SEQUENCE LISTING (57 sequences)

SEQ ID NO: 1 - protein, 8 aa, synthetic construct
  source 1..8: note = 1B1 - VH-CDR1
GFTFSNYA

SEQ ID NO: 2 - protein, 7 aa, synthetic construct
  source 1..7: note = 1B1 - VH-CDR2
ISSGGNT

SEQ ID NO: 3 - protein, 10 aa, synthetic construct
  source 1..10: note = 1B1 - VH-CDR3
ARIDDGYFVF

SEQ ID NO: 4 - protein, 10 aa, synthetic construct
  source 1..10: note = 1B1 - VL-CDR1
QSIDYDGDSY

SEQ ID NO: 5 - skipped

SEQ ID NO: 6 - protein, 9 aa, synthetic construct
  source 1..9: note = 1B1 - VL-CDR3
QQSYEDPPT

SEQ ID NO: 7 - protein, 116 aa, synthetic construct
  source 1..116: note = 1B1 - VH
EVKLVESGGGLVKPGESLKLACAASGFTFSNYAMSWVRQTPEKRLEWVASISSGGNTYFPDSVKGRFTISRDNARNTLYLQMNSLRSEDTAMYYCARIDDGYFVFGGQGTLVTVSA

SEQ ID NO: 8 - protein, 111 aa, synthetic construct
  source 1..111: note = 1B1 - VL
DIVLTQSPASLAVSLGQRATISCKASQSIDYDGDSYMNWYQQKPGQPPKLLIYAASNLESGIPARFSGSGSGTDFTLNIHPVEEEDAATYYCQQSYEDPPTFGGGTKLEIK

SEQ ID NO: 9 - DNA, 348 bp, synthetic construct
  source 1..348: mol_type = other DNA; note = 1B1 - VH Nuc
gaagtgaagctggtggagtctgggggaggcttagtgaagcctggagagtccctgaaactcgcctgtgcagcctctggattcactttcagtaactatgccatgtcttgggttcgccagactccagagaagaggctggagtgggtcgcatccattagtagtggtggtaacacctactttccagacagtgtgaagggccgattcaccatctccagagataatgccaggaacaccctgtacctgcaaatgaacagtctgaggtctgaggacacggccatgtattactgtgcaagaatagacgatggttacttcgttttcgggggccaagggactctggtcactgtctctgca

SEQ ID NO: 10 - DNA, 333 bp, synthetic construct
  source 1..333: mol_type = other DNA; note = 1B1 - VL Nuc
gacattgtgctgacccaatctccagcttctttggctgtgtctctagggcagagggccaccatctcctgcaaggccagccaaagtattgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagttatgaggatcctccgacgttcggtggaggcaccaagctggaaatcaaa

SEQ ID NO: 11 - protein, 8 aa, synthetic construct
  source 1..8: note = 2E7 - VH-CDR1
GFTFSNYA

SEQ ID NO: 12 - protein, 7 aa, synthetic construct
  source 1..7: note = 2E7 - VH-CDR2
ISSGGNT

SEQ ID NO: 13 - protein, 10 aa, synthetic construct
  source 1..10: note = 2E7 - VH-CDR3
ARIDDGYFVF

SEQ ID NO: 14 - protein, 10 aa, synthetic construct
  source 1..10: note = 2E7 - VL-CDR1
QSIDYDGDSY

SEQ ID NO: 15 - skipped

SEQ ID NO: 16 - protein, 9 aa, synthetic construct
  source 1..9: note = 2E7 - VL-CDR3
QQSYEDPPT

SEQ ID NO: 17 - protein, 116 aa, synthetic construct; identical to SEQ ID NO: 7
  source 1..116: note = 2E7 - VH

SEQ ID NO: 18 - protein, 111 aa, synthetic construct; identical to SEQ ID NO: 8
  source 1..111: note = 2E7 - VL

SEQ ID NO: 19 - DNA, 348 bp, synthetic construct; identical to SEQ ID NO: 9
  source 1..348: mol_type = other DNA; note = 2E7 - VH Nuc

SEQ ID NO: 20 - DNA, 333 bp, synthetic construct; identical to SEQ ID NO: 10
  source 1..333: mol_type = other DNA; note = 2E7 - VL Nuc

SEQ ID NO: 21 - protein, 9 aa, synthetic construct
  source 1..9: note = 3C3 - VH-CDR1
GYSITSGYS

SEQ ID NO: 22 - protein, 7 aa, synthetic construct
  source 1..7: note = 3C3 - VH-CDR2
IHYSGST

SEQ ID NO: 23 - protein, 7 aa, synthetic construct
  source 1..7: note = 3C3 - VH-CDR3
ASHDFDY

SEQ ID NO: 24 - protein, 11 aa, synthetic construct
  source 1..11: note = 3C3 - VL-CDR1
QSLLDSDGKTY

SEQ ID NO: 25 - skipped

SEQ ID NO: 26 - protein, 9 aa, synthetic construct
  source 1..9: note = 3C3 - VL-CDR3
WQGTHLPYT

SEQ ID NO: 27 - protein, 114 aa, synthetic construct
  source 1..114: note = 3C3 - VH
DVQLQESGPDLVKPSQSLSLTCTVTGYSITSGYSWPWIRQFPGNKLEWMGYIHYSGSTNYNPSLKSRISITRDTSKNQFFLQLNSVTTEDTATYYCASHDFDYWGQGTTLTVSS

SEQ ID NO: 28 - protein, 112 aa, synthetic construct
  source 1..112: note = 3C3 - VL
DVVMTQTPLTLSVTIGQPASVSCKSSQSLLDSDGKTYLNWLLQRPGQSPKRLIYLVSKLDSGVPDRLTGSGSGTDFTLKISRVEAEDLGVYYCWQGTHLPYTFGGGTKLEIK

SEQ ID NO: 29 - DNA, 342 bp, synthetic construct
  source 1..342: mol_type = other DNA; note = 3C3 - VH Nuc
gatgtgcagcttcaggagtcaggacctgacctggtgaaaccttctcagtcactttcactcacctgcactgtcactggctactccatcaccagtggttatagctggccctggatccggcagtttccaggaaacaaactggaatggatgggctacatacactacagtggtagcactaactacaacccatctctcaaaagtcgaatctctatcactcgagacacatccaagaaccagttcttcctgcaattgaattctgtgactactgaggacacagccacatattactgtgcaagccacgactttgactactggggccaaggcaccactctcacagtctcctca

SEQ ID NO: 30 - DNA, 336 bp, synthetic construct
  source 1..336: mol_type = other DNA; note = 3C3 - VL Nuc
gatgttgtgatgacccagactccactcactttgtcggttaccattggacaaccagcctccgtctcttgcaagtcaagtcagagcctcttagatagtgatggaaagacatatttgaattggttgttacagaggccaggccagtctccaaaacgcctaatatatctggtgtctaaactggactctggagtccctgacaggctcactggcagtggatcagggacagatttcacactgaaaatcagcagagtggaggctgaggatttgggagtttattattgttggcaaggcacacatcttccgtacacgttcggaggggggaccaagctggaaataaaa

SEQ ID NO: 31 - protein, 8 aa, synthetic construct
  source 1..8: note = 5H2 - VH-CDR1
GFNIKDYY

SEQ ID NO: 32 - protein, 8 aa, synthetic construct
  source 1..8: note = 5H2 - VH-CDR2
IDPENGNT

SEQ ID NO: 33 - protein, 11 aa, unidentified
  source 1..11: note = 5H2 - VH-CDR3
TRDGYYMVFAY

SEQ ID NO: 34 - protein, 6 aa, synthetic construct
  source 1..6: note = 5H2 - VL-CDR1
QDINKC

SEQ ID NO: 35 - skipped

SEQ ID NO: 36 - protein, 8 aa, synthetic construct
  source 1..8: note = 5H2 - VL-CDR3
LQYDNLYT

SEQ ID NO: 37 - protein, 118 aa, synthetic construct
  source 1..118: note = 5H2 - VH
EVQLQQSGAELVRPGALVKLSCKASGFNIKDYYMHWVKQRPEQGLEWIGWIDPENGNTIYDPKFQGKASITADTSSNTAYLQLSSLTSGDTAVYYCTRDGYYMVFAYWGQGTLVTVSA

SEQ ID NO: 38 - protein, 106 aa, synthetic construct
  source 1..106: note = 5H2 - VL
DIQMTQSPSSLSASLGGKVTITCKASQDINKCIAWYKHKPGKGPRLLIHYTSILQPGIPSRFSGSGSGRDYSFSISNLEPEDIATYYCLQYDNLYTFGGGTKLEIK

SEQ ID NO: 39 - DNA, 354 bp, synthetic construct
  source 1..354: mol_type = other DNA; note = 5H2 - VH Nuc
gaggttcagctgcagcagtctggggctgagcttgtgaggccaggggccttagtcaagttgtcctgcaaagcttctggcttcaacattaaagactactatatgcactgggtgaagcagaggcctgaacagggcctggagtggattggatggattgatcctgagaatggtaatactatatatgacccgaagttccagggcaaggccagtataacagcagacacatcctccaatacagcctacctgcaactcagcagcctgacatctggggacactgccgtctattactgtactagagatggttactacatggtttttgcttactggggccaagggactctggtcactgtctctgca

SEQ ID NO: 40 - DNA, 318 bp, synthetic construct
  source 1..318: mol_type = other DNA; note = 5H2 - VL Nuc
gacatccagatgacgcagtctccatcctcactgtctgcatctctgggaggcaaagtcaccatcacttgcaaggcaagccaagacattaacaagtgtatagcttggtacaaacacaagcctggaaaaggtcctaggctgctcattcattacacatctatattacagccaggcatcccatcaaggttcagtggaagtgggtctgggagagattattccttcagcatcagcaacctggagcctgaagatattgcaacttattattgtctacagtatgataatctgtacacgttcggaggggggaccaagctggaaataaaa

SEQ ID NO: 41 - protein, 8 aa, synthetic construct
  source 1..8: note = 6B7 - VH-CDR1
GFTFSSYA

SEQ ID NO: 42 - protein, 7 aa, synthetic construct
  source 1..7: note = 6B7 - VH-CDR2
ISSGGST

SEQ ID NO: 43 - protein, 10 aa, synthetic construct
  source 1..10: note = 6B7 - VH-CDR3
ARVGEGAMDY

SEQ ID NO: 44 - protein, 10 aa, synthetic construct
  source 1..10: note = 6B7 - VL-CDR1
QSVDYDGDSY

SEQ ID NO: 45 - skipped

SEQ ID NO: 46 - protein, 9 aa, synthetic construct
  source 1..9: note = 6B7 - VL-CDR3
QQSNEDPPT

SEQ ID NO: 47 - protein, 116 aa, synthetic construct
  source 1..116: note = 6B7 - VH
EVKLVESGGGLVKPGGSLKLSCAASGFTFSSYAMSWVRQTPEKRLEWVASISSGGSTYYPDSVKGRFTISRDNTRNILYLQMSSLRSEDTAMYSCARVGEGAMDYWGQGTSVTVSS

SEQ ID NO: 48 - protein, 111 aa, synthetic construct
  source 1..111: note = 6B7 - VL
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPPTFGGGTKLEIK

SEQ ID NO: 49 - DNA, 348 bp, synthetic construct
  source 1..348: mol_type = other DNA; note = 6B7 - VH Nuc
gaagtgaagctggtggagtctgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataataccaggaacatcctgtacctgcaaatgagcagtctgaggtctgaggacacggccatgtattcctgtgcaagagtgggggaaggagctatggactactggggtcaaggaacctcagtcaccgtctcctca

SEQ ID NO: 50 - DNA, 333 bp, synthetic construct
  source 1..333: mol_type = other DNA; note = 6B7 - VL Nuc
gacattgtgctgacccaatctccagcttctttggctgtatctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatcctccgacgttcggtggaggcaccaagctggaaatcaaa

SEQ ID NO: 51 - protein, 9 aa, synthetic construct
  source 1..9: note = Epitope - WT1 fragment
FKLSHLQMH

SEQ ID NO: 52 - protein, 19 aa, synthetic construct
  source 1..19: note = Epitope - WT1 fragment
CNKRYFKLSHLQMHSRKHT

SEQ ID NO: 53 - protein, 10 aa, synthetic construct
  source 1..10: note = Peptide
AYKRKSRESD

SEQ ID NO: 54 - protein, 21 aa, synthetic construct
  source 1..21: note = Peptide
FNNFTVSFWLRVPKVSASHLE

SEQ ID NO: 55 - protein, 24 aa, synthetic construct
  source 1..24: note = Peptide
LPKPPKPVSKMRMATPLLMQALPM

SEQ ID NO: 56 - protein, 21 aa, synthetic construct
  source 1..21: note = Peptide
PGCNKRYFKLSHLQMHSRKHT

SEQ ID NO: 57 - protein, 15 aa, synthetic construct
  source 1..15: note = Peptide
KLLTQHFVQENYLEY

(19) United States
(12) Patent Application Publication
(10) Pub. No.: US 2026/0042827 A1
(43) Pub. Date: Feb. 12, 2026

(54) ANTI-WT1 ANTIGEN-BINDING PROTEINS AND USES THEREOF

(71) Applicant: UNIVERSITY HEALTH NETWORK, Toronto (CA)

(72) Inventors: Naoto HIRANO, Toronto (CA); Toshiki OCHI, Toronto (CA); Chung-Hsi WANG, Toronto (CA)

(73) Assignee: UNIVERSITY HEALTH NETWORK, Toronto, ON (CA)

(21) Appl. No.: 18/850,455

(22) PCT Filed: Mar. 24, 2023
(86) PCT No.: PCT/IB2023/052959

§ 371 Date: Sep. 24, 2024

Related U.S. Application Data
(60) Provisional application No. 63/323,459, filed on Mar. 24, 2022.

(51) Int. Cl. C07K 16/18; C07K 16/28

(52) U.S. Cl. CPC C07K 16/18; C07K 16/2809; C07K 2317/31; C07K 2317/33; C07K 2317/622; C07K 2317/92

(57) ABSTRACT

The present disclosure is directed to antigen-binding molecules that specifically bind peptide fragments of tumor antigens, wherein the peptide fragment is capable of being presented by more than one type of major histocompatibility complex (MHC) class II molecule. In some aspects, the tumor antigen is a WT1 polypeptide. Other aspects are directed to antibodies, multispecific antibodies, and chimeric antigen receptors, and nucleotides encoding the same. Other aspects are directed to methods of administering the same to a subject in need thereof.

### CROSS REFERENCE TO RELATED APPLICATIONS

This PCT application claims the priority benefit U.S. Provisional Application No. 63/323,459, filed Mar. 24, 2022, which is incorporated herein by reference in its entirety.

### REFERENCE TO SEQUENCE LISTING SUBMITTED ELECTRONICALLY VIA EFS-WEB

The content of the electronically submitted sequence listing (Name: 4706_019PC01_Seglisting_ST26; Size: 60,576 bytes; and Date of Creation: Mar. 20, 2023) is herein incorporated by reference in its entirety.

### FIELD OF THE DISCLOSURE

The present disclosure provides antigen-binding molecules capable of binding tumor antigen fragments displayed by MHC class II molecules.

### BACKGROUND OF THE DISCLOSURE

Immunotherapy has emerged as a critical tool in the battle against a variety of diseases, including cancer. T cell therapies are at the forefront of immunotherapeutic development, and adoptive transfer of antitumor T cells has been shown to induce clinical responses in cancer patients.

The adoptive transfer of T cells redirected by antitumor T cell receptor (TCR) gene transfer is a promising strategy for cancer therapy. Many TCR genes currently tested in the clinic are HLA-class I-restricted. Importantly, cancer cells often express HLA-class II molecules in vivo. Since class II expression is more restricted compared to class I expression, targeting class II may be advantageous to reduce the risk of both on- and off-target toxicities. Moreover, given that the same class II-restricted peptides can promiscuously bind to different class II molecules, TCR gene therapy targeting class II-restricted tumor antigenic peptides could be applied to many cancer patients with diverse class II genotypes. Prevalent class II molecules, HLA-DP2 and HLA-DP4, can constitutively present peptides derived from intracellular tumor associated antigens (TAAs) regardless of invariant chain or HLA-DM expression. Described herein are compositions and methods that target MHC class-II displayed peptide fragments of tumor antigens and the use thereof in treating cancer.

### SUMMARY OF THE DISCLOSURE

Some aspects of the present disclosure are directed to an antibody or an antigen-binding portion thereof that specifically binds a peptide fragment of Wilms' tumor protein (WT1), wherein the peptide fragment is capable of being presented by a major histocompatibility complex (MHC) class II molecule.

Some aspects of the present disclosure are directed to an antibody or an antigen-binding portion thereof that specifically binds a peptide fragment of WT1, wherein the peptide fragment is associated with an MHC class II molecule.

In some aspects, the peptide fragment comprises at least about 10 to about 20 amino acids, at least about 11 to about 20 amino acids, at least about 12 to about 20 amino acids, at least about 13 to about 20 amino acids, at least about 14 to about 20 amino acids, at least about 15 to about 20 amino acids, at least about 16 to about 20 amino acids, at least about 17 to about 20 amino acids, at least about 11 to about 19 amino acids, at least about 12 to about 19 amino acids, at least about 13 to about 19 amino acids, at least about 14 to about 19 amino acids, at least about 15 to about 19 amino acids, at least about 16 to about 19 amino acids, at least about 17 to about 19 amino acids, at least about 11 to about 18 amino acids, at least about 12 to about 18 amino acids, at least about 13 to about 18 amino acids, at least about 14 to about 18 amino acids, at least about 15 to about 18 amino acids, at least about 16 to about 18 amino acids, at least about 17 to about 18 amino acids, at least about 11 to about 17 amino acids, at least about 12 to about 17 amino acids, at least about 13 to about 17 amino acids, at least about 14 to about 17 amino acids, at least about 15 to about 17 amino acids, or at least about 16 to about 17 amino acids.

In some aspects, the antibody or antigen-binding portion thereof does not bind an MHC Class II molecule.

In some aspects, the antibody or antigen-binding portion thereof does not bind full length WT1.

In some aspects, the antibody or antigen-binding portion thereof comprises a heavy chain and a light chain, wherein the heavy chain comprises a heavy chain variable region (VH) comprising a VH complementarity determining region (CDR)-1, a VH-CDR2, and a VH-CDR3; and wherein the light chain comprises a light chain variable region (VL) comprising a VL CDR1, a VL-CDR2, and a VL-CDR3; wherein the VH-CDR3 comprises the amino acid sequence set forth in SEQ ID NO: 3, 13, 23, 33, 43, or 53.

Some aspects of the present disclosure are directed to an antibody or an antigen-binding portion thereof that specifically binds WT1, comprising a heavy chain and a light chain, wherein the heavy chain comprises a heavy chain variable region (VH) comprising a VH complementarity determining region (CDR)-1, a VH-CDR2, and a VH-CDR3; and wherein the light chain comprises a light chain variable region (VL) comprising a VL CDR1, a VL-CDR2, and a VL-CDR3; wherein the VH-CDR3 comprises an amino acid sequence set forth in SEQ ID NO: 3, 13, 23, 33, 43, or 53.

In some aspects, the VH-CDR2 comprises an amino acid sequence set forth in SEQ ID NO: 2, 12, 22, 32, 42, or 52.

In some aspects, the VH-CDR1 comprises an amino acid sequence set forth in SEQ ID NO: 1, 11, 21, 31, 41, or 51.

In some aspects, the VL-CDR1 comprises an amino acid sequence set forth in SEQ ID NO: 4, 14, 24, 34, 44, or 54.

In some aspects, the VL-CDR2 comprises an amino acid sequence set forth in SEQ ID NO: 5, 15, 25, 35, 45, or 55.

In some aspects, the VL-CDR3 comprises an amino acid sequence set forth in SEQ ID NO: 6, 16, 26, 36, 46, or 56.

In some aspects, the antibody or antigen-binding portion thereof comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen-binding portion thereof comprises a VH-CDR1 comprising the amino acid sequence set forth in SEQ ID NO: 11, a VH-CDR2 comprising the amino acid sequence set forth in SEQ ID NO: 12, a VH-CDR3 comprising the amino acid sequence set forth in SEQ ID NO: 13, a VL-CDR1 comprising the amino acid sequence set forth in SEQ ID NO: 14, a VL-CDR2 comprising the amino acid sequence set forth in SEQ ID NO: 15, and a VL-CDR3 comprising the amino acid sequence set forth in SEQ ID NO: 16.

In some aspects, the antibody or antigen-binding portion thereof comprises a VH-CDR1 comprising the amino acid sequence set forth in SEQ ID NO: 21, a VH-CDR2 comprising the amino acid sequence set forth in SEQ ID NO: 22, a VH-CDR3 comprising the amino acid sequence set forth in SEQ ID NO: 23, a VL-CDR1 comprising the amino acid sequence set forth in SEQ ID NO: 24, a VL-CDR2 comprising the amino acid sequence set forth in SEQ ID NO: 25, and a VL-CDR3 comprising the amino acid sequence set forth in SEQ ID NO: 26.

In some aspects, the antibody or antigen-binding portion thereof comprises a VH-CDR1 comprising the amino acid sequence set forth in SEQ ID NO: 31, a VH-CDR2 comprising the amino acid sequence set forth in SEQ ID NO: 32, a VH-CDR3 comprising the amino acid sequence set forth in SEQ ID NO: 33, a VL-CDR1 comprising the amino acid sequence set forth in SEQ ID NO: 34, a VL-CDR2 comprising the amino acid sequence set forth in SEQ ID NO: 35, and a VL-CDR3 comprising the amino acid sequence set forth in SEQ ID NO: 36.

In some aspects, the antibody or antigen-binding portion thereof comprises a VH-CDR1 comprising the amino acid sequence set forth in SEQ ID NO: 41, a VH-CDR2 comprising the amino acid sequence set forth in SEQ ID NO: 42, a VH-CDR3 comprising the amino acid sequence set forth in SEQ ID NO: 43, a VL-CDR1 comprising the amino acid sequence set forth in SEQ ID NO: 44, a VL-CDR2 comprising the amino acid sequence set forth in SEQ ID NO: 45, and a VL-CDR3 comprising the amino acid sequence set forth in SEQ ID NO: 46.

In some aspects, the VH comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 7, 17, 27, 37, 47, or 57.

In some aspects, the VH comprises an amino acid sequence set forth in SEQ ID NOs: 7, 17, 27, 37, 47, or 57.

In some aspects, the VL comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 8, 18, 28, 38, 48, or 58.

In some aspects, the VL comprises an amino acid sequence set forth in SEQ ID NOs: 8, 18, 28, 38, 48, or 58.

In some aspects, the antibody or antigen-binding portion thereof comprises a VH comprising the amino acid sequence set forth in SEQ ID NO: 7, and a VL comprising the amino acid sequence set forth in SEQ ID NO: 8.

In some aspects, the antibody or antigen-binding portion thereof comprises a VH comprising the amino acid sequence set forth in SEQ ID NO: 17, and a VL comprising the amino acid sequence set forth in SEQ ID NO: 18.

In some aspects, the antibody or antigen-binding portion thereof comprises a VH comprising the amino acid sequence set forth in SEQ ID NO: 27, and a VL comprising the amino acid sequence set forth in SEQ ID NO: 28.

In some aspects, the antibody or antigen-binding portion thereof comprises a VH comprising the amino acid sequence set forth in SEQ ID NO: 37, and a VL comprising the amino acid sequence set forth in SEQ ID NO: 38.

In some aspects, the antibody or antigen-binding portion thereof comprises a VH comprising the amino acid sequence set forth in SEQ ID NO: 47, and a VL comprising the amino acid sequence set forth in SEQ ID NO: 48.

In some aspects, the antibody or antigen-binding portion thereof comprises binds a peptide fragment of WT1.

In some aspects, the peptide fragment of WT1 comprises the amino acid sequence set forth in SEQ ID NO: 51.

In some aspects, the peptide fragment is presented by an MHC Class II molecule.

In some aspects, the peptide fragment is presented by an MHC Class II molecule expressed on the surface of a tumor cell.

In some aspects, the MHC Class II molecule comprises (i) a DP beta chain and a DP alpha chain, (ii) a DR beta chain and a DR alpha chain, or (iii) a DQ beta chain and a DQ alpha chain.

In some aspects, the beta chain of the MHC class II molecule is a DP1, DP2, DP3, DP4, DP5, DP6, DP8, or DP9 allele.

In some aspects, the alpha chain of the MHC class II molecule comprises an HLA-DPA1*01, HLA-DPA1*02, HLA-DPA1*03, or HLA-DPA1*04 allele.

In some aspects, the beta chain of the MHC class II molecule comprises a DR2, DR3, DR4, DR5, DR6, DR7, DR8, DR9, DR10, DR11, DR12, DR13, DR14, DR15, or DR16 allele.

In some aspects, the alpha chain of the MHC class II molecule comprises an HLA-DRA1*01 allele.

In some aspects, the beta chain of the MHC class II molecule comprises a DQ2, DQ3, DQ4, DQ5, or DQ6 allele.

In some aspects, the alpha chain of the MHC class II molecule comprises an HLA-DQA1*01, HLA-DQA1*02, HLA-DQA1*03, HLA-DQA1*04, HLA-DQA1*05, or HLA-DQA1*06 allele.

Some aspects of the present disclosure are directed to an antibody or an antigen-binding portion thereof that specifically binds a peptide fragment of a tumor antigen, wherein the peptide fragment is presented by a first major histocompatibility complex class II molecule and a second MHC class II molecule, wherein the first and the second MHC class II molecules are different.

In some aspects, the tumor antigen comprises NY-ESO-1, gp100, Tyr, MAGE-A1, MAGE-A3, SSX2, CCND1, MUC5C, WT1, or any combination thereof.

In some aspects, the first MHC class II molecule comprises (i) a DP beta chain and a DP alpha chain, (ii) a DR beta chain and a DR alpha chain, or (iii) a DQ beta chain and a DQ alpha chain.

In some aspects, the beta chain of the first MHC class II molecule is a DP1, DP2, DP3, DP4, DP5, DP6, DP8, or DP9 allele.

In some aspects, the alpha chain of the first MHC class II molecule comprises an HLA-DPA1*01, HLA-DPA1*02, HLA-DPA1*03, or HLA-DPA1*04 allele.

In some aspects, the beta chain of the first MHC class II molecule comprises a DR2, DR3, DR4, DR5, DR6, DR7, DR8, DR9, DR10, DR11, DR12, DR13, DR14, DR15, or DR16 allele.

In some aspects, the alpha chain of the first MHC class II molecule comprises an HLA-DRA1*01 allele.

In some aspects, the beta chain of the first MHC class II molecule comprises a DQ2, DQ3, DQ4, DQ5, or DQ6 allele.

In some aspects, the alpha chain of the first MHC class II molecule comprises an HLA-DQA1*01, HLA-DQA1*02, HLA-DQA1*03, HLA-DQA1*04, HLA-DQA1*05, or HLA-DQA1*06 allele.

In some aspects, the second MHC class II molecule comprises (i) a DP beta chain and a DP alpha chain, (ii) a DR beta chain and a DR alpha chain, or (iii) a DQ beta chain and a DQ alpha chain.

In some aspects, the beta chain of the second MHC class II molecule is a DP1, DP2, DP3, DP4, DP5, DP6, DP8, or DP9 allele.

In some aspects, the alpha chain of the second MHC class II molecule comprises an HLA-DPA1*01, HLA-DPA1*02, HLA-DPA1*03, or HLA-DPA1*04 allele.

In some aspects, the beta chain of the second MHC class II molecule comprises a DR2, DR3, DR4, DR5, DR6, DR7, DR8, DR9, DR10, DR11, DR12, DR13, DR14, DR15, or DR16 allele.

In some aspects, the alpha chain of the second MHC class II molecule comprises an HLA-DRA1*01 allele.

In some aspects, the beta chain of the second MHC class II molecule comprises a DQ2, DQ3, DQ4, DQ5, or DQ6 allele.

In some aspects, the alpha chain of the second MHC class II molecule comprises an HLA-DQA1*01, HLA-DQA1*02, HLA-DQA1*03, HLA-DQA1*04, HLA-DQA1*05, or HLA-DQA1*06 allele.

Some aspects of the present disclosure are directed to a chimeric antigen receptor (CAR) comprising an antigen-binding domain, wherein the antigen binding domain comprises a antibody or antigen-binding portion thereof disclosed herein.

Some aspects of the present disclosure are directed to a chimeric antigen receptor (CAR) comprising an antigen-binding domain comprising a VH-CDR1, a VH-CDR2, a VH-CDR3, a VL-CDR1, a VL-CDR2, and a VL-CDR3; wherein the VH-CDR3 comprises an amino acid sequence set forth in SEQ ID NO: 3, 13, 23, 33, 43, or 53.

In some aspects, the VH-CDR2 comprises an amino acid sequence set forth in SEQ ID NO: 2, 12, 22, 32, 42, or 52.

In some aspects, the VH-CDR1 comprises an amino acid sequence set forth in SEQ ID NO: 1, 11, 21, 31, 41, or 51.

In some aspects, the VL-CDR1 comprises an amino acid sequence set forth in SEQ ID NO: 4, 14, 24, 34, 44, or 54.

In some aspects, the VL-CDR2 comprises an amino acid sequence set forth in SEQ ID NO: 5, 15, 25, 35, 45, or 55.

In some aspects, the VL-CDR3 comprises an amino acid sequence set forth in SEQ ID NO: 6, 16, 26, 36, 46, or 56.

In some aspects, the chimeric antigen receptor comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the chimeric antigen receptor comprises a VH-CDR1 comprising the amino acid sequence set forth in SEQ ID NO: 11, a VH-CDR2 comprising the amino acid sequence set forth in SEQ ID NO: 12, a VH-CDR3 comprising the amino acid sequence set forth in SEQ ID NO: 13, a VL-CDR1 comprising the amino acid sequence set forth in SEQ ID NO: 14, a VL-CDR2 comprising the amino acid sequence set forth in SEQ ID NO: 15, and a VL-CDR3 comprising the amino acid sequence set forth in SEQ ID NO: 16.

In some aspects, the chimeric antigen receptor comprises a VH-CDR1 comprising the amino acid sequence set forth in SEQ ID NO: 21, a VH-CDR2 comprising the amino acid sequence set forth in SEQ ID NO: 22, a VH-CDR3 comprising the amino acid sequence set forth in SEQ ID NO: 23, a VL-CDR1 comprising the amino acid sequence set forth in SEQ ID NO: 24, a VL-CDR2 comprising the amino acid sequence set forth in SEQ ID NO: 25, and a VL-CDR3 comprising the amino acid sequence set forth in SEQ ID NO: 26.

In some aspects, the chimeric antigen receptor comprises a VH-CDR1 comprising the amino acid sequence set forth in SEQ ID NO: 31, a VH-CDR2 comprising the amino acid sequence set forth in SEQ ID NO: 32, a VH-CDR3 comprising the amino acid sequence set forth in SEQ ID NO: 33, a VL-CDR1 comprising the amino acid sequence set forth in SEQ ID NO: 34, a VL-CDR2 comprising the amino acid sequence set forth in SEQ ID NO: 35, and a VL-CDR3 comprising the amino acid sequence set forth in SEQ ID NO: 36.

In some aspects, the chimeric antigen receptor comprises a VH-CDR1 comprising the amino acid sequence set forth in SEQ ID NO: 41, a VH-CDR2 comprising the amino acid sequence set forth in SEQ ID NO: 42, a VH-CDR3 comprising the amino acid sequence set forth in SEQ ID NO: 43, a VL-CDR1 comprising the amino acid sequence set forth in SEQ ID NO: 44, a VL-CDR2 comprising the amino acid sequence set forth in SEQ ID NO: 45, and a VL-CDR3 comprising the amino acid sequence set forth in SEQ ID NO: 46.

Some aspects of the present disclosure are directed to a bispecific antibody comprising a first antigen-binding domain and a second antigen-binding domain, wherein the first antigen-binding domain, the second antigen-binding domain, or both comprises an antibody or antigen-binding portion thereof disclosed herein.

Some aspects of the present disclosure are directed to a bispecific antibody comprising (i) a first antigen-binding domain and (ii) a second antigen-binding domain; wherein the first antigen-binding domain, the second antigen-binding domain, or both comprises a VH-CDR1, a VH-CDR2, a VH-CDR3, a VL-CDR1, a VL-CDR2, and a VL-CDR3; and wherein the VH-CDR3 comprises an amino acid sequence set forth in SEQ ID NO: 3, 13, 23, 33, 43, or 53.

Some aspects of the present disclosure are directed to a bi-specific T cell engager (BiTE) comprising (i) a first antigen-binding domain that specifically binds CD3 receptor and (ii) a second antigen-binding domain that specifically binds a peptide fragment of WT1, wherein second antigen-binding domain comprises an antibody or antigen-binding portion thereof disclosed herein.

Some aspects of the present disclosure are directed to a bi-specific T cell engager (BiTE) comprising (i) a first antigen-binding domain that specifically binds CD3 receptor and (ii) a second antigen-binding domain; wherein the second antigen-binding domain comprises a VH-CDR1, a VH-CDR2, a VH-CDR3, a VL-CDR1, a VL-CDR2, and a VL-CDR3; and wherein the VH-CDR3 comprises an amino acid sequence set forth in SEQ ID NO: 3, 13, 23, 33, 43, or 53.

In some aspects, the VH-CDR2 comprises an amino acid sequence set forth in SEQ ID NO: 2, 12, 22, 32, 42, or 52.

In some aspects, the VH-CDR1 comprises an amino acid sequence set forth in SEQ ID NO: 1, 11, 21, 31, 41, or 51.

In some aspects, the VL-CDR1 comprises an amino acid sequence set forth in SEQ ID NO: 4, 14, 24, 34, 44, or 54.

In some aspects, the VL-CDR2 comprises an amino acid sequence set forth in SEQ ID NO: 5, 15, 25, 35, 45, or 55.

In some aspects, the VL-CDR3 comprises an amino acid sequence set forth in SEQ ID NO: 6, 16, 26, 36, 46, or 56.

In some aspects, the second antigen-binding domain comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the second antigen-binding domain comprises a VH-CDR1 comprising the amino acid sequence set forth in SEQ ID NO: 11, a VH-CDR2 comprising the amino acid sequence set forth in SEQ ID NO: 12, a VH-CDR3 comprising the amino acid sequence set forth in SEQ ID NO: 13, a VL-CDR1 comprising the amino acid sequence set forth in SEQ ID NO: 14, a VL-CDR2 comprising the amino acid sequence set forth in SEQ ID NO: 15, and a VL-CDR3 comprising the amino acid sequence set forth in SEQ ID NO: 16.

In some aspects, the second antigen-binding domain comprises a VH-CDR1 comprising the amino acid sequence set forth in SEQ ID NO: 21, a VH-CDR2 comprising the amino acid sequence set forth in SEQ ID NO: 22, a VH-CDR3 comprising the amino acid sequence set forth in SEQ ID NO: 23, a VL-CDR1 comprising the amino acid sequence set forth in SEQ ID NO: 24, a VL-CDR2 comprising the amino acid sequence set forth in SEQ ID NO: 25, and a VL-CDR3 comprising the amino acid sequence set forth in SEQ ID NO: 26.

In some aspects, the second antigen-binding domain comprises a VH-CDR1 comprising the amino acid sequence set forth in SEQ ID NO: 31, a VH-CDR2 comprising the amino acid sequence set forth in SEQ ID NO: 32, a VH-CDR3 comprising the amino acid sequence set forth in SEQ ID NO: 33, a VL-CDR1 comprising the amino acid sequence set forth in SEQ ID NO: 34, a VL-CDR2 comprising the amino acid sequence set forth in SEQ ID NO: 35, and a VL-CDR3 comprising the amino acid sequence set forth in SEQ ID NO: 36.

In some aspects, the second antigen-binding domain comprises a VH-CDR1 comprising the amino acid sequence set forth in SEQ ID NO: 41, a VH-CDR2 comprising the amino acid sequence set forth in SEQ ID NO: 42, a VH-CDR3 comprising the amino acid sequence set forth in SEQ ID NO: 43, a VL-CDR1 comprising the amino acid sequence set forth in SEQ ID NO: 44, a VL-CDR2 comprising the amino acid sequence set forth in SEQ ID NO: 45, and a VL-CDR3 comprising the amino acid sequence set forth in SEQ ID NO: 46.

Some aspects of the present disclosure are directed to a tri-specific T cell engager (TriTE) comprising (i) a first antigen-binding domain that specifically binds CD3 receptor, (ii) a second antigen-binding domain that specifically binds a peptide fragment of WT1, and (iii) a third antigen-binding domain; wherein second antigen-binding domain comprises an antibody or antigen-binding portion thereof disclosed herein.

Some aspects of the present disclosure are directed to a tri-specific T cell engager (TriTE) comprising (i) a first antigen-binding domain that specifically binds CD3 receptor, (ii) a second antigen-binding domain, and (iii) a third antigen-binding domain; wherein the second antigen-binding domain comprises a VH-CDR1, a VH-CDR2, a VH-CDR3, a VL-CDR1, a VL-CDR2, and a VL-CDR3; and wherein the VH-CDR3 comprises an amino acid sequence set forth in SEQ ID NO: 3, 13, 23, 33, 43, or 53.

In some aspects, the VH-CDR2 comprises an amino acid sequence set forth in SEQ ID NO: 2, 12, 22, 32, 42, or 52.

In some aspects, the VH-CDR1 comprises an amino acid sequence set forth in SEQ ID NO: 1, 11, 21, 31, 41, or 51.

In some aspects, the VL-CDR1 comprises an amino acid sequence set forth in SEQ ID NO: 4, 14, 24, 34, 44, or 54.

In some aspects, the VL-CDR2 comprises an amino acid sequence set forth in SEQ ID NO: 5, 15, 25, 35, 45, or 55.

In some aspects, the VL-CDR3 comprises an amino acid sequence set forth in SEQ ID NO: 6, 16, 26, 36, 46, or 56.

In some aspects, the second antigen-binding domain comprises: a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the second antigen-binding domain comprises a VH-CDR1 comprising the amino acid sequence set forth in SEQ ID NO: 11, a VH-CDR2 comprising the amino acid sequence set forth in SEQ ID NO: 12, a VH-CDR3 comprising the amino acid sequence set forth in SEQ ID NO: 13, a VL-CDR1 comprising the amino acid sequence set forth in SEQ ID NO: 14, a VL-CDR2 comprising the amino acid sequence set forth in SEQ ID NO: 15, and a VL-CDR3 comprising the amino acid sequence set forth in SEQ ID NO: 16.

In some aspects, the second antigen-binding domain comprises a VH-CDR1 comprising the amino acid sequence set forth in SEQ ID NO: 21, a VH-CDR2 comprising the amino acid sequence set forth in SEQ ID NO: 22, a VH-CDR3 comprising the amino acid sequence set forth in SEQ ID NO: 23, a VL-CDR1 comprising the amino acid sequence set forth in SEQ ID NO: 24, a VL-CDR2 comprising the amino acid sequence set forth in SEQ ID NO: 25, and a VL-CDR3 comprising the amino acid sequence set forth in SEQ ID NO: 26.

In some aspects, the second antigen-binding domain comprises a VH-CDR1 comprising the amino acid sequence set forth in SEQ ID NO: 31, a VH-CDR2 comprising the amino acid sequence set forth in SEQ ID NO: 32, a VH-CDR3 comprising the amino acid sequence set forth in SEQ ID NO: 33, a VL-CDR1 comprising the amino acid sequence set forth in SEQ ID NO: 34, a VL-CDR2 comprising the amino acid sequence set forth in SEQ ID NO: 35, and a VL-CDR3 comprising the amino acid sequence set forth in SEQ ID NO: 36.

In some aspects, the second antigen-binding domain comprises a VH-CDR1 comprising the amino acid sequence set forth in SEQ ID NO: 41, a VH-CDR2 comprising the amino acid sequence set forth in SEQ ID NO: 42, a VH-CDR3 comprising the amino acid sequence set forth in SEQ ID NO: 43, a VL-CDR1 comprising the amino acid sequence set forth in SEQ ID NO: 44, a VL-CDR2 comprising the amino acid sequence set forth in SEQ ID NO: 45, and a VL-CDR3 comprising the amino acid sequence set forth in SEQ ID NO: 46.

Some aspects of the present disclosure are directed to a nucleic acid molecule or a set of nucleic acid molecules encoding an antibody or antigen-binding portion thereof, a CAR of, a bispecific antibody, a BiTE, or a TriTE disclosed herein.

Some aspects of the present disclosure are directed to a vector or a set of vectors comprising a nucleic acid molecule or set of nucleic acid molecules disclosed herein.

In some aspects, the vector is a viral vector, a bacterial vector, a mammalian vector, or any combination thereof.

In some aspects, the viral vector is selected from an adenoviral vector, a lentivirus, a Sendai virus, a baculoviral vector, an Epstein Barr viral vector, a papovaviral vector, a vaccinia viral vector, a herpes simplex viral vector, or an adeno associated virus (AAV) vector.

Some aspects of the present disclosure are directed to a cell comprising an antibody or antigen-binding portion thereof, a CAR, a bispecific antibody, a BiTE, or a TriTE disclosed herein.

In some aspects, the cell is an immune cell.

In some aspects, the cell is selected from a T cell, a B cell, a natural killer (NK) cell, a tumor infiltrating lymphocyte (TIL), a helper T cell, or any combination thereof.

Some aspects of the present disclosure are directed to a pharmaceutical composition comprising an antibody or antigen-binding portion thereof, a CAR, a bispecific antibody, a BiTE, or a TriTE disclosed herein.

Some aspects of the present disclosure are directed to a method of treating a disease or condition in a subject in need thereof, comprising administering to the subject an antibody or antigen-binding portion thereof, a CAR, a bispecific antibody, a BiTE, a TriTE, a nucleic acid molecule or set of nucleic acid molecules, a vector or set of vectors, a cell, or a pharmaceutical composition disclosed herein.

In some aspects, the disease or condition comprises a tumor or an infection.

In some aspects, the tumor is derived from a cancer selected from melanoma, bone cancer, pancreatic cancer, skin cancer, cancer of the head or neck, uterine cancer, ovarian cancer, rectal cancer, stomach cancer, uterine cancer, lung cancer, Hodgkin's Disease, non-Hodgkin's lymphoma (NHL), cancer of the esophagus, cancer of the small intestine, cancer of the urethra, chronic or acute leukemia, acute myeloid leukemia, chronic myeloid leukemia, acute lymphoblastic leukemia (ALL) (including non T cell ALL), chronic lymphocytic leukemia (CLL), cancer of the bladder, cancer of the kidney or ureter, carcinoma of the renal pelvis, glioma, squamous cell cancer, and combinations of said cancers.

In some aspects, the tumor is relapsed or refractory.

In some aspects, the cancer is locally advanced.

In some aspects, the cancer is metastatic.

Some aspects of the present disclosure are directed to a method of generating an antibody or an antigen-binding portion thereof that binds a peptide fragment complexed with an MHC Class II molecule presenting comprising: (i) identifying a peptide fragment associated with an MHC Class II molecule, and (ii) raising an antibody against the peptide fragment; wherein the antibody is not raised against the MHC Class II molecule or a complex of the MHC Class II molecule and the peptide fragment.

Some aspects of the present disclosure are directed to a method of generating an antibody or an antigen-binding portion thereof that binds a tumor antigen comprising: (i) identifying a peptide fragment of the tumor antigen that is associated with an MHC Class II molecule, and (ii) raising an antibody against the peptide fragment; wherein the antibody is not raised against the MHC Class II molecule or a complex of the MHC Class II molecule and the peptide fragment.

Some aspects of the present disclosure are directed to a method of generating an antibody or an antigen-binding portion thereof that binds a tumor antigen comprising: (i) computationally identifying a peptide fragment of the tumor antigen that is likely to be associated with an MHC Class II molecule, and (ii) raising an antibody against the peptide fragment; wherein the antibody is not raised against the MHC Class II molecule or a complex of the MHC Class II molecule and the peptide fragment.

In some aspects, the method further comprises selecting for an antibody or an antigen-binding portion thereof that specifically binds an epitope of the peptide fragment that is exposed on the surface of the MHC Class II molecule-peptide fragment complex.

In some aspects, the peptide fragment is a fragment of a tumor antigen.

In some aspects, the tumor antigen is NY-ESO-1, gp100, Tyr, MAGE-A1, MAGE-A3, SSX2, CCND1, MUC5C, WT1, or any combination thereof.

### BRIEF DESCRIPTION OF THE DRAWINGS

FIGS. 1A-1B are graphical representations illustrating binding of WT1₃₂₈₋₃₄₈ to cells expressing HLA-DP2 (FIG. 1A) or HLA-DP4 (FIG. 1B).

FIGS. 2A-2L present data showing that a DP5-restricted, WT1₃₃₃₋₃₄₇ specific TCR recognizes exogenously pulsed WT1₃₂₈₋₃₄₈ in both DP2 and DP4-restricted manner. FIGS. 2A-2D are graphical representations illustrating surface expression of HLA-DP of K562 transfectants was analyzed by flow cytometric assays after staining with specific mAbs. FIGS. 2E-2L are bar graphs showing data for clone 9 TCR or empty PMX vector (control)-transduced CD4+ T cells stimulated using the indicated peptide-pulsed K562-based aAPCs for 20-24 hours; IFN-γ and IL-2 secretions were measured by ELISPOT. K562 cells transduced with HLA-DP5 were pulsed with WT1₃₂₈₋₃₄₈ or WT1₃₃₂₋₃₄₇ or CLIP (control) and used to stimulate T cells. K562 cells transduced with DP2 or DP4 were pulsed with WT1₃₂₈₋₃₄₈ or TT₉₄₇₋₉₆₇ (control) and used to stimulate T cells. The data shown represent the mean±SD of experiments performed in triplicates. Results are representative of three independent experiments.

FIGS. 3A-3N present data showing that WT1₃₂₈₋₃₄₈ can be naturally processed and presented in both DP2 and DP4-restricted manner. FIGS. 3A-3L are graphical representations illustrating intracellular expression of Ii and surface expression of ΔNGFR of the indicated K562 transfectants measured by flow cytometric analysis after staining with specific mAbs. FIGS. 3M-3N are bar graphs presenting data showing for clone 9 TCR or empty pMX vector (control)-transduced CD4+ T cells stimulated with the indicated K562 transfectants for 20-24 hours; IFN-γ secretion was measured by ELISPOT. The data shown represent the mean±SD of experiments performed in triplicates. Results are representative of three independent experiments. ***p<0.001, ****p<0.0001 by two-way ANOVA with Bonferroni correction.

FIGS. 4A-4C preset data showing that clone 9 TCR-transduced T cells can mediate antitumor responses in vitro and in vivo. FIG. 4A is a graphical representation of cytotoxicity of clone 9 TCR or HA1.7 TCR (control)-transduced T cells against the indicated K562 transfectants as measured by in vitro flow cytometry-based killing assays. Data from three donors were quantified. ****p<0.0001 by two-way ANOVA with Bonferroni correction. FIGS. 4B-4C are graphs showing the results of NSG mice subcutaneously injected with 5×10⁵ indicated K562 transfectants. Two days later, the mice were treated with 07 T cells transduced with clone 9 TCR or HA1.7 TCR (control). The mean tumor size for each group is represented as the average±SD of three mice. Results are representative of two independent experiments.

FIGS. 5A-5S present data showing that WT1-specific mAbs bind to WTT peptide presented by HLA-DP molecules. FIGS. 5A-5B show binding of B7/21 (an anti-HLA-DP mAb) or WT1-specific mAbs to immobilized WT1 peptide or DP4/WT1 monomers as assessed by ELISA. m Abs were added to ELISA plate coated with WT1₃₃₀₋₃₄₈ or CLIP (control) (FIG. 5A). mAbs were added to ELISA plate coated with DP4/WT1₃₃₀₋₃₄₈ monomers or DP4/CLIP monomers (control) (FIG. 5B). The data shown represent the mean±SD of experiments performed in triplicates. Results are representative of two independent experiments. ns, not significant, *p<0.05, **p<0.01, ***p<0.001, ****p<0.0001 by two-way ANOVA with Bonferroni correction. FIG. 5C shows quantification of binding between WT1-specific mAb, 5H2, and WT1₃₃₀₋₃₄₈. Interaction strength was measured by a BLI binding assay. FIGS. 5F-5K show the indicated K562 transfectants pulsed with WT1₃₃₀₋₃₄₈ or CLIP (control) and stained with WT1-specific mAb, 5H2, followed by PE-conjugated anti-mouse secondary Ab and flow cytometric analysis. FIGS. 5L-5S show the indicated K562 transfectants stained with WT1-specific mAb, 5H2, or isotype control mAb, followed by PE-conjugated anti-mouse secondary Ab and flow cytometric analysis.

FIGS. 6A-6V provide comparison between WT1 TCR and CAR-T cells targeting WT1 peptide presented by HLA-DP molecules. FIG. 6A is a schematic representation of the WT1 TCR and WT1-28z CAR constructs. WT1-TCR: Full-length clone 9 TCRα and TCRβ genes were linked by a furin cleavage site, an SGSG spacer sequence, and a P2A sequence. WT1-28z CAR: The single-chain variable fragment (scFv) was formed by connecting the variable regions of heavy (VH) and light chain (VL) derived from WT1-specific mAb, 5H2, via a Whitlow linker. The scFv was linked to human CD28 transmembrane and cytoplasmic domain, followed by cytoplasmic domain of human CD3 zeta. Both TCR and CAR constructs were N-terminally linked to truncated NGFR (ΔNGFR) via a furin cleavage site, an SGSG spacer sequence, and a P2A sequence. FIGS. 6B and 6C are representations of flow cytometry showing human primary T cells transduced with WT1 TCR and WT1-28z CAR stained with Pacific Blue-conjugated anti-NGFR m Ab and biotinylated protein-L, followed by streptavidin-PE. FIG. 6D is a bar graph showing the results of WT1 TCR or CAR-transduced T cells stimulated with the indicated K562 transfectants pulsed with WT1₃₂₉₋₃₄₈ for 20-24 hours; IFN-γ secretion was measured by ELISPOT. The data shown represent the mean±SD of experiments performed in triplicates. Results are representative of two independent experiments. FIGS. 6E-6F are images of Western blot analysis for WT1 (FIG. 6E) or P-actin (FIG. 6F) in T2, K562, and K562/WT1-knockout (KO) lysates. T2 cells do not express endogenous WT1 and was used as a negative control. FIGS. 6G-6I are graphical representations cytokine secretion by WT1 TCR or CAR-transduced T cells stimulated with the indicated K562 transfectants at an E:T ratio of 1:1 for 5 hours; cytokine secretion of CD4+ and CD8+ T cells was measured by intracellular flow cytometric analysis. FIG. 6K is a graphical representation illustrating cytotoxicity of WT1 TCR or CAR-transduced T cells against the indicated K562 transfectants at E:T ratios of 1:1, 5:1, and 15:1 for 18 hours as measured by in vitro flow cytometry-based killing assays. n=3 donors; results are representative of two independent experiments. *p<0.05, **p<0.01, ***p<0.001, ****p<0.0001 by two-way ANOVA with Bonferroni correction. FIGS. 6L-6M show surface expression of CD25 on WT1 TCR or CAR-transduced CD4+ and CD8+ T cells two weeks after retroviral transduction as measured by flow cytometric analysis. FIGS. 6P-6Q show surface expression of PD-1 and TIM-3 on WT1 TCR or CAR-transduced CD4+ and CD8+ T cells simulated with K562/DP4/WT1 for 7 days as measured by flow cytometric analysis. FIG. 6R shows the fold expansion of WT1 TCR or CAR-transduced T cells simulated with K562/DP4/WT1 for 7 days. G-I: n=3 donors; results are representative of two independent experiments. *p<0.05, **p<0.01 by unpaired two-tailed T test. FIGS. 6S-6T show the IFN-γ secretion of WT1 TCR (FIG. 6S) or CAR (FIG. 6T)-transduced T cells stimulated with T2/DP4 pulsed with WT1₃₂₈₋₃₄₈ or WT1₃₂₈₋₃₄₈ substituted with alanine at indicated position, for 20-24 hours. WT1₂₃₅₋₂₄₃ was used as a negative control. IFN-γ secretion was measured by ELISPOT. The data shown represent the mean±SD of experiments performed in triplicates. Results are representative of two independent experiments. *p<0.05, **p<0.01, ***p<0.001, ****p<0.0001 by one-way ANOVA with Dunnett correction. FIGS. 6U-6V show the results of a T2 Cell-based competitive binding assay performed using T2/DP4 pulsed with graded concentrations of WT1₃₂₈₋₃₄₈, or WT1₃₂₈₋₃₄₈ substituted with alanine at indicated position, in the presence of 2 mM biotin-conjugated reference peptide, CLIP. Cells were then stained by phycoerythrin-conjugated streptavidin and analyzed by flow cytometric assays. Percent inhibition of binding by competitor peptides was calculated by Mean Fluorescence Intensity (MFI) using the following formula: Top: % inhibition of representative competitor peptides were shown. % inhibition=[1−(MFI with competitor peptide/MFI without competitor peptide)]×100. Bottom: IC50 values are also shown. N/C, not calculable.

FIGS. 7A-7U present data showing that WT1 CAR-T cells recognize WT1 peptide presented by diverse HLA class II molecules. FIGS. 7A-7B: Cytotoxicity of WT1 TCR (left) or CAR (right)-transduced T cells against the indicated K562 transfectants pulsed with WT1₃₂₈-348 or MAGE-A3₂₄₃₋₂₅₈ at an E:T ratio of 5:1 for 5 hours was measured by in vitro flow cytometry-based killing assays (n=3 donors). FIGS. 7C-7D: Cytotoxicity of WT1 TCR (left) or CAR (right)-transduced T cells against the indicated WT1-overexpressing K562 transfectants at an E:T ratio of 5:1 for 18 hours was measured by in vitro flow cytometry-based killing assays (n=5 donors). Class II-null, WT1-overexpressing K562 (WT) was used as a negative control. FIGS. 7E-7G: WTT TCR or CAR-transduced T cells were stimulated with the indicated WT1-overexpressing K562 transfectants at an E:T ratio of 1:1 for 5 hours; cytokine secretion of CD4+ and CD8+ T cells was measured by intracellular flow cytometric analysis (n=3 donors). FIGS. 7K-7R: WT1 TCR or CAR-transduced T cells were stimulated with the indicated K562 transfectants (WT1+ or WT1-KO) at an E:T ratio of 1:1 for 5 hours; cytokine secretion of CD4+ and CD8+ T cells was measured by intracellular flow cytometric analysis (n=3 donors). FIG. 7S: Cytotoxicity of WT1 CAR-transduced T cells against the indicated K562 transfectants (WT1+ or WT1-KO) at an E:T ratio of 5:1 for 18 hours was measured by in vitro flow cytometry-based killing assays (n=3 donors). Results are representative of two independent experiments. *p<0.05, **p<0.01, ***p<0.001, ****p<0.0001 by two-way ANOVA with Bonferroni correction. FIG. 7T-7U: WT1 CAR-transduced T cells were stimulated with T2/DR15 (FIG. 7T) or T2/DQ9.2 (FIG. 7U) pulsed with WT1₃₂₈₋₃₄₈ or WT1₃₂₈-348 substituted with alanine at indicated position, for 20-24 hours. WT1₂₃₅₋₂₄₃ was used as a negative control. IFN-γ secretion was measured by ELISPOT. The data shown represent the mean±SD of experiments performed in triplicates. Results are representative of two independent experiments. *p<0.05, **p<0.01, ***p<0.001, ****p<0.0001 by one-way ANOVA with Dunnett correction.

FIGS. 8A-8SS present data showing that WT1 CAR-T cells recognize various WT1+/class 11-leukemia and lymphoma cell lines and primary leukemic samples. FIGS. 8A-8P: Surface class II expression of the indicated leukemia and lymphoma cell lines was measured by flow cytometric analysis after staining with isotype control or anti-class II mAb, as indicated. FIGS. 8Q-8FF: Intracellular expression of WT1 of the indicated leukemia and lymphoma cell lines was measured by flow cytometric analysis after staining with isotype control or anti-WT1 mAb, as indicated. FIGS. 8GG-8HH: Cytotoxicity of WT1 TCR or CAR-transduced T cells against the indicated leukemia and lymphoma cell lines at an E:T ratio of 0.2:1 or 1:1 for 18 hours was measured by in vitro flow cytometry-based killing assays (n=3 donors). Results are representative of two independent experiments. FIGS. 8II-8LL: Cytotoxicity of WT1 CAR or CD19 CAR (control)-transduced T cells against OCI-AML5 at an E:T ratio of 1:1, 5:1, 20:1 for 5 hours was measured by in vitro flow cytometry-based killing assays (n=3 donors). FIGS. 8MM-8NN: WT1 CAR or CD19 CAR (control)-transduced T cells were stimulated with K562/DP4/WT1 or OCI-AML5 for 20-24 hours; IFN-γ secretion was measured by ELISPOT analysis. The data shown represent the mean±SD of experiments performed in triplicates. Results are representative of two independent experiments. ***p<0.001, ****p<0.0001 by two-way ANOVA with Bonferroni correction. FIG. 8SS: Cytotoxicity of WT1 CAR or CD19 CAR (control)-transduced T cells against primary ALL or AML samples at an E:T ratio of 5:1 for 18 hours was measured by in vitro flow cytometry-based killing assays (n=3 donors). Results are representative of two independent experiments. ***p<0.001, ****p<0.0001 by two-way ANOVA with Bonferroni correction.

FIGS. 9A-9BB present data showing that WT1 CAR-T cells possess minimal on-target and off-target reactivity. FIGS. 9A-9X: Surface class II expression of the indicated cell lines and primary human CD34+ hemopoietic cells was measured by flow cytometric analysis after staining with isotype control or anti-class II mAb, as indicated; and intracellular expression of WT1 of the indicated cell lines and primary human CD34+ hemopoietic cells was measured by flow cytometric analysis after staining with isotype control or anti-WT1 mAb, as indicated. FIG. 9Y: Cytotoxicity of WT1 CAR or CD19 CAR (control)-transduced T cells against indicated cell lines and human primary CD34+ hemopoietic cells at an E:T ratio of 5:1 for 6 hours was measured by in vitro flow cytometry-based killing assays (n=6 donors). Results are representative of two independent experiments. ****p<0.0001 by two-way ANOVA with Bonferroni correction. FIGS. 9Z-9BB: WT1 CAR-transduced T cells were stimulated with the indicated T2-derived class II transfectants pulsed with WT1₃₃₀₋₃₄₈ or WT1₂₃₅₋₂₄₃ (control) or cross-reactive peptide candidates for 20-24 hours; IFN-γ secretion was measured by ELISPOT. The data shown represent the mean±SD of experiments performed in triplicates.

FIGS. 10-10T present data showing a comparison between WT1 28z and WT1 4-1BBz CAR-T cells. FIG. 10A is a schematic representation of the WT1 28z and WT1 4-TBBz CAR constructs. The single-chain variable fragment (scFv) of both CARs was formed by connecting the variable regions of heavy (VH) and light chain (VL) derived from WT1-specific mAb, 5H2, via a Whitlow linker. For 28z CAR, the scFv was linked to human CD28 transmembrane and cytoplasmic domain, followed by cytoplasmic domain of human CD3 zeta. For 4-1BBz CAR, the scFv was linked to human CD8a transmembrane domain, followed by cytoplasmic domains of human 4-1BB and CD3 zeta. Both CAR constructs were N-terminally linked to truncated NGFR (ΔNGFR) via a furin cleavage site, a SGSG spacer sequence, and a P2A sequence. FIGS. 10B-10C: Human primary T cells transduced with WT1 28z or WT1 4-1BBz CAR were stained with Pacific Blue-conjugated anti-NGFR mAb (left), or biotinylated protein-L followed by streptavidin-PE (right), and analyzed by flow cytometric assays. FIGS. 10D-10G: Surface expression of CD25, CD45RA and CD62L of WTT 28z or WT1 4-1BBz CAR-transduced CD4+ and CD8+ T cells 1 week after retroviral transduction was measured by flow cytometric analysis. FIGS. 10H-10M: WT1 28z or WT1 4-1BBz CAR-transduced T cells were stimulated with the indicated WT1-overexpressing K562 transfectants at an E:T ratio of 1:1 for 5 hours; cytokine secretion of CD4+ and CD8+ T cells was measured by intracellular flow cytometric analysis. WT1 28z or WT1 4-1BBz CAR-transduced T cells were stimulated with the indicated WT1-overexpressing K562 transfectants pulsed with WT1328-348 at an E:T ratio of 1:1 for 5 hours; cytokine secretion of CD4+ and CD8+ T cells was measured by intracellular flow cytometric analysis. FIG. 10T: Cytotoxicity of WT1 28z or WT1 4-1BBz CAR-transduced T cells against the indicated K562 transfectants or OCI-AML5 at an E:T ratio of 5:1 for 18 hours was measured by in vitro flow cytometry-based killing assays. B-F: n=3 donors; results are representative of two independent experiments. *p<0.05, **p<0.01, ***p<0.001, ****p<0.0001 by two-way ANOVA with Bonferroni correction.

FIGS. 11A-11H provide data showing that WT1-specific mAb can recognize exogenously-pulsed WT1₃₂₈₋₃₄₈ peptide presented by different HLA-DP molecules, including DP2 (FIG. 11D), DP4 (FIG. 11F), and DP5 (FIG. 11H). Signal was above background in each case (FIGS. 11A-11C, 11E, and 11G).

FIGS. 12A-12E provide data showing that WT1-specific mAb can recognize naturally processed WT1 peptide presented by HLA-DP2 (FIGS. 12B and 12D) and HLA-DP4 (FIGS. 12C and 12E).

FIGS. 13A-13C provide data showing that WT1 CAR-T cells effectively recognize target cells simultaneously presenting WT1 peptide on multiple class II molecules. FIG. 13A shows surface expression of DP, DR, DQ of K562 transfectants analyzed by flow cytometric assay after staining with specific monoclonal antibodies. FIG. 13B shows cytokine secretion by WT1 CAR-transduced CD4⁺ and CD8⁺ T cells stimulated with the indicated K562 transfectants at an E:T ratio of 1:1 for 5 hours. Cytokine secretion of CD4⁺ and CD8⁺ T cells was measured by intracellular flow cytometric analysis (n=3 donors). FIG. 13C is a bar graph illustrating cytotoxicity of WT1 CAR or CD19 CAR (control)-transduced T cells against the indicated K562 transfectants at an E:T ratio of 5:1 for 5 hours as measured by in vitro flow cytometry-based killing assays (n=4 donors).

FIGS. 14A-14B provide data showing WT1 CAR-T cells demonstrate long-term antitumor response against target cells simultaneously presenting WT1 peptide on multiple class II molecules. FIG. 14 shows long-term cytotoxicity of WT1 CAR-T cells against the indicated K562 transfectants as measured by impedance-based xCELLigence killing assay (n=5 donors). K562 transfectants adhered and expanded for 24 h before WT1 CAR T-cells were added at an E:T ratio of 1:5. FIG. 14B shows surface expression of PD-1, TIM-3 and LAG-3 on WT1 CAR-transduced CD4⁺ and CD8⁺ T cells stimulated with the indicated K562 transfectants as measured by flow cytometric analysis at the end of xCELLigence killing assay (n=4 donors). *p<0.05, **p<0.01, ****p<0.0001 by one-way ANOVA with Bonferroni correction.

FIGS. 15A-15B provide data showing WT1 CAR-T cells recognize a WT1V/class II⁺ leukemic cell line. FIG. 15A shows cytotoxicity of WT1 CAR or CD19 CAR (control) or MSLN CAR (control)-transduced T cells against K562 or PR9 at an E:T ratio of 5:1 for 18 hours as measured by in vitro flow cytometry-based killing assays (n=3 donors). FIG. 15B shows cytokine secretion by WT1 CAR or CD19 CAR (control) or MSLN CAR (control)-transduced CD4⁺ and CD8⁺ T cells stimulated with K562 or PR9 at an E:T ratio of 1:1 for 5 hours. Cytokine secretion of CD4⁺ and CD8⁺ T cells was measured by intracellular flow cytometric analysis (n=4 donors). A, B: *p<0.05, **p<0.01, ****p<0.0001 by two-way ANOVA with Bonferroni correction.

FIGS. 16A-16E provide data showing antitumor activity of WT1 CAR-T cells in vivo. FIG. 16A is a schematic illustration of the experimental design, showing that NSG mice were intravenously infused with 0.1×10⁶ luciferase-expressing PR9 cells (day −3) and were then transplanted with 5×10⁶ CAR-T cells (day 0) or remained untreated. FIG. 16B shows the tumor burden of the untreated or WT1 or MSLN (control) CAR-T treated mice as analyzed by in vivo bioluminescent imaging of luciferase activity and total photon counts were quantified (n=6 mice per group). FIG. 16C shows persistence of PR9 cells in peripheral blood as analyzed at the indicated time points. FIG. 16D is a Kaplan-Meier curve for the overall survival of mice in each treatment cohort (n=6 mice per group; P values were calculated by the log-rank test). FIG. 16E shows persistence of CAR-T cells in peripheral blood as analyzed at the indicated time points.

FIG. 17 provides data showing antitumor activity of WT1 CAR-T cells in vivo. In vivo bioluminescent imaging of luciferase activity in NSG mice in each treatment cohort is shown.

FIGS. 18A-18B provide date showing CyTOF-based analysis of WT1 TCR or CAR-T cells targeting WT1 peptide presented by HLA-DP4. FIG. 18A is a heatmap depicting expression of the indicated cell surface and intracellular markers of CD4-(top) or CD8⁺ (bottom) WT1 TCR or CAR-transduced T cells stimulated with WT1-overexpressing K562/DP4 cells at an E:T ratio of 1:1 for 1 day, 3 days, or 5 days. Results are representative of six donors from two independent experiments. Markers with similar patterns observed across different donors are highlighted in red. FIG. 18B shows expression of the indicated cell surface markers of CD4⁺ (top) or CD8⁺ (bottom) by WT1 TCR- or CAR-transduced T cells stimulated with WT1-overexpressing K562/DP4 cells at an E:T ratio of 1:1 for 1 day, 3 days, or 5 days (n 3 donors). *p<0.05, **p<0.01, ***p<0.001, ****p<0.0001 by two-way ANOVA with Bonferroni correction. Results are representative of two independent experiments.

### DETAILED DESCRIPTION OF THE DISCLOSURE

Some aspects of the present disclosure are directed to antigen-binding molecules that bind a peptide fragment of a tumor antigen, wherein the peptide fragment is capable of being presented by a major histocompatibility complex (MHC) class II molecule. In some aspects, the peptide fragment is a fragment of WT1.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (Si) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, C_{H1}, C_{H2} and C_{H3}. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FRs). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Therefore, the term “anti-WT1 antibody” includes a full antibody having two heavy chains and two light chains that specifically binds to WT1 and antigen-binding portions of the full antibody. Non limiting examples of the antigen-binding portions are shown elsewhere herein.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to WT1 is substantially free of antibodies that bind specifically to antigens other than WT1). An isolated antibody that binds specifically to WT1 may, however, have cross-reactivity to other antigens, such as WT1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-WT1 antibody binds specifically to WT1 or a particular fragment thereof.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

An “antigen” refers to any molecule, e.g., a peptide, that provokes an immune response or is capable of being bound by a TCR. An “epitope,” as used herein, refers to a portion of a polypeptide that provokes an immune response or is capable of being bound by a TCR. The immune response may involve either antibody production, or the activation of specific immunologically-competent cells, or both. A person of skill in the art would readily understand that any macromolecule, including virtually all proteins or peptides, can serve as an antigen. An antigen and/or an epitope can be endogenously expressed, i.e. expressed by genomic DNA, or can be recombinantly expressed. An antigen and/or an epitope can be specific to a certain tissue, such as a cancer cell, or it can be broadly expressed. In addition, fragments of larger molecules can act as antigens. In one aspect, antigens are tumor antigens. An epitope can be present in a longer polypeptide (e.g., in a protein), or an epitope can be present as a fragment of a longer polypeptide. In some aspects, an epitope is complexed with a major histocompatibility complex (MHC; also referred to herein as complexed with an HLA molecule, e.g., an HLA class 1 molecule).

As used herein, the term “chimeric antigen receptor” of “CAR” refers to a non-naturally occurring polypeptide comprising (i) an antigen-binding domain, (ii) a transmembrane domain, and (iii) an intracellular signaling domain. In some aspects, the CAR further comprises a hinge region between the antigen-binding domain and the transmembrane domain. In some aspects, the intracellular signaling domain comprises a costimulatory region and an activation domain. In some aspects, the antigen-binding domain comprises an antibody variable heavy (V-H) domain and an antibody variable light (VL) domain connected by a linker. In some aspects, the transmembrane domain comprises a CD28 transmembrane domain. In some aspects, the hinge domain comprises an IgG hinge domain. In some aspects, the transmembrane domain comprises a CD8α transmembrane domain. In some aspects, the hinge domain comprises a CD8α hinge domain. In some aspects, the costimulatory region comprises a CD28 costimulatory region or a 4-1BB costimulatory region. In some aspects, the activation domain comprises a CD3 zeta activation domain.

As used herein a “multispecific antibody” refers to an antibody that is capable of binding more than one antigen. In some aspects, the multispecific antibody comprises a first antigen-binding domain and a second antigen-binding domain and is capable of bind two antigens (e.g., a “bispecific antibody”). In some aspects, the multispecific antibody comprises a first antigen-binding domain, a second antigen-binding domain, and a third antigen-binding domain and is capable of bind three antigens (e.g., a “trispecific antibody”). In some aspects, the first antigen-binding domain and the second antigen-binding domain bind the same antigen. In some aspects, the first antigen-binding domain and the second antigen-binding domain bind two different epitopes on a single polypeptide. In some aspects, the first antigen-binding domain and the second antigen-binding domain bind different antigens.

The term “BiTE” or “bispecific T cell engager,” as used herein, refers to a bispecific antibody comprising (i) a first antigen-binding domain that binds a particular target, e.g., a fragment of WT1, and (ii) a second antigen-binding domain that binds CD3.

The term “TriTE” or “trispecific T cell engager,” as used herein, refers to a trispecific antibody comprising (i) a first antigen-binding domain that binds a particular target, e.g., a fragment of WT1, (ii) a second antigen-binding domain that binds CD3, and (iii) a third antigen-binding domain that binds a third antigen.

The term “HLA,” as used herein, refers to the human leukocyte antigen. HLA genes encode the major histocompatibility complex (MHC) proteins in humans. MHC proteins are expressed on the surface of cells, and are involved in activation of the immune response. HLA class II genes encode MHC class II proteins which are expressed on the surface of professional antigen presenting cells (APCs). Non-limiting examples of professional APCs include monocytes, macrophages, dendritic cells (DCs), and B lymphocytes. Some endothelial and epithelial cells can also express MI-C class II molecules after inflammatory signals are activated. Humans lacking functional MHC class II molecules are extremely susceptible to an array of infectious diseases and typically die at a young age.

As used herein, an “HLA class II molecule” or “MHC class 11 molecule” refers to a protein product of a wild-type or variant HLA class II gene encoding an MHC class II molecule. Accordingly, “HLA class II molecule” and “MHC class II molecule” are used interchangeably herein. A typical MHC Class II molecule comprises two protein chains: an alpha chain and a beta chain. In general, naturally occurring alpha chains and beta chains each comprise a transmembrane domain, which anchors the alpha/beta chain to the cell surface, and an extracellular domain, which carries the antigen and interacts with a TCR and/or CD4 expressed on a T cell, or, as disclosed herein, a CAR expressed on a T cell.

Both the Nil-IC Class II alpha and beta chains are encoded by the HILA gene complex. The HLA complex is located within the 6p21.3 region on the short arm of human chromosome 6 and contains more than 220 genes of diverse function. The HLA gene complex is highly variant, with over 20,000 HLA alleles and related alleles, including over 830 MHC class II alpha chain alleles and over 8,000 MHC class II beta chain alleles, known in the art, encoding thousands of MHC class II proteins (see, e.g., hla.alleles.org, last visited Mar. 24, 2022, which is incorporated by reference herein in its entirety). For example one such HLA-DP allele, DP4 is the most frequently found allele in many ethnic groups.

Three loci in the HLA complex encode MHC Class II proteins: HLA-DP, HLA-DQ, and LILA-DR. HLA-DO and FILA-DM encode proteins that associate with the M-IC class II molecule and support its configuration and function.

When the MHC class II molecule is complexed with an antigen peptide, the 10-30 amino acid long antigen peptide binds the peptide-binding groove and is presented extracellularly to CD4+ cells. Both the alpha- and beta-chains fold into two separate domains; alpha-1 and alpha-2 for the alpha polypeptide, and beta-1 and beta-2 for the beta polypeptide. The open-ended peptide-binding groove which holds the presented antigen is found between the alpha-1 and beta-1 domains. Upon interaction with a CD4+ T cell, the MHC class II complex interacts with a T cell receptor (TCR) expressed on the surface of the T cell. In addition, the beta chain of the MHC class II molecule weakly interacts (KD>2 mM) with CD4 expressed on the surface of the T cell. As described herein, the antigen peptide can also be used to raise an antibody specific to the antigen peptide. As such, an antibody or a CAR comprising an antigen-binding domain can specifically bind the antigen peptide presented by the M-IC class II molecule.

The term “autologous” refers to any material derived from the same individual to which it is later to be re-introduced. For example, an autologous T cell therapy comprises administering to a subject a T cell that was isolated from the same subject. The term “allogeneic” refers to any material derived from one individual which is then introduced to another individual of the same species. For example, an allogeneic T cell transplantation comprises administering to a subject a T cell that was obtained from a donor other than the subject.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present invention include, but are not limited to, cancers of the immune system including lymphoma, leukemia, and other leukocyte malignancies. In some aspects, the methods of the present invention can be used to reduce the tumor size of a tumor derived from, for example, bone cancer, renal cancer, prostate cancer, breast cancer, colon cancer, lung cancer, cutaneous or intraocular malignant melanoma, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stern glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. The particular cancer can be responsive to chemo- or radiation therapy or the cancer can be refractory.

A refractory cancer refers to a cancer that is not amendable to surgical intervention, and the cancer is either initially unresponsive to chemo- or radiation therapy or the cancer becomes unresponsive over time.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

The term “progression-free survival,” which can be abbreviated as PFS, as used herein refers to the time from the treatment date to the date of disease progression per the revised IWG Response Criteria for Malignant Lymphoma or death from any cause.

“Disease progression” or “progressive disease,” which can be abbreviated as PD, as used herein, refers to a worsening of one or more symptom associated with a particular disease. For example, disease progression for a subject afflicted with a cancer can include an increase in the number or size of one or more malignant lesions, tumor metastasis, and death.

The “duration of response,” which can be abbreviated as DOR, as used herein refers to the period of time between a subject's first objective response to the date of confirmed disease progression, per the revised IWG Response Criteria for Malignant Lymphoma, or death.

The term “overall survival,” which can be abbreviated as OS, is defined as the time from the date of treatment to the date of death.

A “cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

Other examples of analytes and cytokines of the present invention include, but are not limited to chemokine (C-C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, granulocyte colony-stimulating factor (G-CSF), leukemia inhibitory factor (LIF), oncostatin M (OSM). CD154. lymphotoxin (LT) beta, 4-1 BB ligand (4-1BBL), a proliferation-inducing ligand (APRIL), CD70, CD153, CD178, glucocorticoid-induced TNFR-related ligand (GITRL), tumor necrosis factor superfamily member 14 (TNFSF14), OX40L, TNF- and ApoL-related leukocyte-expressed ligand 1 (TALL-1), or TNF-related apoptosis-inducing ligand (TRAIL).

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). T-cell receptors (TCR) differentiate T cells from other lymphocyte types. The thymus, a specialized organ of the immune system, is primarily responsible for the T cell's maturation. There are six types of T-cells, namely: Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+(L-selectin), CD27+, CD28+ and IL-7Ra+, but they also express large amounts of CD95, IL-2RP, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and Gamma Delta T-cells. B-cells, on the other hand, play a principal role in humoral immunity (with antibody involvement). A B cell makes antibodies and antigens and performs the role of antigen-presenting cells (APCs) and turns into memory B-cells after activation by antigen interaction. In mammals, immature B-cells are formed in the bone marrow, where its name is derived from.

The term “genetically engineered” or “engineered” refers to a method of modifying the genome of a cell, including, but not limited to, deleting a coding or non-coding region or a portion thereof or inserting a coding region or a portion thereof. In some aspects, the cell that is modified is a lymphocyte, e.g., a T cell or a modified cell that expresses CD4, which can either be obtained from a patient or a donor. The cell can be modified to express an exogenous construct, such as, e.g., a T cell receptor (TCR) disclosed herein, which is incorporated into the cell's genome. In some aspects, the cell is modified to express CD4.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT), and allogeneic T cell transplantation.

Cells used in an immunotherapy described herein can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety. An immunotherapy can also comprise administering a modified cell to a subject, wherein the modified cell expresses a CAR disclosed herein. In some aspects, the modified cell is not a T cell.

A “patient” as used herein includes any human who is afflicted with a cancer (e.g., a lymphoma or a leukemia). The terms “subject” and “patient” are used interchangeably herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., a CAR, that specifically binds with a cognate stimulatory ligand present on an antigen present cell. A “stimulatory ligand” is a ligand that when present on an antigen presenting cell (e.g., an aAPC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an MHC Class II molecule loaded with a peptide, an anti-CD4 antibody, a superagonist anti-CD2 antibody, a superagonist anti-CD28 antibody, and a superagonist anti-CD3 antibody.

The terms “conditioning” and “pre-conditioning” are used interchangeably herein and indicate preparing a patient in need of a T cell therapy for a suitable condition. Conditioning as used herein includes, but is not limited to, reducing the number of endogenous lymphocytes, removing a cytokine sink, increasing a serum level of one or more homeostatic cytokines or pro-inflammatory factors, enhancing an effector function of T cells administered after the conditioning, enhancing antigen presenting cell activation and/or availability, or any combination thereof prior to a T cell therapy. In one aspect, “conditioning” comprises increasing a serum level of one or more cytokines, e.g., interleukin 7 (IL.-7), interleukin 15 (IL-15), interleukin 10 (IL-10), interleukin 5 (IL-5), gamma-induced protein 10 (IP-10), interleukin 8 (IL-8), monocyte chemotactic protein 1 (MCP-1), placental growth factor (PLGF), C-reactive protein (CRP), soluble intercellular adhesion molecule 1 (sICAM-1), soluble vascular adhesion molecule 1 (sVCAM-1), or any combination thereof. In another aspect, “conditioning” comprises increasing a serum level of IL-7, IL-15, IP-10, MCP-1, PLGF, CRP, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one aspect, “treatment” or “treating” includes a partial remission. In another aspect, “treatment” or “treating” includes a complete remission.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (i.e., ±10%). For example, about 3 mg can include any number between 2.7 mg and 3.3 mg (for 10%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Various aspects of the invention are described in further detail in the following subsections.

### II. Compositions of the Disclosure

Some aspects of the present disclosure are directed to an antibody or an antigen-binding portion thereof that specifically binds a peptide fragment of a tumor antigen, wherein the peptide fragment is presented by an MIC class II molecule. The antibodies and antigen-binding portions thereof disclosed herein are capable of binding peptide fragments that are presented by any type of MHC class II molecules. As such, the antibody or antigen-binding portion thereof is capable of binding a peptide fragment that is presented by a first MI-IC class II molecule and a second MHC class II molecule, wherein the first and the second MHC class II molecules are different. Further, the antibody or antigen-binding portion thereof is specific to the peptide fragment, and the antibody or antigen-binding portion thereof does not specifically bind an epitope on the MHC class II molecule. The promiscuity allows for the antibody or antigen-binding portion thereof to be effective in binding the target peptide fragment regardless of the HLA genotype of the patient.

The methods described herein can be used to generate an antibody or an antigen-binding portion thereof to any peptide fragment that is displayed by an MIC class II molecule. In some aspects, the peptide fragment is a fragment of a tumor antigen. In some aspects, the tumor antigen is ACTN4, BRAF CA SPS, CDC27, CDK4, CTN7NB1, EEF2, EFTUD2, FN1, GP NMB, HHAT, HSPA1B, KRAS, ME1, MUM3, MYO1B, NFYC, NRAS, OS9, PAPOLG, LPGAT1, PRDX5, PTPRK, SIRT2, SNRPD1, TPI1, TRAPPC1, UBXD5, ZUBR1, CASP5, CDKN2A, OGT, CAMEL, MUM1, ABL-BCR chromosomal translocation, BCR-ABL chromosomal translocation, DEK-CAN chromosomal translocation, ETV6-AML1 chromosomal translocation, LDLR-FUT chromosomal translocation, NPM1-ALK1 chromosomal translocation, PAX3-FKHR chromosomal translocation, PML-RARA chromosomal translocation, SYT-SSX1 chromosomal translocation, SYT-SSX2 chromosomal translocation, FLT3, 707-AP, ANKRD30A, DCT, GPR143, KLK3, KLK4, MClR, MLANA, OCA2, RAB38, SCGB2A2, SILV, SOX2, TYR, TYRPI, XAGE1, ABCC3, ACPP, ADAM17, ADFP, AFP, AIM2, ALDHIA1, ALK, AML1, ART4, BCL-2, BCL2L1, BIRC5, BIRC7, BST2, CAS, CCNI, CCNB1, CCND1, CEL, CEACAM5, CLCA2, CPSF1, CSPG4, CSF1, CYPIBI, DDR1, DEK, DKK1, EGFR, ENAH, EPHA2, EPHA3, ERBB2, ETV5, EZH2, FGF5, F4.2, FMENLI, FOLH1, GPC3, HSPA1 A, IL13RA2, KAAG1, MCL1, MIDM2, MMP2, MRPL28, MSLN, MUC1, MUC2, NPM1, PAX3, PPIB, PRAME, RAGE, RGS5, RHAMM, RNF43, SARTI, SART3, SCRNI, SFMBT1, SOX10, SOX11, SOX4, STEAPI, SYNDI, TACSTDI, TERT, TOP2A, TOP2B, TP53, TPBG, TRG, TRIM68, TRPM8, TSPYLI, WDR46, WT1, XBPI, ZNF395, ANXA2, BAGE, CCDClIO, CSAG2, CTAGI, CTAG2, CXORF61, GAGEI, GAGE2, GAGE3, GAGE4, GAGE5, GAGE6, GAGE7, HERV-K-MEL, GAGE8, MAGEAI, MAGEA10, MAGEA12, MAGEA2, MAGEA3, MAGEA4, MAGEA9, MAGEBI, MAGEB2, MAGEC2, MGAT5, SAGEI, SPA17, SSX2, SSX4, SYCPI, TGFBR2, VENTXPI, AB12, ABLI, ACRBP, AKAP13, APC, ARTC1, ATIC, BAAT, BCAP31, BCR, BTBD2, CALR3, CAN, CDC2, CDKNIA, COTL1, CTSH, DNAJC8, EIF4EBP1, ETV6, FMOD, FOXOI, FUTI, H3F3A, HSMD, HMHAI, HMOX1, HPSE, HNRPL, IER3, IGF2BP3, ITGB8, ITPR2, JUP, LCK, LDLR, LGALS3BP, LRP1, LY6K, MAGED4, MET, MFGE8, MFI2, MMP14, OAS3, PA2G4, PAGE4, PAK2, PARP12, PGK1, PMLI, PRTN3, PSCA, PTHLH, PXDNL, RARA, RCVRN, RPAI, RPL1OA, RPS2, RPSA, SDCBP, SEPT2, SLBP, SLC35A4, SLC45A2, SSX1, STATI, SUPT7L, SYT, TAPBP, TOR3A, TPM4, TTK, TYMS, LTBE2A, LTBE2V1, WHSC2, WNK2, RPTOR, USP9X, or any combination thereof. In some aspects, the tumor antigen is any antigen disclosed in Van den Eynde et al., Curr. Opin. Imnunol. 9:684-93 (1997), which is incorporated by reference herein in its entirety.

In some aspects, the tumor antigen is KRAS, IGHV4-28, IGHV3-48, IGHV3-53, IGHV4-61, IGHV3-66, IGKV3D-15, TMPRSS15, GOLGA6L2, GGT5, MIPEP, C1QTNF9B, RHBDF1, IFNGR1, NACA, POTEG, CCT6B, FAT4, SLC9A3R1, RAPGEF2, CTNND1, IGF1R, ZNF33A, KCNH2, PRR36, SPSB4, GLRB, NUP98, SNTG2, SH3RF3, DPP3, BRCA2, HSD17B1, RPL11, SHROOM4, POLR2M, FNDC9, NBEA, ITGB4, PWWP2B, GOLGA8K, WDR7, TOP1MT, MDM1, CDKN2AIP, DZIP1L, OR51H1, APOF, FBLN2, HIST1H3B, RADIL, OR51J1, UTF1, SCML4, MUC16, TARSL2, COL6A5, MUC6, WDR17, KCNMA1, GPD2, RBPJ, HS3ST6, COL6A6, PMS1, DNAH8, ATP2B1, ZNF66, NRXN3, RGPD3, GAS2L2, IRF2, GLP1R, ZNF208, FAM186A, MAP1A, ZNF724, DYNLT3, KDM5C, CRISP3, NPEPPS, CYP24A1, TUBB8P12, UNC13B, RTN2, MIA3, SORBS2, SFXN2, GEMIN2, TTN, RPS6KA1, STK3, KRTAP1O-9, GPR1, ADAM23, SLC17A7, CCAR2, SNX24, MYH2, LRFN5, UCHL1, ZNF717, PTPRM, USP6, SLC5A12, SPAG1IA, OBSCN, KIAA1109, PRAG1, UBALDI, LRP4, ABCC12, SLC25A16, CNTN1, KPNA1, POU1F1, KRTAP4-8, KARS, ZNF700, PITPNM2, KRTAP9-9, ZNF433, KIAA1549L, OTOP1, SORCS3, TNXB, SLC9A6, ADAMTS20, RYR2, FBXW9, GOLGA6B, BEST2, UCP2, TEX13A, SORL1, RNF217, PCSK4, IMIPDH1, ZNF571, GDF5OS, KCNQ5, XIRP2, FAM118A, SLC8A1, IER3, GNAO1, DLGAP3, RASGRP4, NT5C1B, TGFBR1, XYLT1, PRMT6, FBH1, NOTCH3, UHRF1BP1L, SAXO2, GIPC3, GSTM1, PCDHA1, UMOD, PLEKHG6, KCNA2, LILRA3, OR5L1, OR8U8, OR5AP2, NBPF1, HDGFL2, JPH2, CEP295, SLC27A1, KIAA0895L, CACNB3, PDE4DIP, MUC4, SELPLG, BRINP3, INAVA, CACNAlS, IGFN1, ACAP3, Clorfl27, STPG1, MECR, MACF1, KIAA0754, ERMAP, MPL, ELAVL4, FAM69A, PPIAL4G, FGFR1, LCE5A, LCE1C, IQGAP3, COPA, PRG4, MARK1, SFMBT2, C10orf71, CTNNA3, ZFYVE27, SORCS1, KRTAP5-4, OR51A4, FOLH1, SLC22A20P, PLEKHB1, SIDT2, CD4, A2M, TAS2R43, PRB3, PRB1, SLCO1A2, LRRK2, SLC6A15, ANKS1B, NUAK1, ZCCHC8, ATP12A, WBP4, GPC6, TRAV20, SIPA1L1, CEP170B, JAG2, IGHE, CXADRP2, GOLGA8EP, GOLGA8N, SNUPN, LOC645752, CPEB1, ACAN, KIF7, SSTR5-AS1, IFT140, EME2, TMC7, DNAH3, ZKSCAN2, ZNF267, LOC283922, CHST6, CNTNAP4, CDH13, GLTPD2, ELAC2, ADAM11, PCYT2, TXNDC2, SALL3, CD70, ZNF431, ZNF43, CCNE1, SBSN, KMT2B, ZNF146, ZNF585B, A1BG, TRIM28, CPSF3, ATAD2B, ASXL2, LRP1B, CASP8, CARF, NBEAL1, SPHKAP, C20orfl94, RRBP1, RALGAPA2, GDF5, SCANDI, KCNB1, TEKT4P2, COL6A1, POTEH, HIRA, TXNRD2, APOL5, SREBF2, TUBGCP6, GRM2, ROBO2, ROBO1, H1FOO, SMC4, FAM131A, SDHAP2, SLIT2, LIMCH1, MUC7, MGAT4D, HHIP, C4orf51, TUBB7P, DNAH5, FOXD1, ZFYVE16, JADE2, ABLIM3, PIP5K1P1, GCM2, FAM8A1, HFE, MUC22, ORC3, AK9, SLC22A16, TMEM181, TCP10, FAM120B, EIF2AK2, SCIN, CLK2P1, LOC646999, CCDCl46, ASNS, ZAN, SLC12A9, DLD, ING3, AKR1B15, UBE3C, ADAM17, SNTG1, XKR9, DENND3, MROH5, CNTLN, LINGO2, CBWD3, CEP78, FKBP15, ASTN2, SEC16A, ENTPD2, SCML2, RPGR, GNL3L, LPAR4, UBE2DNL, ARMCX4, NKAP, MAGEC1, MUC12, HGC6.3, METTL24, OR8H2, OR5M10, OR2M3, ZNF391, OR13C5, OR5M9, OR4K14, OR4N4, OR13F1, OR8K5, PCDHB11, MAGEC3, PCDHB10, KRTAP5-9, ZNF648, ZFP28, TNN, MLANA, INMT, OR2L3, CTDSPL, H1FNT, PSG3, KRTAP5-3, XKR6, FRG2B, LONRF2, LRIT1, FCGBP, FGFBP2, ZNF493, IQCF1, GRID2IP, SLC44A5, FAM205A, POTEC, ODF3L2, ANKRD30B, ZNF814, ZFP92, PRB2, GALNT9, GOLGA8B, NAV2, DYSF, UHRF2, PCDH8, SPTB, PIK3CA, TP53, CYP2E1, HDAC4, KPNA6, BRD4, IRX1, CHTF18, VCL, DHX15, PRDM4, KCNK10, AGAP11, SEZ6, SF3A2, PIK3C2B, RPS21, COIL, HOXB8, PABPC3, WSCD1, SBNO2, AKAP12, ABCA3, KRTAP4-12, ATP6V1C2, DIAPHI, LRRN3, WWC3, SLC7A4, SKIV2L, AGAP5, CREM, UIMC1, PDLIM5, PPP2R5B, NPDC1, CHD6, INPP5K, AHNAK, PCNX3, FAM49B, CFAP77, HRNR, ICOSLG, HSPH1, CMPK1, SLC34A1, LOXHDI, FAM102A, VANGL2, PAX1, KIF18B, KCNH6, KCNH5, IQCN, PLEC, ORC2, CSPP1, PLEKHG5, SIX4, DDX27, MBD5, MAPKAPK2, SLC25A5, HOMER3, EPB41L4B, KCNA5, LUZP1, WNK3, CUX2, PARL, KLHL14, NUDT9, HNRNPCL2, RNF213, FAM47A, C17orf97, ZNF701, FAM47C, PRRT4, PLD6, ZNF587, ZNF615, LCE1A, ZNF519, C3orfl8, ZNF860, GOLGA8M, IFITM5, CHST2, DENND2C, NAV3, PRAMEF7, GOLGA8H, PRAMEFI, FAM53A, C14orfl80, DDX4, TRABD, VAT1L, OAS3, UGCG, ELP1, PNLIP, TRAPPC9, DMXL1, SHANK1, PLEKHG4, UNC13C, TSHZ3, POTEB2, PCDH17, C9orf72, WASHC4, CACNAlH, NLRP3, CCDCl68, MUC1, RHPN2, MGAT1, ZSCAN1, SSPO, HIST1H2BE, RERE, C2CD6, FYB2, BLNK, TNRC6B, BTN1A1, ZNF236, FAT3, ZNF675, ANKRD52, MGAT5B, MKNK2, SYTL5, STARD9, TIMM21, FRAS1, HIST1H2AE, NACAD, TMEM245, NFIL3, MXRA5, SYT14, TFAM, NBPF12, ARID5B, TUBGCP2, H3F3B, GALNT11, B4GALNT1, ASAP1, SURF4, AKAP2, TUBGCP3, ARRDC1, YTHDF2, HECW1, HIVEP3, TAF3, NELL1, FPR1, ZNF91, GOLGA6C, BCAS1, NFIC, BOC, GRM1, WDPCP, CBLL2, FBN1, BIN1, CFH, ADCY1, DSCAM, GPR149, TMEM150C, ABCB11, ATXN7, NBPF10, SLC6A2, ASPM, WNK1, ARID1A, FOXO3, HDAC9, ATM, PNPLA3, ZBTB38, INPPL1, CACNAlI, CDH10, FUCA1, PFN3, ZDBF2, EBLN1, CASP1, GABRA2, SLITRK1, NOS1, SCUBE2, MLXIPL, ABCB5, FMN2, PGRMC1, RIPOR2, DENND2A, XPO7, STAB2, SNX18, CDT1, KRT6A, MROH2B, ZNF804A, TPR, LAMA2, KRIT1, Clorfl22, ZFP36L2, PCBP1, BIRC8, RBM28, DAG1, AGO3, SEMA3E, RNF43, FLII, EHHADH, SGSM1, SHCBP1L, FAM160B2, FFAR3, Cllorf97, GPR52, VPS35, SPTA1, MUC2, RBMXL1, MUC5B, ADAM9, LIFR, MTA1, TMPRSS4, PCDH19, MYBL2, MICALLI, BMP7, GRIAl, SV2C, TOPAZ1, ZNF827, AHNAK2, USP18, KDM1A, C1QTNF5, FOXP3, MACC1, GRIK4, KAT2B, MACRODI, SOS1, ALKBH8, ANP32B, RFPL4AL1, RTL1, NTF4, DPYSL4, RUFY1, TBCK, MARS2, SLX4, NME4, PRSS27, WFIKKN2, PCDH15, SLITRK5, GPR158, E2F1, ATP10B, ADAM30, ZFP57, DYRKIB, CLIC6, KLHDC7A, CHGB, BAAT, CDH4, RAB37, PKHD1L1, LMTK3, FRG2, RASIPI, ZBED5, GPR50, YAE1D1, ZNF737, SLC35G4, DUSP23, PRR14, DPY19L2, GABRG1, MRPS31, GRK6, PDK3, ABHD16A, PABPC4L, LRRC4C, FOXRED1, KBTBD13, ST7L, PTPRB, LYPLAL1, PILRB, CNRIP1, NRN1, TAS2R50, ANKRD36, ACBD5, GJC2, TPSB2, IGKV3-15, FAM227B, IGLL1, ATP6V1E1, GOLGA8G, CCNC, SYNPR, PRDM9, TTC28, GOLGA8J, TEX38, NUP43, TEX9, TCAF2, UTRN, FOXD2, DCUN1D1, SLC2A4RG, OR2A7, TRIM24, IGSF5, PSMA1, PNKD, GCGR, LRCH1, TNRC18P2, DNM1L, CNKSR1, PEX6, CELF4, IGHMBP2, DUX4L4, CENPI, MYO7B, CSMD1, UAP1L1, CLYBL, GXYLT1, MTMR8, SLC13A1, U2AF1L4, KCNC3, CEP 112, MTREX, PRKCA, DHRS4-AS1, ZMYM4, MAP3K1, ZNF879, C22orf34, FHAD1, CRCP, PRKCB, PGR, ACTR10, ALG6, DCDCl, HYOU1, TUBA1A, DLEU7, ABHD17A, TJP3, FSTL5, DAP, BHMT, LAMA4, EGFR, RUNDC3B, ASZ1, KCP, MATN2, HHIPLI, UBR3, EPB41, C8orf74, CYB5R4, SYDE2, CCNK, PRAMEFI1, FSIP2, DHX29, REEP1, HISTIHIB, MIA2, SLC3A2, IDH1, BRAF, CATB, or any combination thereof.

In some aspects, the antibody or antigen-binding portion thereof does not bind the MHC class II molecule. In some aspects, the antibody or antigen-binding portion thereof does not bind the full length tumor antigen polypeptide. In some aspects, the antibody or antigen-binding portion thereof binds an epitope on the tumor antigen fragment that is surface-exposed when the tumor antigen fragment is associated with the MHC class II molecule. In some aspects, the antibody or antigen-binding portion thereof binds an epitope on the tumor antigen fragment that extends beyond the binding groove of the MH-IC class If molecule.

### IIA. Antibodies

Some aspects of the present disclosure are directed to an antibody or an antigen-binding portion thereof that specifically binds to a fragment of a tumor antigen, wherein the fragment of the tumor antigen can be associated with an MI-IC class II molecule. In some aspects, the tumor antigen is WT1. In some aspects, the fragment comprises the amino acid sequence set forth in SEQ ID NO: 51 or 52.

Some aspects of the present disclosure are directed to an antibody or an antigen-binding portion thereof that specifically binds a peptide fragment of Wilms' tumor protein (WT1), wherein the peptide fragment is capable of being presented by an MHC class II molecule. Any peptide fragment of WT1 that is capable of being presented by an MI-IC class II molecule can be used as a target antigen for the antibody or antigen-binding portion thereof. In some aspects, the peptide fragment comprises at least about 10 to about 20 amino acids, at least about 11 to about 20 amino acids, at least about 12 to about 20 amino acids, at least about 13 to about 20 amino acids, at least about 14 to about 20 amino acids, at least about 15 to about 20 amino acids, at least about 16 to about 20 amino acids, at least about 17 to about 20 amino acids, at least about 11 to about 19 amino acids, at least about 12 to about 19 amino acids, at least about 13 to about 19 amino acids, at least about 14 to about 19 amino acids, at least about 15 to about 19 amino acids, at least about 16 to about 19 amino acids, at least about 17 to about 19 amino acids, at least about 11 to about 18 amino acids, at least about 12 to about 18 amino acids, at least about 13 to about 18 amino acids, at least about 14 to about 18 amino acids, at least about 15 to about 18 amino acids, at least about 16 to about 18 amino acids, at least about 17 to about 18 amino acids, at least about 11 to about 17 amino acids, at least about 12 to about 17 amino acids, at least about 13 to about 17 amino acids, at least about 14 to about 17 amino acids, at least about 15 to about 17 amino acids, or at least about 16 to about 17 amino acids. In some aspects, the peptide fragment is about 10, about 11, about 12, about 13, about 14, about 15, about 16, about 17, about 18, about 19, or about 20 amino acids in length. In some aspects, the peptide fragment is about 12 amino acids in length. In some aspects, the peptide fragment is about 13 amino acids in length. In some aspects, the peptide fragment is about 14 amino acids in length. In some aspects, the peptide fragment is about 15 amino acids in length. In some aspects, the peptide fragment is about 16 amino acids in length. In some aspects, the peptide fragment is about 17 amino acids in length. In some aspects, the peptide fragment comprises FKLSH1LQMH (SEQ ID NO: 51). In some aspects, the peptide fragment comprises WT1₃₃₀₋₃₄₈ (₃₃₀CNKRYFKLSHLQMHSRKHT₃₄₈) (SEQ ID NO: 52).

In some aspects, the antibody or antigen-binding portion thereof does not bind the MHC class II molecule. In some aspects, the antibody or antigen-binding portion thereof does not bind full length WT1. In some aspects, the antibody or antigen-binding portion thereof binds an epitope on the WT1 fragment that is surface-exposed when the WT1 fragment is associated with the MHC class II molecule. In some aspects, the antibody or antigen-binding portion thereof binds an epitope on the WT1 fragment that extends beyond the binding groove of the MHC class II molecule.

In some aspects, the antibody or antigen-binding portion thereof comprises a heavy chain and a light chain, wherein the heavy chain comprises a heavy chain variable region (VH) comprising a VH complementarity determining region (CDR)-1, a VH-CDR2, and a VH-CDR3; and wherein the light chain comprises a light chain variable region (VL) comprising a VL CDR1, a VL-CDR2, and a VL-CDR3; wherein the VH-CDR3 comprises the amino acid sequence set forth in SEQ ID NO: 3, 13, 23, 33, 43, or 53. In some aspects, the VH-CDR2 comprises an amino acid sequence set forth in SEQ ID NO: 2, 12, 22, 32, 42, or 52. In some aspects, the VH-CDR1 comprises an amino acid sequence set forth in SEQ ID NO: 1, 11, 21, 31, 41, or 51. In some aspects, the VL-CDR1 comprises an amino acid sequence set forth in SEQ ID NO: 4, 14, 24, 34, 44, or 54. In some aspects, the VL-CDR2 comprises an amino acid sequence set forth in SEQ ID NO: 5, 15, 25, 35, 45, or 55. In some aspects, the VL-CDR3 comprises an amino acid sequence set forth in SEQ ID NO: 6, 16, 26, 36, 46, or 56.

In some aspects, the antibody or antigen-binding portion thereof comprises a VII-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen-binding portion thereof comprises a VH-CDR1 comprising the amino acid sequence set forth in SEQ ID NO: 11, a VH-CDR2 comprising the amino acid sequence set forth in SEQ ID NO: 12, a VH-CDR3 comprising the amino acid sequence set forth in SEQ ID NO: 13, a VL-CDR1 comprising the amino acid sequence set forth in SEQ ID NO: 14, a VL-CDR2 comprising the amino acid sequence set forth in SEQ ID NO: 15, and a VL-CDR3 comprising the amino acid sequence set forth in SEQ ID NO: 16.

In some aspects, the antibody or antigen-binding portion thereof comprises a VH-CDR1 comprising the amino acid sequence set forth in SEQ ID NO: 21, a VH-CDR2 comprising the amino acid sequence set forth in SEQ ID NO: 22, a VH-CDR3 comprising the amino acid sequence set forth in SEQ ID NO: 23, a VL-CDR1 comprising the amino acid sequence set forth in SEQ ID NO: 24, a VL-CDR2 comprising the amino acid sequence set forth in SEQ ID NO: 25, and a VL-CDR3 comprising the amino acid sequence set forth in SEQ ID NO: 26.

In some aspects, the antibody or antigen-binding portion thereof comprises a VH-CDR1 comprising the amino acid sequence set forth in SEQ ID NO: 31, a VH-CDR2 comprising the amino acid sequence set forth in SEQ ID NO: 32, a VH-CDR3 comprising the amino acid sequence set forth in SEQ ID NO: 33, a VL-CDR1 comprising the amino acid sequence set forth in SEQ ID NO: 34, a VL-CDR2 comprising the amino acid sequence set forth in SEQ ID NO: 35, and a VL-CDR3 comprising the amino acid sequence set forth in SEQ ID NO: 36.

In some aspects, the antibody or antigen-binding portion thereof comprises a VH-CDR1 comprising the amino acid sequence set forth in SEQ ID NO: 41, a VH-CDR2 comprising the amino acid sequence set forth in SEQ ID NO: 42, a VH-CDR3 comprising the amino acid sequence set forth in SEQ ID NO: 43, a VL-CDR1 comprising the amino acid sequence set forth in SEQ ID NO: 44, a VL-CDR2 comprising the amino acid sequence set forth in SEQ ID NO: 45, and a VL-CDR3 comprising the amino acid sequence set forth in SEQ ID NO: 46.

In some aspects, the antibody or antigen-binding portion thereof comprises a VH-CDR1 comprising the amino acid sequence set forth in SEQ ID NO: 51, a VH-CDR2 comprising the amino acid sequence set forth in SEQ ID NO: 52, a VH-CDR3 comprising the amino acid sequence set forth in SEQ ID NO: 53, a VL-CDR1 comprising the amino acid sequence set forth in SEQ ID NO: 54, a VL-CDR2 comprising the amino acid sequence set forth in SEQ ID NO: 55, and a VL-CDR3 comprising the amino acid sequence set forth in SEQ ID NO: 56.

In some aspects, the antibody or antigen-binding portion thereof comprises a VTH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 7, 17, 27, 37, 47, or 57. In some aspects, the VH comprises an amino acid sequence set forth in SEQ HD NOs: 7, 17, 27, 37, 47, or 57.

In some aspects, the antibody or antigen-binding portion thereof comprises a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 8, 18, 28, 38, 48, or 58. In some aspects, the VL comprises an amino acid sequence set forth in SEQ ID NOs: 8, 18, 28, 38, 48, or 58.

In some aspects, the antibody or antigen-binding portion thereof comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 7, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 8. In some aspects, the antibody or antigen-binding portion thereof comprises a VH comprising the amino acid sequence set forth in SEQ ID NO: 7, and a VL comprising the amino acid sequence set forth in SEQ ID NO: 8.

In some aspects, the antibody or antigen-binding portion thereof comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 17, and a VL, comprising an amino acid having at least about 80%, at least about 85%, at least about 90%, at least about 95%, at least about 96%, at least about 97%, at least about 98%, or at least about 99% sequence identity to the sequence set forth in SEQ ID NO: 18. In some aspects, the antibody or antigen-binding portion thereof comprises a VH comprising the amino acid sequence set forth in SEQ ID NO: 17, and a VL comprising the amino acid sequence set forth in SEQ ID NO: 18.

In some aspects, the antibody or antigen-binding portion thereof comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 27, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 28. In some aspects, the antibody or antigen-binding portion thereof comprises a VH comprising the amino acid sequence set forth in SEQ ID NO: 27, and a VL comprising the amino acid sequence set forth in SEQ ID NO: 28.

In some aspects, the antibody or antigen-binding portion thereof comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 37, and a VL. comprising an amino acid having at least about 80%, at least about 85%, at least about 90%, at least about 95%, at least about 96%, at least about 97%, at least about 98%, or at least about 99% sequence identity to the sequence set forth in SEQ ID NO: 38. In some aspects, the antibody or antigen-binding portion thereof comprises a VH comprising the amino acid sequence set forth in SEQ ID NO: 37, and a VL comprising the amino acid sequence set forth in SEQ ID NO: 38.

In some aspects, the antibody or antigen-binding portion thereof comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 47, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 48. In some aspects, the antibody or antigen-binding portion thereof comprises a VH comprising the amino acid sequence set forth in SEQ ID NO: 47, and a VL comprising the amino acid sequence set forth in SEQ ID NO: 48.

In some aspects, the antibody or antigen-binding portion thereof comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 57, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 58. In some aspects, the antibody or antigen-binding portion thereof comprises a VH comprising the amino acid sequence set forth in SEQ ID NO: 57, and a VL comprising the amino acid sequence set forth in SEQ ID NO: 58.

In some aspects, the antibody or antigen-binding portion thereof comprises a heavy chain constant region. In some aspects, the heavy chain constant region is an IgG constant region. In some aspects, the heavy chain constant region is an IgG constant region, IgG2 constant region, IgG3 constant region, IgG4 constant region, or a variant thereof.

### IIB. Chimeric Antigen Receptors and Polynucleotides Encoding the Same

Some aspects of the present disclosure are directed to a polynucleotide encoding a chimeric antigen receptors (CAR), wherein the CAR comprises an antigen-binding domain that specifically binds to a fragment of a tumor antigen, wherein the fragment of the tumor antigen can be associated with an MHC class II molecule. Some aspects of the present disclosure are directed to a CAR comprising an antigen-binding domain that specifically binds to a fragment of a tumor antigen, wherein the fragment of the tumor antigen can be associated with an MHC class II molecule. In some aspects, the tumor antigen is WT1. In some aspects, the fragment comprises the amino acid sequence set forth in SEQ ID NO: 51 or 52.

In some aspects, the antigen-binding domain of the CAR does not bind the MHC class II molecule. In some aspects, the antigen-binding domain does not bind full length WT1. In some aspects, the antigen-binding domain binds an epitope on the WTT fragment that is surface-exposed when the WT1 fragment is associated with the MHC class II molecule. In some aspects, antigen-binding domain binds an epitope on the WT1 fragment that extends beyond the binding groove of the MHC class II molecule.

In some aspects, the antigen-binding domain comprises a heavy chain and a light chain, wherein the heavy chain comprises a heavy chain variable region (V-H) comprising a VH complementarity determining region (CDR)-1, a VH-CDR2, and a VH-CDR3; and wherein the light chain comprises a light chain variable region (VL) comprising a VL CDR1, a ML-CDR2, and a VL-CDR3; wherein the VH-CDR3 comprises the amino acid sequence set forth in SEQ ID NO: 3, 13, 23, 33, 43, or 53. In some aspects, the VH-CDR2 comprises an amino acid sequence set forth in SEQ ID NO: 2, 12, 22, 32, 42, or 52. In some aspects, the VH-CDR1 comprises an amino acid sequence set forth in SEQ ID NO: 1, 11, 21, 31, 41, or 51. In some aspects, the VL-CDR1 comprises an amino acid sequence set forth in SEQ ID NO: 4, 14, 24, 34, 44, or 54. In some aspects, the VL-CDR2 comprises an amino acid sequence set forth in SEQ ID NO: 5, 15, 25, 35, 45, or 55. In some aspects, the VL-CDR3 comprises an amino acid sequence set forth in SEQ ID NO: 6, 16, 26, 36, 46, or 56.

In some aspects, the antigen-binding domain comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antigen-binding domain comprises a VH-CDR1 comprising the amino acid sequence set forth in SEQ ID NO: 11, a VH-CDR2 comprising the amino acid sequence set forth in SEQ ID NO: 12, a VH-CDR3 comprising the amino acid sequence set forth in SEQ ID NO: 13, a VL-CDR1 comprising the amino acid sequence set forth in SEQ ID NO: 14, a VL-CDR2 comprising the amino acid sequence set forth in SEQ ID NO: 15, and a VL-CDR3 comprising the amino acid sequence set forth in SEQ ID NO: 16.

In some aspects, the antibody or antigen-binding portion thereof comprises a VH-CDR1 comprising the amino acid sequence set forth in SEQ ID NO: 21, a VH-CDR2 comprising the amino acid sequence set forth in SEQ ID NO: 22, a VH-CDR3 comprising the amino acid sequence set forth in SEQ ID NO: 23, a VL-CDR1 comprising the amino acid sequence set forth in SEQ ID NO: 24, a VL-CDR2 comprising the amino acid sequence set forth in SEQ ID NO: 25, and a VL-CDR3 comprising the amino acid sequence set forth in SEQ ID NO: 26.

In some aspects, the antigen-binding domain comprises a VH-CDR1 comprising the amino acid sequence set forth in SEQ ID NO: 31, a VH-CDR2 comprising the amino acid sequence set forth in SEQ ID NO: 32, a VH-CDR3 comprising the amino acid sequence set forth in SEQ ID NO: 33, a VL-CDR1 comprising the amino acid sequence set forth in SEQ ID NO: 34, a VL-CDR2 comprising the amino acid sequence set forth in SEQ ID NO: 35, and a VL-CDR3 comprising the amino acid sequence set forth in SEQ ID NO: 36.

In some aspects, the antigen-binding domain comprises a VH-CDR1 comprising the amino acid sequence set forth in SEQ ID NO: 41, a VH-CDR2 comprising the amino acid sequence set forth in SEQ ID NO: 42, a VH-CDR3 comprising the amino acid sequence set forth in SEQ ID NO: 43, a VL-CDR1 comprising the amino acid sequence set forth in SEQ ID NO: 44, a VL-CDR2 comprising the amino acid sequence set forth in SEQ ID NO: 45, and a VL-CDR3 comprising the amino acid sequence set forth in SEQ ID NO: 46.

In some aspects, the antigen-binding domain comprises a VH-CDR1 comprising the amino acid sequence set forth in SEQ ID NO: 51, a VH-CDR2 comprising the amino acid sequence set forth in SEQ ID NO: 52, a VH-CDR3 comprising the amino acid sequence set forth in SEQ ID NO: 53, a VL-CDR1 comprising the amino acid sequence set forth in SEQ ID NO: 54, a VL-CDR2 comprising the amino acid sequence set forth in SEQ ID NO: 55, and a VL-CDR3 comprising the amino acid sequence set forth in SEQ ID NO: 56.

In some aspects, the antigen-binding domain thereof comprises a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 7, 17, 27, 37, 47, or 57. In some aspects, the VH comprises an amino acid sequence set forth in SEQ ID NOs: 7, 17, 27, 37, 47, or 57.

In some aspects, the antigen-binding domain comprises a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 8, 18, 28, 38, 48, or 58. In some aspects, the VL comprises an amino acid sequence set forth in SEQ ID NOs: 8, 18, 28, 38, 48, or 58.

In some aspects, the antigen-binding domain comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 7, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 8. In some aspects, the antigen-binding domain comprises a VH comprising the amino acid sequence set forth in SEQ ID NO: 7, and a VL comprising the amino acid sequence set forth in SEQ ID NO: 8.

In some aspects, the antigen-binding domain comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 17, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 18. In some aspects, the antigen-binding domain comprises a VH comprising the amino acid sequence set forth in SEQ ID NO: 17, and a VL comprising the amino acid sequence set forth in SEQ ID NO: 18.

In some aspects, the antigen-binding domain comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 27, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 28. In some aspects, the antigen-binding domain thereof comprises a VH comprising the amino acid sequence set forth in SEQ ID NO: 27, and a VL comprising the amino acid sequence set forth in SEQ ID NO: 28.

In some aspects, the antigen-binding domain comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 37, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 38. In some aspects, the antigen-binding domain thereof comprises a VH comprising the amino acid sequence set forth in SEQ ID NO: 37, and a VL comprising the amino acid sequence set forth in SEQ ID NO: 38.

In some aspects, the antigen-binding domain comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 47, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 48. In some aspects, the antigen-binding domain comprises a VTH comprising the amino acid sequence set forth in SEQ ID NO: 47, and a VL comprising the amino acid sequence set forth in SEQ ID NO: 48.

In some aspects, the antigen-binding domain thereof comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 57, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 58. In some aspects, the antigen-binding domain thereof comprises a VH comprising the amino acid sequence set forth in SEQ ID NO: 57, and a VL comprising the amino acid sequence set forth in SEQ ID NO: 58.

In some aspects, the CAR comprises (i) an antigen binding domain comprising a VEH-CDR1 comprising the amino acid sequence set forth in SEQ ID NO: 1, a VEH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a transmembrane domain; and (iii) an intracellular signaling domain.

In some aspects, the CAR comprises (i) an antigen binding domain comprising a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a CD8α transmembrane domain or a CD28 transmembrane domain; and (iii) an intracellular signaling domain.

In some aspects, the CAR comprises (i) an antigen binding domain comprising a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a transmembrane domain; and (iii) an intracellular signaling domain comprising a CD28 costimulatory region or a 4-1BB costimulatory region and a CD3-zeta signaling domain.

In some aspects, the CAR comprises (i) an antigen binding domain comprising a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a CD8α transmembrane domain or a CD28 transmembrane domain; and (iii) an intracellular signaling domain comprising a CD28 costimulatory region or a 4-1BB costimulatory region and a CD3-zeta signaling domain.

In some aspects, the CAR comprises (i) an antigen binding domain comprising a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a CD8α transmembrane domain; and (iii) an intracellular signaling domain comprising a CD28 costimulatory region and a CD3-zeta signaling domain.

In some aspects, the CAR comprises (i) an antigen binding domain comprising a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a CD8x transmembrane domain; and (iii) an intracellular signaling domain comprising a 4-1BB costimulatory region and a CD3-zeta signaling domain.

In some aspects, the CAR comprises (i) an antigen binding domain comprising a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a CD28 transmembrane domain; and (iii) an intracellular signaling domain comprising a CD28 costimulatory region and a CD3-zeta signaling domain.

In some aspects, the CAR comprises (i) an antigen binding domain comprising a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6; (ii) a CD28 transmembrane domain; and (iii) an intracellular signaling domain comprising a 4-1IBB costimulatory region and a CD3-zeta signaling domain.

In some aspects, the CAR further comprises a hinge region (sometimes referred to as a spacer region) between the antigen binding domain and the transmembrane domain. In some aspects, the hinge region comprises an IgG hinge region. In some aspects, the hinge region is an IgG1, IgG2, IgG3, or IgG4 hinge region or a portion thereof.

### II.C. Multispecific Antibodies

Some aspects of the present disclosure are directed to multispecific antibodies that comprise a first antigen-binding domain and a second antigen-binding domain, wherein the first antigen-binding domain, the second antigen-binding domain, or both specifically binds to a fragment of a tumor antigen, wherein the fragment of the tumor antigen can be associated with an MHC class II molecule.

In some aspects, the multispecific antibody is a bispecific antibody, wherein the first antigen-binding domain specifically binds a fragment of a tumor antigen, wherein the fragment of the tumor antigen can be associated with an MI-IC class II molecule. In some aspects, the second antigen-binding domain specifically binds an antigen expressed on the surface of an immune cell, e.g., a T cell, an NK cell, or a tumor infiltrating lymphocyte (TIL). In some aspects, the second antigen-binding domain specifically binds CD3 receptor. In some aspects, the bispecific antibody is a bi-specific T cell engager (BiTE) comprising (i) a first antigen-binding domain that specifically binds CD3 receptor and (ii) a second antigen-binding domain that specifically binds a peptide fragment of a tumor antigen, wherein the fragment of the tumor antigen can be associated with an MHC class II molecule. In some aspects, the second antigen-binding domain specifically binds a peptide fragment of WT1. In some aspects, the second antigen-binding domain comprises an antigen-binding portion of an antibody disclosed herein.

In some aspects, the multispecific antibody is a trispecific antibody, comprising (i) a first antigen-binding domain that specifically binds CD3 receptor; (ii) a second antigen-binding domain that specifically binds a fragment of a tumor antigen, wherein the fragment of the tumor antigen can be associated with an MHC class II molecule; and (iii) a third antigen-binding domain. In some aspects, the second antigen-binding domain specifically binds a peptide fragment of WT1. In some aspects, the second antigen-binding domain comprises an antigen-binding portion of an antibody disclosed herein.

In some aspects, the multispecific antibody further comprises a fourth antigen-binding domain. In some aspects, the multispecific antibody further comprises a fifth antigen-binding domain.

In some aspects, the tumor antigen is WT1. In some aspects, the fragment comprises the amino acid sequence set forth in SEQ ID NO: 51 or 52. In some aspects, the multispecific antibody further comprises a third antigen-binding domain.

In some aspects, the multispecific antibody comprises at least one antigen-binding domain that specifically binds a peptide fragment of WT1, wherein the peptide fragment of the WT1 can be associated with an MHC class II molecule. In some aspects, the multispecific antibody, e.g., bispecific antibody, BiTE, and TriTE, comprises a VH-CDR1, a VH-CDR2, a VH-CDR3, a VL-CDR1, a VL-CDR2, and a VL-CDR3; wherein the VH-CDR3 comprises the amino acid sequence set forth in SEQ ID NO: 3, 13, 23, 33, 43, or 53. In some aspects, the VH-CDR2 comprises an amino acid sequence set forth in SEQ ID NO: 2, 12, 22, 32, 42, or 52. In some aspects, the VH-CDR1 comprises an amino acid sequence set forth in SEQ ID NO: 1, 11, 21, 31, 41, or 51. In some aspects, the VL-CDR1 comprises an amino acid sequence set forth in SEQ ID NO: 4, 14, 24, 34, 44, or 54. In some aspects, the VL-CDR2 comprises an amino acid sequence set forth in SEQ ID NO: 5, 15, 25, 35, 45, or 55. In some aspects, the VL-CDR3 comprises an amino acid sequence set forth in SEQ ID NO: 6, 16, 26, 36, 46, or 56.

In some aspects, the multispecific antibody, e.g., bispecific antibody, BiTE, or TriTE, comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the multispecific antibody, e.g., bispecific antibody, BiTE, or TriTE, comprises a VH-CDR1 comprising the amino acid sequence set forth in SEQ ID NO: 11, a VH-CDR2 comprising the amino acid sequence set forth in SEQ ID NO: 12, a VH-CDR3 comprising the amino acid sequence set forth in SEQ ID NO: 13, a VL-CDR1 comprising the amino acid sequence set forth in SEQ ID NO: 14, a VL-CDR2 comprising the amino acid sequence set forth in SEQ ID NO: 15, and a VL-CDR3 comprising the amino acid sequence set forth in SEQ ID NO: 16.

In some aspects, the multispecific antibody, e.g., bispecific antibody, BiTE, or TriTE, comprises a VH-CDR1 comprising the amino acid sequence set forth in SEQ ID NO: 21, a VH-CDR2 comprising the amino acid sequence set forth in SEQ ID NO: 22, a VH-CDR3 comprising the amino acid sequence set forth in SEQ ID NO: 23, a VL-CDR1 comprising the amino acid sequence set forth in SEQ ID NO: 24, a VL-CDR2 comprising the amino acid sequence set forth in SEQ ID NO: 25, and a VL-CDR3 comprising the amino acid sequence set forth in SEQ ID NO: 26.

In some aspects, the multispecific antibody, e.g., bispecific antibody, BiTE, or TriTE, comprises a VH-CDR1 comprising the amino acid sequence set forth in SEQ ID NO: 31, a VH-CDR2 comprising the amino acid sequence set forth in SEQ ID NO: 32, a VH-CDR3 comprising the amino acid sequence set forth in SEQ ID NO: 33, a VL-CDR1 comprising the amino acid sequence set forth in SEQ ID NO: 34, a VL-CDR2 comprising the amino acid sequence set forth in SEQ ID NO: 35, and a VL-CDR3 comprising the amino acid sequence set forth in SEQ ID NO: 36.

In some aspects, the multispecific antibody, e.g., bispecific antibody, BiTE, or TriTE, comprises a VH-CDR1 comprising the amino acid sequence set forth in SEQ ID NO: 41, a VH-CDR2 comprising the amino acid sequence set forth in SEQ ID NO: 42, a VH-CDR3 comprising the amino acid sequence set forth in SEQ ID NO: 43, a VL-CDR1 comprising the amino acid sequence set forth in SEQ ID NO: 44, a VL-CDR2 comprising the amino acid sequence set forth in SEQ ID NO: 45, and a VL-CDR3 comprising the amino acid sequence set forth in SEQ ID NO: 46.

In some aspects, the multispecific antibody, e.g., bispecific antibody, BiTE, or TriTE, comprises a VH-CDR1 comprising the amino acid sequence set forth in SEQ ID NO: 51, a VH-CDR2 comprising the amino acid sequence set forth in SEQ ID NO: 52, a VH-CDR3 comprising the amino acid sequence set forth in SEQ ID NO: 53, a VL-CDR1 comprising the amino acid sequence set forth in SEQ ID NO: 54, a VL-CDR2 comprising the amino acid sequence set forth in SEQ ID NO: 55, and a VL-CDR3 comprising the amino acid sequence set forth in SEQ ID NO: 56.

In some aspects, the multispecific antibody, e.g., bispecific antibody, BiTE, or TriTE, comprises a VII comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 7, 17, 27, 37, 47, or 57. In some aspects, the V-H comprises an amino acid sequence set forth in SEQ ID NOs: 7, 17, 27, 37, 47, or 57.

In some aspects, the multispecific antibody, e.g., bispecific antibody, BiTE, or TriTE, comprises a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 8, 18, 28, 38, 48, or 58. In some aspects, the VL comprises an amino acid sequence set forth in SEQ ID NOs: 8, 18, 28, 38, 48, or 58.

In some aspects, the multispecific antibody, e.g., bispecific antibody, BiTE, or TriTE, comprises a V-H comprising an amino acid having at least about 80%, at least about 85%, at least about 90%, at least about 95%, at least about 96%, at least about 97%, at least about 98%, or at least about 99% sequence identity to the sequence set forth in SEQ ID NO: 7, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 8. In some aspects, the antigen-binding domain comprises a VH comprising the amino acid sequence set forth in SEQ ID NO: 7, and a VL comprising the amino acid sequence set forth in SEQ ID NO: 8.

In some aspects, the multispecific antibody, e.g., bispecific antibody, BiTE, or TriTE, comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 17, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 18. In some aspects, the antigen-binding domain comprises a VH comprising the amino acid sequence set forth in SEQ ID NO: 17, and a VL comprising the amino acid sequence set forth in SEQ ID NO: 18.

In some aspects, the multispecific antibody, e.g., bispecific antibody, BiTE, or TriTE, comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 27, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 28. In some aspects, the antigen-binding domain thereof comprises a VH comprising the amino acid sequence set forth in SEQ ID NO: 27, and a VL comprising the amino acid sequence set forth in SEQ ID NO: 28.

In some aspects, the multispecific antibody, e.g., bispecific antibody, BiTE, or TriTE, comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 37, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 38. In some aspects, the antigen-binding domain thereof comprises a VH comprising the amino acid sequence set forth in SEQ ID NO: 37, and a VL comprising the amino acid sequence set forth in SEQ ID NO: 38.

In some aspects, the multispecific antibody, e.g., bispecific antibody, BiTE, or TriTE, comprises a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 47, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 48. In some aspects, the antigen-binding domain comprises a VH comprising the amino acid sequence set forth in SEQ ID NO: 47, and a VL comprising the amino acid sequence set forth in SEQ ID NO: 48.

In some aspects, the multispecific antibody, e.g., bispecific antibody, BiTE, or TriTE, comprises a VH comprising an amino acid having at least about 80%, at least about 85%, at least about 90%, at least about 95%, at least about 96%, at least about 97%, at least about 98%, or at least about 99% sequence identity to the sequence set forth in SEQ ID NO: 57, and a VL comprising an amino acid having at least about 80%, at least about 85%, at least about 90%, at least about 95%, at least about 96%, at least about 97%, at least about 98%, or at least about 99% sequence identity to the sequence set forth in SEQ ID NO: 58. In some aspects, the antigen-binding domain thereof comprises a VH comprising the amino acid sequence set forth in SEQ ID NO: 57, and a VL comprising the amino acid sequence set forth in SEQ ID NO: 58.

### II.D. HLA Class II Molecules

As described herein, the antibodies or antigen-binding portions thereof specifically recognize a peptide fragment of tumor antigen that is presented by an MHC class II molecule. As such, the peptide fragment can be presented by any MI-IC class II molecule. In some aspects, the peptide fragment is presented by an MHC Class 11 molecule expressed on the surface of an antigen presenting cell. In some aspects, the peptide fragment is presented by an MHC Class II molecule expressed on the surface of a tumor cell.

In some aspects, the HLA Class II molecule is an HLA-DR, HLA-DP, or an HLA-DQ allele. In some aspects, the HLA class II molecule is any HLA allele disclosed at hla.alleles.org/(last visited on Mar. 24, 2022)

In some aspects, the HLA Class II molecule comprises an alpha chain and a beta chain. In some aspects, the sequence of the alpha chain is selected from any of the HLA alpha chain protein sequences available at hla.alleles.org (last visited Mar. 24, 2022).

### II.D.1. HLA-DP Class II Molecules

In some aspects, the MHC class II molecule comprises a DP beta chain and a DP alpha chain. In some aspects, the beta chain of the MHC class II molecule is a DP1, DP2, DP3, DP4, DP5, DP6, DP8, or DP9 allele.

In some aspects, the alpha chain is an HLA-DP alpha chain. Any HLA-DP alpha chain allele known in the art can be used in the compositions and methods disclosed herein. In some aspects, the alpha chain is selected from an HLA-DPA1*01, HLA-DPA1*02, HLA-DPA1*03, and HLA-DPA1*04 allele. In certain aspects, the DP alpha chain comprises an HLA-DPA1*01 allele. In certain aspects, the DP alpha chain comprises an HLA-DPA1*02 allele. In certain aspects, the DP alpha chain comprises an HLA-DPA1*03 allele. In certain aspects, the DP alpha chain comprises an HLA-DPA1*04 allele.

In certain aspects, the DP alpha chain is selected from DPA1*01:03:01:01, DPA1*01:03:01:02, DPA1*01:03:01:03, DPA1*01:03:01:04, DPA1*01:03:01:05, DPA1*01:03:01:06, DPA1*01:03:01:07, DPA1*01:03:01:08, DPA1*01:03:01:09, DPA1*01:03:01:10, DPA1*01:03:01:11, DPA1*01:03:01:12, DPA1*01:03:01:13, DPA1*01:03:01:14, DPA1*01:03:01:15, DPA1*01:03:01:16, DPA1*01:03:01:17, DPA1*01:03:01:18Q. DPA1*01:03:01:19, DPA1*01:03:01:20, DPA1*01:03:01:21, DPA1*01:03:01:22, DPA1*01:03:01:23, DPA1*01:03:02, DPA1*01:03:03, DPA1*01:03:04, DPA1*01:03:05, DPA1*01:03:06, DPA*01:03:07, DPA1*01:03:08, DPA1*01:03:09, DPA1*01:04, DPA1*01:05, DPA1*01:06:01, DPA1*01:06:02, DPA1*01:07, DPA1*01:08, DPA1*01:09, DPA1*01:10, DPA1*01:11, DPA*01:12, DPA*01:13, DPA1*01:14, DPA1*01:15, DPA1*01:16, DPA1*01:17, DPA1*01:18, DPA1*01:19, DPA1*02:01:01:01, DPA1*02:01:01:02, DPA1*02:01:01:03, DPA1*02:01:01:04, DPA1*02:01:01:05, DPA1*02:01:01:06, DPA1*02:01:01:07, DPA1*02:01:01:08, DPA1*02:01:01:09, DPA1*02:01:01:10, DPA1*02:01:01:11, DPA1*02:01:02:01, DPA 1*02:01:02:02, DPA1*02:01:03, DPA1*02:01:04, DPA1*02:01:05, DPA1*02:01:06, DPA1*02:01:07, DPA1*02:01:08:01, DPA1*02:01:08:02, DPA1*02:02:02:01, DPA1*02:02:02:02, DPA1*02:02:02:03, DPA1*02:02:02:04, DPA1*02:02:02:05, DPA1*02:02:03, DPA1*02:02:04, DPA1*02:02:05, DPA1*02:02:06, DPA1*02:03, DPA1*02:04, DPA1*02:05, DPA1*02:06, DPA1*02:07:01:01, DPA1*02:07:01:02, DPA1*02:07:01:03, DPA1*02:08, DPA1*02:09, DPA1*02:10, DPA1*02:11, DPA1*02:12, DPA1*02:13N, DPA1*02:14, DPA1*02:15, DPA1*02:16, DPA1*03:01:01:01, DPA1*03:01:01:02, DPA1*03:01:01:03, DPA1*03:01:01:04, DPA1*03:01:01:05, DPA1*03:01:02, DPA1*03:02, DPA1*03:03, DPA1*03:04, DPA1*04:01:01:01, DPA1*04:01:01:02, DPA1*04:01:01:03, DPA1*04:02, or any combination thereof.

In some aspects, the beta chain is an HLA-DP beta chain. Any HLA-DP beta chain allele known in the art can be used in the compositions and methods disclosed herein. In certain aspects, the DP beta chain comprises an allele selected from an DPB1*01, DPB1*02, DPB1*03, DPB1*04, DPB1*05, DPB1*06, DPB1*08, DPB1*09, DPB1*10, DPB1*100, DPB1*101, DPB1*102, DPB1*103, DPB1*104, DPB1*105, DPB1*106, DPB1*107, DPB1*108, DPB1*109, DPB1*11, DPB1*110, DPB1*111, DPB1*112, DPB1*113, DPB1*114, DPB1*115, DPB1*116, DPB1*117, DPB1*118, DPB1*119, DPB1*120, DPB1*121, DPB1*122, DPB1*123, DPB1*124, DPB1*125, DPB1*126, DPB1*127, DPB1*128, DPB1*129, DPB1*13, DPB1*130, DPB1*131, DPB1*132, DPB1*133, DPB1*134, DPB1*135, DPB1*136, DPB1*137, DPB1*138, DPB1*139, DPB1*14, DPB1*140, DPB1*141, DPB1*142, DPB1*143, DPB1*144, DPB1*145, DPB1*146, DPB1*147, DPB1*148, DPB1*149, DPB1*15, DPB1*150, DPB1*151, DPB1*152, DPB1*153, DPB1*154, DPB1*155, DPB1*156, DPB1*157, DPB1*158, DPB1*159, DPB1*16, DPB1*160, DPB1*161, DPB1*162, DPB1*163, DPB1*164, DPB1*165. DPB1*166, DPB1*167, DPB1*168, DPB1*169, DPB1*17, DPB1*170, DPB1*171, DPB1*172, DPB1*173, DPB1*174, DPB1*175, DPB1*176, DPB1*177, DPB1*178, DPB1*179, DPB1*18, DPB1*180, DPB1*181, DPB1*182, DPB1*183, DPB1*184, DPB1*185, DPB1*186, DPB1*187, DPB1*188, DPB1*189, DPB1*19, DPB1*190, DPB1*191, DPB1*192, DPB1*193, DPB1*194, DPB1*195, DPB1*196, DPB1*197. DPB1*198, DPB1*199, DPB1*20, DPB1*200, DPB1*201, DPB1*202, DPB1*203, DPB 1*204, DPB1*205, DPB1*206, DPB1*207, DPB1*208, DPB1*209, DPB1*21, DPB1*210, DPB1*211, DPB1*212, DPB1*213, DPB1*214, DPB1*215, DPB1*216, DPB1*217, DPB1*218, DPB1*219, DPB1*22, DPB1*220, DPB1*221, DPB1*222, DPB1*223, DPB1*224, DPB1*225, DPB1*226, DPB1*227, DPB1*228, DPB1*229 DPB1*23, DPB1*230, DPB1*231, DPB1*232, DPB1*233, DPB1*234, DPB1*235, DPB1*236, DPB1*237, DPB1*238, DPB1*239, DPB1*24, DPB1*240, DPB1*241, DPB1*242, DPB1*243, DPB1*244, DPB1*245, DPB1*246, DPB1*247, DPB1*248, DPB1*249, DPB1*25, DPB1*250, DPB1*251, DPB1*252, DPB1*253, DPB1*254, DPB1*255, DPB1*256, DPB1*257, DPB1*258, DPB1*259, DPB1*26, DPB1*260 DPB1*261, DPB1*262, DPB1*263, DPB1*264, DPB1*265, DPB1*266, DPB1*267, DPB1*268, DPB1*269, DPB1*27, DPB1*270, DPB1*271, DPB1*272, DPB1*273 DPB1*274, DPB1*275, DPB1*276, DPB1*277, DPB1*278, DPB1*279, DPB1*28, DPB1*280, DPB1*281, DPB1*282, DPB1*283, DPB1*284, DPB1*285, DPB1*286 DPB1*287, DPB1*288, DPB1*289, DPB1*29, DPB1*290, DPB1*291, DPB1*292, DPB1*293, DPB1*294, DPB1*295, DPB1*296, DPB1*297, DPB1*298, DPB1*299, DPB1*30, DPB1*300, DPB1*301, DPB1*302, DPB1*303, DPB1*304, DPB1*305, DPB1*306, DPB1*307, DPB1*308, DPB1*309, DPB1*31, DPB1*310, DPB1*311, DPB1*312, DPB1*313, DPB1*314, DPB1*315, DPB1*316, DPB1*317, DPB1*318, DPB1*319, DPB1*32, DPB1*320, DPB1*321, DPB1*322, DPB1*323, DPB1*324, DPB1*325, DPB1*326, DPB1*327, DPB1*328, DPB1*329, DPB1*33, DPB1*330, DPB1*331, DPB1*332, DPB1*333, DPB1*334, DPB1*335, DPB1*336, DPB1*337, DPB1*338, DPB1*339, DPB1*34, DPB1*340, DPB1*341, DPB1*342, DPB1*343, DPB1*344, DPB1*345, DPB1*346, DPB1*347, DPB1*348, DPB1*349, DPB1*35, DPB1*350, DPB1*351, DPB1*352, DPB1*353, DPB1*354, DPB1*355, DPB1*356, DPB1*357, DPB1*358, DPB1*359, DPB1*36, DPB1*360, DPB1*361, DPB1*362, DPB1*363, DPB1*364, DPB1*365, DPB1*366, DPB1*367, DPB1*368, DPB1*369, DPB1*37, DPB1*370, DPB1*371, DPB1*372, DPB1*373, DPB1*374, DPB1*375, DPB1*376, DPB1*377, DPB1*378, DPB1*379, DPB1*38, DPB1*380, DPB1*381, DPB1*382, DPB1*383, DPB1*384, DPB1*385, DPB1*386, DPB1*387, DPB1*388, DPB1*389, DPB1*39, DPB1*390, DPB1*391, DPB1*392, DPB1*393, DPB1*394, DPB1*395, DPB1*396, DPB1*397, DPB1*398, DPB1*399, DPB1*40, DPB1*400. DPB1*401, DPB1*402, DPB1*403, DPB1*404, DPB1*405, DPB1*406, DPB1*407, DPB1*408, DPB1*409, DPB1*41, DPB1*410, DPB1*411, DPB1*412, DPB1*413, DPB1*414, DPB1*415, DPB1*416, DPB1*417, DPB1*418, DPB1*419, DPB1*420, DPB1*421, DPB1*422, DPB1*423, DPB1*424, DPB1*425, DPB1*426, DPB1*427. DPB1*428, DPB1*429, DPB1*430, DPB1*431, DPB1*432, DPB1*433, DPB1*434, DPB1*435, DPB1*436, DPB1*437, DPB1*438, DPB1*439, DPB1*44, DPB1*440, DPB1*441, DPB1*442, DPB1*443, DPB1*444, DPB1*445, DPB1*446, DPB1*447, DPB1*448, DPB1*449, DPB1*45, DPB1*450, DPB1*451, DPB1*452, DPB1*453, DPB1*454, DPB1*455, DPB1*456, DPB1*457, DPB1*458, DPB1*459, DPB1*46, DPB1*460, DPB1*461, DPB1*462, DPB1*463, DPB1*464, DPB1*465, DPB1*466, DPB 1*467, DPB1*468, DPB1*469, DPB1*47, DPB1*470, DPB1*471, DPB1*472, DPB1*473, DPB1*474, DPB1*475, DPB1*476, DPB1*477, DPB 1*478, DPB1*479 DPB1*48, DPB1*480, DPB1*481, DPB1*482, DPB1*483, DPB1*484, DPB1*485, DPB1*486, DPB1*487, DPB1*488, DPB1*489, DPB1*49, DPB1*490, DPB1*491 DPB1*492, DPB1*493, DPB1*494, DPB1*495, DPB1*496, DPB1*497, DPB1*498, DPB1*499, DPB1*50, DPB1*500, DPB1*501, DPB1*502, DPB1*503, DPB1*504, DPB1*505, DPB1*506, DPB1*507, DPB1*508, DPB1*509, DPB1*51, DPB1*510, DPB1*511, DPB1*512, DPB1*513, DPB1*514, DPB1*515, DPB1*516, DPB1*517, DPB1*518, DPB1*519, DPB1*52, DPB1*520, DPB1*521, DPB1*522, DPB1*523, DPB1*524, DPB1*525, DPB1*526, DPB1*527, DPB1*528, DPB1*529, DPB1*53, DPB1*530, DPB1*531, DPB1*532, DPB1*533, DPB1*534, DPB1*535, DPB1*536, DPB1*537, DPB1*538, DPB1*539, DPB1*54, DPB1*540, DPB1*541, DPB1*542, DPB1*543, DPB1*544, DPB1*545, DPB1*546, DPB1*547, DPB1*548, DPB1*549 DPB1*55, DPB1*550, DPB1*551, DPB1*552, DPB1*553, DPB1*554, DPB1*555, DPB1*556, DPB1*557, DPB1*558, DPB1*559, DPB1*56, DPB1*560, DPB1*561, DPB1*562, DPB1*563, DPB1*564, DPB1*565, DPB1*566, DPB1*567, DPB1*568, DPB1*569, DPB1*57, DPB1*570, DPB1*571, DPB1*572, DPB1*573, DPB1*574, DPB1*575, DPB1*576, DPB1*577, DPB1*578, DPB1*579, DPB1*58, DPB1*580, DPB1*581, DPB1*582, DPB1*583, DPB1*584, DPB1*585, DPB1*586, DPB1*587, DPB1*588, DPB1*589, DPB1*59, DPB1*590, DPB1*591, DPB1*592, DPB1*593, DPB1*594, DPB1*595, DPB1*596, DPB1*597, DPB1*598, DPB1*599, DPB1*60, DPB1*600, DPB1*601, DPB1*602, DPB1*603, DPB1*604, DPB1*605, DPB1*606. DPB1*607, DPB1*608, DPB1*609, DPB1*61, DPB1*610, DPB1*611, DPB1*612, DPB1*613, DPB1*614, DPB1*615, DPB1*616, DPB1*617, DPB1*618, DPB1*619, DPB1*62, DPB1*620, DPB1*621, DPB1*622, DPB1*623, DPB1*624, DPB1*625, DPB1*626, DPB1*627, DPB1*628, DPB1*629, DPB1*63, DPB1*630, DPB1*631, DPB1*632, DPB1*633, DPB1*634, DPB1*635, DPB1*636, DPB1*637, DPB1*638, DPB1*639, DPB1*64, DPB1*640, DPB1*641, DPB1*642, DPB1*643, DPB1*644, DPB1*645, DPB1*646, DPB1*647, DPB1*648, DPB1*649, DPB1*65, DPB1*650, DPB1*651, DPB1*652, DPB1*653, DPB1*654, DPB1*655, DPB1*656, DPB1*657, DPB1*658, DPB1*659, DPB1*66, DPB1*660, DPB1*661, DPB1*662, DPB1*663, DPB1*664, DPB1*665, DPB1*666, DPB1*667, DPB1*668, DPB1*669, DPB1*67, DPB1*670, DPB1*671, DPB1*672, DPB1*673, DPB1*674, DPB1*675, DPB1*676, DPB1*677, DPB1*678, DPB1*679, DPB1*68, DPB1*680, DPB1*681, DPB1*682, DPB1*683, DPB1*684, DPB1*685, DPB1*686, DPB1*687, DPB1*688, DPB1*689, DPB1*69, DPB1*690, DPB1*691, DPB1*692, DPB1*693, DPB1*694, DPB1*695 DPB1*696, DPB1*697, DPB1*698, DPB1*699, DPB1*70, DPB1*700, DPB1*701, DPB1*702, DPB1*703, DPB1*704, DPB1*705, DPB1*706, DPB1*707, DPB1*708, DPB1*709, DPB1*71, DPB1*710, DPB1*711, DPB1*712, DPB1*713, DPB1*714, DPB1*715, DPB1*716, DPB1*717, DPB1*718, DPB1*719, DPB1*72, DPB1*720, DPB1*721, DPB1*722, DPB1*723, DPB1*724, DPB1*725, DPB1*726, DPB1*727, DPB1*728, DPB1*729, DPB1*73, DPB1*730, DPB1*731, DPB1*732, DPB1*733, DPB1*734, DPB1*735, DPB1*736, DPB1*737, DPB1*738, DPB1*739, DPB1*74, DPB1*740, DPB1*741, DPB1*742, DPB1*743, DPB1*744, DPB1*745, DPB1*746, DPB1*747, DPB1*748, DPB1*749, DPB1*75, DPB1*750, DPB1*751, DPB1*752, DPB1*753, DPB1*754, DPB1*755, DPB1*756, DPB1*757, DPB1*758, DPB1*759, DPB1*76, DPB1*760, DPB1*761, DPB1*762, DPB1*763, DPB1*764, DPB1*765, DPB1*766, DPB1*767, DPB1*768, DPB1*769, DPB1*77, DPB1*770, DPB1*771, DPB11*772, DPB1*773, DPB1*774, DPB1*775, DPB1*776, DPB1*777, DPB1*778, DPB1*779, DPB1*78, DPB1*780, DPB1*781, DPB1*782, DPB1*783, DPB1*784, DPB1*785, DPB1*786, DPB1*787, DPB1*788, DPB1*789, DPB1*79, DPB1*790, DPB1*791, DPB1*792, DPB1*794, DPB1*795, DPB1*796, DPB1*797, DPB1*798, DPB1*799, DPB1*80, DPB1*800, DPB1*801, DPB1*802, DPB1*803, DPB1*804, DPB1*805, DPB1*806, DPB1*807, DPB1*808, DPB1*809, DPB1*81, DPB1*810, DPB1*811, DPB1*812, DPB1*813, DPB1*814, DPB1*815, DPB1*816, DPB1*817, DPB1*818, DPB1*819, DPB1*82, DPB1*820, DPB1*821, DPB1*822, DPB1*823, DPB1*824, DPB1*825, DPB1*826, DPB1*827, DPB1*828, DPB1*829, DPB1*83, DPB1*830, DPB1*831, DPB1*832, DPB1*833, DPB1*834, DPB1*835, DPB1*836, DPB1*837, DPB1*838, DPB1*839, DPB1*84, DPB1*840, DPB1*841, DPB1*842, DPB1*843, DPB1*844, DPB1*845, DPB1*846, DPB1*847, DPB1*848, DPB1*849, DPB1*85, DPB1*850, DPB1*851, DPB1*852, DPB1*853, DPB1*854, DPB1*855, DPB1*856, DPB1*857, DPB1*858, DPB1*859, DPB1*86, DPB1*860, DPB1*861, DPB1*862, DPB1*863, DPB1*864, DPB1*865, DPB1*866, DPB1*867, DPB1*868, DPB1*869, DPB1*87, DPB1*870, DPB1*871, DPB1*872, DPB1*873, DPB1*874, DPB1*875, DPB1*876, DPB1*877, DPB1*878, DPB1*879, DPB1*88, DPB1*880, DPB1*881, DPB1*882, DPB1*883, DPB1*884, DPB1*885, DPB1*886, DPB1*887, DPB1*888, DPB1*889, DPB1*89, DPB1*890, DPB1*891, DPB1*892, DPB1*893, DPB1*894, DPB1*895, DPB1*896, DPB1*897, DPB1*898, DPB1*899, DPB1*90, DPB1*900, DPB1*901, DPB1*902, DPB1*903, DPB1*904, DPB1*905, DPB1*906, DPB1*907, DPB1*908, DPB1*909, DPB1*91, DPB1*910, DPB1*911, DPB1*912, DPB1*913, DPB1*914, DPB1*915, DPB1*916, DPB1*917, DPB1*918, DPB1*919, DPB1*92, DPB1*920, DPB1*921, DPB1*922, DPB1*923, DPB1*924, DPB1*925, DPB1*926, DPB1*927, DPB1*928, DPB1*929, DPB1*93, DPB1*930, DPB1*931, DPB1*932, DPB1*933, DPB1*934, DPB1*935, DPB1*936, DPB1*937, DPB1*938, DPB1*939, DPB1*94, DPB1*940, DPB1*941, DPB1*942, DPB1*943, DPB1*944, DPB1*945, DPB1*946, DPB1*947, DPB1*948, DPB1*949, DPB1*95, DPB1*950, DPB1*951, DPB1*952, DPB1*953, DPB1*954, DPB1*955, DPB1*956, DPB1*957, DPB1*958, DPB1*959, DPB1*96, DPB1*960, DPB1*961, DPB1*962, DPB1*963, DPB1*964, DPB1*965, DPB1*97, DPB1*98, and DPB1*99. In some aspects, the DP beta chain comprises an HLA-DPB1*01, HLA-DPB1*02, HLA-DPB1*01. HLA-DPB1*03, HLA-DPB1*04, HLA-DPB1*05, HLA-DPB1*06, HLA-DPB1*08, HLA-DPB1*09 allele, and any combination thereof. In certain aspects, the DP beta chain comprises an IL A-DPB1*04 allele. In particular aspects, the DP beta chain comprises an HLA-DPB1*04:01 allele.

In some aspects, the DP beta chain comprises an allele selected from DPB1*01:01:01:01, DPB1*01:01:01:02, DPB1*01:01:01:03, DPB1*01:01:01:04, DPB1*01:01:01:05, DPB1*01:01:01:06, DPB1*01:01:01:07, DPB1*01:01:01:08, DPB1*01:01:01:09, DPB1*01:01:01:10, DPB1*01:01:02:01, DPB1*01:01:02:02, DPB1*01:01:03, DPB1*01:01:04, DPB1*01:01:05, DPB1*01:01:06, DPB1*02:01:02:01, DPB1*02:01:02:02, DPB1*02:01:02:03, DPB1*02:01:02:04, DPB1*02:01:02:05, DPB1*02:01:02:06, DPB1*02:01:02:07, DPB1*02:01:02:08, DPB1*02:01:02:09, DPB1*02:01:02:10, DPB1*02:01:02:11, DPB1*02:01:02:12, DPB1*02:01:02:13, DPB1*02:01:02:14, DPB1*02:01:02:15, DPB1*02:01:02:16, DPB1*02:01:02:17, DPB1*02:01:02:18, DPB1*02:01:02:19, DPB1*02:01:02:20, DPB1*02:01:02:21, DPB1*02:01:02:22, DPB1*02:01:02:23, DPB1*02:01:02:24, DPB1*02:01:02:25, DPB1*02:01:02:26, DPB1*02:01:02:27, DPB1*02:01:02:28, DPB1*02:01:02:29, DPB1*02:01:02:30, DPB1*02:01:02:31, DPB1*02:01:02:32, DPB1*02:01:02:33, DPB1*02:01:02: 34, DPB1*02:01:02:35, DPB1*02:01:02:36, DPB1*02:01:02:37, DPB1*02:01:02:38, DPB1*02:01:02:39, DPB1*02:01:02:40, DPB1*02:01:02:41, DPB1*02:01:02:42, DPB1*02:01:02:43, DPB1*02:01:03, DPB1*02:01:04, DPB1*02:01:05, DPB1*02:01:06, DPB1*02:01:07, DPB1*02:01:08, DPB1*02:01:09, DPB1*02:01:10, DPB1*02:01:11, DPB1*02:01:12, DPB1*02:01:13, DPB1*02:01:14, DPB1*02:01:15, DPB1*02:01:16, DPB1*02:01:17, DPB1*02:01:18, DPB1*02:01:19, DPB1*02:01:20, DPB1*02:01:21, DPB1*02:01:22, DPB1*02:01:23, DPB1*02:01:24, DPB1*02:01:25, DPB1*02:01:26, DPB1*02:01:27, DPB1*02:01:28, DPB1*02:01:29, DPB1*02:01:30, DPB1*02:01:31, DPB1*02:01:32, DPB1*02:01:33, DPB1*02:01:34, DPB1*02:01:35, DPB1*02:01:36, DPB1*02:01:37, DPB1*02:01:38, DPB1*02:01:39, DPB1*02:01:40, DPB1*02:01:41, DPB1*02:01:42, DPB1*02:01:43, DPB1*02:02:01:01, DPB1*02:02:01:02, DPB1*02:02:01:03, DPB1*02:02:01:04, DPB1*02:02:01:05, DPB1*02:02:01:06, DPB1*02:02:01:07, DPB1*02:02:02, DPB1*02:02:03, DPB1*03:01:01:01, DPB1*03:01:01: 02, DPB1*03:01: 01:03, DPB1*03:01:01:04, DPB1*03:01:01:05, DPB1*03:01: 01:06, DPB1*03:01:01:07, DPB1*03:01:01:08, DPB1*03:01:01:09, DPB1*03:01:01:10, DPB1*03:01:01:11, DPB1*03:01:02, DPB1*03:01:03, DPB1*03:01:04, DPB1*03:01:05, DPB1*03:01:06, DPB1*03:01:07, DPB1*03:01:08, DPB1*03:01:09, DPB1*03:01:10, DPB1*03:01:11, DPB1*03:01:12, DPB1*04:01:01:01, DPB1*04:01: 01:02, DPB1*04:01:01:03, DPB1*04:01:01:04, DPB1*04:01:01:05, DPB1*04:01:01:06, DPB1*04:01:01:07, DPB1*04:01: 01:08, DPB1*04:01:01:09, DPB1*04:01:01:10, DPB1*04:01:01:11, DPB1*04:01:01:12, DPB1*04:01:01:13, DPB1*04:01:01:14, DPB1*04:01:01:15, DPB1*04:01:01:16, DPB1*04:01:01:17, DPB1*04:01:01:18, DPB1*04:01:01:19, DPB1*04:01:01:20, DPB1*04:01:01:21, DPB1*04:01:01:22, DPB1*04:01:01:23, DPB1*04:01:01:24N, DPB1*04:01:01:25, DPB1*04:01:01:26, DPB1*04:01:01:27, DPB1*04:01:01:28, DPB1*04:01:01:29, DPB1*04:01:01:30, DPB1*04:01:01:31, DPB1*04:01:01:32, DPB1*04:01:01:33, DPB1*04:01:01:34, DPB1*04:01:02, DPB1*04:01:03, DPB1*04:01:04:01, DPB1*04:01:04:02, DPB1*04:01:05, DPB1*04:01:06, DPB1*04:01:07, DPB1*04:01:08, DPB1*04:01:09, DPB1*04:01:10, DPB1*04:01:11, DPB1*04:01:12, DPB1*04:01:13, DPB1*04:01:14, DPB1*04:01:15, DPB1*04:01:16, DPB1*04:01:17, DPB1*04:01:18, DPB1*04:01:19, DPB1*04:01:20, DPB1*04:01:21, DPB1*04:01:22, DPB1*04:01:23, DPB1*04:01:24, DPB1*04:01:25, DPB1*04:01:26, DPB1*04:01:27, DPB1*04:01:28, DPB1*04:01:29, DPB1*04:01:30, DPB1*04:01:31, DPB1*04:01:32, DPB1*04:01:33, DPB1*04:01:34, DPB1*04:01:35, DPB1*04:01:36, DPB1*04:01:37, DPB1*04:01:38, DPB1*04:01:39, DPB1*04:01:40, DPB1*04:02:01:01, DPB1*04:02:01:02, DPB1*04:02:01:03, DPB1*04:02:01:04, DPB1*04:02:01:05, DPB1*04:02:01:06, DPB1*04:02:01:07, DPB1*04:02:01:08, DPB1*04:02:01:09, DPB1*04:02:01:10, DPB1*04:02:01:11, DPB1*04:02:01:12, DPB1*04:02:01:13, DPB1*04:02:01:14, DPB1*04:02:02, DPB1*04:02:03, DPB1*04:02:04, DPB1*04:02:05, DPB1*04:02:06, DPB1*04:02:07, DPB1*04:02:08, DPB1*04:02:09, DPB1*04:02:10, DPB1*04:02:11, DPB1*04:02:12, DPB1*04:02:13, DPB1*04: 02:14, DPB1*05:01:01:01, DPB1*05:01:01:02, DPB1*05:01:01:03, DPB1*05:01:01:04, DPB1*05:01:01:05, DPB1*05:01:01:06, DPB1*05:01:01:07, DPB1*05:01:01:08, DPB1*05:01:01:09, DPB1*05:01:01:10, DPB1*05:01:02, DPB1*05:01:03, DPB1*05:01:04, DPB1*05:01:05, DPB1*05:01:06, DPB1*05:01:07, DPB1*05:01:08, DPB1*05:01:09, DPB1*06:01:01:01, DPB1*06:01:01:02, DPB1*06:01:01:03, DPB1*06:01:02, DPB1*06:01:03, DPB1*06:01:04, DPB1*06:01:05, DPB1*08:01, DPB1*09:01:01, DPB1*09:01:02, DPB1*09:01:03, DPB1*09:01:04, DPB1*100:01, DPB1*101:01, DPB1*102:01, DPB1*103:01, DPB1*104:01:01:01, DPB1*104:01:01:02 DPB1*104:01:01:03, DPB1*104:01:01:04, DPB1*104:01:01:05, DPB1*104:01:01:06, DPB1*104:01:02, DPB1*105:01:01:01, DPB1*105:01:01:02, DPB1*105:01:01:03, DPB1*105:01:01:04, DPB1*105:01:01:05, DPB1*105:01:01:06, DPB1*105:01:01:07, DPB1*105:01:01:08, DPB1*105:01:01:09, DPB1*105:01:01:10, DPB1*106:01, DPB1*107:01, DPB1*108:01, DPB1*109:01, DPB1*10:01:01:01, DPB1*10:01:01:02, DPB1*10:01:02, DPB1*10:01:03, DPB1*10:01:04, DPB1*110:01, DPB1*111:01, DPB1*112:01, DPB1*113:01, DPB1*114:01, DPB1*115:01, DPB1*116:01, DPB1*117:01, DPB1*118:01, DPB1*119:01, DPB1*11:01:01:01, DPB1*11:01:01:02, DPB1*11:01:02, DPB1*11:01:03, DPB1*11:01:04, DPB1*120:0IN, DPB1*121:01, DPB1*122:01, DPB1*123:01, DPB1*124:01:01:01, DPB1*124:01:01:02, DPB1*124:01:02:01, DPB1*124:01:02:02, DPB1*125:01, DPB1*126:01:01:01, DPB1*126:01:01:02, DPB1*127:01, DPB1*128:01, DPB1*129:01, DPB1*130:01, DPB1*131:01:01:01, DPB1*131:01:01:02, DPB1*131:01:02, DPB1*131:01:03, DPB1*132:01, DPB1*133:01, DPB1*134:01, DPB1*135:01, DPB1*136:01, DPB1*137:01, DPB1*138:01, DPB1*139:01, DPB1*13:01:01:01, DPB1*13:01:01:02, DPB1*13:01:01:03, DPB1*13:01:01:04, DPB1*13:01:01:05, DPB1*13:01:01:06, DPB1*13:01:01:07, DPB1*13:01:01:08, DPB1*13:01:02, DPB1*13:01:03, DPB1*140:01, DPB1*141:01, DPB1*142:01, DPB1*143:01, DPB1*144:01, DPB1*145:01, DPB1*146:01, DPB1*147:01, DPB1*148:01, DPB1*149:01, DPB1*14:01:01:01, DPB1*14:01:01:02, DPB1*14:01:01:03, DPB1*14:01:02, DPB1*14:01:03, DPB1*14:01:04, DPB1*14:01:05, DPB1*14:01:06, DPB1*14:01:07, DPB1*14:01:08, DPB1*14:01:09, DPB1*150:01, DPB1*151:01, DPB1*152:01, DPB1*153:01, DPB1*154:01N, DPB1*155:01:01, DPB1*155:01:02, DPB1*156:01, DPB1*157:01, DPB1*158:01, DPB1*159:0IN, DPB1*15:01:01:01, DPB1*15:01:01:02, DPB1*15:01:01:03, DPB1*15:01:01:04, DPB1*15:01:02, DPB1*15:01:03, DPB1*160:01, DPB1*161:0IN, DPB1*162:01:01, DPB1*162:01:02, DPB1*163:01, DPB1*164:01, DPB1*165:01, DPB1*166:01, DPB1*167:01, DPB1*168:01, DPB1*169:01, DPB1*16:01:01:01, DPB1*16:01:01:02, DPB1*16:01:02, DPB1*16:01:03, DPB1*170:01, DPB1*171:01, DPB1*172:01, DPB1*173:01, DPB1*174:01, DPB1*175:01, DPB1*176:01, DPB1*177:01, DPB1*178:01, DPB1*179:01, DPB1*17:01:01:01, DPB1*17:01:01:02, DPB1*17:01:02, DPB1*17:01:03, DPB1*180:01, DPB1*181:01, DPB1*182:01, DPB1*183:01, DPB1*184:01, DPB1*185:01, DPB1*186:01, DPB1*187:01, DPB1*188:01, DPB1*189:01, DPB1*18:01:01:01, DPB1*18:01:01:02, DPB1*18:01:01:03, DPB1*18:01:02, DPB1*18:01:03, DPB1*190:01, DPB1*191:01, DPB1*192:01, DPB1*193:01, DPB1*194:01, DPB1*195:01, DPB1*196:01, DPB1*197:01, DPB1*198:01, DPB1*199:01 DPB1*19:01:01:01, DPB1*19:01:01:02, DPB1*19:01:01:03, DPB1*200:01, DPB1*201:01, DPB1*202:01, DPB1*203:01:01, DPB1*203:01:02, DPB1*204:01, DPB1*205:01, DPB1*206:01, DPB1*207:01, DPB1*208:01, DPB1*209:01, DPB1*20:01:01:01, DPB1*20:01:01:02, DPB1*20:01:02, DPB1*20:01:03, DPB1*20:01:04, DPB1*210:01, DPB1*211:01, DPB1*212:01, DPB1*213:01:01, DPB1*213:01:02, DPB1*214:01, DPB1*215:01, DPB1*216: IN, DPB1*217:01, DPB1*218: IN, DPB1*219:01, DPB1*21:01, DPB1*220:01, DPB1*221:01, DPB1*222:01, DPB1*223:01, DPB1*224:01, DPB1*225:01, DPB1*226:01, DPB1*227:01:01, DPB1*227:01:02, DPB1*228:01, DPB1*229:01, DPB1*22:01:01:01, DPB1*22:01:01:02, DPB1*230:01, DPB1*231:01, DPB1*232:01, DPB1*233:01, DPB1*234:01, DPB1*235:01, DPB1*236:01:01, DPB1*236:01:02, DPB1*237:01, DPB1*238:01, DPB1*239:01, DPB1*23:01:01:01, DPB1*23:01:01:02, DPB1*23:01:02, DPB1*240:01, DPB1*241:01, DPB1*242:01, DPB1*243:01, DPB1*244:01, DPB1*245:01, DPB1*246:01, DPB1*247:01, DPB1*248:01, DPB1*249:01, DPB1*24:01, DPB1*250:01, DPB1*251:01, DPB1*252:01, DPB1*253:01, DPB1*254:01, DPB1*255:01, DPB1*256:01, DPB1*257:01, DPB1*258:01, DPB1*259:01, DPB1*25:01, DPB1*260:01, DPB1*261:01, DPB1*262:01, DPB1*263:01, DPB1*264:01, DPB1*265:01, DPB1*266:01, DPB1*267:01, DPB1*268:01, DPB1*269:01, DPB1*26:01:01 DPB1*26:01:02, DPB1*26:01:03, DPB1*270:01, DPB1*271:01, DPB1*272:01, DPB1*273:01, DPB1*274:01, DPB1*275:01, DPB1*276:01, DPB1*277:01, DPB1*278:01, DPB1*279:01:01, DPB1*279:01:02, DPB1*27:01, DPB1*280:01, DPB1*281:01, DPB1*282:01, DPB1*283:01, DPB1*284:01, DPB1*285:01, DPB1*286:01, DPB1*287:01, DPB1*288:01, DPB1*289:01, DPB1*28:01, DPB1*290:01, DPB1*291:01, DPB1*292:01, DPB1*293:01, DPB1*294:01, DPB1*295:01, DPB1*296:01, DPB1*297:01, DPB1*298:01, DPB1*299:01, DPB1*29:01, DPB1*300:01, DPB1*301:01, DPB1*302:01, DPB1*303:01, DPB1*304:01, DPB1*305:01, DPB1*306:01, DPB1*307:01, DPB1*308:01, DPB1*309:01, DPB1*30:01:01:01, DPB1*30:01:01:02, DPB1*310:01, DPB1*311:01, DPB1*312:01, DPB1*313:01, DPB1*314:01, DPB1*315:01, DPB1*316:01, DPB1*317:01, DPB1*318:01, DPB1*319:01, DPB1*31:01:01:01, DPB1*31:01:01:02, DPB1*320:01, DPB1*321:01, DPB1*322:01, DPB1*323:01, DPB1*324:01, DPB1*325:01, DPB1*326:01, DPB1*327:01, DPB1*328:01N, DPB1*329:01, DPB1*32:01, DPB1*330:01, DPB1*331:01, DPB1*332:01, DPB1*333:01, DPB1*334:01, DPB1*335:01, DPB1*336:01, DPB1*337:01, DPB1*338:01, DPB1*339:01, DPB1*33:01:01:01, DPB1*33:01:01:02, DPB1*33:01:01:03, DPB1*33:01:01:04, DPB1*33:01:01:05, DPB1*340:01, DPB1*341:01, DPB1*342:01, DPB1*343:01, DPB1*344:01, DPB1*345:01, DPB1*346:01, DPB1*347:01, DPB1*348:01:01, DPB1*348:01:02, DPB1*349:01, DPB1*34:01:01:01, DPB1*34:01:01:02, DPB1*34:01:02, DPB1*350:01, DPB1*351:01, DPB1*352:01:01, DPB1*352:01:02, DPB1*353:01, DPB1*354:01:01, DPB1*354:01:02, DPB1*355:01, DPB1*356:01, DPB1*357:01N, DPB1*358:01, DPB1*359:01, DPB1*35:01:01, DPB1*360:01, DPB1*361:01, DPB1*362:01, DPB1*363:01, DPB1*364:01, DPB1*365:01, DPB1*366:01, DPB1*367:01, DPB1*368:01, DPB1*369:01, DPB1*36:01, DPB1*370:01, DPB1*371:01, DPB1*372:01, DPB1*373:01, DPB1*374:01, DPB1*375:01, DPB1*376:01, DPB1*377:01, DPB1*378:01, DPB1*379:01, DPB1*37:01, DPB1*380:01, DPB1*381:01, DPB1*382:0IN, DPB1*383:01, DPB1*384:01, DPB1*385:01, DPB1*386:01, DPB1*387:01, DPB1*388:01, DPB1*389:01, DPB1*38:01, DPB1*390:01, DPB1*391:01, DPB1*392:01, DPB1*393:01, DPB1*394:01, DPB1*395:01, DPB1*396:01, DPB1*397:01, DPB1*398:01, DPB1*399:01 DPB1*39:01:01:01, DPB1*39:01:01:02, DPB1*39:01:01:03, DPB1*39:01:01:04, DPB1*39:01:02, DPB1*39:01:03, DPB1*400:01, DPB1*401:01N, DPB1*402:01, DPB1*403:01N, DPB1*404:01, DPB1*405:01, DPB1*406:01, DPB1*407:01, DPB1*408:01, DPB1*409:01, DPB1*40:01:01:01, DPB1*40:01:01:02, DPB1*40:01:01:03, DPB1*40:01:02, DPB1*410:01, DPB1*411:01, DPB1*412:01, DPB1*413:01, DPB1*414:01:01:01, DPB1*414:01:01:02, DPB1*415:01, DPB1*416:01:01:01, DPB1*416:01:01:02, DPB1*416:01:01:03, DPB1*416:01:02, DPB1*417:01:01, DPB1*417:01:02, DPB1*418:01, DPB1*419:01, DPB1*41:01:01:01, DPB1*41:01:01:02, DPB1*41:01:02, DPB1*420:01, DPB1*421:01, DPB1*422:01, DPB1*423:01:01, DPB1*423:01:02, DPB1*424:01, DPB1*425:01, DPB1*426:01, DPB1*427:01, DPB1*428:01, DPB1*429:01, DPB1*430:01, DPB1*431:01, DPB1*432:01, DPB1*433:01, DPB1*434:01, DPB1*435:01, DPB1*436:01, DPB1*437:01, DPB1*438:01, DPB1*439:01, DPB1*440:01, DPB1*441:01, DPB1*442:01, DPB1*443:01, DPB1*444:01, DPB1*445:01, DPB1*446:01, DPB1*447:01, DPB1*448:01, DPB1*449:01, DPB1*44:01, DPB1*450:01N, DPB1*451:01, DPB1*452:01, DPB1*453:01, DPB1*454:01, DPB1*455:01N, DPB1*456:01, DPB1*457:01, DPB1*458:01, DPB1*459:01, DPB1*45:01, DPB1*460:01, DPB1*461:01, DPB1*462:01, DPB1*463:01:01:01, DPB1*463:01:01:02, DPB1*463:01:01:03, DPB1*464:01, DPB1*465:01, DPB1*466:01, DPB1*467:01, DPB1*468:01, DPB1*469:01, DPB1*46:01:01, DPB1*46:01:02, DPB1*470:01, DPB1*471:01, DPB1*472:01, DPB1*473:01, DPB1*474:01, DPB1*475:01, DPB1*476:01, DPB1*477:01, DPB1*478:01, DPB1*479:01, DPB1*47:01:01:01, DPB1*47:01:01:02, DPB1*47:01:01:03, DPB1*480:01, DPB1*481:01, DPB1*482:01, DPB1*483:01, DPB1*484:01, DPB1*485:01, DPB1*486:01, DPB1*487:01, DPB1*488:01, DPB1*489:01, DPB1*48:01, DPB1*490:01, DPB1*491:01, DPB1*492:01, DPB1*493:01, DPB1*494:01, DPB1*495:01, DPB1*496:01, DPB1*497:01, DPB1*498:01, DPB1*499:01, DPB1*49:01:01:01, DPB1*49:01:01:02, DPB1*49:01:01:03, DPB1*500:01, DPB1*501:01, DPB1*502:01, DPB1*503:01, DPB1*504:01, DPB1*505:01, DPB1*506:01, DPB1*507:01N, DPB1*508:01, DPB1*509:01, DPB1*50:01, DPB1*510:01, DPB1*511:01, DPB1*512:01, DPB1*513:01, DPB1*514:01, DPB1*515:01, DPB1*516:01, DPB1*517:01, DPB1*518:01, DPB1*519:01, DPB1*51:01:01:01, DPB1*51:01:01:02, DPB1*520:01, DPB1*521:01, DPB1*522:01, DPB1*523:01:01, DPB1*523:01:02, DPB1*524:01, DPB1*525:01, DPB1*526:01, DPB1*527:01, DPB1*528:01, DPB1*529:01, DPB1*52:01, DPB1*530:01, DPB1*531:01, DPB1*532:01, DPB1*533:01, DPB1*534:01, DPB1*535:01, DPB1*536:01, DPB1*537:01, DPB1*538:01, DPB1*539:01, DPB1*53:01, DPB1*540:01, DPB1*541:01, DPB1*542:01, DPB1*543:01, DPB1*544:01, DPB1*545:01, DPB1*546:01, DPB1*547:01, DPB1*548:01, DPB1*549:01, DPB1*54:01, DPB1*550:01, DPB1*551:01N, DPB1*552:01, DPB1*553:01, DPB1*554:01, DPB1*555:01, DPB1*556:01, DPB1*557:01, DPB1*558:01, DPB1*559:01, DPB1*55:01:01:01, DPB1*55:01:01:02, DPB1*55:01:01:03, DPB1*55:01:01:04, DPB1*55:01:01:05, DPB1*55:01:02, DPB1*560:01, DPB1*561:01, DPB1*562:01, DPB1*563:01, DPB1*564:01, DPB1*565:01, DPB1*566:01, DPB1*567:01, DPB1*568:01, DPB1*569:01, DPB1*56:01, DPB1*570:01N, DPB1*571:01, DPB1*572:01, DPB1*573:01, DPB1*574:01, DPB1*575:01, DPB1*576:01, DPB1*577:01, DPB1*578:01, DPB1*579:01, DPB1*57:01, DPB1*580:01, DPB1*581:01, DPB1*582:01, DPB1*583:01, DPB1*584:01:01:01, DPB1*584:01:01:02, DPB1*584:01:01:03, DPB1*584:01:02:01, DPB1*584:01:02:02, DPB1*585:01:01:01, DPB1*585:01:01:02, DPB1*586:01, DPB1*587:01, DPB1*588:01, DPB1*589:01, DPB1*58:01, DPB1*590:01, DPB1*591:01, DPB1*592:01, DPB1*593:01, DPB1*594:01, DPB1*595:01, DPB1*596:01, DPB1*597:01, DPB1*598:01N, DPB1*599:01, DPB1*59:01, DPB1*600:01, DPB1*601:01, DPB1*602:01, DPB1*603:01, DPB1*604:01, DPB1*605:01, DPB1*606:01, DPB1*607:01, DPB1*608:01, DPB1*609:01, DPB1*60:01, DPB1*610:01, DPB1*611:01, DPB1*612:01, DPB1*613:01, DPB1*614:01, DPB1*615:01, DPB1*616:01, DPB1*617:01, DPB1*618:01, DPB1*619:01, DPB1*61:01N, DPB11*620:01, DPB1*621:01, DPB1*622:01, DPB1*623:01, DPB1*624:01, DPB1*625:01, DPB1*626:01, DPB1*627:01, DPB1*628:01, DPB1*629:01, DPB1*62:01, DPB1*630:01, DPB1*631:01, DPB1*632:01, DPB1*633:01, DPB1*634:01, DPB1*635:01, DPB1*636:01, DPB1*637:01, DPB1*638:01, DPB1*639:01, DPB1*63:01, DPB1*640:01, DPB1*641:01, DPB1*642:01, DPB1*643:01, DPB1*644:01, DPB1*645:01, DPB1*646:01, DPB1*647:01, DPB1*648:01:01:01, DPB1*648:01:01:02, DPB1*649:01, DPB1*64:01N, DPB1*650:01, DPB1*651:01, DPB1*652:01, DPB1*653:01, DPB1*654:01, DPB1*655:01, DPB1*656:01, DPB1*657:0IN, DPB1*658:01, DPB1*659:01, DPB1*65:01:01, DPB1*65:01:02, DPB1*660:01, DPB1*661:01N, DPB1*662:01, DPB1*663:01, DPB1*664:01, DPB1*665:01, DPB1*666:01, DPB1*667:01, DPB1*668:01:01:01, DPB1*668:01:01:02, DPB1*669:01, DPB1*66:01, DPB1*670:01, DPB1*671:01, DPB1*672:01, DPB1*673:01, DPB1*674:01, DPB1*675:01, DPB1*676:01, DPB1*677:01, DPB1*678:01, DPB1*679:01, DPB1*67:01, DPB1*680:01, DPB1*681:01, DPB1*682:01, DPB1*683:01, DPB1*684:01, DPB1*685:01, DPB1*686:01, DPB1*687:01, DPB1*688:01, DPB1*689:01, DPB1*68:01, DPB1*690:01, DPB1*691:01N, DPB1*692:01, DPB1*693:01N, DPB1*694:01, DPB1*695:01, DPB1*696:01N, DPB1*697:01Q, DPB1*698:01, DPB1*699:01, DPB1*69:01:01:01, DPB1*69:01:01:02, DPB1*700:01N, DPB1*701:01, DPB1*702:01, DPB1*703:01, DPB1*704:01, DPB1*705:01, DPB1*706:01, DPB1*707:01, DPB1*708:01, DPB1*709:01, DPB1*70:01, DPB1*710:01, DPB1*711:01, DPB1*712:01N, DPB1*713:01, DPB1*714:01, DPB1*715:01, DPB1*716:01, DPB1*71 7:01, DPB1*718:01, DPB1*719:01, DPB1*71:01:01, DPB1*71:01:02, DPB1*720:01, DPB1*721:01, DPB1*722:01, DPB1*723:01, DPB1*724:01N, DPB1*725:01, DPB1*726:01, DPB1*727:01, DPB1*728:01, DPB1*729:01, DPB1*72:01:01:01, DPB1*72:01:01:02, DPB1*72:01:01:03, DPB1*730:01, DPB1*731:01, DPB1*732:01N, DPB1*733:01, DPB1*734:01, DPB1*735:01, DPB1*736:01, DPB1*737:01, DPB1*738:01N, DPB1*739:01, DPB1*73:01, DPB1*740:01, DPB1*741:01, DPB1*742:01, DPB1*743:01N, DPB1*744:01, DPB1*745:01, DPB1*746:01, DPB1*747:01, DPB1*748:01N, DPB1*749:01, DPB1*74:01, DPB1*750:01, DPB1*751:01, DPB1*752:01, DPB1*753:01, DPB1*754:01N, DPB1*755:01, DPB1*756:01N, DPB1*757:01, DPB1*758:01, DPB1*759:01, DPB1*75:01, DPB1*760:01, DPB1*761:01, DPB1*762:01, DPB1*763:01, DPB1*764:01, DPB1*765:01, DPB1*766:01, DPB1*767:01, DPB1*768:01, DPB1*769:01, DPB1*76:01, DPB1*770:01, DPB1*771:01, DPB1*772:01, DPB1*773:01, DPB1*774:01, DPB1*775:01, DPB1*776:01, DPB1*777:01N DPB1*778:01, DPB1*779:01, DPB1*77:01, DPB1*780:01, DPB1*781:01, DPB1*782:01, DPB1*783:01, DPB1*784:01, DPB1*785:01, DPB1*786:01:01N, DPB1*786:01:02N, DPB1*787:01, DPB1*788:01, DPB1*789:01, DPB1*78:01, DPB1*790:01, DPB1*791:01, DPB1*792:01N, DPB1*794:01N, DPB1*795:01, DPB1*796:01, DPB1*797:01, DPB1*798:01, DPB1*799:01, DPB1*79:01, DPB1*800:01N, DPB1*801:01, DPB1*802:01, DPB1*803:01, DPB1*804:01, DPB1*805:01, DPB1*806:01:01:01, DPB1*806:01:01:02, DPB1*807:01, DPB1*808:01, DPB1*809:01, DPB1*80:01, DPB1*810:01, DPB1*811:01, DPB1*812:01, DPB1*813:01, DPB1*814:01, DPB1*815:01, DPB1*816:01, DPB1*817:01, DPB1*818:01, DPB1*819:01, DPB1*81:01:01:01, DPB1*81:01:01:02, DPB1*81:01:02, DPB1*820:01, DPB1*821:01N, DPB1*822:01, DPB1*823:01, DPB1*824:01, DPB1*825:01, DPB1*826:01, DPB1*827:01, DPB1*828:01, DPB1*829:01, DPB1*82:01, DPB1*830:01, DPB1*831:01N, DPB1*832:01, DPB1*833:01, DPB1*834:01, DPB1*835:01, DPB1*836:01, DPB1*837:01, DPB1*838:01N, DPB1*839:01, DPB1*83:01, DPB1*840:01, DPB1*841:01, DPB1*842:01, DPB1*843:01, DPB1*844:01N, DPB1*845:01, DPB1*846:01, DPB1*847:01, DPB1*848:01, DPB1*849:01, DPB1*84:01, DPB1*850:01, DPB1*851:01, DPB1*852:01, DPB1*853:01, DPB1*854:01, DPB1*855:01, DPB1*856:01, DPB1*857:01, DPB1*858:01, DPB1*859:01, DPB1*85:01:01:01, DPB1*85:01:01:02, DPB1*85:01:02, DPB1*860:01, DPB1*861:01, DPB1*862:01N, DPB1*863:01, DPB1*864:01, DPB1*865:01N, DPB1*866:01N, DPB1*867:01N, DPB1*868:01N, DPB1*869:01N, DPB1*86:01, DPB1*870: IN, DPB1*871:01N, DPB1*872:01N, DPB1*873:01N, DPB1*874:01N, DPB1*875:01N, DPB1*876:01N, DPB1*877:01N, DPB1*878:01N, DPB1*879:01:01:01, DPB1*879:01:01:02, DPB1*879:01:01:03, DPB1*87:01, DPB1*880:01, DPB1*881:01, DPB1*882:01, DPB1*883:01, DPB1*884:01, DPB1*885:01, DPB1*886:01, DPB1*887:01, DPB1*888:01, DPB1*889:01, DPB1*88:01, DPB1*890:01, DPB1*891:01, DPB1*892:01, DPB1*893: 01, DPB1*894:0IN, DPB1*895: 01, DPB1*896:01, DPB1*897:01, DPB1*898:01, DPB1*899:01, DPB1*89:01, DPB1*900:01, DPB1*901:01, DPB1*902:01, DPB1*903:01, DPB1*904:01, DPB1*905:01, DPB1*906:01, DPB1*907:01, DPB1*908:01, DPB1*909:01, DPB1*90:01:01, DPB1*90:01:02, DPB1*910:01, DPB1*911:01N, DPB1*912:01, DPB1*913:01, DPB1*914:01, DPB1*915:01, DPB1*916:01, DPB1*917:01N, DPB1*918:01, DPB1*919:01N, DPB1*91:01:01:01, DPB1*91:01:01:02, DPB1*920:01, DPB1*921:01, DPB1*922:01, DPB1*923:01, DPB1*924:01, DPB1*925:01N, DPB1*926:01, DPB1*927:01, DPB1*928:01, DPB1*929:01, DPB1*92:01, DPB1*930:01, DPB1*931:01, DPB1*932:01, DPB1*933:01, DPB1*934:01Q, DPB1*935:01Q, DPB1*936:01Q, DPB1*937:01, DPB1*938:01, DPB1*939:0IN, DPB1*93:01, DPB1*940:01, DPB1*941:01N, DPB1*942:01, DPB1*943:01, DPB1*944:01, DPB1*945:01, DPB1*946:01, DPB1*947:01, DPB1*948:01, DPB1*949:01, DPB1*94:01, DPB1*950:01N, DPB1*951:01, DPB1*952:01, DPB1*953:01, DPB1*954:01, DPB1*955:01, DPB1*956:01, DPB1*957:01, DPB1*958:01, DPB1*959:01N, DPB1*95:01, DPB1*960:0IN, DPB1*961:01, DPB1*962:01, DPB1*963:01, DPB1*964:01, DPB1*965:01:01:01, DPB1*965:01:01:02, DPB1*96:01, DPB1*97:01, DPB1*98:01, DPB1*99:01, and any combination thereof.

### II.D.2. HLA-DQ Class II Molecules

In some aspects, the alpha chain is an HLA-DQ alpha chain. Any HLA-DQ alpha chain allele known in the art can be used in the compositions and methods disclosed herein. In some aspects, the alpha chain is selected from an HLA-DQA1*01, HLA-DQA1*02, HLA-DQA1*03, HLA-DQA1*04, HLA-DQA1*05, and HLA-DQA1*06 allele. In some aspects, the alpha chain is an I-ILA-DQA1 allele selected from *01:01:01:01, *01:01:01:02, *01:01:01:03, *01:01:01:05, *01:01:01:06, *01:01:02, *01:01:03, *01:01:04, *01:01:05, *01:02:01:01, *01:02:01:02, *01:02:01:03, *01:02:01:04, *01:02:01:05, *01:02:01:06, *01:02:01:07, *01:02:01:08, *01:02:01:09, *01:02:01:10, *01:02:01:11, *01:02:01:12, *01:02:02:01, *01:02:02:02, *01:02:02:03, *01:02:02:04, *01:02:03, *01:02:04, *01:03:01:01, *01:03:01:02, *01:03:01:03, *01:03:01:04, *01:03:01:05, *01:03:01:06, *01:03:01:07, *01:03:01:08, *01:03:01:09, *01:04:01:01, *01:04:01:02, *01:04:01:03, *01:04:01:04, *01:04:02, *01:05:01, *01:05:02, *01:06, *01:07Q, *01:08, *01:09, *01:10, *01:11, *01:12, *01:13, *01:14, *01:15N, *01:16N, *01:17, *01:18, *01:19, *01:20, *01:21, *01:22, *01:23, *01:24, *01:25, *01:26, *02:01:01:01, *02:01:01:02, *02:01:02, *02:02N, *02:03, *03:01:01, *03:01:03. *03:02:01:01, *03:02:01:02, *03:03:01:01, *03:03:01:02, *03:03:01:03, *03:03:01:04, *03:03:01:05, *03:03:01:06, *03:03:01:07, *03:03:02, *03:04, *03:05, *03:06, *03:07, *04:01:01:01, *04:01:01:02, *04:01:01:03, *04:01:01:04, *04:01:01:05, *04:01:01:06, *04:01:01:07, *04:01:01:08, *04:01:02:01, *04:01:02:02, *04:01:03, *04:02, *04:03N, *04:04, *04:05, *05:01:01:01, *05:01:01:02, *05:01:01:03, *05:01:01:04, *05:01:02, *05:01:04, *05:01:05, *05:01:06, *05:02, *05:03:01:01, *05:03:01:02, *05:04, *05:05:01:01, *05:05:01:02, *05:05:01:03, *05:05:01:04, *05:05:01:05, *05:05:01:06, *05:05:01:07, *05:05:01:08, *05:05:01:09, *05:05:01:10, *05:05:01:11, *05:05:01:12 *05:05:01:13, *05:05:01:14, *05:05:01:15, *05:05:01:16, *05:05:01:17, *05:05:01:18, *05:05:01:19, *05:05:01:20, *05:06:01:01, *05:06:01:02, *05:07, *05:08, *05:09, *05:10, *05:11, *05:12, *05:13, *05:14, *05:15N, *06:01:01:01, *06:01:01:02, *06:01:01:03, *06:01:01:04, *06:01:02, *06:02, and any combination thereof.

In some aspects, the beta chain is an HLA-DQ beta chain. Any HLA-DQ beta chain allele known in the art can be used in the compositions and methods disclosed herein. In some aspects the beta chain is selected from an HLA-DQB1*02, HLA-DQB1*03, HLA-DQB1*04, HLA-DQB1*05, and HLA-DQB1*06 allele.

In certain aspects, the DQ beta chain comprises an allele selected from DQB1*02:01:01, DQB1*02:01:02, DQB1*02:01:03, DQB1*02:01:04, DQB1*02:01:05, DQB1*02:01:06, DQB1*02:01:07, DQB1*02:01:08, DQB1*02:01:09, DQB1*02:01:10, DQB1*02:01: 11, DQB1*02:01:12, DQB1*02:01:13, DQB1*02:01:14, DQB1*02:01:15, DQB1*02:01:16, DQB1*02:01:17, DQB1*02:01:18, DQB1*02:01:19, DQB1*02:01:20, DQB1*02:01:21, DQB1*02:01:22, DQB1*02:01:23, DQB1*02:01:24, DQB1*02:01:25, DQB1*02:01:26, DQB1*02:01:27, DQB1*02:01:28, DQB1*02:01:29, DQB1*02:01:30, DQB1*02:01:31, DQB1*02:02:01:01, DQB1*02:02:01:02, DQB1*02:02:01:03, DQB1*02:02:01:04, DQB1*02:02:02, DQB1*02:02:03, DQB1*02:02:04, DQB1*02:02:05, DQB1*02:02:06, DQB1*02:02:07, DQB1*02:02:08, DQB1*02:02:09, DQB1*02:03:01, DQB1*02:03:02, DQB1*02:04, DQB1*02:05, DQB1*02:06, DQB1*02:07:01, DQB1*02:07:02, DQB1*02:08, DQB11*02:09, DQB1*02:10, DQB1*02:100, DQB1*02:101, DQB1*02:102, DQB1*02:103, DQB1*02:104, DQB1*02:105, DQB1*02:106, DQB1*02:107, DQB1*02:108, DQB1*02:109, DQB1*02:11, DQB1*02:110, DQB1*02:111, DQB1*02:112, DQB1*02:113, DQB1*02:114, DQB1*02:115, DQB1*02:116, DQB1*02:117, DQB1*02:118, DQB1*02:119, DQB1*02:12, DQB1*02:120, DQB1*02:121, DQB1*02:122, DQB1*02:123, DQB1*02:124, DQB1*02:125, DQB1*02:126, DQB1*02:127, DQB1*02:128, DQB1*02:129N, DQB1*02:13, DQB1*02:130, DQB1*02:131, DQB1*02:132N, DQB1*02:133, DQB1*02:134N, DQB1*02:135, DQB1*02:136, DQB1*02:137, DQB1*02:138, DQB1*02:139, DQB1*02:140, DQB1*02:141, DQB1*02:142, DQB1*02:14:01, DQB1*02:14:02, DQB1*02:15, DQB1*02:16, DQB1*02:17, DQB1*02:18N, DQB1*02:19, DQB1*02:20N, DQB1*02:21, DQB1*02:22, DQB1*02:23, DQB1*02:24, DQB1*02:25, DQB1*02:26, DQB1*02:27, DQB1*02:28, DQB1*02:29, DQB1*02:30, DQB1*02:31, DQB1*02:32, DQB1*02:33, DQB1*02:34, DQB1*02:35, DQB1*02:36, DQB1*02:37, DQB1*02:38, DQB1*02:39, DQB1*02:40, DQB1*02:41, DQB1*02:42, DQB1*02:43, DQB1*02:44, DQB1*02:45, DQB1*02:46, DQB1*02:47, DQB1*02:48, DQB1*02:49, DQB1*02:50, DQB1*02:51, DQB1*02:52, DQB1*02:53Q, DQB1*02:54, DQB1*02:55, DQB1*02:56, DQB1*02:57, DQB1*02:58N, DQB1*02:59, DQB1*02:60, DQB1*02:61, DQB1*02:62, DQB1*02:63, DQB1*02:64, DQB1*02:65, DQB1*02:66, DQB1*02:67NX, DQB1*02:68, DQB1*02:69, DQB1*02:70, DQB1*02:71, DQB1*02:72, DQB1*02:73, DQB1*02:74, DQB1*02:75, DQB1*02:76, DQB1*02:77, DQB1*02:78, DQB1*02:79, DQB1*02:80, DQB1*02:81, DQB1*02:82, DQB1*02:83, DQB1*02:84, DQB1*02:85, DQB1*02:86, DQB1*02:87, DQB1*02:88, DQB1*02:89:01, DQB1*02:89:02, DQB1*02:90, DQB1*02:91, DQB1*02:92, DQB1*02:93, DQB1*02:94, DQB1*02:95, DQB1*02:96N, DQB1*02:97, DQB1*02:98, DQB1*02:99, DQB1*03:01:01:01, DQB1*03:01:01:02, DQB1*03:01:01:03, DQB1*03:01:01:04, DQB1*03:01:01:05, DQB1*03:01:01:06, DQB1*03:01:01:07, DQB1*03:01:01:08, DQB1*03:01:01:09, DQB1*03:01:01:10, DQB1*03:01:01:11, DQB1*03:01:01:12, DQB1*03:01:01:14, DQB1*03:01:01:15, DQB1*03:01:01:16, DQB1*03:01:01:17, DQB1*03:01:01:18, DQB1*03:01:01:19, DQB1*03:01:01:20, DQB1*03:01:02, DQB1*03:01:03, DQB1*03:01:04, DQB1*03:01:05, DQB1*03:01:06, DQB1*03:01:07, DQB1*03:01:08, DQB1*03:01:09, DQB1*03:01:10, DQB1*03:01:11, DQB1*03:01:12, DQB1*03:01:13, DQB1*03:01:14, DQB1*03:01:15, DQB1*03:01:16, DQB1*03:01:17, DQB1*03:01:18, DQB1*03:01:19, DQB1*03:01:20, DQB1*03:01:21, DQB1*03:01:22, DQB1*03:01:23, DQB1*03:01:24, DQB1*03:01:25, DQB1*03:01:26, DQB1*03:01:27, DQB1*03:01:28, DQB1*03:01:29, DQB1*03:01:30, DQB1*03:01:31, DQB1*03:01:32, DQB1*03:01:33, DQB1*03:01:34, DQB1*03:01:35, DQB1*03:01:36, DQB1*03:01:37, DQB1*03:01:38, DQB1*03:01:39, DQB1*03:01:40, DQB1*03:01:41, DQB1*03:01:42, DQB1*03:01:43, DQB1*03:01:44, DQB1*03:01:45, DQB1*03:01:46, DQB1*03:02:01:01, DQB1*03:02:01:02, DQB1*03:02:01:03, DQB1*03:02:01:04, DQB1*03:02:01:05, DQB1*03:02:01:06, DQB1*03:02:01:07, DQB1*03:02:01:08, DQB1*03:02:02, DQB1*03:02:03, DQB1*03:02:04, DQB1*03:02:05, DQB1*03:02:06, DQB1*03:02:07, DQB1*03:02:08, DQB1*03:02:09, DQB1*03:02:10, DQB1*03:02:11, DQB1*03:02:12, DQB1*03:02:13, DQB1*03:02:14, DQB1*03:02:15, DQB1*03:02:16, DQB1*03:02:17, DQB1*03:02:18, DQB1*03:02:19, DQB1*03:02:20, DQB1*03:02:21, DQB1*03:02:22, DQB1*03:02:23, DQB1*03:02:24, DQB1*03:02:25, DQB1*03:02:26, DQB1*03:02:27, DQB1*03:02:28, DQB1*03:02:29, DQB1*03:02:30, DQB1*03:03:02:01, DQB1*03:03:02:02, DQB1*03:03:02:03, DQB1*03:03:02:04, DQB1*03:03:02:05, DQB1*03:03:03, DQB1*03:03:04, DQB1*03:03:05, DQB1*03:03:06, DQB1*03:03:07, DQB1*03:03:08, DQB1*03:03:09, DQB1*03:03:10, DQB1*03:03:11, DQB1*03:03:12, DQB1*03:03:13, DQB1*03:03:14, DQB1*03:03:15, DQB1*03:03:16, DQB1*03:03:17, DQB1*03:03:18, DQB1*03:03:19, DQB1*03:03:20, DQB1*03:03:21, DQB1*03:04:01, DQB1*03:04:02, DQB1*03:04:03, DQB1*03:04:04, DQB1*03:05:01, DQB1*03:05:02, DQB1*03:05:03, DQB1*03:05:04, DQB1*03:06, DQB1*03:07, DQB1*03:08, DQB1*03:09, DQB1*03:100, DQB1*03:101, DQB1*03:102, DQB1*03:103, DQB1*03:104, DQB1*03:105, DQB1*03:106, DQB1*03:107, DQB1*03:108, DQB1*03:109, DQB1*03:10:01, DQB1*03:10:02:01, DQB1*03:10:02:02, DQB1*03:11, DQB1*03:110, DQB1*03:111, DQB1*03:112, DQB1*03:113, DQB1*03:114, DQB1*03:115, DQB1*03:116, DQB1*03:117, DQB1*03:118N, DQB1*03:119, DQB1*03:12, DQB1*03:120, DQB1*03:121, DQB1*03:122, DQB1*03:123, DQB1*03:124, DQB1*03:125, DQB1*03:126, DQB1*03:127, DQB1*03:128, DQB1*03:129, DQB1*03:13, DQB1*03:130, DQB1*03:131, DQB1*03:132, DQB1*03:133, DQB1*03:134, DQB1*03:135, DQB1*03:136, DQB1*03:137, DQB1*03:138, DQB1*03:139, DQB1*03:140, DQB1*03:141, DQB1*03:142, DQB1*03:143, DQB1*03:144, DQB1*03:145, DQB1*03:146, DQB1*03:147, DQB1*03:148, DQB1*03:149, DQB1*03:14:01, DQB1*03:14:02, DQB1*03:15, DQB1*03:150, DQB1*03:151, DQB1*03:152, DQB1*03:153, DQB1*03:154, DQB1*03:155, DQB1*03:156, DQB1*03:157, DQB1*03:158, DQB1*03:159, DQB1*03:16, DQB1*03:160, DQB1*03:161, DQB1*03:162, DQB1*03:163, DQB1*03:164, DQB1*03:165, DQB1*03:166, DQB1*03:167, DQB1*03:168, DQB1*03:169, DQB1*03:170, DQB1*03:171, DQB1*03:172, DQB1*03:173, DQB1*03:174, DQB1*03:175, DQB1*03:176, DQB1*03:177, DQB1*03:178, DQB1*03:179, DQB1*03:17:01, DQB1*03:17:02, DQB1*03:18, DQB1*03:180, DQB1*03:181, DQB1*03:182, DQB1*03:183, DQB1*03:184, DQB1*03:185, DQB1*03:186, DQB1*03:187, DQB1*03:188, DQB1*03:189, DQB1*03:190, DQB1*03:191, DQB1*03:192, DQB1*03:193, DQB1*03:194 DQB1*03:195, DQB1*03:196, DQB1*03:197Q, DQB1*03:198:01, DQB1*03:198:02, DQB1*03:199, DQB1*03:19:01, DQB1*03:19:02, DQB1*03:19:03, DQB1*03:19:04, DQB1*03:20, DQB1*03:200, DQB1*03:201, DQB1*03:202, DQB1*03:203, DQB1*03:204, DQB1*03:205, DQB1*03:206, DQB1*03:207, DQB1*03:208, DQB1*03:209, DQB1*03:21, DQB1*03:210, DQB1*03:211, DQB1*03:212, DQB1*03:213NX, DQB1*03:214, DQB1*03:215, DQB1*03:216, DQB1*03:217, DQB1*03:218, DQB1*03:219, DQB1*03:220, DQB1*03:221, DQB1*03:222, DQB1*03:223, DQB1*03:224, DQB1*03:225, DQB1*03:226, DQB1*03:227, DQB1*03:228, DQB1*03:229, DQB1*03:22:01, DQB1*03:22:02, DQB1*03:230, DQB1*03:231, DQB1*03:232, DQB1*03:233, DQB1*03:234, DQB1*03:235, DQB1*03:236, DQB1*03:237N DQB1*03:238, DQB1*03:239, DQB1*03:23:01, DQB1*03:23:02, DQB1*03:23:03, DQB1*03:24, DQB1*03:240, DQB1*03:241, DQB1*03:242, DQB1*03:243, DQB1*03:244, DQB1*03:245, DQB1*03:246, DQB1*03:247, DQB1*03:248, DQB1*03:249, DQB1*03:250, DQB1*03:251, DQB1*03:252, DQB1*03:253, DQB1*03:254, DQB1*03:255, DQB1*03:256, DQB1*03:257, DQB1*03:258, DQB1*03:259, DQB1*03:25:01, DQB1*03:25:02, DQB1*03:26, DQB1*03:260, DQB1*03:261, DQB1*03:262, DQB1*03:263, DQB1*03:264, DQB1*03:265, DQB1*03:266, DQB1*03:267, DQB1*03:268, DQB1*03:269N, DQB1*03:27, DQB1*03:270, DQB1*03:271, DQB1*03:272, DQB1*03:273, DQB1*03:274, DQB1*03:275, DQB1*03:277, DQB1*03:278, DQB1*03:279, DQB1*03:28, DQB1*03:280, DQB1*03:281, DQB1*03:282N, DQB1*03:283, DQB1*03:284, DQB1*03:285, DQB1*03:286, DQB1*03:287, DQB1*03:288, DQB1*03:289, DQB1*03:29, DQB1*03:290, DQB1*03:291, DQB1*03:292, DQB1*03:293, DQB1*03:294, DQB1*03:295, DQB1*03:296, DQB1*03:297, DQB1*03:298, DQB1*03:299, DQB1*03:30, DQB1*03:300, DQB1*03:301, DQB1*03:302, DQB1*03:303N, DQB1*03:304, DQB1*03:305, DQB1*03:306, DQB1*03:307, DQB1*03:308, DQB1*03:309, DQB1*03:31, DQB1*03:310N, DQB1*03:311, DQB1*03:312, DQB1*03:313, DQB1*03:314, DQB1*03:315, DQB1*03:316, DQB1*03:317:01, DQB1*03:317:02, DQB1*03:318, DQB1*03:319, DQB1*03:32, DQB1*03:320, DQB1*03:321, DQB1*03:322, DQB1*03:323, DQB1*03:324, DQB1*03:326, DQB1*03:327, DQB1*03:328, DQB1*03:329, DQB1*03:33, DQB1*03:330, DQB1*03:331, DQB1*03:332, DQB1*03:333, DQB1*03:334N4 bp, DQB1*03:335, DQB1*03:336, DQB1*03:337, DQB1*03:338N, DQB1*03:339N, DQB1*03:34, DQB1*03:340N, DQB1*03:341, DQB1*03:342, DQB1*03:343, DQB1*03:344, DQB1*03:345, DQB1*03:346, DQB1*03:347, DQB1*03:348, DQB1*03:349, DQB1*03:35, DQB1*03:350, DQB1*03:351, DQB1*03:352, DQB1*03:353, DQB1*03:354N, DQB1*03:355, DQB1*03:356NX, DQB1*03:357N, DQB1*03:358N, DQB1*03:36, DQB1*03:37, DQB1*03:38:01, DQB1*03:38:02, DQB1*03:39, DQB1*03:40, DQB1*03:41, DQB1*03:42, DQB1*03:43, DQB1*03:44, DQB1*03:45, DQB1*03:46, DQB1*03:47, DQB1*03:48, DQB1*03:49, DQB1*03:50, DQB1*03:51, DQB1*03:52, DQB1*03:53, DQB1*03:54, DQB1*03:55, DQB1*03:56, DQB1*03:57, DQB1*03:58, DQB1*03:59, DQB1*03:60, DQB1*03:61, DQB1*03:62, DQB1*03:63, DQB1*03:64, DQB1*03:65, DQB1*03:66N, DQB1*03:67, DQB1*03:68, DQB1*03:69, DQB1*03:70, DQB1*03:71, DQB1*03:72, DQB1*03:73, DQB1*03:74, DQB1*03:75, DQB1*03:76, DQB1*03:77, DQB1*03:78, DQB1*03:79, DQB1*03:80, DQB1*03:81, DQB1*03:82, DQB1*03:83, DQB1*03:84N, DQB1*03:85, DQB1*03:86, DQB1*03:87, DQB1*03:88, DQB1*03:89, DQB1*03:90N, DQB1*03:91Q, DQB1*03:92, DQB1*03:93, DQB1*03:94, DQB1*03:95N, DQB1*03:96, DQB1*03:97, DQB1*03:98, DQB1*03:99Q, DQB1*04:01:01:01, DQB1*04:01:01:02, DQB1*04:01:02, DQB1*04:01:03, DQB1*04:01:04, DQB1*04:01:05, DQB1*04:02:01:01, DQB1*04:02:01:04, DQB1*04:02:01:05, DQB1*04:02:01:06, DQB1*04:02:01:07, DQB1*04:02:01:08, DQB1*04:02:01:09, DQB1*04:02:01:10, DQB1*04:02:02, DQB1*04:02:03, DQB1*04:02:04, DQB1*04:02:05, DQB1*04:02:06, DQB1*04:02:07, DQB1*04:02:08, DQB1*04:02:09, DQB1*04:02:10, DQB1*04:02:11, DQB1*04:02:12, DQB1*04:02:13, DQB1*04:02:14, DQB1*04:02:15, DQB1*04:02:16, DQB1*04:02:17, DQB1*04:02:18, DQB1*04:03:01, DQB1*04:03:02, DQB1*04:03:03, DQB1*04:04, DQB1*04:05, DQB1*04:06, DQB1*04:07, DQB1*04:08, DQB1*04:09 DQB1*04:10, DQB1*04:11, DQB1*04:12, DQB1*04:13, DQB1*04:14, DQB1*04:15, DQB1*04:16, DQB1*04:17, DQB1*04:18, DQB1*04:19, DQB1*04:20, DQB1*04:21, DQB1*04:22, DQB1*04:23, DQB1*04:24, DQB1*04:25N, DQB1*04:26, DQB1*04:27, DQB1*04:28, DQB1*04:29, DQB1*04:30, DQB1*04:31, DQB1*04:32, DQB1*04:33, DQB1*04:34, DQB1*04:35, DQB1*04:36N, DQB1*04:37, DQB1*04:38, DQB1*04:39, DQB1*04:40, DQB1*04:41N, DQB1*04:42, DQB1*04:43, DQB1*04:44, DQB1*04:45, DQB1*04:46N, DQB1*04:47, DQB1*04:48, DQB1*04:49, DQB1*04:50, DQB1*04:51, DQB1*04:52, DQB1*04:53, DQB1*04:54, DQB1*04:55, DQB1*04:56, DQB1*04:57, DQB1*04:58, DQB1*04:59N, DQB1*04:60, DQB1*04:61, DQB1*04:62, DQB1*05:01:01:01, DQB1*05:01:01:02, DQB1*05:01:01:03, DQB1*05:01:01:04, DQB1*05:01:01:05, DQB1*05:01:02, DQB1*05:01:03, DQB1*05:01:04, DQB1*05:01:05, DQB1*05:01:06, DQB1*05:01:07, DQB1*05:01:08, DQB1*05:01:09, DQB1*05:01:10, DQB1*05:01:11, DQB1*05:01:12, DQB1*05:01:13, DQB1*05:01:14, DQB1*05:01:15, DQB1*05:01:16, DQB1*05:01:17, DQB1*05:01:18, DQB1*05:01:19, DQB1*05:01:20, DQB1*05:01:21, DQB1*05:01:22, DQB1*05:01:23, DQB1*05:01:24:01, DQB1*05:01:24:02, DQB1*05:01:25, DQB1*05:01:26, DQB1*05:01:27, DQB1*05:01:28, DQB1*05:01:29, DQB1*05:01:30, DQB1*05:01:31, DQB1*05:01:32, DQB1*05:01:33, DQB1*05:01:34, DQB1*05:02:01:01, DQB1*05:02:01:02, DQB1*05:02:01:03, DQB1*05:02:01:04, DQB1*05:02:01:05, DQB1*05:02:01:06, DQB1*05:02:02, DQB1*05:02:03, DQB1*05:02:04, DQB1*05:02:05, DQB1*05:02:06, DQB1*05:02:07, DQB1*05:02:08, DQB1*05:02:09, DQB1*05:02:10, DQB1*05:02:11, DQB1*05:02:12, DQB1*05:02:13, DQB1*05:02:14, DQB1*05:02:15, DQB1*05:02:16, DQB1*05:02:17, DQB1*05:02:18, DQB1*05:02:19, DQB1*05:03:01:01, DQB1*05:03:01:02, DQB1*05:03:01:03, DQB1*05:03:02, DQB1*05:03:03, DQB1*05:03:04, DQB1*05:03:05, DQB1*05:03:06, DQB1*05:03:07, DQB1*05:03:08, DQB1*05:03:09, DQB1*05:03:10, DQB1*05:03:11, DQB1*05:03:12, DQB1*05:03:13, DQB1*05:03:14, DQB1*05:03:15, DQB1*05:03:16, DQB1*05:03:17, DQB1*05:03:18, DQB1*05:03:19, DQB1*05:03:20, DQB1*05:04, DQB1*05:05:01, DQB1*05:05:02, DQB1*05:06:01, DQB1*05:06:02, DQB1*05:07, DQB1*05:08, DQB1*05:09, DQB1*05:10, DQB1*05:100, DQB1*05:101, DQB1*05:102, DQB1*05:103, DQB1*05:104, DQB1*05:105, DQB1*05:106, DQB1*05:107, DQB1*05:108, DQB1*05:109, DQB1*05:11ON, DQB1*05:111, DQB1*05:112, DQB1*05:113, DQB1*05:114, DQB1*05:115, DQB1*05:116, DQB1*05:117, DQB1*05:118, DQB1*05:119, DQB1*05:11:01, DQB1*05:11:02, DQB1*05:12, DQB1*05:120, DQB1*05:121, DQB1*05:122, DQB1*05:123, DQB1*05:124, DQB1*05:125, DQB1*05:126, DQB1*05:127, DQB1*05:128N, DQB1*05:129, DQB1*05:13, DQB1*05:130, DQB1*05:131, DQB1*05:132Q, DQB1*05:133, DQB1*05:134, DQB1*05:135, DQB1*05:136, DQB1*05:137, DQB1*05:138, DQB1*05:139, DQB1*05:14, DQB1*05:140, DQB1*05:141, DQB1*05:142, DQB1*05:143, DQB1*05:144, DQB1*05:145, DQB1*05:146, DQB1*05:147, DQB1*05:148, DQB1*05:149, DQB1*05:15, DQB1*05:150, DQB1*05:151, DQB1*05:152, DQB1*05:153, DQB1*05:154, DQB1*05:155, DQB1*05:156, DQB1*05:157, DQB1*05:158, DQB1*05:159, DQB1*05:16, DQB1*05:160, DQB1*05:161, DQB1*05:162, DQB1*05:163, DQB1*05:164, DQB1*05:165, DQB1*05:166, DQB1*05:167, DQB1*05:168, DQB1*05:169, DQB1*05:17, DQB1*05:170, DQB1*05:171, DQB1*05:172, DQB1*05:173, DQB1*05:174, DQB1*05:175, DQB1*05:176, DQB1*05:177, DQB1*05:178, DQB1*05:179, DQB1*05:18, DQB1*05:180, DQB1*05:181, DQB1*05:182, DQB1*05:183, DQB1*05:184, DQB1*05:185N, DQB1*05:186, DQB1*05:187, DQB1*05:188, DQB1*05:189, DQB1*05:19, DQB1*05:190, DQB1*05:191, DQB1*05:192, DQB1*05:193, DQB1*05:194, DQB1*05:195, DQB1*05:196, DQB1*05:197, DQB1*05:198, DQB1*05:199, DQB1*05:20, DQB1*05:200, DQB1*05:201, DQB1*05:202, DQB1*05:203, DQB1*05:204, DQB1*05:205, DQB1*05:206N, DQB1*05:207, DQB1*05:208N5 bp DQB1*05:209, DQB1*05:21, DQB1*05:210, DQB1*05:211, DQB1*05:212, DQB1*05:213, DQB1*05:214, DQB1*05:215N, DQB1*05:216, DQB1*05:217, DQB1*05:22, DQB1*05:23, DQB1*05:24, DQB1*05:25, DQB1*05:26, DQB1*05:27, DQB1*05:28, DQB1*05:29, DQB1*05:30, DQB1*05:31, DQB1*05:32, DQB1*05:33, DQB1*05:34, DQB1*05:35, DQB1*05:36, DQB1*05:37, DQB1*05:38, DQB1*05:39, DQB1*05:40, DQB1*05:41N, DQB1*05:42, DQB1*05:43:01, DQB1*05:43:02, DQB1*05:44, DQB1*05:45, DQB1*05:46, DQB1*05:47, DQB1*05:48, DQB1*05:49, DQB1*05:50, DQB1*05:51, DQB1*05:52, DQB1*05:53, DQB1*05:54, DQB1*05:55, DQB1*05:56, DQB1*05:57, DQB1*05:58, DQB1*05:59, DQB1*05:60, DQB1*05:61, DQB1*05:62, DQB1*05:63, DQB1*05:64, DQB1*05:65, DQB1*05:66:01, DQB1*05:66:02, DQB1*05:67, DQB1*05:68, DQB1*05:69, DQB1*05:70, DQB1*05:71, DQB1*05:72, DQB1*05:73, DQB1*05:74, DQB1*05:75, DQB1*05:76, DQB1*05:77, DQB1*05:78, DQB1*05:79, DQB1*05:80, DQB1*05:81, DQB1*05:82, DQB1*05:83, DQB1*05:84, DQB1*05:85, DQB1*05:86, DQB1*05:87Q, DQB1*05:88, DQB1*05:89:01, DQB1*05:89:02, DQB1*05:90N, DQB1*05:91, DQB1*05:92, DQB1*05:93, DQB1*05:94, DQB1*05:95, DQB1*05:96, DQB1*05:97, DQB1*05:98, DQB1*05:99, DQB1*06:01:01:01, DQB1*06:01:01:02, DQB1*06:01:02, DQB1*06:01:03, DQB1*06:01:04, DQB1*06:01:05, DQB1*06:01:06, DQB1*06:01:07, DQB1*06:01:08, DQB1*06:01:09, DQB1*06:01:10, DQB1*06:01:11, DQB1*06:01:12, DQB1*06:01:13, DQB1*06:01:14, DQB1*06:01:15, DQB1*06:01:16, DQB1*06:01:17, DQB1*06:01:18, DQB1*06:01:19, DQB1*06:01:20, DQB1*06:01:21, DQB1*06:02:01:01, DQB1*06:02:01:02, DQB1*06:02:01:03, DQB1*06:02:01:04, DQB1*06:02:02, DQB1*06:02:03, DQB1*06:02:04, DQB1*06:02:05, DQB1*06:02:06, DQB1*06:02:07, DQB1*06:02:08, DQB1*06:02:09, DQB1*06:02:10, DQB1*06:02:11, DQB1*06:02:12, DQB1*06:02:13, DQB1*06:02:14, DQB1*06:02:15, DQB1*06:02:16, DQB1*06:02:17, DQB1*06:02:18, DQB1*06:02:19, DQB1*06:02:20, DQB1*06:02:21, DQB1*06:02:22, DQB1*06:02:23, DQB1*06:02:24, DQB1*06:02:25, DQB1*06:02:26, DQB1*06:02:27, DQB1*06:02:28, DQB1*06:02:29, DQB1*06:02:30, DQB1*06:02:31, DQB1*06:02:32, DQB1*06:02:33, DQB1*06:02:34, DQB1*06:02:35, DQB1*06:02:36, DQB1*06:02:37, DQB1*06:02:38, DQB1*06:03:01:01, DQB1*06:03:01:02, DQB1*06:03:01:03, DQB1*06:03:02, DQB1*06:03:03, DQB1*06:03:04, DQB1*06:03:05, DQB1*06:03:06, DQB1*06:03:07, DQB1*06:03:08, DQB1*06:03:09, DQB1*06:03:10, DQB1*06:03:11, DQB1*06:03:12, DQB1*06:03:13, DQB1*06:03:14, DQB1*06:03:15, DQB1*06:03:16, DQB1*06:03:17, DQB1*06:03:18, DQB1*06:03:19, DQB1*06:03:20, DQB1*06:03:21, DQB1*06:03:22, DQB1*06:03:23, DQB1*06:03:24, DQB1*06:03:25, DQB1*06:03:26, DQB1*06:03:27, DQB1*06:03:28, DQB1*06:03:29, DQB1*06:03:30, DQB1*06:03:31, DQB1*06:03:32, DQB1*06:03:33, DQB1*06:03:34, DQB1*06:03:35, DQB1*06:04:01, DQB1*06:04:02, DQB1*06:04:03, DQB1*06:04:04, DQB1*06:04:05, DQB1*06:04:06, DQB1*06:04:07, DQB1*06:04:08, DQB1*06:04:09, DQB1*06:04:10, DQB1*06:04:11, DQB1*06:04:12, DQB1*06:05:01, DQB1*06:05:02, DQB1*06:06, DQB1*06:07:01, DQB1*06:07:02, DQB1*06:08:01, DQB1*06:08:02, DQB1*06:08:03, DQB1*06:09:01:01 DQB1*06:09:01:02, DQB1*06:09:02, DQB1*06:09:03, DQB1*06:09:04, DQB1*06:09:05 DQB1*06:09:06, DQB1*06:09:07, DQB1*06:09:08, DQB1*06:09:09, DQB1*06:09:10, DQB1*06:10, DQB1*06:100, DQB1*06:101, DQB1*06:102N, DQB1*06:103, DQB1*06:104, DQB1*06:105, DQB1*06:106, DQB1*06:107, DQB1*06:108, DQB1*06:109, DQB1*06:110, DQB1*06:111, DQB1*06:112N, DQB1*06:113, DQB1*06:114, DQB1*06:115, DQB1*06:116, DQB1*06:117, DQB1*06:118:01, DQB1*06: 118:02, DQB1*06:118:03, DQB1*06:119, DQB1*06:11:01, DQB1*06: 11:02, DQB1*06:11:03, DQB1*06:11:04, DQB1*06:12, DQB1*06:120, DQB1*06:121, DQB1*06:122, DQB1*06:123, DQB1*06:124, DQB1*06:125, DQB1*06:126, DQB1*06:127, DQB1*06:128, DQB1*06:129, DQB1*06:130, DQB1*06:131, DQB1*06:132, DQB1*06:133, DQB1*06:134, DQB1*06:135, DQB1*06:136, DQB1*06:137, DQB1*06:138, DQB1*06:139, DQB1*06:13:01, DQB1*06:13:02, DQB1*06:13:03, DQB1*06:140, DQB1*06:141, DQB1*06:142, DQB1*06:143, DQB1*06:144N, DQB1*06:145, DQB1*06:146:01, DQB1*06:146:02, DQB1*06:147, DQB1*06:148, DQB1*06:149, DQB1*06:14:01, DQB1*06:14:02, DQB1*06:14:03, DQB1*06:150, DQB1*06:151, DQB1*06:152, DQB1*06:153:01, DQB1*06:153:02, DQB1*06:154, DQB1*06:155, DQB1*06:156, DQB1*06:157, DQB1*06:158N, DQB1*06:159, DQB1*06:15:01, DQB1*06:15:02, DQB1*06:16, DQB1*06:160, DQB1*06:161, DQB1*06:162, DQB1*06:163, DQB1*06:164, DQB1*06:165, DQB1*06:166, DQB1*06:167, DQB1*06:168, DQB1*06:169, DQB1*06:17, DQB1*06:170, DQB1*06:171, DQB1*06:172, DQB1*06:173, DQB1*06:174, DQB1*06:175, DQB1*06:176, DQB1*06:177, DQB1*06:178, DQB1*06:179N, DQB1*06:180, DQB1*06:181, DQB1*06:182, DQB1*06:183, DQB1*06:184, DQB1*06:185, DQB1*06:186, DQB1*06:187, DQB1*06:188, DQB1*06:189, DQB1*06:18:01, DQB1*06:18:02, DQB1*06:190:01, DQB1*06:190:02, DQB1*06:191, DQB1*06:192, DQB1*06:193N, DQB1*06:194, DQB1*06:195, DQB1*06:196, DQB1*06:197, DQB1*06:198, DQB1*06:199, DQB1*06:19:01, DQB1*06:19:02, DQB1*06:20, DQB1*06:200, DQB1*06:201, DQB1*06:202, DQB1*06:203, DQB1*06:204, DQB1*06:205, DQB1*06:206:01, DQB1*06:206:02, DQB1*06:207, DQB1*06:208, DQB1*06:209, DQB1*06:21, DQB1*06:210, DQB1*06:211, DQB1*06:212, DQB1*06:213, DQB1*06:214, DQB1*06:215, DQB1*06:216N, DQB1*06:217, DQB1*06:218, DQB1*06:219, DQB1*06:221, DQB1*06:222, DQB1*06:223, DQB1*06:224, DQB1*06:225, DQB1*06:226, DQB1*06:227, DQB1*06:228, DQB1*06:229, DQB1*06:22:01, DQB1*06:22:02, DQB1*06:22:03, DQB1*06:23, DQB1*06:230, DQB1*06:231, DQB1*06:232, DQB1*06:233, DQB1*06:234, DQB1*06:235, DQB1*06:236, DQB1*06:237, DQB1*06:238, DQB1*06:239, DQB1*06:24, DQB1*06:240, DQB1*06:241, DQB1*06:242, DQB1*06:243, DQB1*06:244, DQB1*06:245, DQB1*06:246, DQB1*06:247, DQB1*06:248, DQB1*06:249, DQB1*06:25, DQB1*06:250, DQB1*06:251, DQB1 06:252N, DQB1*06:253, DQB1*06:254, DQB1*06:255, DQB1*06:256, DQB1*06:257, DQB1*06:258, DQB1*06:259, DQB1*06:260, DQB1*06:261, DQB1*06:262, DQB1*06:263, DQB1*06:264, DQB1*06:265, DQB1*06:266, DQB1*06:267, DQB1*06:268, DQB1*06:269, DQB1*06:26N, DQB1*06:270:01, DQB1*06:270:02, DQB1*06:271, DQB1*06:272, DQB1*06:273, DQB1*06:274, DQB1*06:275, DQB1*06:276, DQB1*06:277, DQB1*06:278, DQB1*06:279, DQB1*06:27:01, DQB1*06:27:02, DQB1*06:28, DQB1*06:280, DQB1*06:281, DQB1*06:282, DQB1*06:283, DQB1*06:284, DQB1*06:285, DQB1*06:286, DQB1*06:287, DQB1*06:288, DQB1*06:289, DQB1*06:29, DQB1*06:290, DQB1*06:291, DQB1*06:292, DQB1*06:293, DQB1*06:294, DQB1*06:295, DQB1*06:296, DQB1*06:297, DQB1*06:298, DQB1*06:299, DQB1*06:30, DQB1*06:300, DQB1*06:301, DQB1*06:302, DQB1*06:303N, DQB1*06:304N, DQB1*06:305, DQB1*06:306N, DQB1*06:307, DQB1*06:308N, DQB1*06:309, DQB1*06:31, DQB1*06:310, DQB1*06:311, DQB1*06:312, DQB1*06:313, DQB1*06:314, DQB1*06:315, DQB1*06:316, DQB1*06:317N, DQB1*06:318, DQB1*06:319, DQB1*06:320, DQB1*06:321, DQB1*06:322, DQB1*06:323, DQB1*06:324, DQB1*06:325, DQB1*06:326, DQB1*06:32:01, DQB1*06:32:02, DQB1*06:33, DQB1*06:34, DQB1*06:35, DQB1*06:36, DQB1*06:37, DQB1*06:38, DQB1*06:39, DQB1*06:40, DQB1*06:41, DQB1*06:42, DQB1*06:43, DQB1*06:44, DQB1*06:45, DQB1*06:46, DQB1*06.47, DQB1*06:48:01, DQB1*06:48:02, DQB1*06:49, DQB1*06:50, DQB1*06:51:01, DQB1*06:51:02, DQB1*06:52, DQB1*06:53:01, DQB1*06:53:02, DQB1*06:54N, DQB1*06:55, DQB1*06:56, DQB1*06:57, DQB1*06:58, DQB1*06:59, DQB1*06:60, DQB1*06:61, DQB1*06:62, DQB1*06:63, DQB1*06:64, DQB1*06:65, DQB1*06:66, DQB1*06:67, DQB1*06:68, DQB1*06:69:01, DQB1*06:69:02, DQB1*06:70, DQB1*06:71, DQB1*06:72, DQB1*06:73, DQB1*06:74, DQB1*06:75NX, DQB1*06:76, DQB1*06:77N, DQB1*06:78, DQB1*06:79:01, DQB1*06:79:02, DQB1*06:80, DQB1*06:81, DQB1*06:82, DQB1*06:83, DQB1*06:84, DQB1*06:85, DQB1*06:86, DQB1*06:87, DQB1*06:88, DQB1*06:89, DQB1*06:90, DQB1*06:91, DQB1*06:92:01, DQB1*06:92:02, DQB1*06:93, DQB1*06:94, DQB1*06:95, DQB1*06:96:01, DQB1*06:96:02, DQB1*06:97, DQB1*06:98, DQB1*06:99:01, DQB1*06:99:02, and any combination thereof.

### II.D.3. HLA-DR Class II Molecules

In some aspects, the alpha chain is an HLA-DR alpha chain. Any HLA-DR alpha chain allele known in the art can be used in the compositions and methods disclosed herein. In some aspects, the alpha chain is an HLA-DRA*01 allele. In some aspects, the alpha chain is an HLA-DRA1 allele selected from *01:01:01:01, *01:01:01:02, *01:01:01:03, *01:01:02, *01:02:01, *01:02:02, *01:02:03, and any combination thereof.

In some aspects, the beta chain is an HLA-DR beta chain. Any HLA-DR beta chain allele known in the art can be used in the compositions and methods disclosed herein. In some aspects the beta chain is selected from an HLA-DRB1*01, HLA-DRB1*03, HLA-DRB1*04, HLA-DRB1*07, HLA-DRB1*08, HLA-DRB1*09, HLA-DRB1*10, HLA-DRB1*11, HLA-DRB1*12, HLA-DRB1*13, HLA-DRB1*14, HLA-DRB1*15, and HLA-DRB1*16 allele. In some aspects, the beta chain is a DRB3 allele. In some aspects, the beta chain is a DRB4 allele. In some aspects, the beta chain is a DRB5 allele.

In some aspects the beta chain is selected from DRB1*01:01:01, DRB1*01:01:02, DRB1*01:01:03, DRB1*01:01:04, DRB1*01:01:05, DRB1*01:01:06, DRB1*01:01:07, DRB1*01:01:08, DRB1*01:01:09, DRB1*01:01:10, DRB1*01:01:11, DRB1*01:01:12, DRB1*01:01:13, DRB1*01:01:14, DRB1*01:01:15, DRB1*01:01:16, DRB1*01:01:17, DRB1*01:01:18, DRB1*01:01:19, DRB1*01:01:20, DRB1*01:01:21, DRB1*01:01:22, DRB1*01:01:23, DRB1*01:01:24, DRB1*01:01:25, DRB1*01:01:26, DRB1*01:01:27, DRB1*01:01:28, DRB1*01:01:29, DRB1*01:01:30, DRB1*01:01:31, DRB1*01:01:32, DRB1*01:01:33, DRB1*01:02:01:01, DRB1*01:02:01:02, DRB1*01:02:02, DRB1*01:02:03, DRB1*01:02:04, DRB1*01:02:05, DRB1*01:02:06, DRB1*01:02:07, DRB1*01:02:08, DRB1*01:02:09, DRB1*01:02:10, DRB1*01:02:11, DRB1*01:02:12, DRB1*01:02:13, DRB1*01:03:01, DRB1*01: 03:02, DRB1*01:03:03, DRB1*01:03:04, DRB1*01:04, DRB1*01:05, DRB1*01:06, DRB1*01:07, DRB1*01:08, DRB1*01:09, DRB1*01:10, DRB1*01:100, DRB1*01:11:01, DRB1*01:11:02, DRB1*01:12, DRB1*01:13, DRB1*01:14, DRB1*01:15, DRB1*01:16, DRB1*01:17, DRB1*01:18:01, DRB1*01:18:02, DRB1*01:19 DRB1*01:20:01, DRB1*01:20:02, DRB1*01:21, DRB1*01:22, DRB1*01:23, DRB1*01:24:01, DRB1*01:24:02, DRB1*01:25, DRB1*01:26, DRB1*01:27, DRB1*01:28, DRB1*01:29:01, DRB1*01:29:02, DRB1*01:30, DRB1*01:31, DRB1*01:32, DRB1*01:33N, DRB1*01:34, DRB1*01:35, DRB1*01:36, DRB1*01:37, DRB1*01:38, DRB1*01:39N, DRB1*01:40N, DRB1*01:41, DRB1*01:42, DRB1*01:43, DRB1*01:44:01, DRB1*01:44:02, DRB1*01:45, DRB1*01:46, DRB1*01:47, DRB1*01:48, DRB1*01:49, DRB1*01:50, DRB1*01:51, DRB1*01:52N, DRB1*01:53, DRB1*01:54, DRB1*01:55, DRB1*01:56, DRB1*01:57, DRB1*01:58, DRB1*01:59, DRB1*01:60, DRB1*01:61, DRB1*01:62N, DRB1*01:63, DRB1*01:64, DRB1*01:65:01, DRB1*01:65:02, DRB1*01:66, DRB1*01:67, DRB1*01:68N, DRB1*01:69, DRB1*01:70, DRB1*01:71, DRB1*01:72, DRB1*01:73, DRB1*01:74, DRB1*01:75, DRB1*01:76, DRB1*01:77, DRB1*01:78, DRB1*01:79, DRB1*01:80, DRB1*01:81, DRB1*01:82, DRB1*01:83, DRB1*01:84, DRB1*01:85, DRB1*01:86, DRB1*01:87, DRB1*01:88, DRB1*01:89 DRB1*01:90, DRB1*01:91Q, DRB1*01:92, DRB1*01:93, DRB1*01:94, DRB1*01:95, DRB1*01:96, DRB1*01:97, DRB1*01:98, DRB1*01:99, DRB1*03:01:01:01, DRB1*03:01:01:02, DRB1*03:01:01:03, DRB1*03:01:02, DRB1*03:01:03, DRB1*03:01:04, DRB1*03:01:05, DRB1*03:01:06, DRB1*03:01:07, DRB1*03:01: 08, DRB1*03:01:09, DRB1*03:01:10, DRB1*03:01:11, DRB1*03:01:12, DRB1*03:01:13, DRB1*03:01:14, DRB1*03:01:15, DRB1*03:01:16, DRB1*03:01:17, DRB1*03:01:18, DRB1*03:01:19, DRB1*03:01:20, DRB1*03:01:21, DRB1*03:01:22, DRB1*03:01:23, DRB1*03:01:24, DRB1*03:01:25, DRB1*03:01:26, DRB1*03:01:27, DRB1*03:01:28, DRB1*03:02:01, DRB1*03:02:02, DRB1*03:02:03, DRB1*03:03, DRB1*03:04:01, DRB1*03:04:02, DRB1*03:05:01, DRB1*03:05:02, DRB1*03:05:03, DRB1*03:06, DRB1*03:07:01, DRB1*03:07:02, DRB1*03:08, DRB1*03:09, DRB1*03:10, DRB1*03:100:01, DRB1*03:100:02, DRB1*03:101, DRB1*03:102, DRB1*03:103, DRB1*03:104, DRB1*03:105, DRB1*03: 106, DRB 1*03:107, DRB1*03:108, DRB1*03:109, DRB1*03:110, DRB1*03:111, DRB1*03:112, DRB1*03:113, DRB1*03:114, DRB1*03:115, DRB1*03:116, DRB1*03:117, DRB1*03:118, DRB1*03:119, DRB1*03:11:01, DRB1*03:12, DRB1*03:120, DRB1*03:121, DRB1*03:122, DRB1*03:123, DRB1*03:124, DRB1*03:125, DRB1*03:126, DRB1*03:127, DRB1*03:128, DRB1*03:129, DRB1*03:130, DRB1*03:131, DRB1*03:132, DRB1*03:133, DRB1*03:134, DRB1*03:135, DRB1*03:136, DRB1*03:137, DRB1*03:138, DRB1*03:139, DRB1*03:13:01, DRB1*03:13:02, DRB1*03:14, DRB1*03:140, DRB1*03:141, DRB1*03:142, DRB1*03:143, DRB1*03:144, DRB1*03:145, DRB1*03:146, DRB1*03:147, DRB1*03:148, DRB1*03:149, DRB1*03:150, DRB1*03:151, DRB*03:152, DRB1*03:153, DRB1*03:154, DRB1*03:155, DRB1*03:156N, DRB1*03:157, DRB1*03:158, DRB1*03:15:01, DRB1*03:15:02, DRB1*03:16, DRB1*03:17, DRB1*03:18, DRB1*03:19, DRB1*03:20, DRB1*03:21, DRB1*03:22, DRB1*03:23, DRB1*03:24, DRB1*03:25:01, DRB1*03:25:02, DRB1*03:26, DRB1*03:27, DRB1*03:28, DRB1*03:29, DRB1*03:30, DRB1*03:31, DRB1*03:32, DRB1*03:33, DRB1*03:34, DRB1*03:35, DRB1*03:36, DRB1*03:37, DRB1*03:38, DRB1*03:39 DRB1*03:40, DRB1*03:41:01, DRB1*03:41:02, DRB1*03:42, DRB1*03:43, DRB1*03:44, DRB1*03:45, DRB1*03:46, DRB1*03:47, DRB1*03:48, DRB1*03:49, DRB1*03:50, DRB1*03:51, DRB1*03:52, DRB1*03:53, DRB1*03:54, DRB1*03:55, DRB1*03:56, DRB1*03:57, DRB1*03:58, DRB1*03:59, DRB1*03:60, DRB1*03:61, DRB1*03:62, DRB1*03:63, DRB1*03:64, DRB1*03:65, DRB1*03:66, DRB1*03:67N, DRB1*03:68N, DRB1*03:69, DRB1*03:70, DRB1*03:71:01, DRB1*03:71:02, DRB1*03:72, DRB1*03:73, DRB1*03:74, DRB1*03:75, DRB1*03:76, DRB1*03:77, DRB1*03:78, DRB1*03:79, DRB1*03:80, DRB1*03:81, DRB1*03:82, DRB1*03:83, DRB1*03:84, DRB1*03:85, DRB1*03:86, DRB1*03:87, DRB1*03:88, DRB1*03:89, DRB1*03:90, DRB1*03:91, DRB1*03:92, DRB1*03: 93, DRB1*03:94, DRB1*03:95, DRB1*03:96, DRB1*03:97, DRB1*03:98, DRB1*03:99, DRB1*04: 01:01:01, DRB1*04:01:01:02, DRB1*04:01:01:03, DRB1*04:01:02, DRB1*04:01:03, DRB1*04:01:04, DRB1*04:01:05, DRB1*04:01:06, DRB1*04:01:07, DRB1*04:01:08, DRB1*04:01:09, DRB*04:01:10, DRB1*04:01:11, DRB1*04:01:12, DRB1*04:01:13, DRB1*04:01:14, DRB1*04:01:15, DRB1*04:01:16, DRB1*04:01:17, DRB1*04:01:18, DRB1*04:01:19, DRB1*04:01:20, DRB1*04:01:21, DRB1*04:02:01, DRB1*04:02:02, DRB1*04:02:03, DRB1*04:02:04, DRB1*04:02:05, DRB1*04:02:06, DRB1*04:03::01, DRB*04:03:01:02, DRB*04:03:02, DRB1*04:03:03, DRB1*04:03:04, DRB1*04:03:05, DRB1*04:03:06, DRB1*04:03:07, DRB1*04:03:08, DRB1*04:03:09, DRB1*04:03:10, DRB1*04:03:11, DRB1*04:03:12, DRB1*04:03:13, DRB1*04:03:14, DRB1*04:03:15, DRB1*04:04:01, DRB1*04:04:02, DRB1*04:04:03, DRB1*04:04:04, DRB1*04:04:05, DRB1*04:04:06, DRB1*04:04:07, DRB1*04:04:08, DRB1*04:04:09, DRB1*04:04:10, DRB1*04:04:11, DRB1*04:04:12, DRB1*04:04:13, DRB1*04:04:14, DRB1*04:04:15, DRB1*04:05:01:01, DRB1*04:05:01:02, DRB1*04:05:01:03, DRB1*04:05:02, DRB1*04:05:03, DRB1*04:05:04, DRB1*04:05:05, DRB1*04:05:06, DRB1*04:05:07, DRB1*04:05:08, DRB1*04:05:09, DRB1*04:05:10, DRB1*04:05:11, DRB1*04:05:13, DRB1*04:05:14, DRB1*04:05:15, DRB1*04:05:16, DRB1*04:05:17, DRB1*04:05:18, DRB1*04:05:19, DRB1*04:05:20, DRB1*04:06:01, DRB1*04:06:02, DRB1*04:06:03, DRB1*04:06:04, DRB1*04:06:05, DRB1*04:06:06, DRB1*04:06:07, DRB1*04:07:01:01, DRB1*04:07:01:02, DRB1*04:07:02, DRB1*04:07:03, DRB1*04:07:04, DRB1*04:07:05, DRB1*04:07:06, DRB1*04:08:01, DRB1*04:08:02, DRB1*04:08:03, DRB1*04:08:04, DRB1*04:09, DRB1*04:100, DRB1*04:101, DRB1*04:102, DRB1*04:103, DRB1*04:104, DRB1*04:105:01, DRB1*04:105:02, DRB1*04:106, DRB1*04:107, DRB1*04:108, DRB1*04:109, DRB1*04:10:01, DRB1*04:10:02, DRB1*04:10:03, DRB1*04:110, DRB1*04:111, DRB1*04:112, DRB1*04:113, DRB1*04:114, DRB1*04:115, DRB1*04:116, DRB1*04:117, DRB1*04:118, DRB1*04:119N, DRB1*04:11:01, DRB1*04:11:02, DRB1*04:11:03, DRB1*04:11:04, DRB1*04:11:05, DRB1*04:12, DRB1*04:120N, DRB1*04:121, DRB1*04:122, DRB1*04:123, DRB1*04:124, DRB1*04:125, DRB1*04:126, DRB1*04:127, DRB1*04:128, DRB1*04:129, DRB1*04:13, DRB1*04:130, DRB1*04:131:01, DRB1*04:131:02, DRB1*04:132, DRB1*04:133, DRB1*04:134, DRB1*04:135, DRB1*04:136, DRB1*04:137, DRB1*04:138, DRB1*04:139, DRB1*04:14, DRB1*04:140, DRB1*04:141, DRB1*04:142N, DRB1*04:143, DRB1*04:144, DRB1*04:145, DRB1*04:146, DRB1*04:147, DRB1*04:148, DRB1*04:149, DRB1*04:15, DRB1*04:150, DRB1*04:151, DRB1*04:152, DRB1*04:153, DRB1*04:154, DRB1*04:155, DRB1*04:156, DRB1*04:157N, DRB1*04:158N, DRB1*04:159, DRB1*04:16, DRB1*04:160, DRB1*04:161, DRB1*04:162, DRB1*04:163, DRB1*04:164, DRB1*04:165, DRB1*04:166, DRB1*04:167, DRB1*04:168, DRB1*04:169, DRB1*04:170, DRB1*04:171, DRB1*04:172, DRB1*04:173, DRB1*04:174, DRB1*04:175, DRB1*04:176, DRB1*04:177, DRB1*04:178N, DRB1*04:179, DRB1*04:17:01, DRB1*04:17:02, DRB1*04:18, DRB1*04:180, DRB1*04:181, DRB1*04:182, DRB1*04:183, DRB1*04:184, DRB1*04:185, DRB1*04:186N, DRB1*04:187, DRB1*04:188, DRB1*04:189, DRB1*04:19, DRB1*04:190, DRB1*04:191, DRB1*04:192, DRB1*04:193, DRB1*04:194, DRB1*04:195, DRB1*04:196, DRB1*04:197, DRB1*04:198, DRB1*04:199, DRB1*04:20, DRB1*04:200, DRB1*04:201, DRB1*04:202, DRB1*04:203, DRB1*04:204, DRB1*04:205, DRB1*04:206, DRB1*04:207, DRB1*04:208, DRB1*04:209, DRB1*04:21, DRB1*04:210, DRB1*04:211, DRB1*04:212N, DRB1*04:213, DRB1*04:214N, DRB1*04:215, DRB1*04:216, DRB1*04:217, DRB1*04:218, DRB1*04:219, DRB1*04:22, DRB1*04:220, DRB1*04:221, DRB1*04:222, DRB1*04:223, DRB1*04:224, DRB1*04:225, DRB1*04:226:01, DRB1*04:226:02, DRB1*04:227, DRB1*04:228, DRB1*04:229, DRB1*04:23, DRB1*04:230, DRB1*04:231, DRB1*04:232, DRB1*04:233, DRB1*04:234, DRB1*04:235, DRB1*04:236, DRB1*04:237, DRB1*04:238, DRB1*04:239, DRB1*04:24, DRB1*04:240, DRB1*04:241, DRB1*04:242, DRB1*04:243, DRB1*04:244, DRB1*04:245, DRB1*04:246, DRB1*04:247N, DRB1*04:248, DRB1*04:249, DRB1*04:25, DRB1*04:250, DRB1*04:251, DRB1*04:252, DRB1*04:253, DRB1*04:254, DRB1*04:255, DRB1*04:256, DRB1*04:257, DRB1*04:258, DRB1*04:259, DRB1*04:26, DRB1*04:260, DRB1*04:261, DRB1*04:262, DRB1*04:263, DRB1*04:264N, DRB1*04:265, DRB1*04:266N, DRB1*04:267N, DRB1*04:268, DRB1*04:269, DRB1*04:27, DRB1*04:270, DRB1*04:271, DRB1*04:272, DRB1*04:28, DRB1*04:29, DRB1*04:30, DRB1*04:31, DRB1*04:32, DRB1*04:33, DRB1*04:34, DRB1*04:35, DRB1*04:36, DRB1*04:37, DRB1*04:38, DRB1*04:39, DRB1*04:40, DRB1*04:41, DRB1*04:42, DRB1*04:43, DRB1*04:44:01, DRB1*04:44:02, DRB1*04:45, DRB1*04:46, DRB1*04:47, DRB1*04:48, DRB1*04:49, DRB1*04:50, DRB1*04:51, DRB1*04:52, DRB1*04:53:01, DRB1*04:53:02, DRB1*04:54, DRB 1*04:55, DRB1*04:56:01, DRB1*04:56:02, DRB1*04:57, DRB1*04:58, DRB1*04:59, DRB1*04:60, DRB1*04:61, DRB1*04:62, DRB1*04:63, DRB1*04:64, DRB1*04:65, DRB1*04:66, DRB1*04:67, DRB1*04:68, DRB1*04:69, DRB1*04:70, DRB1*04:71, DRB1*04:72:01, DRB1*04:72:02, DRB1*04:73:01, DRB1*04:73:02, DRB1*04:74, DRB1*04:75, DRB1*04:76, DRB1*04:77, DRB1*04:78, DRB1*04:79, DRB1*04:80, DRB1*04:81N, DRB1*04:82, DRB1*04:83, DRB1*04:84, DRB1*04:85, DRB1*04: 86, DRB1*04: 87, DRB1*04: 88, DRB1*04: 89, DRB1*04: 90, DRB1*04:91 DRB1*04:92, DRB1*04: 93, DRB1*04:94:01N, DRB1*04:95:01, DRB1*04: 95:02, DRB1*04:96, DRB1*04:97, DRB1*04:98:01, DRB1*04:98:02, DRB1*04:99, DRB1*07:01:01:01, DRB1*07:01:01:02, DRB1*07:01:01:03, DRB1*07:01:01:04, DRB1*07:01: 02, DRB1*07:01:03, DRB1*07:01:04, DRB1*07:01:05, DRB*07:01:06, DRB1*07:01:07, DRB1*07:01:08, DRB1*07:01:09, DRB1*07:01:10, DRB1*07:01:11, DRB1*07:01:12, DRB1*07:01:13, DRB1*07:01:14, DRB1*07:01:15, DRB1*07:01:16, DRB1*07:01:17, DRB1*07:01:18, DRB1*07:01:19, DRB1*07:01:20, DRB1*07:01:21, DRB1*07:01:22, DRB1*07:03, DRB1*07:04, DRB1*07:05, DRB1*07:06, DRB1*07:07, DRB1*07:08, DRB1*07:09, DRB1*07:100, DRB1*07:101N, DRB1*07:1ON, DRB1*07:11, DRB1*07:12, DRB1*07:13, DRB1*07:14, DRB1*07:15, DRB1*07:16, DRB1*07:17, DRB1*07:18, DRB1*07:19, DRB1*07:20, DRB1*07:21, DRB1*07:22, DRB1*07:23, DRB1*07:24, DRB1*07:25, DRB1*07:26N, DRB1*07:27, DRB1*07:28, DRB1*07:29, DRB1*07:30, DRB1*07:31, DRB1*07:32, DRB1*07:33, DRB1*07:34, DRB1*07:35, DRB1*07:36, DRB1*07:37, DRB1*07:38, DRB1*07:39, DRB1*07:40, DRB1*07:41, DRB1*07:42, DRB1*07:43, DRB1*07:44, DRB1*07:45, DRB1*07:46, DRB1*07:47, DRB1*07:48, DRB1*07:49, DRB1*07:50, DRB1*07:51, DRB1*07:52, DRB1*07:53, DRB1*07:54, DRB1*07:55, DRB1*07:56, DRB1*07:57, DRB1*07:58N, DRB1*07:59 DRB1*07:60, DRB1*07:61, DRB1*07:62, DRB1*07:63, DRB1*07:64, DRB1*07:65, DRB1*07:66, DRB1*07:67, DRB1*07:68N, DRB1*07:69, DRB1*07:70, DRB1*07:71, DRB1*07:72, DRB1*07:73, DRB1*07:74, DRB1*07:75, DRB1*07:76, DRB1*07:77, DRB1*07:78, DRB1*07:79, DRB1*07:80, DRB1*07:81, DRB1*07:82, DRB1*07:83, DRB1*07: 84, DRB1*07: 85, DRB1*07:86, DRB1*07:87N, DRB1*07:88, DRB1*07:89 DRB1*07:90, DRB1*07: 91, DRB1*07: 92, DRB1*07: 93, DRB1*07:94, DRB1*07:95, DRB1*07:96, DRB1*07:97, DRB1*07:98, DRB1*07:99, DRB1*08:01:01, DRB1*08:01:02, DRB1*08:01:04, DRB1*08:01:05, DRB1*08:01:06, DRB1*08:01:07, DRB1*08:02:01:01, DRB1*08:02:01:02, DRB1*08:02:02, DRB1*08:02:03, DRB1*08:02:04, DRB1*08:03:02:01, DRB1*08:03:02:02, DRB1*08:03:03, DRB1*08:03:04, DRB1*08:03:05, DRB1*08:03:06, DRB1*08:03:07, DRB1*08:03:08, DRB1*08:03:09, DRB1*08:04:01, DRB1*08:04:02, DRB1*08:04:03, DRB1*08:04:04, DRB1*08:04:05, DRB1*08:04:06, DRB1*08:04:07, DRB1*08:05, DRB1*08:06, DRB1*08:07, DRB1*08:08, DRB1*08:09, DRB1*08:10, DRB1*08:11, DRB1*08:12, DRB1*08:13, DRB1*08:14, DRB1*08:15, DRB1*08:16, DRB1*08:17, DRB1*08:18, DRB1*08:19, DRB1*08:20, DRB1*08:21, DRB1*08:22, DRB1*08:23, DRB1*08:24:01, DRB1*08:24:02, DRB1*08:25, DRB1*08:26, DRB1*08:27, DRB1*08:28, DRB1*08:29, DRB1*08:30:01, DRB1*08:30:02, DRB1*08:30:03, DRB1*08:31, DRB1*08:32, DRB1*08:33, DRB1*08:34, DRB1*08:35, DRB1*08:36:01, DRB1*08:36:02, DRB1*08:37, DRB1*08:38, DRB1*08:39, DRB1*08:40, DRB1*08:41, DRB1*08:42, DRB1*08:43, DRB1*08:44, DRB1*08:45:01, DRB1*08:45:02, DRB1*08:46, DRB1*08:47, DRB1*08:48, DRB1*08:49, DRB1*08:50, DRB1*08:51, DRB1*08:52, DRB1*08:53, DRB1*08:54, DRB1*08:55, DRB1*08:56, DRB1*08:57, DRB1*08:58, DRB1*08:59, DRB1*08:60N, DRB1*08:61, DRB1*08:62, DRB1*08:63, DRB1*08:64, DRB1*08:65, DRB1*08:66, DRB1*08:67, DRB1*08:68:01, DRB1*08:68:02, DRB1*08:69 DRB1*08:70, DRB1*08:71, DRB1*08:72, DRB1*08:73, DRB1*08:74, DRB1*08:75, DRB1*08:76, DRB1*08:77, DRB1*08:78N, DRB1*08:79, DRB1*08:80, DRB1*08:81, DRB1*08:82, DRB1*08:83, DRB1*08:84, DRB1*08:85, DRB1*08:86, DRB1*08:87, DRB1*08:88, DRB1*08:89N, DRB1*08:90, DRB1*09:01:02:01, DRB1*09:01:02:02, DRB1*09:01:03, DRB1*09:01:04, DRB1*09:01:05, DRB1*09:01:06, DRB1*09:01:07, DRB1*09:01:08, DRB1*09:01:09, DRB1*09:01:10, DRB1*09:01:11, DRB1*09:02:01, DRB1*09:02:02, DRB1*09:03, DRB1*09:04, DRB1*09:05, DRIB*09:06, DRB1*09:07, DRB1*09:08, DRB1*09:09, DRB1*09:10, DRB1*09:11, DRB1*09:12, DRB1*09:13 DRB1*09:14, DRB1*09:15, DRB1*09:16, DRB1*09:17, DRB1*09:18, DRB1*09:19, DRB1*09:20, DRB1*09:21, DRB1*09:22, DRB1*09:23, DRB1*09:24, DRB1*09:25, DRB1*09:26, DRB1*09:27, DRB1*09:28, DRB1*09:29, DRB1*09:30, DRB1*09:31, DRB1*09:32, DRB1*09:33, DRB1*09:34, DRB1*09:35, DRB1*09:36, DRB1*09:37N, DRB1*09: 38, DRB1*09: 39, DRB1*09: 40, DRB1*10:01:01:01, DRB1*10:01:01:02, DRB1*10:01:01:03, DRB1*10:01:02, DRB1*10:01:03, DRB1*10:01:04, DRB1*10:01:05, DRB1*10:01:06, DRB1*10:01:07, DRB1*10:01:08, DRB1*10:01:09, DRB1*10:01:10, DRB1*10:01:11, DRB1*10:01:12, DRB1*10:02, DRB1*10:03, DRB1*10:04, DRB1*10:05, DRB1*10:06, DRB1*10:07, DRB1*10:08, DRB1*10:09, DRB1*10:10, DRB1*10:11, DRB1*10:12, DRB1*10:13, DRB1*10:14, DRB1*10:15, DRB1*10:16, DRB1*10:17, DRB1*10:18, DRB1*10:19, DRB1*10:20, DRB1*10:21, DRB1*10:22, DRB1*10:23, DRB1*10:24, DRB1*10:25, DRB1*10:26, DRB1*10:27, DRB1*10:28, DRB1*10:29, DRB1*10:30, DRB1*10:31, DRB1*10:32, DRB1*10:33, DRB1*11:01:01:01, DRB1*11:01:01:02, DRB1*11:01:01:03, DRB1*11:01:01:04, DRB1*11:01:02, DRB1*11:01:03, DRB1*11:01:04, DRB1*11:01:05, DRB1*11:01:06, DRB1*11:01:07, DRB1*11:01:08, DRB1*11:01:09, DRB1*11:01:10, DRB1*11:01:11, DRB1*11:01:12, DRB1*11:01:13, DRB1*11:01:14, DRB1*11:01:15, DRB1*11:01:16, DRB1*11:01:17, DRB1*11:01:18, DRB1*11:01:19, DRB1*11:01:20, DRB1*11:01:21, DRB1*11:01:22, DRB1*11:01:23, DRB1*11:01:24, DRB1*11:01:25, DRB1*11:01:26, DRB1*11:01:27, DRB1*11:01:28, DRB1*11:01:29, DRB1*11:01:30, DRB1*11:01:31, DRB1*11:01:32, DRB1*11:01:33, DRB1*11:02:01, DRB1*11:02:02, DRB1*11:02:03, DRB1*11:02:04, DRB1*11:02:05, DRB1*11:03:01, DRB1*11:03:02, DRB1*11:03:03, DRB1*11:03:04, DRB1*11:04:01, DRB1*11:04:02, DRB1*11:04:03, DRB1*11:04:04, DRB1*11:04:05, DRB1*11: 04:06, DRB1*11: 04:07, DRB1*11:04:08, DRB1*11:04:09, DRB1*11:04:10, DRB1*11:04:11, DRB1*11:04:12, DRB1*11:04:13, DRB1*11:04:14, DRB1*11:04:15, DRB1*11:04:16, DRB1*11:04:17, DRB1*11:04:18, DRB1*11:05, DRB1*11:06:01, DRB1*11:06:02, DRB1*11:06:03, DRB1*11:07:01, DRB1*11:07:02, DRB1*11:08:01, DRB1*11:08:02, DRB1*11:08:03, DRB1*11:09, DRB1*11:100, DRB1*11:101:01, DRB1*11:101:02, DRB1*1:102:01, DRB1*11:102:02, DRB1*11:103:01, DRB1*11:103:02, DRB1*11:104, DRB1*11:105, DRB1*11:106, DRB1*11:107, DRB1*11:108, DRB1*11:109, DRB1*11:10:01, DRB1*11:10:02, DRB1*11:110, DRB1*11:111, DRB1*11:112, DRB1*11:113, DRB1*11:114, DRB1*11:115, DRB1*11:116, DRB1*11:117:01, DRB1*11:117:02, DRB1*11:118, DRB1*11:119, DRB1*11:11:01, DRB1*11: 11:03, DRB1*11:120, DRB1*11:121, DRB1*11:122, DRB1*11:123, DRB1*11:124:01, DRB1*11:124:02, DRB1*11:125, DRB1*11:126, DRB1*11:127, DRB1*11:128, DRB1*11:129, DRB1*11:12:01, DRB1*11:12:02, DRB1*11:12:03, DRB1*11:130, DRB1*11:131, DRB1*11:132, DRB1*11:133, DRB1*11:134, DRB1*11:135, DRB1*11:136, DRB1*11:137, DRB1*11:138, DRB1*11:139, DRB1*11:13:01, DRB1*11:13:02, DRB1*11:140, DRB1*L 141, DRB1*11:142, DRB1*11:143, DRB1*11:144, DRB1*11:145, DRB1*11:146, DRB1*11:147:01, DRB1*11:147:02, DRB1*11:148, DRB1*11:149, DRB1*11:14:01, DRB1*11:14:02, DRB1*11:15, DRB1*11:150, DRB1*11:151, DRB1*11:152, DRB1*11:153, DRB111:154, DRB1*11:155, DRB1*11:156, DRB1*11:157, DRB1*11:158, DRB1*11:159, DRB1*11:16, DRB1*11:160, DRB1*11:161, DRB1*11:162, DRB1*11:163, DRB1*11:164, DRB1*11:165:01, DRB1*11:165:02, DRB1*11:166, DRB1*11:167, DRB1*11:168, DRB1*11:169N, DRB1*11:17, DRB1*11:170, DRB1*11:171, DRB1*11:172, DRB1*11:173, DRB1*11:174, DRB1*11:175, DRB1*11:176, DRB1*11:177, DRB1*11:178, DRB1*11:179, DRB1*11:18, DRB1*11:180, DRB1*11:181, DRB1*11:182, DRB1*11:183, DRB1*11:184, DRB1*11:185, DRB1*11:186, DRB1*11:187, DRB1J*11:188, DRB1*11:189, DRB1*11:190, DRB1*11:191, DRB1*11:192, DRB1*11:193:01, DRB1*11:193:02, DRB1*11:194, DRB1*11:195, DRB1*11:196, DRB1*11:197, DRB1*11:198, DRB1*11:199, DRB1*11:19:01, DRB1*11:19:02 DRB1*11:19:03, DRB1*11:20, DRB1*11:200, DRB1*11:201, DRB1*11:202, DRB1*11:203, DRB1*11:204, DRB1*11:205, DRB1*11:206, DRB 1*11:207, DRB1*11:208, DRB1*11:209, DRB1*11:21, DRB1*11:210, DRB1*11:211, DRB1*11:212, DRB1*11:213, DRB1*11:214, DRB1*11:215, DRB1*11:216, DRB1*11:217N, DRB1*11:218, DRB1*11: 219, DRB1*11:22, DRB*11:220, DRB*11:221, DRB1*11: 222, DRB1*11:223, DRB1*11:224, DRB1*11:225, DRB1*11:226, DRB1*11:227, DRB1*11:228, DRB1*11:229, DRB1*11:230, DRB1*l 1:231, DRB1*11:232, DRB1*11:233, DRB1*11:234, DRB1*11:235, DRB1*11:236, DRB1*11:237, DRB*11:238, DRB1*11:239, DRB1*11:23:01, DRB1*11: 23:02, DRB*11:240, DRB*11:241, DRB*11:242, DRB1*11:243, DRB1*11:244, DRB1*11:245, DRB1*11:246N, DRB1*11:247, DRB1*11:248Q, DRB1*11:249, DRB1*11:24:01, DRB1*11:24:02, DRB1*11:25, DRB1*11:250N DRB1*11:251, DRB1*11:252, DRB1*11:253, DRB1*11:254, DRB1*11:26, DRB 1*11:27:01, DRB1*11:27:02, DRB1*11:27:03, DRB1*11:28:01, DRB1*11:28:02, DRB1*11:29:01, DRB1*11:29:02, DRB1*11:30, DRB1*11:31, DRB*l11:32, DRB1*11:33, DRB1*11:34, DRB1*11:35, DRB1*11:36, DRB1*11:37:01, DRB1*11:37:02, DRB1*11:38, DRB1*11:39, DRB1*11:40, DRB1*11:41, DRB1*11:42:01, DRB1*11:42:02, DRB1*11:43, DRB1*11:44, DRB1*11:45, DRB1*11:46:01, DRB1*11:46:02, DRB1*11:47, DRB1*11:48, DRB1*11:49:01, DRB1*11:49:02, DRB1*11:50, DRB1*11:51, DRB1*11:52, DRB1*11:53, DRB1*11:54:01, DRB1*11:54:02, DRB1*11:55, DRB1*11:56, DRB1*11:57, DRB1*11:58:01, DRB1*11:58:02, DRB1*11:59, DRB1*11:60, DRB1*11:61, DRB1*11:62:01, DRB1*11:62:02, DRB1*11:63:01, DRB1*11:63:02, DRB1*11:64, DRB1*11:65:01, DRB1*11:65:02, DRB1*11:66:01, DRB1*11:66:02, DRB1*11:67, DRB1*11:68, DRB1*11:69, DRB1*11:70, DRB1*11:72, DRB1*11:73, DRB1*11:74:01, DRB1*11:74:02, DRB1*11:75, DRB1*11:76, DRB1*11:77, DRB1*11:78, DRB1*11:79, DRB1*11:80, DRB1*11:81, DRB1*11:82, DRB1*11:83, DRB1*11:84:01, DRB1*11:84:02, DRB1*11:84:03, DRB1*11:85, DRB1*11:86, DRB1*11:87, DRB1*11:88, DRB1*11:89, DRB1*11:90, DRB1*11:91, DRB1*11:92, DRB1*11:93, DRB1*11:94, DRB1*11:95, DRB1*11: 96, DRB1*11:97, DRB1*11: 98, DRB1*11:99, DRB1*12:01:01:01, DRB1*12:01:01:02, DRB1*12:01:01:03, DRB1*12:01:01:04, DRB1*12:01:01:05, DRB1*12:01:01:06, DRB1*12:01:02, DRB1*12:01:03, DRB1*12:01:04, DRB1*12:01:05, DRB1*12:01:06, DRB1*12:01:07, DRB1*12:01:08, DRB1*12:01:09, DRB1*12:02:01:01, DRB1*12:02:01:02, DRB1*12:02:01:03, DRB1*12:02:01:04, DRB1*12:02:02, DRB1*12:02:03, DRB1*12:02:04, DRB1*12:02:05, DRB1*12:02:06, DRB1*12:02:07, DRB1*12:02:08, DRB1*12:02:09, DRB1*12:03:02, DRB1*12:03:03, DRB1*12:04, DRB1*12:05, DRB1*12:06, DRB1*12:07, DRB1*12:08, DRB1*12:09, DRB1*12:10, DRB1*12:11, DRB1*12:12, DRB1*12:13, DRB1*12:14, DRB1*12:15, DRB1*12:16:01, DRB1*12:16:02, DRB1*12:16:03, DRB1*12:17, DRB1*12:18, DRB1*12:19, DRB1*12:20, DRB1*12:21, DRB1*12:22, DRB1*12:23, DRB1*12:24N, DRB1*12:25, DRB1*12:26, DRB1*12:27, DRB1*12:28, DRB1*12:29, DRB1*12:30, DRB1*12:31N, DRB1*12:32, DRB1*12:33, DRB1*12:34, DRB1*12:35, DRB1*12:36, DRB1*12:37, DRB1*12:38, DRB1*12 39, DRB1*12:40, DRB1*12:41, DRB1*12:42, DRB1*12:43, DRB1*12:44, DRB1*12:45, DRB1*12:46, DRB1*12:47, DRB1*12:48, DRB1*12:49, DRB1*12:50, DRB1*12:51, DRB1*12:52, DRB1*12:53, DRB1*12:54, DRB1*12:55, DRB1*12:56, DRB1*12:57, DRB1*12:58, DRB1*12:59, DRB1*12:60N, DRB1*12:61, DRB1*12:62, DRB1*12:63, DRB1*12:64, DRB1*12:65, DRB1*12:66, DRB1*12:67, DRB1*12:68, DRB1*12:69, DRB1*12:70, DRB1*12:71, DRB1*12:72N, DRB1*12:73, DRB1*12:74N, DRB1*12:75, DRB1*13:01:01:01, DRB1*13:01:01:02, DRB1*13:01:02, DRB1*13:01:03, DRB1*13:01:04, DRB1*13:01:05, DRB1*13:01:06, DRB1*13:01:07, DRB1*13:01:08, DRB1*13:01:09, DRB1*13:01:10, DRB1*13:01:11, DRB1*13:01:12, DRB1*13:01:13, DRB1*13:01:14, DRB1*13:01:15, DRB1*13:01:16, DRB1*13:01:17, DRB1*13:01:18, DRB1*13:01:19, DRB1*13:01:20, DRB1*13:01:21, DRB1*13:01:22, DRB1*13:01:23, DRB1*13:01:24, DRB1*13:01:25, DRB1*13:01:26, DRB1*13:02:01:01, DRB1*13:02:01:02, DRB1*13:02:01:03, DRB1*13:02:02, DRB1*13:02:03, DRB1*13:02:04, DRB1*13:02:05, DRB1*13:02:06, DRB1*13:02:07, DRB1*13:02:08, DRB1*13:02:09, DRB1*13:02:10 DRB1*13:02:11, DRB1*13:02:12, DRB*13:02:13, DRB*13:02:14, DRB1*13:02:15, DRB1*13:02:16, DRB1*13:02:17, DRB1*13:03:01, DRB1*13:03:02, DRB1*13:03:03, DRB1*13:03:04, DRB1*13:03:05, DRB1*13:03:06, DRB1*13:03:07, DRB1*13:03:08, DRB1*13:03:09, DRB1*13:04, DRB1*13:05:01, DRB1*13:05:02, DRB1*13:05:03, DRB1*13:06, DRB1*13:07:01, DRB1*13:07:02, DRB1*13:08, DRB1*13:09, DRB1*13:10, DRB1*13: 100, DRB1*13: 101, DRB1*13:102, DRB1*13:103, DRB1*13:104, DRB1*13:105, DRB1*13:106, DRB1*13:107, DRB1*13:108, DRB1*13:109, DRB1*13:110, DRB1*13:111, DRB1*13:112, DRB1*13:113N, DRB1*13:114, DRB1*13:115, DRB1*13:116, DRB1*13:117, DRB1*13:118, DRB1*13:119, DRB1*13:11:01, DRB1*13:11:02, DRB1*13:120, DRB1*13:121, DRB1*13:122, DRB1*13:123, DRB1*13:124, DRB1*13:125, DRB1*13:126, DRB1*13:127, DRB1*13:128, DRB1*13:129, DRB1*13:12:01, DRB1*13:12:02, DRB1*13:12:03, DRB1*13:12:04, DRB1*13:13, DRB1*13:130, DRB1*13:131, DRB1*13:132, DRB1*13:133, DRB1*13:134, DRB1*13:135, DRB1*13:136, DRB1*13:137N. DRB1*13:138, DRB1*13:139, DRB1*13:140, DRB1*13:141, DRB1*13:142N, DRB1*13:143, DRB1*13:144, DRB1*13:145, DRB1*13:146, DRB1*13:147, DRB1*13:148, DRB1*13:149, DRB1*13:14:01, DRB1*13:14:02, DRB1*13:14:03, DRB1*13:15, DRB1*13:150, DRB1*13:151, DRB1*13:152, DRB1*13:153, DRB1*13:154, DRB1*13:155, DRB1*13:156, DRB1*13:157, DRB1*13:158, DRB1*13:159, DRB1*13:16, DRB1*13:160, DRB1*13:161, DRB1*13:162, DRB1*13:163, DRB1*13:164, DRB1*13:165, DRB1*13:166, DRB1*13:167, DRB1*13:168, DRB1*13:169, DRB1*13:17, DRB1*13:170, DRB1*13:171:01, DRB1*13:171:02, DRB1*13:172, DRB1*13:173, DRB1*13:174, DRB1*3:175, DRB1*13:176, DRB1*1 3:177, DRB1*13:178, DRB1*13:179, DRB1*13:18, DRB1*13:180, DRB1*13:181, DRB1*13:182, DRB1*13:183, DRB1*13:184, DRB1*13:185N, DRB1*13:186, DRB1*13:187, DRB1*13:188, DRB1*13:189, DRB1*13:19, DRB1*13:190, DRB1*13:191, DRB1*13:192, DRB1*13:193, DRB1*13:194, DRB1*13:195, DRB1*3:196, DRB1*13:197, DRB1*13:198, DRB1*13:199, DRB1*13:20, DRB1*13:200N, DRB1*13:201, DRB1*13:202, DRB1*13:203, DRB1*13:204, DRB1*13:205, DRB1*13:206, DRB1*13:207, DRB1*13:208, DRB1*13:209, DRB1*13:210, DRB1*13:211, DRB1*13:212, DRB1*13:213, DRB1*13:214, DRB1*13:215, DRB1*13:216, DRB1*13:217, DRB1*13:218, DRB1*13:219, DRB1*13:21:01, DRB1*13:21:02, DRB1*13:220, DRB1*13:221, DRB1*13:222, DRB1*13:223, DRB1*13:224, DRB1*13:225, DRB1*1*13:226, DRB1*13:227, DRB1*13:228, DRB1*13:229, DRB1*13:22:01, DRB1*13:22:02, DRB1*13:230, DRB1*13:231, DRB1*13:232, DRB1*13:233, DRB1*13:234, DRB1*13:235, DRB1*13:236, DRB1*13:237, DRB1*13:238, DRB1*13:239, DRB1*13:23:01, DRB1*13:23:02, DRB1*13:24, DRB1*13:240, DRB1*13:241, DRB1*13:242:01, DRB1*13:242:02, DRB1*13:243, DRB1*13:244, DRB1*13:245, DRB1*13:246, DRB1*13:247, DRB1*13:248, DRB1*13:249N, DRB1*13:25, DRB1*13:250, DRB1*13:251, DRB1*13:252N, DRB1*13:253, DRB1*13:254, DRB1*13:255N, DRB1*13:256, DRB1*13:257, DRB1*13:258, DRB1*13:259, DRB1*13:260, DRB11*3:261, DRB1*13:262, DRB11*13:263, DRB1*13:264, DRB1*13:265, DRB1*13:266, DRB1*13:267, DRB1*13:268N, DRB1*13:269, DRB1*13:26:01, DRB1*13:26:02, DRB1*13:27, DRB1*13:270, DRB1*13:271, DRB1*13:272, DRB11*13:273, DRB1*13:274, DRB1*3:275, DRB1*13:276, DRB11*13:277, DRB1*13:278Q, DRB1*13:279, DRB1*13:28:01, DRB1*13:28:02, DRB1*13:29, DRB1*13:30, DRB1*13:31, DRB1*13:32, DRB1*13:33:01, DRB1*13:33:02, DRB1*13:33:03, DRB1*13:34, DRB1*13:35, DRB1*13:36, DRB1*13:37, DRB1*13:38, DRB11*13:39, DRB1*13:40, DRB1*13:41, DRB1*13:42, DRB1*13:43, DRB1*13:44, DRB1*13:45, DRB1*13:46, DRB1*13:47, DRB1*13:48, DRB1*13:49, DRB1*13:50:01, DRB1*13:50:02, DRB1*13:50:03, DRB1*13:51, DRB1*13:52, DRB1*13:53, DRB1*13:54, DRB1*13:55, DRB1*13:56, DRB1*13:57, DRB1*13:58, DRB1*13:59, DRB1*13:60, DRB1*13:61: 01, DRB1*13: 61:02, DRB1*13:62, DRB1*13:63, DRB1*13:64, DRB1*13:65, DRB1*13:66:01, DRB1*13:66:02, DRB1*13:67, DRB1*13:68, DRB1*13:69, DRB1*13:70, DRB1*13:71, DRB1*13:72, DRB1*13:73, DRB1*13:74, DRB1*13:75, DRB1*13:76, DRB1*13:77, DRB1*13:78, DRB1*13:79, DRB1*13:80, DRB1*13:81, DRB1*13:82, DRB1*13:83, DRB1*13:84, DRB1*13:85, DRB1*13:86, DRB1*13:87, DRB1*13:88, DRB1*13:89:01, DRB1*13:89:02, DRB1*13:90, DRB1*13:91, DRB1*13:92, DRB1*13:93, DRB1*13:94:01, DRB1*13:94:02, DRB1*13:95, DRB1*13:96:01, DRB1*13:96:02, DRB1*13:97:01, DRB1*13:97:02, DRB1*13:98, DRB1*13:99, DRB1*14:01:01, DRB1*14:01:02, DRB1*14:01:03, DRB1*14:01:04, DRB1*14:02:01:01, DRB1*14:02:01:02, DRB1*14:02:02, DRB1*14:02:03, DRB1*14:02:04, DRB1*14:02:05, DRB1*14:02:06, DRB1*14:02:07, DRB1*14:03:01, DRB1*14:03:02, DRB1*14:04:01, DRB1*14:04:02, DRB1*14:04:03, DRB1*14:04:04, DRB1*14:04:05, DRB1*14:04:06, DRB1*14:05:01:01, DRB1*14:05:01:02, DRB1*14:05:02, DRB1*14:05:03, DRB1*14:05:04, DRB1*14:06:01, DRB1*14:06:02, DRB1*14:06:03, DRB1*14:06:04, DRB1*14:07:01, DRB1*14:07:02, DRB1*14:08, DRB1*14:09, DRB1*14:10, DRB1*14:100, DRB1*14:101, DRB1*14:102, DRB11*14:103, DRB11*14:104, DRB1*14:105, DRB1*4:106, DRB1*14:107, DRB11*14:108, DRB1*14:109, DRB1*14:11. DRB1*14:110. DRB1*14:111. DRB1*14:112, DRB1*14:113, DRB1*14:114, DRB1*14:115, DRB1*4:116, DRB1*14:117, DRB1*14:118, DRB1*14:119, DRB1*14:120, DRB1*14:121, DRB11*14:122, DRB1*14:123, DRB1*14:124, DRB1*14:125, DRB1*14:126:01, DRB*14:126:02, DRB1*14:127:01, DRB1*14:127:02, DRB1*14:128, DRB1*14:129, DRB1*14:12:01, DRB1*14:12:02, DRB1*14:13, DRB1*14:130, DRB11*14:131, DRB1*14:132, DRB1*14:133, DRB1*14:134, DRB1*14:135, DRB11*14:136, DRB1*14:137N, DRB1*14:138, DRB1*14:139, DRB1*14:14, DRB1*14:140, DRB1*14:141, DRB1*14:142, DRB1*14:143, DRB1*14:144, DRB1*14:145, DRB1*14:146, DRB1*14:147, DRB1*14:148, DRB1*14:149, DRB1*14:15, DRB1*14:150, DRB1*14:151, DRB1*14:152N, DRB1*14:153, DRB1*14:154, DRB1*14:155, DRB1*14:156, DRB11*14:157, DRB1*14:158, DRB1*14:159, DRB11*14:16, DRB1*14:160, DRB11*14:161, DRB1*14:162, DRB1*14:163, DRB1*14:164, DRB1*14:165, DRB1*14:166N, DRB1*14:167, DRB11*14:168, DRB11*14:169, DRB11*14:17, DRB1*14:170, DRB11*14:171, DRB1*14:172, DRB1*14:173, DRB11*14:174, DRB1*14:175, DRB1*14:176, DRB1*14:177, DRB1J*14:178, DRB1*14:179, DRB1*14:18, DRB1*14:180, DRB1J*14:181, DRB1*14:182, DRB1*14:183, DRB1*14:184, DRB1*14:185, DRB11*14:186, DRB1*14:187, DRB1*14:188N, DRB1*14:189, DRB1*14:19, DRB11*14:190, DRB1*14:191 DRB11*14:192, DRB1*14:193, DRB1*14:194, DRB1*14:195N, DRB11*14:196, DRB1*14:197N, DRB1*14:198, DRB1*14:199, DRB1*14:20, DRB1*14:200, DRB1*14:201, DRB1*14:202, DRB1*14:203, DRB1*14:204, DRB1*1 4:205, DRB1*14:206, DRB1*14:207, DRB1*14:208, DRB1*14:209, DRB1*14:21, DRB1*14:210Q, DRB1*14:211, DRB1*14:22, DRB1*14:23:01, DRB*14:23:02, DRB1*14:23:03, DRB1*14:23:04, DRB1*14:24, DRB1*14:25:01, DRB1*14:25:02, DRB1*14:26, DRB1*14:27:01, DRB1*14:27:02, DRB1*14:28, DRB1*14:29, DRB1*14:30, DRB1*14:31, DRB1*14:32:01, DRB1*14:32:02, DRB1*14:32:03, DRB1*14:33, DRB1*14:34, DRB1*14:35, DRB1*14:36, DRB1*14:37, DRB1*14:38:01, DRB1*14:38:02, DRB1*14:39 DRB1*14:40, DRB11*14:41, DRB1*14:42, DRB1*14:43, DRB1*14:44:01, DRB1*14:44:02, DRB1*14:44:03, DRB1*14:45, DRB1*14:46, DRB1*14:47, DRB1*14:48, DRB1*14:49, DRB1*14:50, DRB1*14:51, DRB1*14:52, DR31*14:53, DRB1*14:54:01:01, DRB1*14:54:01:02, DRB1*14:54:01:03, DRB1*14:54:01:04, DRB1*14:54:02, DRB1*14:54:03, DRB1*14:54:04, DRB1*14:54:05, DRB1*14:54:06, DRB1*14:54:07, DRB1*14:55, DRB1*14:56, DRB1*14:57, DRB1*14:58, DRB1*14:59, DRB1*14:60, DRB1*14:61, DRB1*14:62, DRB1*14:63, DRB1*14:64, DRB1*14:65, DRB1*14:67, DRB1*14:68:01, DRB1*14:68:02, DRB1*14:69, DRB1*14:70, DRB1*14:71, DRB1*14:72, DRB1*14:73, DRB*14:74, DRB1*14:75, DRB1*14:76, DRB1*14:77, DRB1*14:78, DRB1*14:79, DRB1*14:80, DRB1*14:81, DRB1*14:82, DRB1*14:83, DRB1*14:84, DRB1*14:85, DRB1*14:86, DRB1*14:87, DRB1*14:88, DRB1*14:89, DRB1*14:90, DRB1*14:91, DRB1*14:92N, DRB1*14:93, DRB1*14:94, DRB1*14:95, DRB1*14:96, DRB1*14:97, DRB1*14:98, DRB1*14:99, DRB1*15:01:01:01, DRB1*15:01:01:02, DRB1*15:01:01:03, DRB*15:01:01:04, DRB1*15:01:01:05, DRB1*15:01:02, DRB1*15:01:03, DRB1*15:01:04, DRB1*15:01:05, DRB1*15:01:06, DRB1*15:01:07, DRB1*15:01:08, DRB1*15:01:09, DRB1*15:01:10, DRB1*15:01:11, DRB1*15:01:12, DRB1*15:01:13, DRB1*15:01:14, DRB1*15:01:15, DRB1*15:01:16, DRB1*15:01:17, DRB1*15:01:18, DRB1*15:01:19, DRB1*15:01:20, DRB1*15:01:21, DRB1*15:01:22, DRB1*15:01: 23, DRB1*15:01:24, DRB1*15:01:25, DRB1*15:01:26, DRB1*15:01:27, DRB1*15:01:28, DRB1*15:01:29, DRB1*15:01:30, DRB1*15:01:31, DRB1*15:01:32, DRB1*15:01:33, DRB1*15:01:34, DRB1*15:01:35, DRB1*15:01:36, DRB1*15:01:37, DRB1*15:01:38, DRB1*15:01:39, DRB1*15:01:40, DRB1*15:01:41, DRB1*15:02:01:01, DRB1*15:02:01:02, DRB1*15:02:01:03, DRB1*15:02:02, DRB 1*15:02:03, DRB1*15:02:04, DRB1*15:02:05, DRB1*15:02:06, DRB1*15:02:07, DRB1*15:02:08, DRB1*15:02:09, DRB1*15:02:10, DRB1*15:02:11, DRB1*15:02:12, DRB1*15:02:13, DRB1*15:02:14, DRB1*15:02:15, DRB1*15:02:16, DRB1*15:02:17, DRB1*15:02:18, DRB11*15:02:19, DRB1*15:03:01:01, DRB1*15:03:01:02, DRB1*15:03:01:03, DRB1*15:03:02, DRB1*15:03:03, DRB1*15:03:04, DRB1*15:04, DRB1*15:05, DRB1*15:06:01, DRB1*15:06:02, DRB1*15:06:03, DRB1*15:06:04, DRB1*15:07:01, DRB1*15:07:02, DRB1*15:07:03, DRB1*15:08, DRB1*15:09, DRB1*15:10, DRB1*15:100, DRB1*15:101, DRB1*15:102, DRB1*15:103, DRB1*15:104:01, DRB11*15:104:02, DRB1*15:104:03, DRB1*15:105:01, DRB1*15:105:02, DRB1*15:106, DRB1*15:107, DRB1*15:108, DRB1*15:109, DRB1*15:110, DRB1*15:111, DRB1*15:112, DRB1*15:113N, DRB1*15:114, DRB1*15:115N, DRB1*15:116, DRB1*15:117, DRB1*15:118, DRB1*15:119, DRB1*15:11:01, DRB1*15:11:02, DRB1*15:12, DRB1*15:120, DRB1*15:121, DRB1*15:122, DRB 1 15:123, DRB1*15:124, DRB1*15:125, DRB1*15:126, DRB1*15:127, DRB1*15:128, DRB1*15:129N, DRB1*15:13, DRB1*15:130, DRB1*15:131, DRB1*15:132, DRB1*15:133, DRB1*15:134N, DRB1*15:135, DRB1*15:136, DRB1*15:137N, DRB1*15:138N, DRB1*15:139, DRB1*15:14, DRB1*15:140, DRB1*15:141, DRB115:142, DRB1*15:143, DRB1*1 5:144, DRB1*15:145, DRB1*15:146, DRB1*15:147, DRB1*15:148N, DRB1*15:149, DRB1*15:150, DRB1*15:151, DRB1*15:152, DRB1*15:153, DRB1*15:154N, DRB1*15:155, DRB1*15:156, DRB1*15:157, DRB1*15:158, DRB1*15:159N, DRB1*15:15:01, DRB1*15:15:02, DRB1*15:15:03, DRB1*15:16, DRB1*15:160, DRB1*15:161, DRB1*15:162, DRB1*15:163N, DRB1*15:164Q. DRB1*15:165, DRB1*15:166, DRB1*15:167, DRB1*15:168, DRB1*15:169, DRB1*15:170, DRB1*15:17N, DRB1*15:18, DRB1*15:19, DRB1*15:20, DRB1*15:21, DRB1*15:22, DRB1*15:23, DRB1*15:24, DRB1*15:25, DRB1*15:26, DRB1*15:27, DRB1*15:28, DRB1*15:29, DRB1*15:30, DRB1*15:31:01, DRB1*15:31:02, DRB1*15:32, DRB1*15:33, DRB1*15:34, DRB1*15:35, DRB1*15:36, DRB1*15:37:01, DRB1*15:37:02, DRB1*15:38, DRB1*15:39, DRB1*15:40, DRB1*15:41, DRB1*15:42, DRB1*15:43, DRB1*15:44, DRB1*15:45, DRB1*15:46, DRB1*15:47, DRB1*15:48, DRB1*15:49, DRB1*15:50N, DRB1*15:51, DRB1*15:52, DRB1*15:53, DRB1*15:54, DRB1*15:55, DRB1*15:56, DRB1*15:57, DRB11*15:58, DRB1*15:59, DRB1*15:60, DRB1*15:61, DRB1*15:62, DRB1*15:63, DRB1*15:64, DRB1*15:65, DRB1*15:66:01, DRB1*15:66:02, DRB1*15:67, DRB1*15:68, DRB1*15:69 DRB1*15:70, DRB1*15:71, DRB1*15:72, DRB11*15:73, DRB1*15:74, DRB1*15:75, DRB1*15:76, DRB1*15:77, DRB1*15:78, DRB1*15:79, DRB1*15:80N, DRB1*15:81, DRB1*15:82, DRB1*15:83, DRB1*15:84, DR11*15:85, DRB1*15:86, DRB1*15:87, DRB1*15:88, DRB1*15:89, DRB1*15:90, DRB1*15:91, DRB1*15:92, DRB1*15:93, DRB1*15:94, DRB1*15:95, DRB1*15:96, DRB1*15:97, DRB1*15:98, DRB1*15:99, DRB1*16:01:01, DRB1*16:01:02, DRB1*16:01:03, DRB1*16:01:04, DRB1*16:01:05, DRB1*16:01:06, DRB1*16:01:07, DRB1*16:01:08, DRB1*16:01:09, DRB1*16:01:10, DRB1*16:01:11, DRB1*16:01:12, DRB1*16:01:13, DRB1*16:01:14, DRB1*16:01:15, DRB1*16:01:16, DRB1*16:02:01:01, DRB1*16:02:01:02, DRB1*16:02:01:03, DRB1*16:02:02, DRB1*16:02:03, DRB1*16:02:04, DRB1*16:02:05, DRB1*16:02:06, DRB1*16:02:07, DRB1*16:02:08, DRB1*16:03, DRB1*16:04:01, DRB1*16:04:02, DRB1*16:05:01, DRB1*16:05:02, DRB1*16:07, DRB1*16:08, DRB1*16:09:01, DRB1*16:09:02, DRB1*16:10:01, DRB1*16:10:02, DRB1*16:11, DRB1*16:12, DRB1*16:13N, DRB1*16:14, DRB1*16:15, DRB1*16:16, DRB1*16:17, DRB1*16:18, DRB1*16:19, DRB1*16:20, DRB1*16:21N, DRB1*16:22, DRB1*16:23, DRB1*16:24, DRB1*16:25, DRB1*16:26, DRB1*16:27, DRB1*16:28, DRB1*16:29, DRB1*16:30, DRB1*16:31, DRB1*16:32, DRB1*16:33, DRB1*16:34, DRB1*16:35, DRB1*16:36, DRB1*16:37, DRB1*16:38:01, DRB1*16:38:02, DRB1*16:39, DRB1*16:40, DRB1*16:41N, DRB1*16:42, DRB1*16:43, DRB1*16:44, DRB1*16:45, DRB1*16:46, DRB1*16:47, DRB1*16:48, DRB1*16:49, DRB1*16:50, DRB1*16:51, DRB1*16:52, DRB1*16:53, DRB1*16:54, DRB1*16:55N, DRB1*16:56, DRB3*01:01:02:01, DRB3*01:01:02:02, DRB3*01:01:02:03, DRB3*01:01:03, DRB3*01:01:04, DRB3*01:01:05, DRB3*01:01:06, DRB3*01:01:07, DRB3*01:01:08, DRB3*01:01:09, DRB3*01:01:10, DRB3*01:02, DRB3*01:03, DRB3*01:04, DRB3*01:05, DRB3*01:06, DRB3*01:07, DRB3*01:08, DRB3*01:09, DRB3*01:10, DRB3*01:11, DRB3*01:12, DRB3*01:13, DRB3*01:14, DRB3*01:15, DRB3*01:16, DRB3*01:17, DRB3*01:18, DRB3*01:19, DRB3*01:20, DRB3*01:21, DRB3*01:22, DRB3*01:23, DRB3*01:24, DRB3*01:25, DRB3*01:26N, DRB3*01:27, DRB3*01:28, DRB3*01:29, DRB3*01:30, DRB3*01:31, DRB3*01:32, DRB3*01:33, DRB3*01:34, DRB3*01:35, DRB3*01:36, DRB3*01:37, DRB3*01:38, DRB3*01:39, DRB3*01:40:0IN, DRB3*01:40:02N, DRB3*01:41, DRB3*01:42, DRB3*01:43, DRB3*01:44, DRB3*01:45, DRB3*01:46, DRB3*01:47, DRB3*01:48, DRB3*01:49, DRB3*01:50, DRB3*01:51, DRB3*01:52, DRB3*01:53, DRB3*01:54, DRB3*01:55, DRB3*01:56, DRB3*01:57, DRB3*01:58, DRB3*01:59, DRB3*01:60, DRB3*01:61, DRB3*01:62, DRB3*02:01, DRB3*02:02:01:01, DRB3*02:02:01:02, DRB3*02:02:01:03, DRB3*02:02:01:04, DRB3*02:02:02, DRB3*02:02:03, DRB3*02:02:04, DRB3*02:02:05, DRB3*02:02:06, DRB3*02:02:07, DRB3*02:02:08, DRB3*02:02:09, DRB3*02:02:10, DRB3*02:02:11, DRB3*02:02:12, DRB3*02:02:13, DRB3*02:02:14, DRB3*02:02:15, DRB3*02:02:16, DRB3*02:02:17, DRB3*02:02:18, DRB3*02:02:19, DRB3*02:02:20, DRB3*02:02:21, DRB3*02:03, DRB3*02:04, DRB3*02:05, DRB3*02:06, DRB3*02:07, DRB3*02:08, DRB3*02:09, DRB3*02:10, DRB3*02:11, DRB3*02:12, DRB3*02:13, DRB3*02:14, DRB3*02:15, DRB3*02:16, DRB3*02:17, DRB3*02:18, DRB3*02:19, DRB3*02:20, DRB3*02:21, DRB3*02:22:01, DRB3*02:22:02, DRB3*02:23, DRB3*02:24, DRB3*02:25, DRB3*02:26, DRB3*02:27, DRB3*02:28, DRB3*02:29N, DRB3*02:30, DRB3*02:31:01, DRB3*02:31:02, DRB3*02:32, DRB3*02:33, DRB3*02:34, DRB3*02:35, DRB3*02:36, DRB3*02:37, DRB3*02:38, DRB3*02:39, DRB3*02:40, DRB3*02:41, DRB3*02:42, DRB3*02:43, DRB3*02:44, DRB3*02:45, DRB3*02:46, DRB3*02:47, DRB3*02:48, DRB3*02:49, DRB3*02:50, DRB3*02:51, DRB3*02:52, DRB3*02:53, DRB3*02:54, DRB3*02:55N, DRB3*02:56, DRB3*02:57, DRB3*02:58, DRB3*02:59, DRB3*02:60, DRB3*02:61Q, DRB3*02:62, DRB3*02:63, DRB3*02:64, DRB3*02:65, DRB3*02:66, DRB3*02:67N, DRB3*02:68, DRB3*02:69, DRB3*02:70, DRB3*02:71, DRB3*02:72, DRB3*02:73, DRB3*02:74, DRB3*02:75, DRB3*02:76, DRB3*02:77, DRB3*02:78, DRB3*02:79, DRB3*02:80N, DRB3*02:81, DRB3*02:82, DRB3*02:83, DRB3*02:84, DRB3*02:85, DRB3*02:86, DRB3*02:87, DRB3*02:88, DRB3*02:89, DRB3*02:90, DRB3*02:91, DRB3*02:92, DRB3*02:93, DRB3*02:94, DRB3*02:95N, DRB3*03:01:01:01, DRB3*03:01:01:02, DRB3*03:01:02, DRB3*03:01:03, DRB3*03:01:04, DRB3*03:01:05, DRB3*03:01:06, DRB3*03:01:07, DRB3*03:02, DRB3*03:03, DRB3*03:04, DRB3*03:05, DRB3*03:06, DRB3*03:07, DRB3*03:08, DRB3*03:09, DRB3*03:10, DRB3*03:11, DRB3*03:12, DRB3*03:13, DRB3*03:14, DRB3*03:15, DRB3*03:16, DRB3*03:17, DRB3*03:18, DRB3*03:19, DRB3*03:20, DRB3*03:21, DRB3*03:22, DRB3*03:23, DRB3*03:24, DRB3*03:25, DRB4*01:01:01:01, DRB4*01:01:02, DRB4*01:01:03, DRB4*01:01:04, DRB4*01:01:05, DRB4*01:01:06, DRB4*01:02, DRB4*01:03:01:01, DRB4*01:03:01:02N, DRB4*01:03:01:03, DRB4*01:03:01:04, DRB4*01:03:01:05, DRB4*01:03:01:06, DRB4*01:03:01:07, DRB4*01:03:01:08, DRB4*01:03:01:09, DRB4*01:03:01:10, DRB4*01:03:01:11, DRB4*01:03:02, DRB4*01:03:03, DRB4*01:03:04, DRB4*01:03:05, DRB4*01:03:06, DRB4*01:03:07, DRB4*01:03:08, DRB4*01:03:09, DRB4*01:03:10, DRB4*01:03:11, DRB4*01:04, DRB4*01:05, DRB4*01:06, DRB4*01:07:01, DRB4*01:07:02, DRB4*01:08, DRB4*01:09, DRB4*01:10, DRB4*01:11, DRB4*01:12, DRB4*01:13, DRB4*01:14, DRB4*01:15, DRB4*01:16N, DRB4*01:17, DRB4*01:18, DRB4*01:19, DRB4*01:20, DRB4*01:21, DRB4*01:22, DRB4*01:23, DRB4*01:24, DRB4*01:25, DRB4*01:26, DRB4*01:27, DRB4*01:28, DRB4*01:29, DRB4*01:30, DRB4*01:31, DRB4*01:32, DRB4*01:33, DRB4*01:34, DRB4*01:35, DRB4*01:36, DRB4*01:37, DRB4*01:38N, DRB4*01:39, DRB4*01:40, DRB4*01:41, DRB4*01:42, DRB4*01:43, DRB4*01:44, DRB4*01:45, DRB4*01:46, DRB4*01:47, DRB4*01:48, DRB4*01:49, DRB4*01:50, DRB4*01:51, DRB4*01:52, DRB4*01:53, DRB4*01:54N, DRB4*01:55, DRB4*01:56N, DRB4*01:57N, DRB4*01:58, DRB4*01:59, DRB4*01:60, DRB4*01:61N, DRB4*01:62, DRB4*01:63, DRB4*01:64, DRB4*01:65N, DRB4*01:66, DRB4*01:67, DRB4*01:68, DRB4*01:69, DRB4*01:70, DRB4*01:71N, DRB4*01:72, DRB4*01:73, DRB4*01:74, DRB4*01:75, DRB4*01:76, DRB4*01:77, DRB4*01:78, DRB4*01:79, DRB4*01:80N, DRB4*01:81, DRB4*01:82, DRB4*01:83, DRB4*01:84N, DRB4*01:85, DRB4*01:86, DRB4*01:87, DRB4*01:88, DRB4*01:89, DRB4*01:90, DRB4*01:91, DRB4*01:92, DRB4*01:93, DRB4*02:01N, DRB5*01:01:01:01, DRB5*01:01:01:02, DRB5*01:01:02, DRB5*01:01:03, DRB5*01:01:04, DRB5*01:02, DRB5*01:03, DRB5*01:04, DRB5*01:05, DRB5*01.06, DRB5*01:07, DRB5*01:08N, DRB5*01:09, DRB5*01:10N, DRB5*01:11, DRB5*01:12, DRB5*01:13, DRB5*01:14, DRB5*01:15, DRB5*01:16, DRB5*01:17, DRB5*01:18, DRB5*01:19, DRB5*01:20, DRB5*01:21, DRB5*01:22:01, DRB5*01:22:02, DRB5*01:23, DRB5*01:24, DRB5*01:25, DRB5*01:26, DRB5*01:27N, DRB5*01:28, DRB5*01:29, DRB5*01:30, DRB5*01:31, DRB5*01:32, DRB5*01:33, DRB5*01:34, DRB5*01:35, DRB5*01:36, DRB5*01:37, DRB5*01:38, DRB5*01:39, DRB5*01:40, DRB5*01:41, DRB5*01:42, DRB5*01:43, DRB5*01:44, DRB5*01:45, DRB5*01:46, DRB5*01:47, DRB5*01:48N, DRB5*01:49N, DRB5*01:50, DRB5*01:51, DRB5*01:52N, DRB5*01:53N, DRB5*01:54, DRB5*01:55, DRB5*02:02:01, DRB5*02:02:02, DRB5*02.02:03, DRB5*02:03, DRB5*02:04, DRB5*02:05, DRB5*02:06, DRB5*02:07, DRB5*02:08, DRB5*02:09, DRB5*02:10, DRB5*02:11, DRB5*02:12, DRB5*02:13, DRB5*02:14, DRB5*02:15, DRB5*02:16, DRB5*02:17, DRB5*02:18, DRB5*02:19N, DRB5*02:20, DRB5*02:21, DRB5*02:22, DRB5*02:23, DRB5*02:24 and any combination thereof.

In some aspects, the HLA class II molecule is a monomer. In some aspects, the HILA class II molecule is a dimer. In some aspects, the HLA class II molecule is a multimer. In some aspects, the HLA class II molecule is a trimer. In some aspects, the HLA class II molecule is a tetramer. In some aspects, the HLA class II molecule is a pentamer.

### II.E. Nucleic Acid Molecules, Vectors, and Cells

Some aspects of the present disclosure are directed to a nucleic acid molecule or a set of nucleic acid molecules encoding the antibody or antigen-binding portion thereof, the CAR, the multispecific antibody, or any combination thereof. In some aspects, the nucleic acid molecule or the set of nucleic acid molecules further encodes a signal peptide.

Some aspects of the present disclosure are directed to a vector or a set of vectors comprising a nucleic acid molecule or a set of nucleic acid molecules disclosed herein. In some aspects, the vector is a viral vector. In some aspects, the vector is a viral particle or a virus. In some aspects, the vector is a mammalian vector. In some aspects, the vector is a bacterial vector.

In certain aspects, the vector is a retroviral vector. In some aspects, the vector is selected from the group consisting of an adenoviral vector, a lentivirus, a Sendai virus, a baculoviral vector, an Epstein Barr viral vector, a papovaviral vector, a vaccinia viral vector, a herpes simplex viral vector, and an adeno associated virus (AAV) vector. In particular aspects, the vector is an AAV vector. In some aspects, the vector is a lentivirus. In particular aspects, the vector is an AAV vector. In some aspects, the vector is a Sendai virus. In some aspects, the vector is a hybrid vector. Examples of hybrid vectors that can be used in the present disclosure can be found in Huang and Kamihira, Biotechnol. Adv. 31(2):208-23 (2103), which is incorporated by reference herein in its entirety.

Some aspects of the present disclosure are directed to cells comprising a nucleic acid molecule disclosed herein, a vector disclosed herein, a CAR disclosed herein, a multispecific antibody disclosed herein, or any combination thereof. Any cell can be used in the present disclosure.

In certain aspects, the cell expresses CD4. CD4 expression can be naturally occurring, e.g., the CD4 is expressed from a nucleic acid sequence that is endogenously expressed by the cell. For example, T cells, monocytes, macrophages, dendritic cells, and natural killer (NK) cells naturally express CD4. Thus, in some aspects, the cell is a T cell, a monocyte, a macrophage, a dendritic cell, or a natural killer cell. In certain aspects, the cell is a T cell selected from a natural killer T (NKT) cell and an innate lymphoid cell (ILC). In some aspects, the cell is a monocyte. In some aspects, the cell is a macrophage. In some aspects, the cell is a dendritic cell.

In some aspects, the T cell is isolated from a human subject. In some aspects, the human subject is the same subject that will ultimately receive the T cell therapy. In other aspects, the subject is a donor subject, wherein the donor subject is not the same subject that will receive the T cell therapy.

In some aspects, the cell is a cell that does not naturally express CD4, wherein the cell has been modified to express CD4. In some aspects, the cell comprises a transgene encoding CD4, wherein the transgene is expressed by the cell. In some aspects, the cell comprises a transgene encoding a protein that activates expression of endogenous CD4 by the cell. In some aspects, the cell comprises a transgene encoding a protein or siRNA that inhibits an inhibitor of CD4 expression in the cell. In some aspects, the transgene is incorporated into the genome of the cell. In some aspects, the transgene is not incorporated into the genome of the cell.

In some aspects, the cell that is modified to express CD4 is isolated from a human subject. In some aspects, the human subject is the same subject that will ultimately receive the cell therapy. In other aspects, the subject is a donor subject, wherein the donor subject is not the same subject that will receive the cell therapy.

### III. Methods of the Disclosure

Some aspects of the present disclosure are directed to methods of treating a disease or condition in a subject in need thereof. Some aspects of the present disclosure are directed to methods of generating an antigen-binding molecule that can specifically bind a peptide fragment of a tumor antigen, wherein the peptide fragment is associated with an MHC class II molecule, and which is capable of associating with more than one MHC class II molecule.

### III.A. Methods of Treating Cancer

Some aspects of the present disclosure are directed to methods of treating a disease or condition in a subject in need thereof. In some aspects, the disease or condition comprises a cancer. In some aspects, the method comprises administering to the subject an antibody or antigen-binding portion thereof disclosed herein, a CAR disclosed herein (e.g., a cell expressing a CAR disclosed herein), a nucleic acid molecule disclosed herein (e.g., encoding a CAR disclosed herein), a multispecific antibody disclosed herein, or a vector or cell comprising any of the above.

In some aspects, the cancer is selected from melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, a cancer of unknown origin or primary, and combinations of said cancers. In some aspects, the cancer melanoma.

In some aspects, the cancer is relapsed. In some aspects, the cancer is refractory. In some aspects, the cancer is advanced. In some aspects, the cancer is metastatic.

In some aspects, the methods disclosed herein treat a cancer in a subject. In some aspects, the methods disclosed herein reduce the severity of one or more symptom of the cancer. In some aspects, the methods disclosed herein reduce the size or number of a tumor derived from the cancer. In some aspects, the methods disclosed herein increase the overall survival of the subject, relative to a subject not provided the methods disclosed herein. In some aspects, the methods disclosed herein increase the progressive-free survival of the subject, relative to a subject not provided the methods disclosed herein. In some aspects, the methods disclosed herein lead to a partial response in the subject. In some aspects, the methods disclosed herein lead to a complete response in the subject.

In some aspects, the methods disclosed herein comprise treating a cancer in a subject in need thereof, comprising administering to the subject a cell described herein, wherein the cell comprises a nucleic acid molecule disclosed herein, a vector disclosed herein, a CAR disclosed herein, and/or a multispecific antibody disclosed herein. In some aspects, the cell is a T cell. In some aspects, the cell is a cell that is modified to express CD4.

In some aspects, the cell, e.g., the T cell, is obtained from the subject. In some aspects, the cell, e.g., the T cell, is obtained from a donor other than the subject.

In some aspects, the subject is preconditioned prior to receiving the cells. The preconditioning can comprise any substance that promotes T cell function and/or survival. In some aspects, the preconditioning comprises administering to the subject a chemotherapy, a cytokine, a protein, a small molecule, or any combination thereof. In some aspects, the preconditioning comprises administering an interleukin. In some aspects, the preconditioning comprises administering IL-2, IL-4, IL-7, IL-9, IL-15, IL-21, or any combination thereof. In some aspects, the preconditioning comprises administering cyclophosphamide, fludarabine, or both (i.e., a lymphodepleting chemotherapy). In some aspects, the preconditioning comprises administering vitamin C, an AKT inhibitor, ATRA (vesanoid, tretinoin), rapamycin, or any combination thereof.

### III.B. Methods of Engineering an Antigen-Targeting Cell

Certain aspects of the present disclosure are directed to methods of engineering an antigen-targeting cell. In some aspects, the antigen is a CCND1 antigen. In some aspects, the method comprises transducing a cell with a nucleic acid molecule disclosed herein or a vector disclosed herein. The cell can be any cell described herein. In some aspects, the cell is a T cell described herein. In some aspects, the cell is a cell that is modified to express CD4, as described herein. In some aspects, the cell, e.g., the T cell, is obtained from a subject in need of a T cell therapy. In some aspects, the cell is obtained from a donor other than the subject in need of the T cell therapy. In some aspects, the cell is a T cell or a natural killer cell.

### III.C. Methods of Generating Antigen-Binding Molecules

Some aspects of the present disclosure are directed to methods of generating an antigen-binding molecule that can specifically bind a peptide fragment of a tumor antigen, wherein the peptide fragment is associated with an MHC class II molecule, and which is capable of associating with more than one MHC class II molecule. Some aspects of the present disclosure are directed to a method of generating an antibody or an antigen-binding portion thereof that binds a peptide fragment complexed with an MHC Class II molecule presenting comprising: (i) identifying a peptide fragment associated with an MHC Class II molecule, and (ii) raising an antibody against the peptide fragment; wherein the antibody is not raised against the MHC Class II molecule or a complex of the MHC Class II molecule and the peptide fragment.

Some aspects of the present disclosure are directed to a method of generating an antibody or an antigen-binding portion thereof that binds a tumor antigen comprising: (i) identifying a peptide fragment of the tumor antigen that is associated with an MHC Class II molecule, and (ii) raising an antibody against the peptide fragment; wherein the antibody is not raised against the MHC Class II molecule or a complex of the MHC Class II molecule and the peptide fragment.

Some aspects of the present disclosure are directed to a method of generating an antibody or an antigen-binding portion thereof that binds a tumor antigen comprising: (i) computationally identifying a peptide fragment of the tumor antigen that is likely to be associated with an MHC Class II molecule, and (ii) raising an antibody against the peptide fragment; wherein the antibody is not raised against the MHC Class II molecule or a complex of the MHC Class II molecule and the peptide fragment.

In some aspects, the method further comprises selecting for an antibody or an antigen-binding portion thereof that specifically binds an epitope of the peptide fragment that is exposed on the surface of the MHC Class II molecule-peptide fragment complex.

In some aspects, the method comprises identifying a peptide fragment associated with an MHC class II molecule, isolating the peptide, and raising an antibody against the isolated peptide. In some aspects, the peptide fragment is identified by isolating an MHC class II complex with a tumor antigen peptide fragment and isolating and/or characterizing the peptide fragment. In some aspects, the peptide fragment is identified using an MHC class II peptide fragment prediction algorithm.

In some aspects, the method comprises contacting the T cells with an HLA class II molecule disclosed herein. In some aspects, the method comprises contacting the T cells with an APC disclosed herein. In some aspects, following the contacting, the enriched population of T cells comprises a higher number of T cells capable of binding the HLA class II molecule relative to the number of T cells capable of binding the HLA class II molecule prior to the contacting.

In some aspects, the method comprises contacting the T cells in vitro with a peptide, wherein the peptide comprises the amino acid sequence set forth in SEQ ID NO: 13. In some aspects, the method comprises contacting the T cells in vitro with a peptide, wherein the peptide consists of the amino acid sequence set forth in SEQ ID NO: 13. In some aspects, following the contacting, the enriched population of T cells comprises a higher number of T cells capable of binding the HLA class II molecule relative to the number of T cells capable of binding the HLA class II molecule prior to the contacting.

Some aspects of the present disclosure are directed to a method of selecting a T cell capable of targeting a tumor cell. In some aspects, the method comprises contacting a population of isolated T cells in vitro with a peptide, wherein the peptide consists of an amino acid sequence as set forth in SEQ ID NO: 13. In some aspects, the T cells are obtained from a human subject.

The T cells obtained from the human subject can be any T cells disclosed herein. In some aspects, the T cells obtained from the human subject are tumor infiltrating lymphocytes (TIL).

In some aspects, the method further comprises administering to the human subject the enriched T cells. In some aspects, the subject is preconditioned prior to receiving the T cells, as described herein.

All of the various aspects, aspects, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1—Identification of WT1 Peptide Presented By HLA-DP Molecules

### Methods

Cells and cDNA

Blood samples were obtained from healthy donors following Institutional Review Board approval. Written, informed consent was collected from all donors who provided the samples. K562 is a human erythroleukemic cell line defective of HLA class II and Ii expression. T2 is a T-cell leukemia/B-cell large cell leukemia cell line which expresses Ii but lack HLA class II expression. K562 and T2 cells were cultured in RPMI1640 supplemented with 10% fetal calf serum and gentamycin antibiotic (Life Technologies, Carlsbad, CA). All cell lines were obtained from American Type Culture Collection (ATCC).

All cDNAs were cloned into the pMX retroviral vector and all transfectants were generated using retrovirus produced by 293GPG cells or PG13 cells as previously published (Butler et al., 2007; Hirano et al., 2006). K562 transfectants expressing the indicated HLA-class II in conjunction with Ii were generated as previously described (Butler et al., 2010; Tanaka et al., 2011). Full-length WT1 cDNA was fused with a truncated version of human nerve growth factor receptor (ΔNGFR) via an optimized intervening sequence consisting of a furin cleavage site, an SGSG spacer sequence, and a P2A sequence and ΔNGFR positive cells were isolated using anti-NGFR monoclonal antibody. Clone 9 TCRα and TCRβ genes were codon-optimized according to sequences reported by Lin et al (Lin et al., 2013) and were fused by a furin cleavage site, an SGSG spacer sequence, and an F2A sequence.

TCR Transduction into Primary T Cells

CD3⁺ T cells were purified using the Pan T Cell Isolation kit (Miltenyi Biotec). Purified T cells were stimulated with aAPC/mOKT3 (aAPC expressing a membrane-bound form of anti-CD3 mAb (clone OKT3) and the co-stimulatory molecules CD80 and CD83) irradiated with 200 Gy at an effector:target (E:T) ratio of 5:1 (Sugata et al., 2021). 48 hours later, activated T cells were retrovirally transduced with cloned TCR genes via centrifugation at 2,000 g for 2 h at 32° C. for two consecutive days using a Retronectin-coated plate (Takara Bio) and 100 IU ml⁻¹ IL-2 and 10 ng ml⁻¹ IL-15 were added to the TCR-transduced T cells. The culture medium was replenished every 2-3 d. TCR-transduced CD4⁺ T cells were purified using the CD4⁺ T Cell Isolation kit (Miltenyi Biotec).

### Flow Cytometric Analysis

Monoclonal antibodies recognizing the following surface antigens were used: pan HLA class II (6604366, 1:500, Beckman Coulter), HLA-DP (ab21119-100, 1:100, Abcam), HLA-DR (555561, 1:500, BD Biosciences), Ii (555540, 1:500, BD Biosciences), CLIP (555981, 1:200, BD Biosciences), HLA-DM (555983, 1:250, BD Biosciences), NGFR (557196, 1:200, BD Biosciences). Mouse isotype controls were from BD Biosciences and each was used at 1:500. Surface and intracellular molecular staining was carried out as described elsewhere (Butler et al., 2010).

### Elispot Assays

Human CD4⁺ T cells transduced with either the clone 9 TCR or empty PMX vector were positively purified using magnetic beads (Miltenyi Biotec). IL-2 and interferon (IFN)-g ELISPOT were performed as previously described (Hirano et al., 2003; Hirano et al., 2006; Nakatsugawa et al., 2015). Briefly, for the IL-2 ELISPOT assay, PVDF plates (Millipore) were coated with capture monoclonal antibody (SEL002; R&D Systems, Minneapolis, MN). T cells were incubated with 2×10\hat{ }4 stimulator cells for 20-24 h at 37° C. in the presence or absence of indicated peptides. Plates were washed and incubated with biotin-conjugated detection monoclonal antibody (SEL002; R&D Systems). After washing, alkaline phosphatase-conjugated streptavidin (Jackson ImmunoResearch) was added. Plates were washed and incubated with nitroblue tetrazolium/5-bromo-4-chloro-3-indolyl phosphate (Promega) and IL-2 spots were developed. For the IFN-g ELISPOT assay, polyvinylidene difluoride (PVDF) plates (Millipore) were coated with capture monoclonal antibody (1D1K; MABTECH, Mariemont, OH). T cells were incubated with 2×10\hat{ }4 stimulator cells for 20-24 h at 37′C in the presence or absence of indicated peptides. Plates were washed and incubated with biotin-conjugated detection monoclonal antibody (7-B6-1; MABTECH). HRP-conjugated Streptavidin (DAKO, Carpenteria, CA) was then added, and IFN-g spots were subsequently developed. The peptides used were Plexin A4₁₂₅₉₋₁₂₆₈ (PLXNA4₁₂₅₉₋₁₂₆₈) (₁₂₅₉AYKRKSRESD₁₂₆₈) (SEQ ID NO: 53), tetanus toxin₉₄₇₋₉₆₇ (TT₉₄₇₋₉₆₇) (₉₄₇FNNFTVSFWLRVPKVSASHLE₉₆₇) (SEQ ID NO: 54), CLIP (Ii₉₇₋₁₂₀) (₉₇LPKPPKPVSKMRMATPLLMQALPM₁₂₀) (SEQ ID NO: 55) and WT1₃₂₈₋₃₄₈ (₃₂₈ PGCNKRYFKLSHLQMHSRKHI₃₄₈) (SEQ ID NO: 56).

### In Vitro Cytotoxicity Assays

Ten thousand K562 transfectants were labelled with 5 M florescent Vybrant™ DiO in PBS (Thermo Fisher Scientific) for 15 mins at 37° C. After washing, the DiO-labelled targets were added to 96-well plates in 100 μl RPMI with 10% FBS. 5×10\hat{ }4 TCR-transduced T cells were added at 5:1 E:T ratio. After 18 h co-culture, cells were transferred to a new microtiter plate. 3 M TO-PRO-3 (Thermo Fisher Scientific) was added to cell suspension to stain for dead cells and cells were analyzed by flow cytometric assays to determine the frequency of live and dead DiO*target cells. non-adherent cells were collected and transferred to a new microtiter plate. The % cytotoxicity is calculated by: % DiO⁺TO-PRO-3⁺ (with T cells)−% DiO⁺ TO-PRO-3⁺ (without T cells).

### In Vivo T-Cell Assays

NSG mice (n=3 per group) were subcutaneously inoculated with 5×10≡K562 cells stably expressing DP4/WT1/DNGFR or Ii/DP4/WT1/DNGFR. Two days later, the mice were infused with 1×10\hat{ }7 TCR-transduced T cells. Tumor volume was calculated using the formula: tumor volume (mm³)=length×width×height×0.52. Data are shown as means±s.e.m.'s for each group (n=3). There was no significant difference in the tumorigenicity of the two K562 transfectants. Mice were monitored, at minimum, once every 2 days and tumors were not to exceed 1.5 cm in diameter, nor 1,500 mm³ in volume.

### Statistical Analysis

Statistical analysis was performed using GraphPad Prism 9.0. Two-way ANOVA tests followed by Bonferroni post hoc analysis were employed. P values less than 0.05 were considered significant. No statistical method was used to predetermine sample size. The investigators were not blinded to allocation during experiments and outcome assessment. The experiments were not randomized.

### Research Results

WT1₃₂₈₋₃₄₈BINDS TO HLA-DP2 AND HLA-DP4 MOLECULES

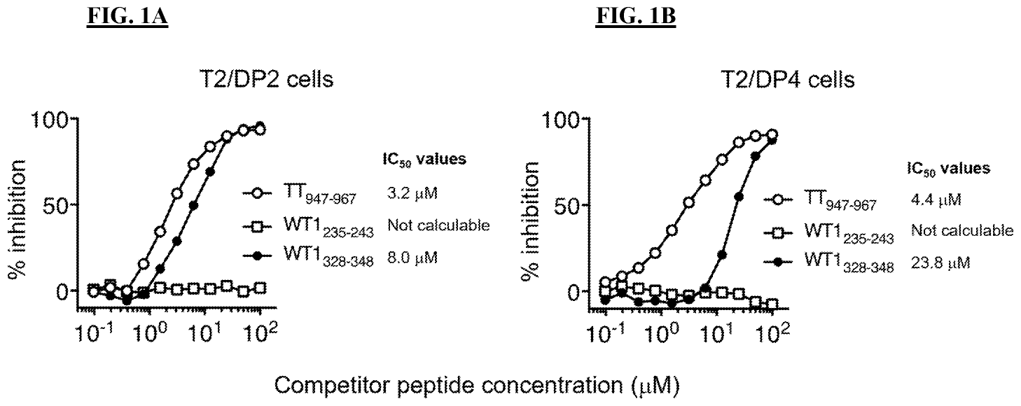

We first searched WT1-derived peptides which can potentially bind to DP2 and DP4 molecules through an algorithmic prediction tool, NetMHC 3.2. WT1₃₂₈₋₃₄₈, a 21-mer peptide, was among the best binders for both DP2 and DP4 molecules, which demonstrated the highest predicted binding affinity compared to other WT1-derived peptides of similar length (Table 1). Known strong binders to DP4, tetanus toxin (TT)₉₄₇₋₉₆₇ and Oxy₂₇₁₋₂₈₇, were also predicted by the algorithm to have strong affinity for DP4 as well as DP2. To confirm whether WT1₃₂₈₋₃₄₈ can bind to DP2 and DP4 in vitro, we performed a cell-based competitive binding assay using T2 cell, which is an HLA class II deficient cell line expressing endogenous Ii and is widely used to study antigen presentation by HLA class II (Denzin et al., 1994; Henne, Schwenk, Koch, & Moller, 1995; Riberdy & Cresswell, 1992; Tanaka et al., 2011). T2 cells individually transduced with DP2 or DP4 were pulsed with graded concentration of WT1₃₂₈₋₃₄₈ in the presence of reference peptide Oxy₂₇₁₋₂₈₇. Binding strength of WT1₃₂₈₋₃₄₈ to DP2 and DP4 was determined based on its ability to outcompete and displace the reference peptide (Castelli et al., 2002). WT1₃₂₈₋₃₄₈ demonstrated IC50 value comparable to TT₉₄₇₋₉₆₇, whereas the known HLA-A2 restricted peptide, WT1₂₃₅₋₃₄₃, failed to displace reference peptide at any tested concentration (FIGS. 1A-1B). These results suggest that WT1₃₂₈₋₃₄₈ binds to both DP2 and DP4 strongly in vitro.

TABLE 1
Predicted peptide binding affinity for DP2 and DP4 in vitro. Full
length of WT1 protein sequence was searched to identify peptides
which can bind to DP2 and DP4 molecules using NetMHCII 3.2 server
(services.healthtech.dtu.dk/service.php?NetMHCIIpan-3.2) in silico.
TT₉₄₇₋₉₆₇, Oxy₂₇₁₋₂₈₇, WT1₂₃₅₋₂₄₃ were included as controls. Predicted
binding affinity and binding core sequences of searched
peptides are shown.

| | | | Calculated |
|---|---|---|---|
| Peptide | Allele | Predicted binding core sequence | affinity (nM) |
| WT1₃₂₈₋₃₄₈ | DP2 | ₃₃₅FKLSHLQMH₃₄₃ | 125.5 |
| | DP4 | ₃₃₅FKLSHLQMH₃₄₃ | 138.35 |
| TT₉₄₇₋₉₆₇ | DP2 | ₉₅₀FTVSFWLRV₉₅₈ | 1.7 |
| | DP4 | ₉₅₀FTVSFWLRV₉₅₈ | 1.9 |
| Oxy₂₇₁₋₂₈₇ | DP2 | ₂₇₅FAATQFEPL₂₈₃ | 2.56 |
| | DP4 | ₂₇₅FAATQFEPL₂₈₃ | 2.2 |
| WT1₂₃₅₋₂₄₃ | DP2 | ₂₃₅CMTWNQMNL₂₄₃ | 14825.13 |
| | DP4 | ₂₃₅CMTWNQMNL₂₄₃ | 18033.61 |

A DP5-Restricted, WT1₃₃₂₋₃₄₇-Specific TCR Recognize WT1₃₂₈₋₃₄₈ in the Context of DP2 and DP4

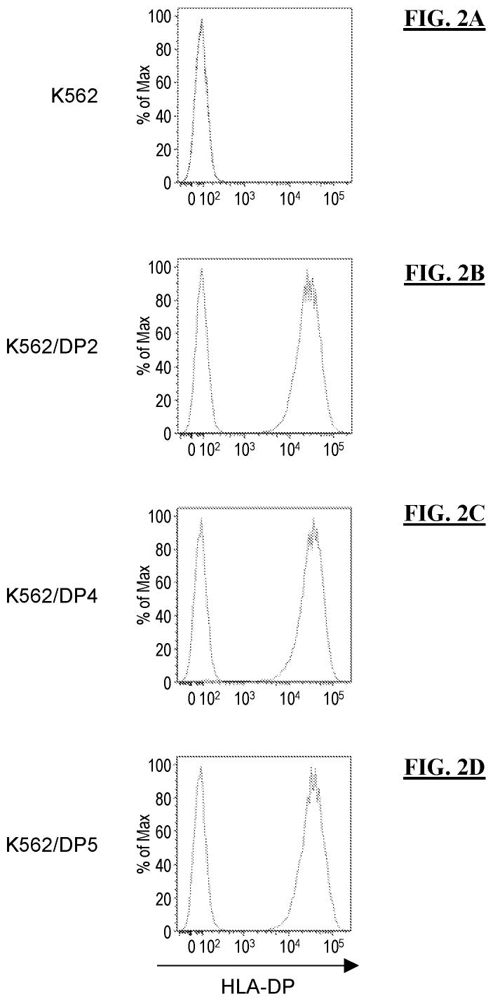

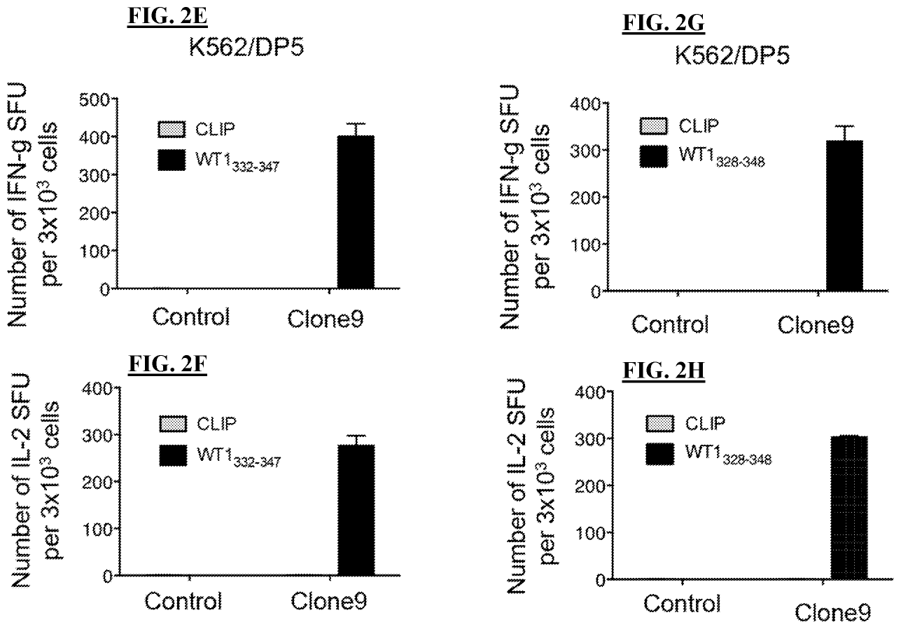

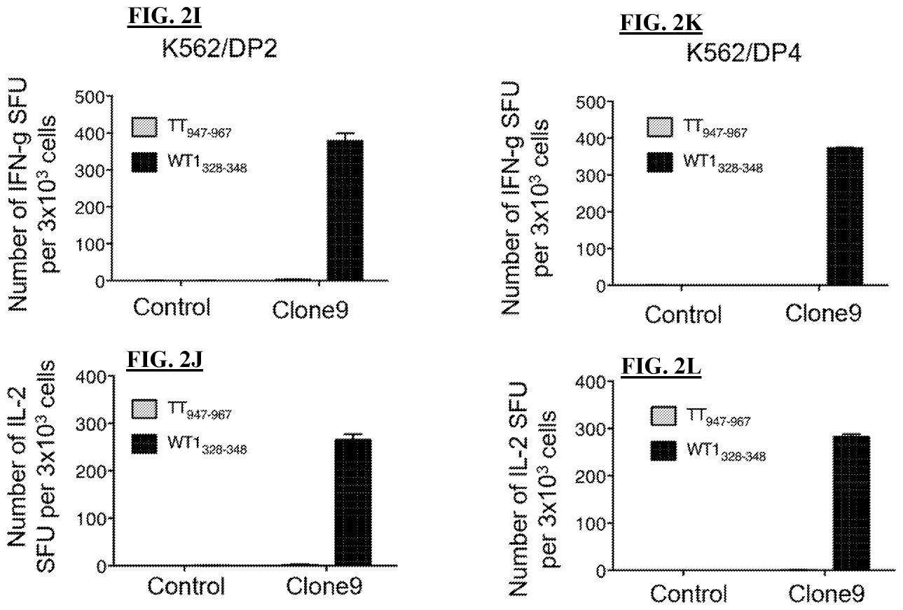

Having identified WT1₃₂₈₋₃₄₈ as a strong DP2 and DP4 binder, we studied whether WT1₃₂₈₋₃₄₈ is naturally processed from WT1 protein and subsequently presented on the cell surface by DP2 and DP4. A TCR capable of recognizing DP2/WT1₃₂₈₋₃₄₈ and DP4/WT1₃₂₈₋₃₄₈ complexes can be employed as a probe to address this question. Lin et al have generated a DP5-restricted WT1₃₃₂₋₃₄₇-specific CD4⁺ T cell clone, designated as clone 9, and cloned its TCR (Lin et al., 2013). It is widely known that HLA class II-restricted TCRs can promiscuously recognize different but overlapping peptides presented by the same class II molecules, or the same peptides presented by different class II molecules (Mohan & Unanue, 2012; Panina-Bordignon et al., 1989). We therefore speculated that the clone 9 TCR can also recognize WT1₃₂₈₋₃₄₈ in the context of DP2 and DP4. To test our hypothesis, we transduced codon-optimized clone 9 TCR genes into primary human CD4⁺ T cells and assessed their antigen-specific responses in IL-2 and IFN-γ ELISPOT assays. HLA and Ii-deficient, K562-based artificial antigen-presenting cells (aAPCs) (Butler et al., 2007; Hirano et al., 2006) were individually transduced with DP2, DP4 and DP5 (FIGS. 2A-2D), pulsed with WT1₃₂₈₋₃₄₈ or control peptide and used as target cells in the assays. We confirmed that clone 9 TCR-transduced CD4⁺ T cells were able to recognize K562/DP5 cells pulsed with not only WT1₃₃₂-347 but also WT1₃₂₈₋₃₄₈ (FIGS. 2E-2H). We then showed that clone 9 TCR-transduced CD4⁺ T cells also recognized K562/DP2 and K562/DP4 cells pulsed with WT1₃₂₈₋₃₄₈ (FIGS. 2I-L). These results indicate that the DP5-restricted WT1₃₃₂₋₃₄₇-specific TCR can promiscuously recognize WT1₃₂₈₋₃₄₈ presented by DP2, DP4 and DP5, which allowed us to utilize clone 9 TCR to investigate whether WT1₃₂₈₋₃₄₈ is a naturally processed DP2 and DP4-restricted epitope.

### WT1₃₂₈₋₃₄₈ Can be Naturally Processed and Presented in a DP2-Restricted and DP4-Restricted Manner

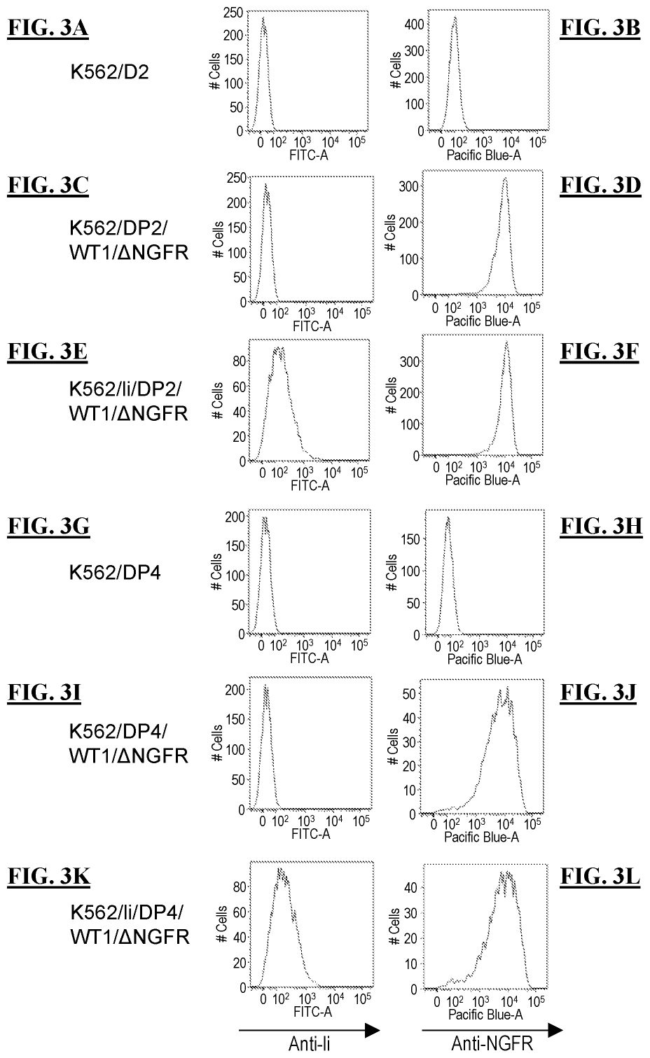

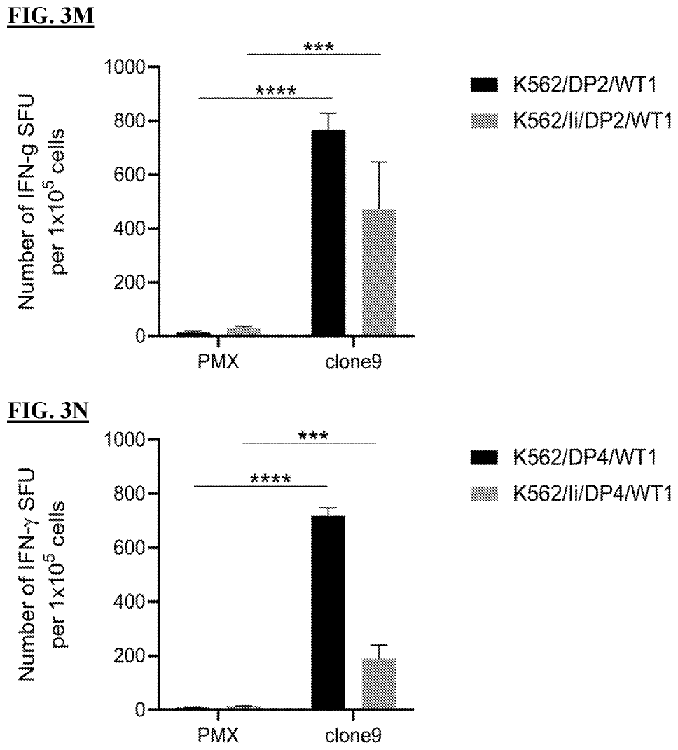

We have established that clone 9 TCR recognized DP2/WT1₃₂₈₋₃₄₈ and DP4/WT1₃₂₈₋₃₄₈ complexes. If clone 9 TCR-transduced CD4⁺ T cells can also recognize DP2⁺/WT1⁺ or DP4⁺/WT1⁺ target cells, it would suggest that WT1₃₂₈₋₃₄₈ can be naturally processed from intracellular WT1 protein and presented by DP2 and DP4 on cell surface. As a leukemic cell line, K562 cells express endogenous WT1 (Svedberg, Chylicki, & Gullberg, 1999). The clone 9 TCR was originally isolated in a DP5-restricted manner. Therefore, it is possible that the clone 9 TCR-transduced T cells may not have sufficient avidity to recognize target cells which present peptides derived from endogenous WT1 protein in the context of DP2 and/or DP4. To ensure that clone 9 TCR-transduced CD4⁺ T cells would possess sufficient avidity to recognize naturally processed and presented WT1₃₂₈₋₃₄₈, we further increased WT1 expression by transducing ΔNGFR-tagged, full-length WT1 gene into K562/DP2 and K562/DP4 cells (FIGS. 3A-3L). We have previously shown that although Ii can bind to both DP2 and DP4 molecules, it does so via a non-CLIP region without occupying the peptide binding cleft, thereby allowing DP2 and DP4 to present intracellularly-derived antigenic peptides (Anczurowski & Hirano, 2018; Anczurowski et al., 2018; Yamashita et al., 2017). Given this finding, we posited that if endogenously derived WT1₃₂₈₋₃₄₈ can be loaded onto DP2 and DP4 molecules, the loading would occur even in the presence of Ii. Since K562 cells are Ii-deficient, we ectopically expressed Ii in the indicated K562 transfectants (FIGS. 3A-3L). In IFN-γ ELISPOT assays, clone 9 TCR-transduced CD4⁺ T cells were able to recognize all the indicated DP2⁺ and DP4⁺ transfectants overexpressing WT1 regardless of the expression of Ii. (FIGS. 3M-N). However, the presence of Ii appeared to reduce the loading of naturally derived WT1₃₂₈₋₃₄₈ onto DP2 and DP4, as demonstrated by lower level of IFN-γ secreted from T cells when stimulated with Ii+ transfectants compared to their Ii⁻ counterparts. Nonetheless, the results suggest that WT 1328-348 is a natural epitope in the context of DP2 and DP4.

### Clone 9 TCR-Transduced T Cells Mediate Antitumor Responses in Vitro and in Vivo

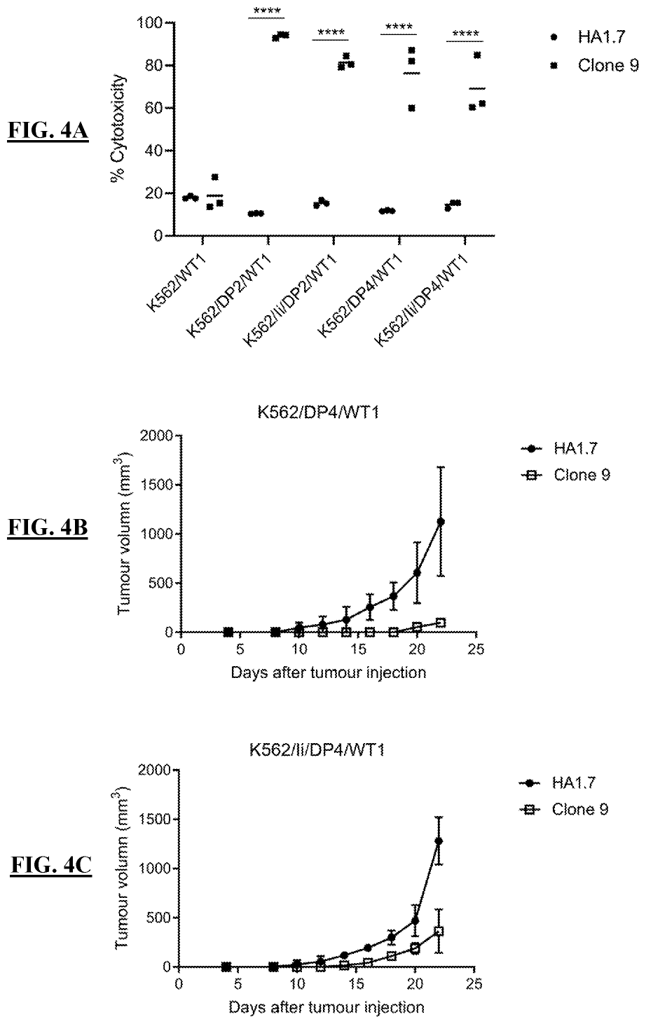

We further explored the therapeutic potential of clone 9 TCR-transduced T cells, such as their ability to directly kill WT1-expressing target cells in vitro and control tumor growth in vivo. In an in vitro cytotoxicity assay, we included a 1:1 mixture of clone 9 TCR-transduced CD4⁺ and CD8⁺ T cells and showed that these T cells were able to kill K562/DP2 and K562/DP4 cells overexpressing WT1, and the presence of Ii did not affect the extent of cytotoxicity (FIG. 4A). T cells transduced with a hemagglutinin-specific DP1-restricted TCR, clone HA1.7, were utilized as a negative control, which exhibited significantly weaker cytotoxicity against DP2⁺/WT 1⁺ and DP4⁺/WT 1⁺ target cells. We also examined the antitumor effect of clone 9 TCR-transduced T cell against DP4⁺/WT1⁺ target cells in vivo. NSG mice were first inoculated with WT1-transduced K562/DP4 cells or K562/Ii/DP4 cells. Since both antitumor CD4⁺ T cells and CD8⁺ T cells are required for optimal tumor control in vivo, we injected HA1.7 TCR or clone 9 TCR-transduced T cells with a CD4⁺: CD8⁺ ratio of 1:1 into NSG mice, two days post tumor injection. Tumor-bearing mice treated with clone 9 TCR-transduced T cells showed superior growth control of DP4⁺/WT1*and DP4*/WT1*/Ii+ tumor cells (FIGS. 4B-4C). These results demonstrated the antitumor efficacy of clone 9 TCR-transduced T cells against DP2⁺/WT1⁺ and DP4⁺/WT1⁺ tumor cells regardless of the expression of Ii.

### Conclusion

In this present study, we demonstrated that WT1₃₂₈₋₃₄₈ peptide can be naturally processed and presented by DP2 and the most frequent HLA class II molecule, DP4. In a cell-based competitive binding assay, we first showed that WT1₃₂₈₋₃₄₈ peptide bound to both DP2 and DP4 molecules, which aligned with the results obtained in silico. CD4⁺ T cells transduced with an DP5-restricted, WT1₃₃₂₋₃₄₇-specific TCR, clone 9, promiscuously recognized DP2⁺ and DP4⁺ target cells pulsed with WT1₃₂₈₋₃₄₈ peptide as well as DP2⁺ and DP4⁺ target cells expressing WT1 protein, suggesting that WT1₃₂₈₋₃₄₈ is a natural epitope of WT1 in DP2 and DP4-restricted manner. Results from in vitro killing assay and in vivo mouse experiments further demonstrate the therapeutic potential and feasibility of utilizing clone 9 for TCR gene therapy against DP4⁺/WT 1 tumors.

The present study extensively utilized K562 cells, which is frequently used as a backbone of aAPC as they do not express endogenous HLA class I/II molecules and can be genetically modified at ease (Butler & Hirano, 2014). K562 cells also lack endogenous Ii. Thus, we ectopically expressed Ii in our K562-based aAPCs to recapitulate antigen presentation in Ii⁺ cells. While clone 9 TCR-transduced T cells were able to recognize endogenously derived WT1₃₂₈₋₃₄₈ presented by DP2 and DP4, T cell responses were weaker in the presence of Ii, suggesting that K562/DP2 and K562/DP4 cells presented endogenously derived WT1₃₂₈₋₃₄₈ more efficiently when Ii was absent. This was consistent with our previous findings, as the presence of Ii seemed to reduce the presentation of endogenously derived MAGE-A3 peptides by DP4 (Yamashita et al., 2017). We have previously shown that Ii can facilitate egress of DP4 from the ER towards early endosomes/lysosomes, which can possibly result in insufficient loading of intracellular peptide onto DP4 within the ER. Using cell lines beside K562, others have previously identified WT1₃₃₂₋₃₄₇ as a natural epitope shared by multiple HLA class II molecules, including DR4 (HLA-DRB1*04:05), DR15 (DRB1*15:01, HLA-DRB1*15:02), and DP5 (DPA1*02:01/DPB1*05:01) (Fujiki et al., 2008; Fujiki et al., 2007; Lin et al., 2013), though it was unknown if those cell lines express Ii or not.

It was evident in the present study that clone 9 TCR-transduced T cells have sufficient avidity to recognize DP2⁺ and DP4⁺ target cells overexpressing full-length WT1, as demonstrated by cytokine secretion, in vitro cytotoxicity, and control of tumor growth in mice. Generally, the avidity of T cells can be determined by the density of target HLA/peptide complexes on cell surface, and the affinity of individual TCR towards its cognate HLA/peptide complex. Clone 9 TCR was originally isolated from an HLA-DP5⁺ donor and selected based on its specificity towards WT1₃₃₂₋₃₄₇ in a DP5-restricted manner. Thus, it is possible that clone 9 TCR-transduced T cells might not have sufficient avidity to recognize DP2⁺ or DP4⁺ target cells if the level of WT1 expression is below the threshold for T cell activation. Without knowing the affinity of clone 9 TCR towards DP2/WT1₃₂₈₋₃₄₈ or DP4/WT1₃₂₈₋₃₄₈, we ectopically expressed WT1 in K562/DP2 and K562/DP4 cells to increase the level of DP2/WT1₃₂₈₋₃₄₈ and DP4/WT1₃₂₈₋₃₄₈ complexes on cell surface, thereby enhancing avidity of clone 9 TCR-transduced T cells sufficient to recognize naturally processed and presented WT1₃₂₈₋₃₄₈ in the context of DP2 and DP4. WT1₃₂₈₋₃₄₈-pulsed K562/DP2 and K562/DP4 cells likely presented even higher level of DP2/WT1₃₂₈₋₃₄₈ and DP4/WT1₃₂₈₋₃₄₈ complexes on cell surface, contributing to greater IFN-γ secretion from clone 9 TCR-transduced T cells stimulated by these peptide-pulsed target cells. However, clone 9 TCR-transduced T cells might not have enough avidity to target WT1-untransduced K562/DP2 and K562/DP4 cells if the level of endogenous WT1 in K562 cells is not sufficient. Compared to K562 cells, some other cancer cell lines and primary leukemic blasts express higher level of endogenous WT1 protein (May et al., 2007). If WT1₃₂₈₋₃₄₈ can also be naturally processed and presented in DP2 and DP4-restricted manner by these cancer cell lines and primary blasts, it is likely that clone 9 TCR-transduced T cells would be able to directly recognize them when level of DP2/WT1₃₂₈-348 and DP4/WT1₃₂₈₋₃₄₈ complexes on the cell surface exceeds the threshold for stimulation. In addition to HLA/peptide level, enhancing TCR affinity towards its cognate HLA/peptide complex can also increase T cell avidity. Most antitumor TCRs cloned from peripheral T cells possess low to intermediate TCR affinity against tumor antigens derived from self-proteins, due to central and peripheral tolerance. Various strategies have been employed by others to enhance affinity of TCR for greater antitumor efficacy (Y. Li et al., 2005; Robbins et al., 2008; Schmitt et al., 2013). We have previous reported that, by exploiting TCR chain centricity, we were able to isolate TCRs with a wide range of affinity while possessing minimal cross-reactivity (Nakatsugawa et al., 2015; Ochi et al., 2015). Through generation of thymically unselected TCR repertoires, we have successfully isolated TCR with optimal affinity against A2/MART127-35 and A24/WT1₂₃₅₋₂₄₃ complexes. We will employ this strategy to first determine chain centricity of clone 9 TCR, generate a pool of TCR counter chains, and identify the TCR pairs with optimal affinity against DP2/WT1₃₂₈₋₃₄₈ and DP4/WT1₃₂₈₋₃₄₈ complexes. This would allow us to obtain antitumor T cells with optimal avidity towards DP2⁺ and DP4⁺ tumor cells with a wide range of WT1 expression.

The identification of natural DP2 and DP4-restricted WT1 epitope represents a new avenue for treating patients with DP2⁺/WT1⁺ and DP4⁺/WT1⁺ cancer with adoptive T cell therapies. To date, majority of adoptive T cell therapies redirected by antitumor receptors target tumor antigenic peptides presented by HLA class I molecules. Despite limited expression in normal tissues, HLA class II molecules including DP can be expressed in several types of cancer, either constitutively or inducible upon exposure to IFN-γ (Axelrod et al., 2018). Although the presence of HLA class II molecules expressed by tumor cells can potentially induce T cell anergy in the absence of costimulatory molecules, or promote immunosuppressive response from regulatory T cells, many studies have reported positive correlation between HLA class II expression and favorable prognosis. For example, expression of HLA class II has been associated with greater progression-free and overall survival in patients with melanoma or lymphoma after anti PD-1/PD-L1 treatment (D. B. Johnson et al., 2016; Rodig et al., 2018; Roemer et al., 2018). Certain subsets of triple-negative breast cancer patients with high HLA class II expression also had better clinical response following chemotherapy or radiotherapy (Forero et al., 2016; I. A. Park et al., 2017). The enhanced antitumor responses observed in these cases might be attributable to direct recognition by antitumor CD4⁺ T cells. CD4⁺ T cells are known to provide help to CD8⁺ T cells, but they can also eradicate tumor cells through the secretion of tumoricidal IFN-γ, or through cytolytic mechanisms by directly targeting HLA class II⁺ tumor cells (Lin et al., 2013; Quezada et al., 2010). The promiscuity of HLA class II-restricted peptides offers another advantage to interventions against HLA class II tumors. Strategies targeting HLA class I-restricted peptides are often only applicable to certain subsets of patients with compatible HLA genotypes. Conversely, many HLA class II-restricted peptides, such as those derived from WT1, can promiscuously bind to multiple HLA class II molecules (Fujiki et al., 2008; Y. Hu et al., 2014; Kobayashi et al., 2000; Panina-Bordignon et al., 1989). Thus, patients with various HLA class II genotypes can benefit from therapies directed against the same peptides promiscuously presented by different class II molecules.

In summary, we have shown that WT1₃₂₈₋₃₄₈ peptide is a natural epitope presented by DP2 and DP4, and T cells transduced with DP5-restricted WT1₃₃₂₋₃₄₇-specific TCR, clone 9, were able to recognize DP2⁺/WT1 and DP4⁺/WT1 tumors. Considering that WT1 is a well-established TAA and the high prevalence of DP2 and DP4, immunotherapeutic strategies targeting this epitope, such as TCR and TCR-like CAR T cell therapy, serve as promising treatment alternatives for many cancer patients.

### Example 2—Development of CAR-T Cell Therapy Targeting WT1 Peptide Presented By Diverse HLA Class II Molecules

In the current study, we describe the identification of an antibody capable of recognizing WT1 peptide promiscuously presented by diverse class II molecules. T cells transduced with second generation CAR derived from this antibody show class II-restricted, WT1-specific antitumor response by recognizing and killing WT1⁺ cancer cell lines and primary leukemic samples expressing various class II molecules. The data presented here provides the first proof-of-concept for a peptide-centric, HLA class II-agnostic antibody-based technology that can potentially be expanded to many other cancer antigens beyond WT1.

### Methods

Cells and cDNA

Peripheral blood samples were obtained from healthy donors following Institutional Review Board approval. Written, informed consent was collected from all donors who provided the samples. K562 is a human erythroleukemic cell line defective of HLA class II and Ii expression. T2 is a T-cell leukemia/B-cell large cell leukemia cell line which expresses Ii but lack HLA class II expression. K562, T2, HL60, Daudi, Ramos, U937 cells were cultured in RPMI1640 supplemented with 10% fetal calf serum and gentamycin antibiotic. KG1a cells were cultured in IMDM supplemented with 20% fetal calf serum and gentamycin antibiotic. OCI-AML5 were cultured in alpha-MEM supplemented with 20% fetal calf serum and gentamycin antibiotic and 10 ng/ml human GM-CSF. Daudi, Ramos, HL60, T2, K562 were obtained from American Type Culture Collection (ATCC). KG1a, OCI-AML5, U937 and primary AML/ALL samples were provided by Dr. Mark Minden and through the Leukemia Tissue Bank at Princess Margaret Cancer Centre/University Health Network. Primary human CD34⁺ hematopoietic cells purified from cord blood samples were obtained from STEMCELL Technologies.

### Genes

Clone 9 TCRα and TCRβ genes were codon-optimized according to sequences reported by Lin et al (Lin et al., 2013) and were fused by a furin cleavage site, an SGSG spacer sequence, and an F2A sequence. Immunoglobulin genes of 5H2 mAb were cloned via 5′-rapid amplification of cDNA ends (RACE) PCR as previously described (Nakatsugawa et a., 2015). The single-chain variable fragment (scFv) of CAR was formed by connecting the variable regions of heavy (VH) and light chain (VL) derived from 5H2, via a Whitlow linker. For 28z CAR, the scFv was linked to human CD28 transmembrane and cytoplasmic domain, followed by cytoplasmic domain of human CD3ξ. For 4-1BBz CAR, the scFv was linked to human CD8α transmembrane domain, followed by cytoplasmic domains of human 4-1BB and CD3ξ. Both TCR and CAR constructs were N-terminally linked to human nerve growth factor receptor (ΔNGFR) via a furin cleavage site, a SGSG spacer sequence, and a P2A sequence. Full-length WT1 cDNA was fused with ΔNGFR via an optimized intervening sequence consisting of a furin cleavage site, an SGSG spacer sequence, and an P2A sequence.

### Transfectants

All cDNAs were cloned into the pMX retroviral vector and all transfectants were generated using retrovirus produced by 293GPG cells or PG13 cells as previously published (Butler et al., 2007; Hirano et al., 2006). To generate K562/class II⁺ transfectants, K562 were individually transduced with the following HLA-class II genes: DPA1*01:03/DPB1*01:01 (DP1), DPA1*01:03/DPB1*02:01 (DP2), DPA1*01:03/DPB1*03:01 (DP3), DPA1*01:03/DPB1*04:01 (DP4-0401), DPA1*02:01/DPB1*04:02 (DP4-0402), DPA1*02:01/DPB1*05:01 (DP5), DPA1*02:01/DPB1*13:01 (DP13), DPA1*02:06/DPB1*104:01 (DP104), DQA1*02:01/DQB1*02:02 (DQ2.2), DQA1*01:02/DQB1*05:02 (DQ5.2), DQA1*01:04/DQB1*05:03 (DQ5.3), DQA1*02:01/DQB1*03:03 (DQ9.2), DRB1*01:01, DRB1*04:01, DRB1*07:01, DRB1*13:01, DRB1*14:54, DRB1*15:01, DRB1*15:02, DRB1*16:01, DRB5*02:02 (DR51b), DRB3*02:02 (DR52b), To ectopically express WT1, K562/class II⁺ transfectants were further transduced WT1/ΔNGFR. ΔNGFR positive cells were isolated using anti-NGFR beads (Miltenyi Biotec).

CD3-T cells were purified using the Pan T Cell Isolation kit (Miltenyi Biotec). Purified T cells were stimulated with aAPC/mOKT3 (aAPC expressing a membrane-bound form of anti-CD3 mAb (clone OKT3) and the co-stimulatory molecules CD80 and CD83) irradiated with 200 Gy at an effector: target (E: I) ratio of 5:1 (Sugata et al, 2021). 48 hours later, activated T cells were retrovirally transduced with CAR or TCR genes via centrifugation at 2,000 g for 2 b at 32° C. for two consecutive days using a Retronectin-coated plate (Takara Bio) and 100 IU ml-1 IL-2 and 10 ng ml-1 IL-15 were added to the CAR or TCR-transduced T cells. The culture medium was replenished every 2-3 days. ΔNGFR positive cells were isolated using anti-NGFR beads (Miltenyi Biotec).

### ELISA

ELISA plates were coated with anti-His antibodies and incubated at 4° C. overnight. Next day, the plates were washed and coated with DP4/CLIP or DP4/WT1 329-349 monomers, or CLIP (₉₇LPKPPKPVSKMRMATPLLMQALPM₁₂₀) (SEQ ID NO: 55) or WT1₃₃₀₋₃₄₈ (₃₃₀ CNKRYFKLSHLQMHSRKHT₃₄₈) (SEQ ID NO: 52) and incubated at 4° C. overnight. The monomers were kindly provided by the NIH Tetramer Core Facility. Next day, the plates were washed extensively before adding WT1-specific or control mAb. After incubation at room temperature for two hours, plates were washed extensively and incubated with ALP-anti-mouse IgG/IgM at room temperature for 30 minutes. Finally, the plates were washed and incubated with p-nitrophenyl phosphate (PNPP) substrate (Pierce, Rockford, IL) at room temperature. The reaction was terminated by adding 1 mol/L NaOH. The optic density (OD) (405 nm) was read (Spectramax 190 Microplate Reader; Molecular Devices, Sunnyvale, CA).

### Biolayer Interferometry (BLI)

BLI experiments were performed using an Octet Red96 instrument with Streptavidin (SA, cat. number 18-5019)-coated biosensor tips (Sartorius). All experiments were performed at 25 C with black flat bottom 96-well microtiter plates (Greiner Bio-One GmbH, Germany). SA tips were hydrated in HPS-EP+ buffer (10 mM HEPES, pH 7.4, 150 mM NaCl, 3 mM. EDTA, 0.005% Tween-20; Cytiva, Sweden AB) for 10 min. Following sensor check measurements in HPS-EP+ buffer, tips were dipped into 200 ul of 50 nM of N-linked biotinylated WT1₃₂₈₋₃₄₈ peptide (Genscript, Piscataway, NJ) diluted in HPS-EP+ buffer until 2.0 nm response was achieved. Peptide-loaded SA tips where then quenched in 100 nM solution of free biotin (Sigma-Aldrich) for 120 s, followed by baseline measurements in buffer to remove excess peptide for additional 120 s. Association step with 5H2 antibody at various concentrations were performed for 600 s followed by dissociation for 600 s in buffer only. Data were analyzed in Octet Data Analysis software and steady state affinity measurements at all concentrations were globally fit using a Langmuir (1:1) model

### Flow Cytometry

For surface staining, cells were pelleted and resuspended in a mAb master mix diluted in a PBS/2% FBS buffer. Cells were incubated at 4° C. for 15 minutes and washed prior to analysis. Monoclonal antibodies recognizing the following targets were used for surface staining: CD4 (300505, 1:40, BioLegend), CD8 (301007, 1:100, BioLegend), pan HLA class II (361703, 1:100, BioLegend), NGFR (345131, 1:40, BioLegend), CD25 (301007, 1:100, BioLegend), CD62L (304805, 1:25, BioLegend), CD45RA (304111, 1:10, BioLegend), PD-1 (329905, 1:40, BioLegend), Tim-3 (345007, 1:20, BioLegend). Mouse isotype controls were purchased from BioLegend. Biotinylated Protein-L was purchased from GenScript. For intracellular cytokine staining, TCR or CAR-T cells were stimulated with indicated target cells at an E:T ratio of 1:1. Brefeldin A (BioLegend) was added to the culture media after 2 hours, and the cells were further cultured for 4 hours. Following the surface marker staining, the cells were fixed and permeabilized using the Cytofix/Cytoperm Kit (BD Biosciences) and stained with FITC-anti-IL-2 (500305, 1:20, BioLegend), APC-anti-TNF-α (502913, 1:100, BioLegend), and PE-Cy7-anti-IFN-7 (506517, 1:200, BioLegend). The frequency of cytokine-producing cells within the CD8⁺ or CD4⁺ T cell population was determined by flow cytometric analysis. For intracellular WT1 staining, cells were fixed and permeabilized using the Cytofix/Cytoperm Kit (BD Biosciences) and stained with mouse anti-WT1 mAb (NB110-60011SS, 1:200, Novus Biologicals), followed by BV421-anti-mouse IgG (405317, 1:20, BioLegend).

### Immunoblotting

Equal amounts of proteins were separated on 12% gels by SDS-PAGE and transferred to Immobilon-P PVDF membranes (Millipore). The membranes were probed with the primary antibodies at 4° C. overnight. The membranes were then washed and incubated with HRP-conjugated anti-mouse IgG (Promega) or anti-rabbit IgG (Santa Cruz Biotechnology) secondary antibody at room temperature for 1 hr. The following antibodies were used: anti-WT1 mAb (83535S, 1:1000, CST) and anti-β-actin mAb (Santa Cruz Biotechnology). The signal was detected by Amersham ECL Prime Western Blotting Detection Reagent (GE Healthcare).

### Elispot Assays

Interferon (IFN)-γ ELISPOT were performed as previously described (Hirano et al., 2003; Hirano et al., 2006; Nakatsugawa et al., 2015). Briefly, polyvinylidene difluoride (PVDF) plates (Millipore) were coated with capture monoclonal antibody (1D1K; MABTECH, Mariemont, OH). Indicated number of T cells were incubated with 2×10\hat{ }4 stimulator cells for 20-24 h at 37° C. in the presence or absence of indicated peptides. Plates were washed and incubated with biotin-conjugated detection monoclonal antibody (7-B6-1; MABTECH). HIRP-conjugated Streptavidin (DAKO, Carpenteria, CA) was then added, and IFN-g spots were subsequently developed. The peptides used were MAGE-A3243-258 (243KLLTQHFVQENYLEY258) (SEQ ID NO: 57), WT1₃₂₈₋₃₄₈ (328 PGCNKRYFKLSHLQMHSRKHT348) (SEQ ID NO: 58) and cross-reactive peptide candidates listed in Table 5. All peptides were purchased from GenScript (Piscataway, NJ).

### In Vitro Cytotoxicity Assays

Ten thousand K562 transfectants were labelled with 5 M florescent Vybrant™ DiO in PBS (Thermo Fisher Scientific) for 15 mins at 37° C. After washing, the DiO-labelled targets were added to 96-well plates in 100 l RPMI with 10% FBS. 5×10\hat{ }4 TCR-transduced T cells were added at the indicated E:T ratio. After 6 h/18 h co-culture, cells were transferred to a new microtiter plate. 3 M TO-PRO-3 (Thermo Fisher Scientific) was added to cell suspension to stain for dead cells and cells were analyzed by flow cytometric assays to determine the frequency of live and dead DiO*target cells. non-adherent cells were collected and transferred to a new microtiter plate. The % cytotoxicity is calculated by: % DiO⁺ TO-PRO-3⁺ (with T cells)−% DiO⁺ TO-PRO-3⁺ (without T cells).

### Statistical Analysis

Statistical analysis was performed using GraphPad Prism 9.0. Statistically significant differences between two groups were assessed using a two-tailed unpaired t-test. Comparisons between more than two groups were carried out by an ANOVA test followed by Bonferroni post hoc analysis were employed. P values less than 0.05 were considered significant. No statistical method was used to predetermine sample size.

### Results

Generation of mABS Targeting WT1 Peptide Presented by HLA Class II Molecules

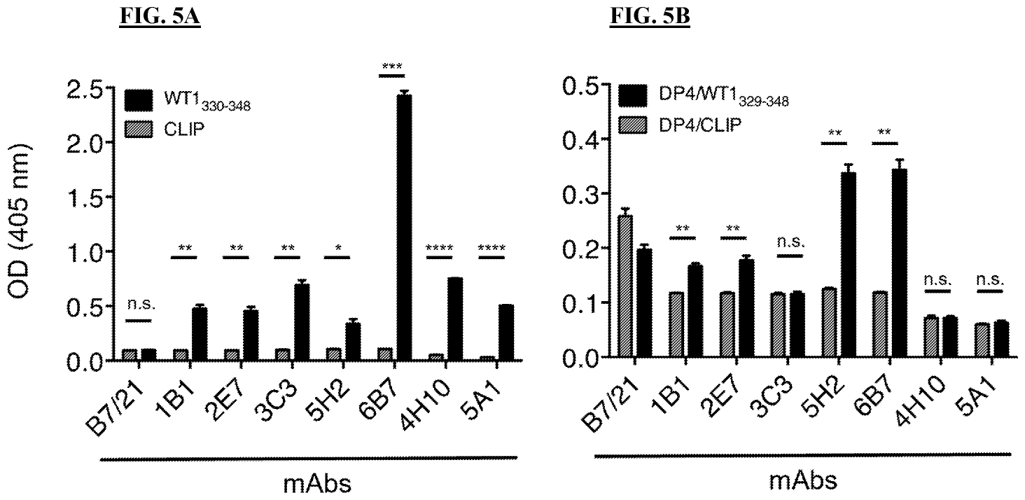

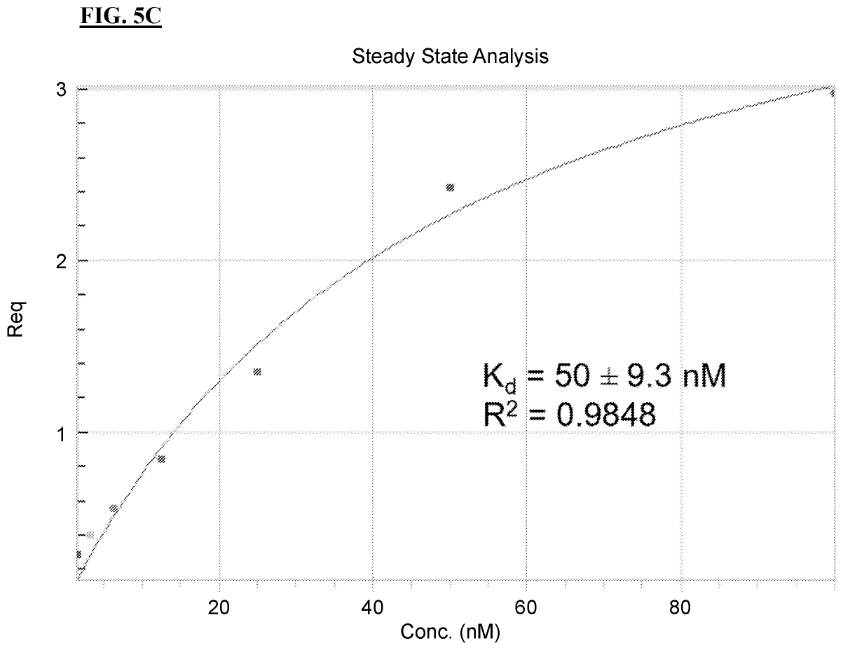

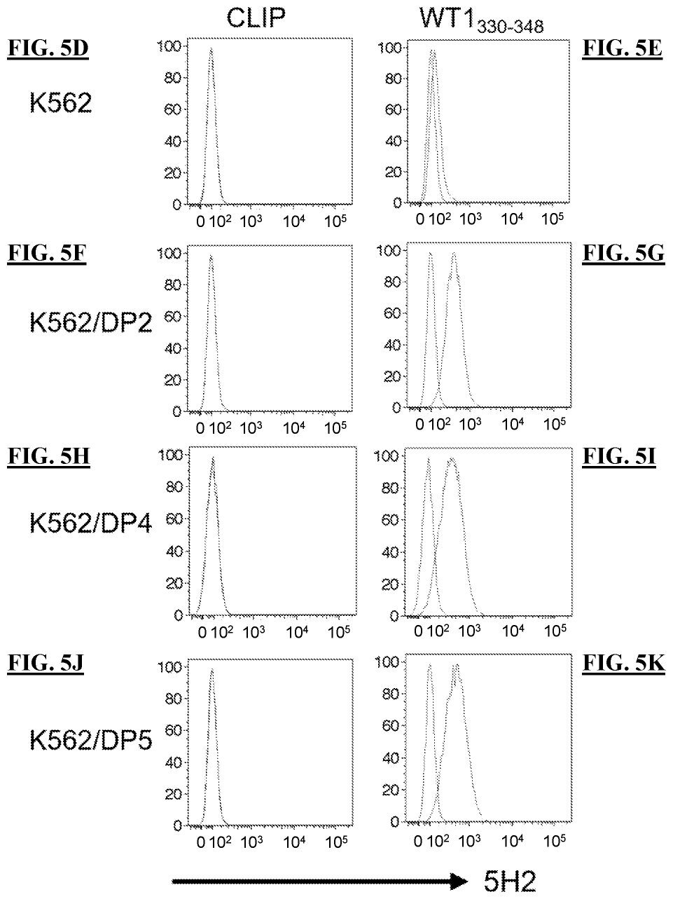

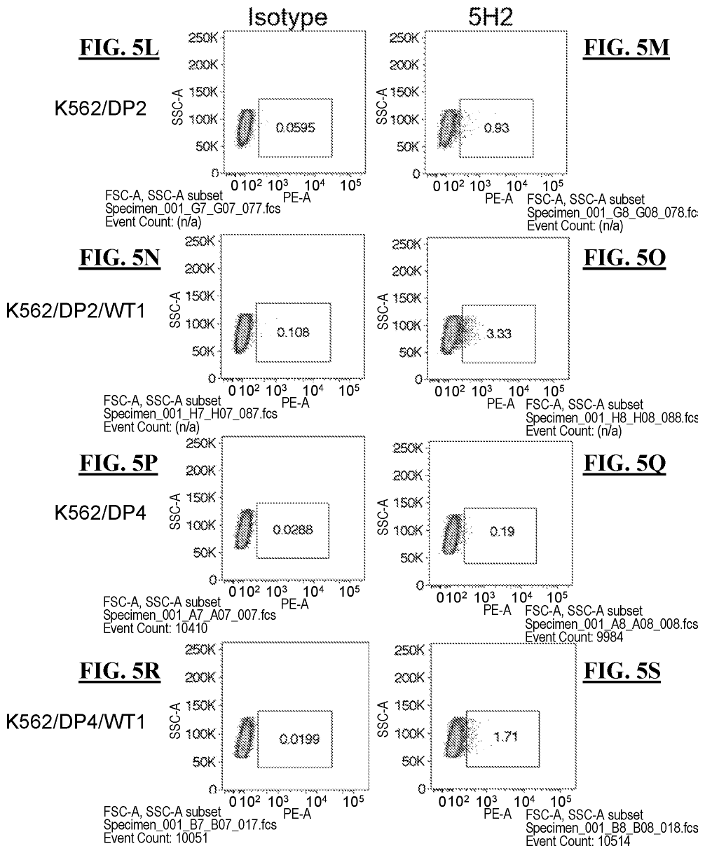

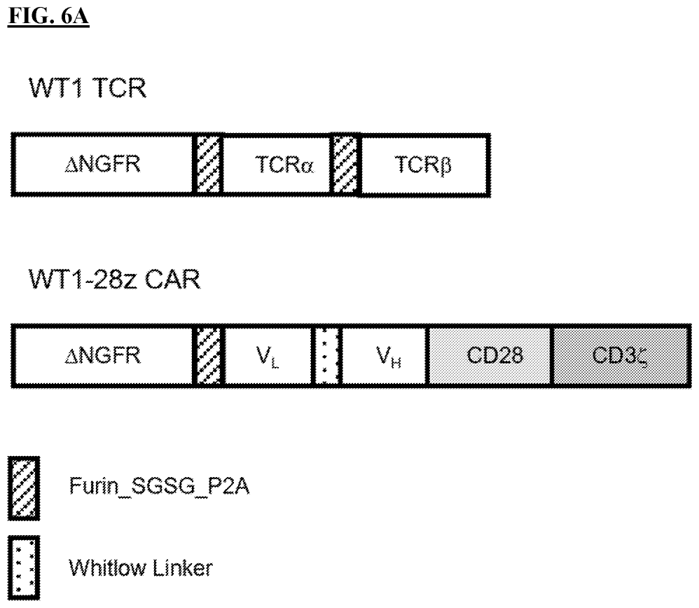

As shown in herein, we have identified WT1₃₂₈₋₃₄₈ peptide as a DP2 and DP4-restricted natural WT1 epitope. We immunized mice with WT1₃₃₀₋₃₄₈peptide and raised multiple mouse hybridoma clones specific for the peptide. The shorter peptide was chosen for immunization since the Cysteine residue at position 330 is critical for N-terminal conjugation with adjuvant to enhance immunogenicity. In an ELISA assay, these clones were able to recognize plate-bound WT1₃₃₀₋₃₄₈ peptide but not the negative control CLIP peptide (FIG. 5A). More importantly, some of these clones were able to bind to plate-bound DP4/WT1₃₃₀₋₃₄₈ monomers, but not DP4/CLIP monomers, indicating their capabilities to recognize the WT1 peptide presented by DP4 molecules (FIG. 5B). Using a biolayer interferometry (BLI) assay, we measured the affinity of the clone, 5H2, and showed that 5H2 bound to WT1₃₃₀₋₃₄₈ with a KD of approximately 50 nM (FIG. 5C). To confirm that the WT1-specific mAb can recognize WT1₃₃₀₋₃₄₈ presented by DP4 molecules on the cell surface, we pulsed K562 cells expressing different DP molecules with WT1₃₃₀₋₃₄₈, followed by incubation with clone 5H2 and flow cytometric analysis. Unless specified otherwise, DP4 tested herein comprised of DPA1*01:03/DPB1*04:01. Interestingly, the data showed that 5H2 can recognize exogenously-pulsed WT1₃₃₀₋₃₄₈ presented by multiple DP molecules such as DP2 and DP5 in addition to DP4 (FIGS. 5D-5K), demonstrating the potential of 5H2 to overcome HLA barrier and target WT1 peptide on multiple class II molecules. We further showed that 5H2 can bind to K562/DP2 and K562/DP4 cells which express endogenous WT1, and to a greater extent, the target cells that ectopically expressed WT1 (FIGS. 5L-5S). These data suggest that 5H2 can also recognize naturally processed and presented WT1 peptide in the context of various DP molecules.

### Comparison Between WT1 TCR and CAR-T Cells Targeting WT1 Peptide Presented by HLA-DP

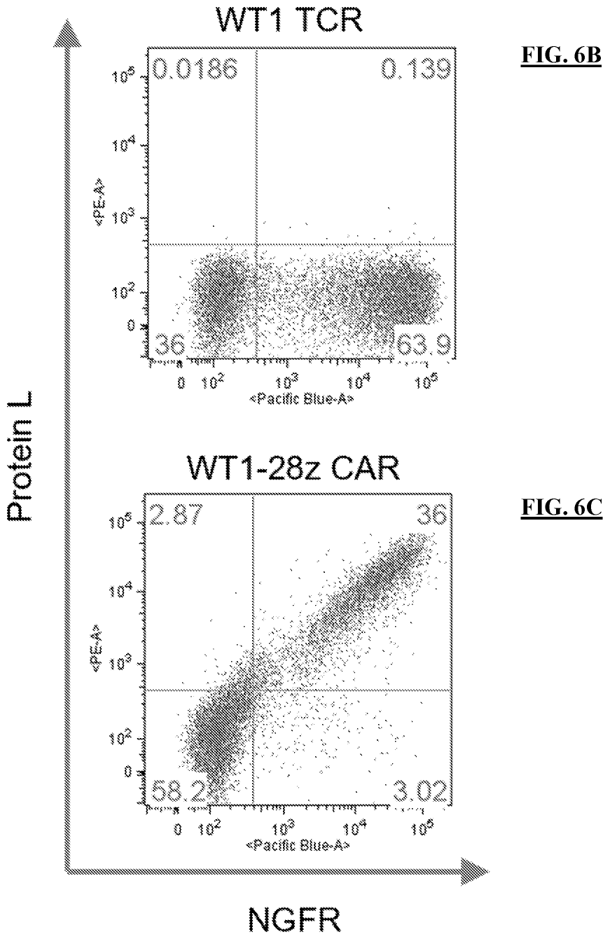

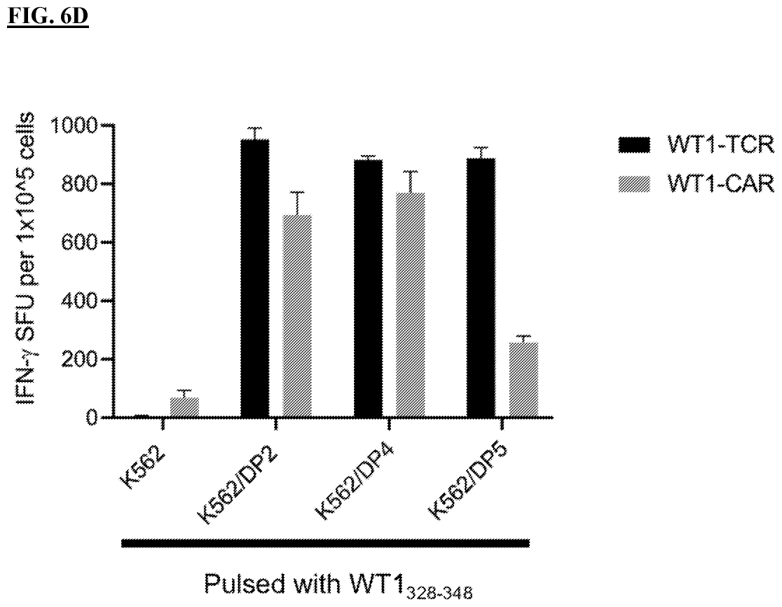

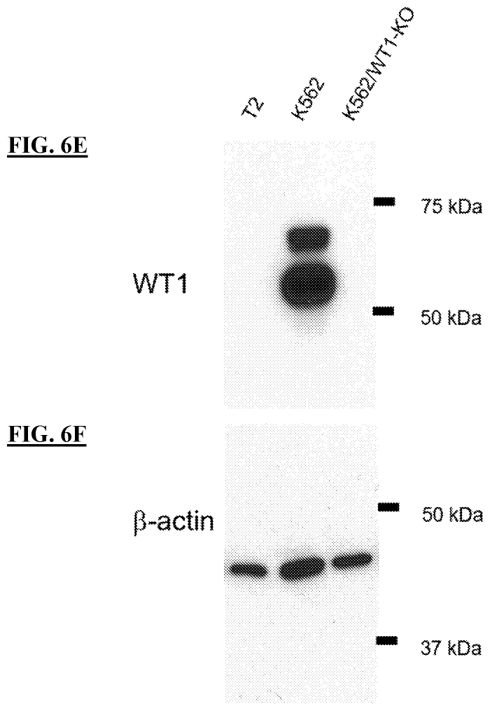

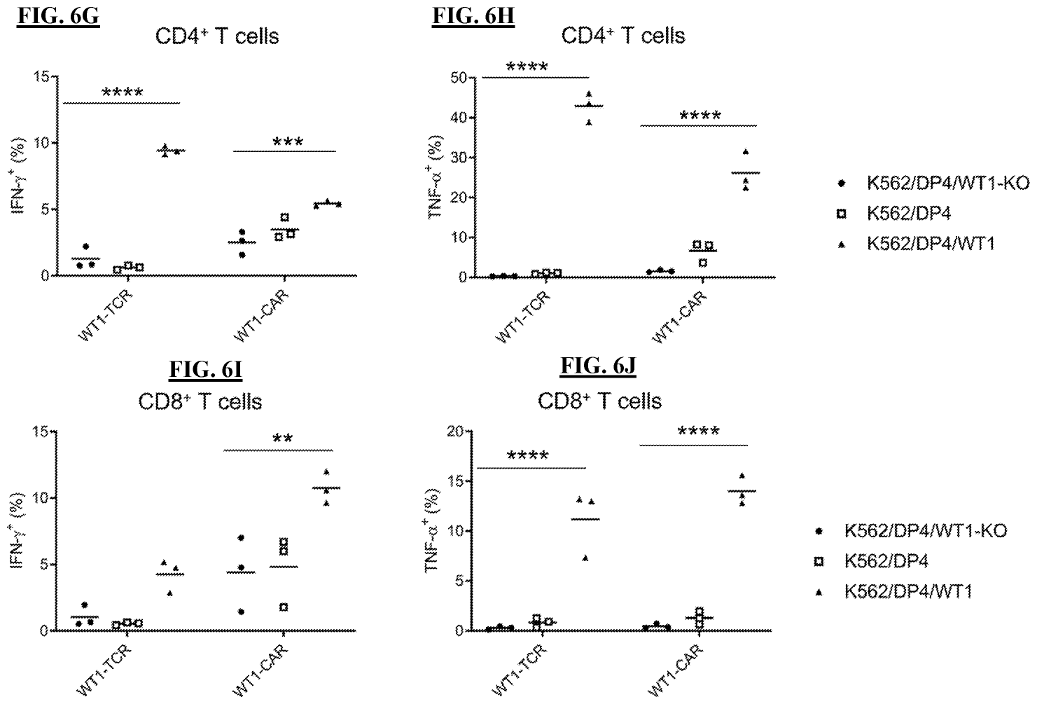

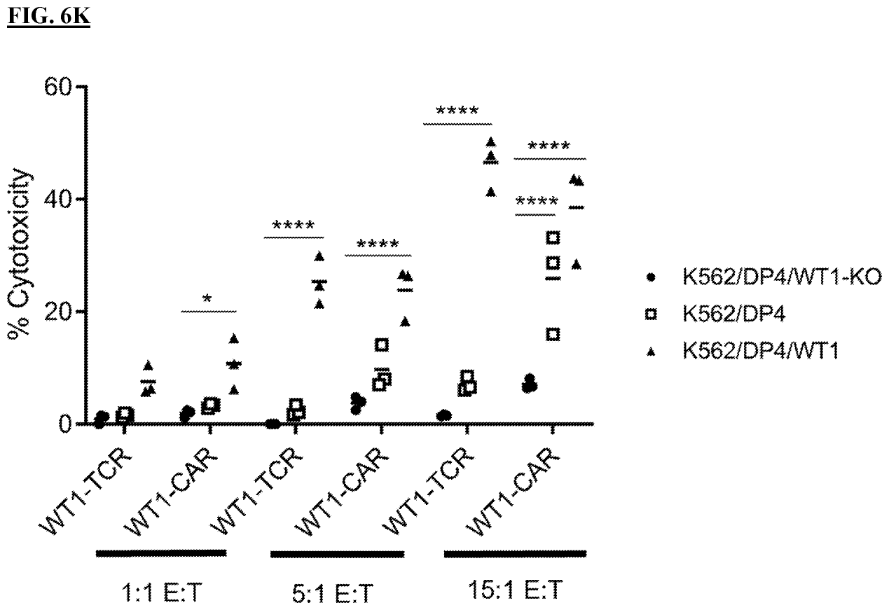

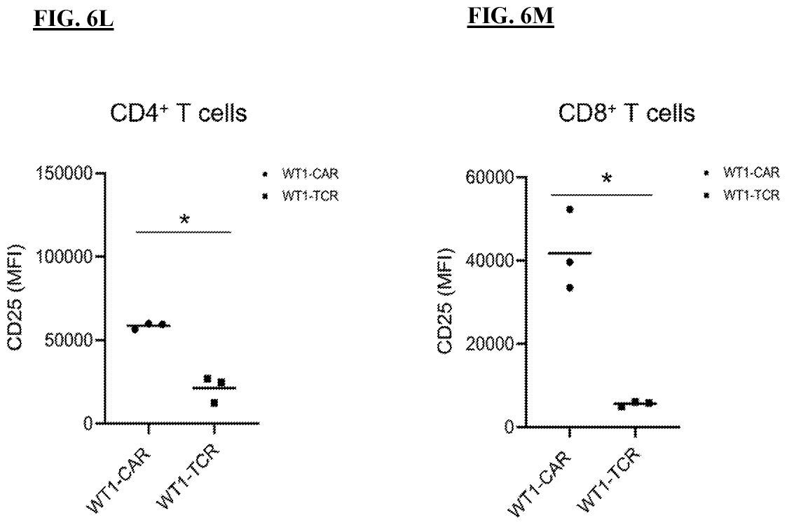

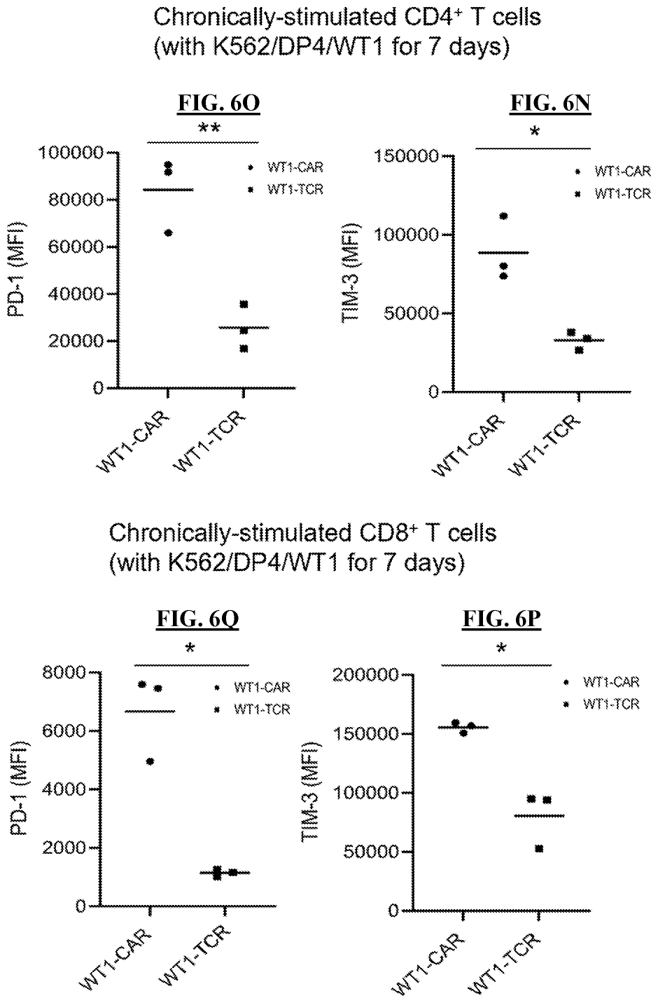

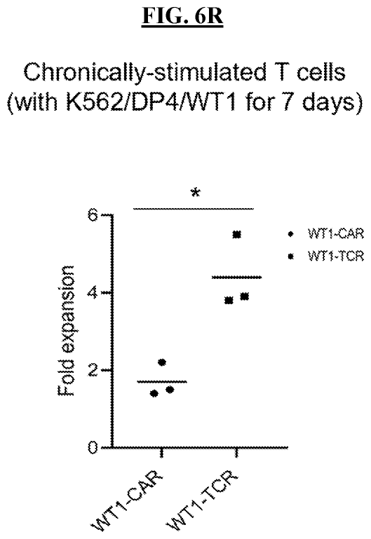

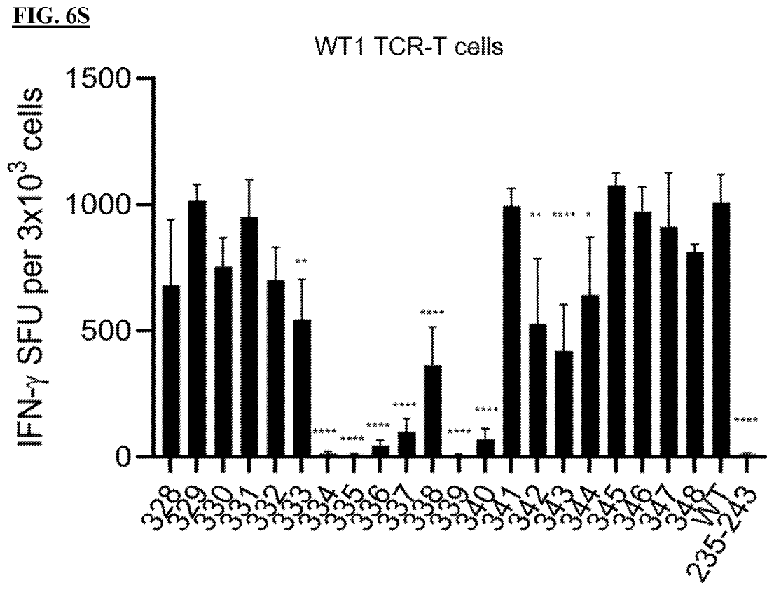

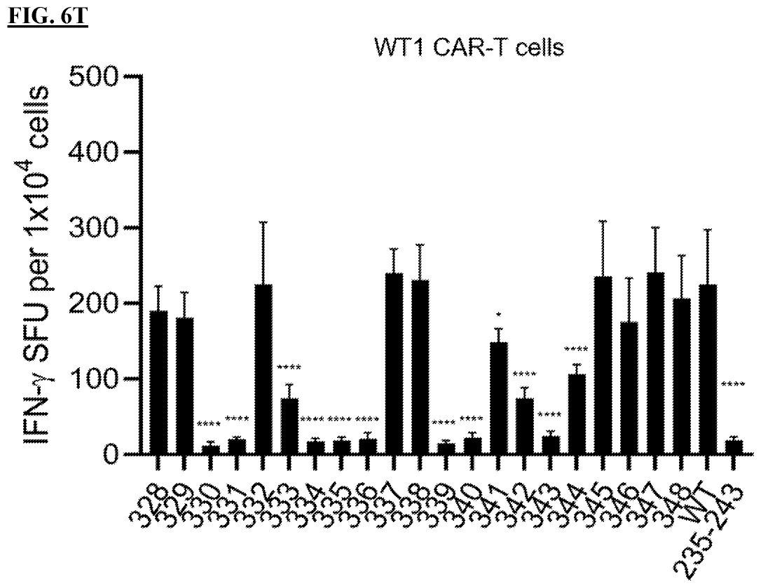

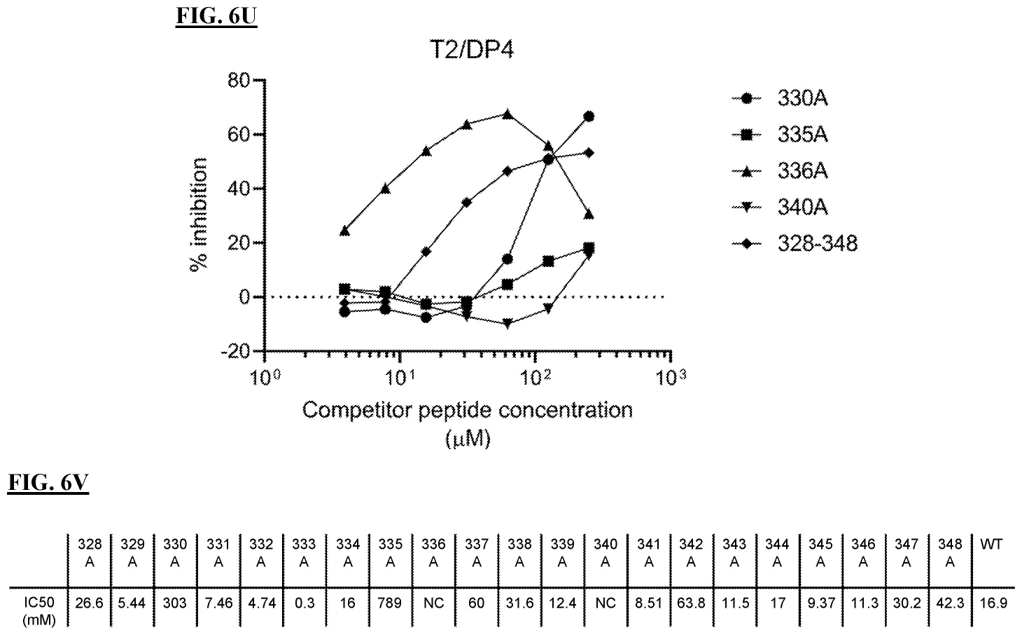

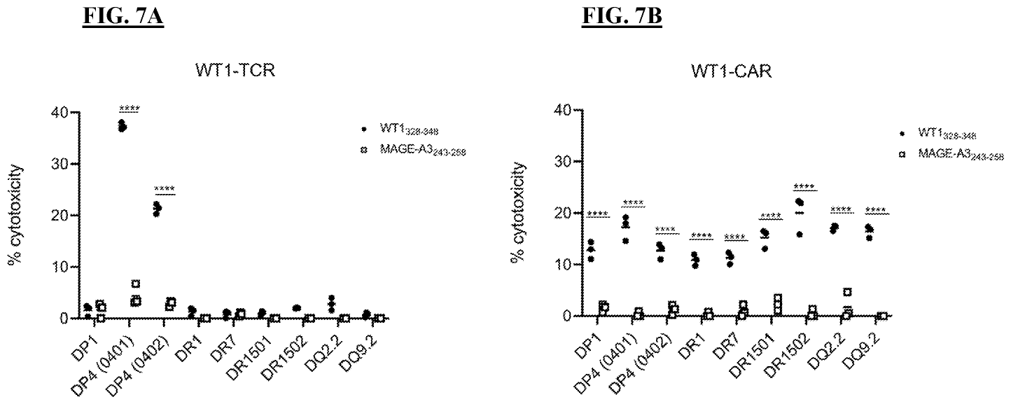

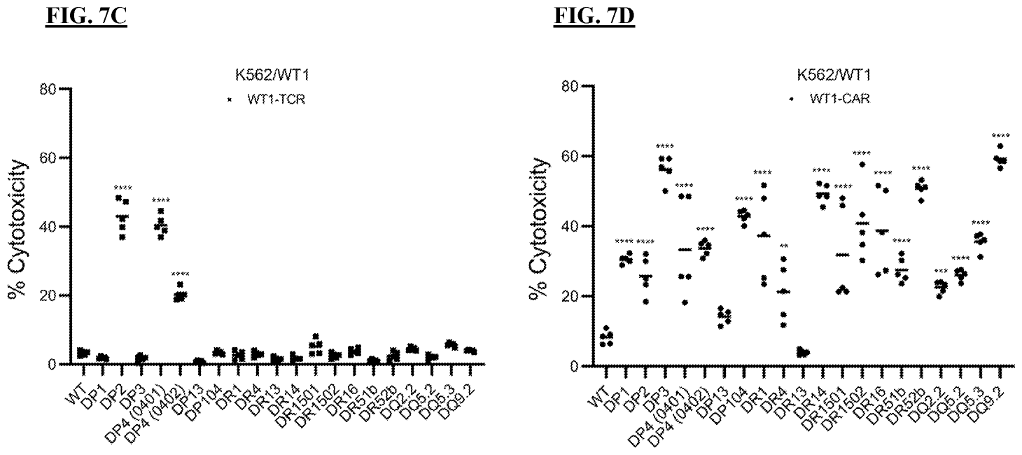

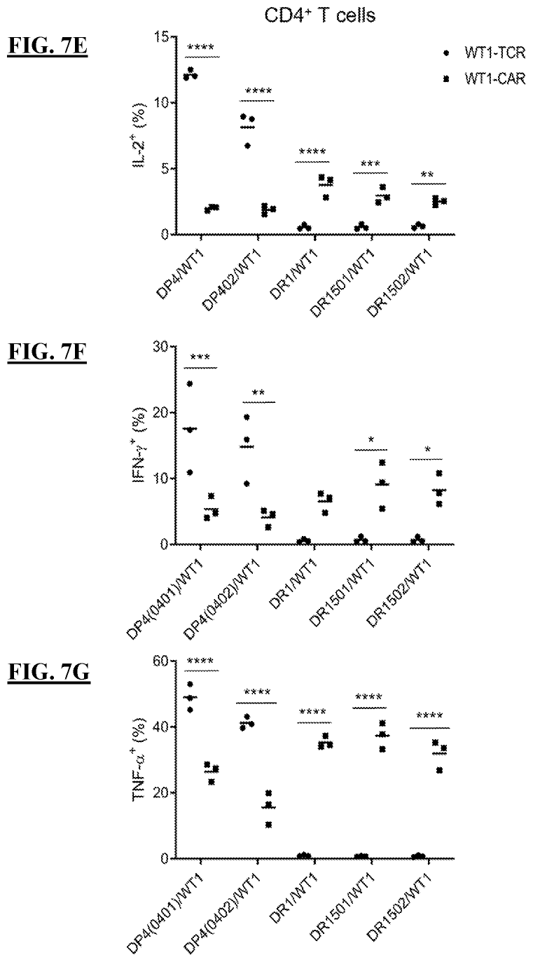

To generate WT1-specific CAR-T cells, we cloned the immunoglobulin heavy and light chain genes from 5H2 mAb. 5H2-derived scFv was constructed by linking the variable domains of 5H2 heavy and light chain sequences through Whitlow linker (Cooper et al., 2003), which was then ligated upstream to transmembrane and cytoplasmic domain of CD28 and cytoplasmic domain of CD3ξ (FIG. 6A) to generate the widely used 2^{nd} generation 28z CAR construct (designated as WT1 CAR herein, unless specified otherwise). For comparison, construct of DP2/4-restricted WT1-specific TCR, clone 9, was also shown (designated as WT1 TCR herein, unless specified otherwise). Both CAR and TCR constructs were N-terminally linked to ΔNGFR to monitor transduction efficiency and for subsequent purification. Following retroviral transduction into primary T cells, the majority of the transduced, ΔNGFR⁺ cells expressed the CAR efficiently on the cell surface (FIGS. 6B-6C), which was detected by protein L, a bacterial protein commonly used for the detection of CAR on the cell surface due to its ability to bind to the variable region of immunoglobulin light chains (Zheng, Chinnasamy, & Morgan, 2012).

To compare the functionality of WT1 TCR and WT1 CAR-T cells, we first purified both with anti-NGFR beads to enrich transduced T cells. In an ELISPOT assay, WT1 CAR-T cells secreted similar level of IFN-γ as WT1 TCR-T cells when stimulated with K562/DP2 and K562/DP4 cells pulsed with WT1₃₂₈₋₃₄₈, and to a lesser extent, peptide-pulsed K562/DP5 cells (FIG. 6D). This confirmed that WT1 CAR-T cells were able to recognize WT1 peptide presented by various DP molecules like 5H2. We next investigated the ability of WT1 CAR-T cells to target naturally processed and presented WT1 peptide in the context of class II molecules. We found that both WT1 TCR and CAR-T cells secreted cytokines such as IFN-γ and TNF-α when stimulated with K562/DP4 cells ectopically expressing WT1 at low E/T ratios. K562 cells with WT1 knockout (KO) (FIGS. 6E-6F) were generated and used as a negative control, which induced significantly lower cytokine release from WT1 CAR and TCR-T cells (FIGS. 6G-6J). Both WT1 CAR and TCR-T cells also demonstrated similar level of cytotoxicity towards K562/DP4 cells ectopically expressing WT1 (FIG. 6K). Interestingly, at a higher ET ratio, WT1 CAR-T cells were also cytotoxic against K562/DP4 cells expressing endogenous WT1. This suggest that WT1 CAR-T cells are more sensitive than WT1 TCR-T cells against lower WT1 peptide density presented by DP4 molecules on the cell surface, which might be caused by different affinity between CAR and TCR for WT1 peptide presented by DP4. Since class II-bound peptides can possess ragged ends at both terminuses, it is also possible that WT1 CAR and TCR-T cells target different natural WT1 epitopes in the context of DP4, resulting in differential responses.

CAR-T cells are known to exhibit tonic signaling in a ligand-independent manner, which leads to T cell exhaustion and impaired antitumor efficacy (Ajina & Maher, 2018). Two weeks following retroviral transduction, both CD4⁺ and CD8⁺ WT1 CAR-T cells expressed higher level of T cell activation marker, CD25, compared to WT1 TCR-T cells (FIGS. 6L-6M). To examine if higher level of tonic signaling can lead to T cell exhaustion, WT1 TCR and CAR-T cells were co-cultured with K562/DP4 cells ectopically expressing WT1 for 7 days. Following prolonged culture, WT1 CAR-T cells showed higher level of markers associated with T cell exhaustion such as PD-1 and TIM-3 (FIGS. 6N-6Q). In addition, WT1 CAR-T cells also expanded significantly less compared to WT1 TCR-T cells (FIG. 6R). These results show that, although both WT1 CAR-T and TCR-T cells recognized DP4⁺/WT1⁺ target cells, higher tonic signaling in CAR-T cells can lead to greater T cell exhaustion and reduced proliferation upon chronic antigen stimulation.

Although both WT1 CAR and TCR-T cells reacted to WT1 peptide presented by DP4, they may manifest different modes of recognition, given that WT1 CAR was derived from a peptide-centric mAb. Alanine scanning was performed, in which WT1 CAR and TCR-T cells were stimulated with T2/DP4 cells individually pulsed with a series of alanine-substituted WT1₃₂₈₋₃₄₈ peptides (FIGS. 6S-6T). A residue at a given position was considered critical if alanine substitution of which resulted in a significant reduction of IFN-γ secretion from WT1 TCR or CAR-T cells, where we defined as having a p value less than 0.0001 compared to T cell response induced by nonmutated peptide. The results revealed that the amino acid residues at positions 334, 335, 336, 337, 338, 339, 340, and 343 were critical for recognition by WT1 TCR-T cells. On the other hand, residues at positions 330, 331, 333, 334, 335, 336, 339, 340, 342, 343, and 344 were essential for WT1 CAR-T cells. Since the lack of recognition by T cells following alanine substitution can be a result of reduced binding of mutant peptides to DP4, a T2/DP4-based competitive binding assay as described herein was performed and residues at positions 330, 335, 336, and 340, which were among the residues essential for recognition by both WT1 TCR and CAR-T cells, were identified to be critical for DP4 loading (FIGS. 6U-6V). The remaining critical residues may directly interact with WT1 TCR and CAR-T cells or help to maintain the peptide conformation for optimal recognition. Interestingly, the data demonstrated that the core residues of WT1₃₂₈₋₃₄₈ presented by DP4 were crucial for recognition by WT1 TCR-T cells, whereas for WT1 CAR-T cells, additional residues near the N and C-terminus of WT1₃₂₈₋₃₄₈ were also essential. Despite some overlapping critical residues at the core of WT1₃₂₈₋₃₄₈, the results suggest that WT1 CAR and TCR-T recognize WT1 peptide presented by DP4 in different manners.

### WT1 CAR-T Cells Recognize WT1 Peptide Presented by Diverse HLA Class II Molecules

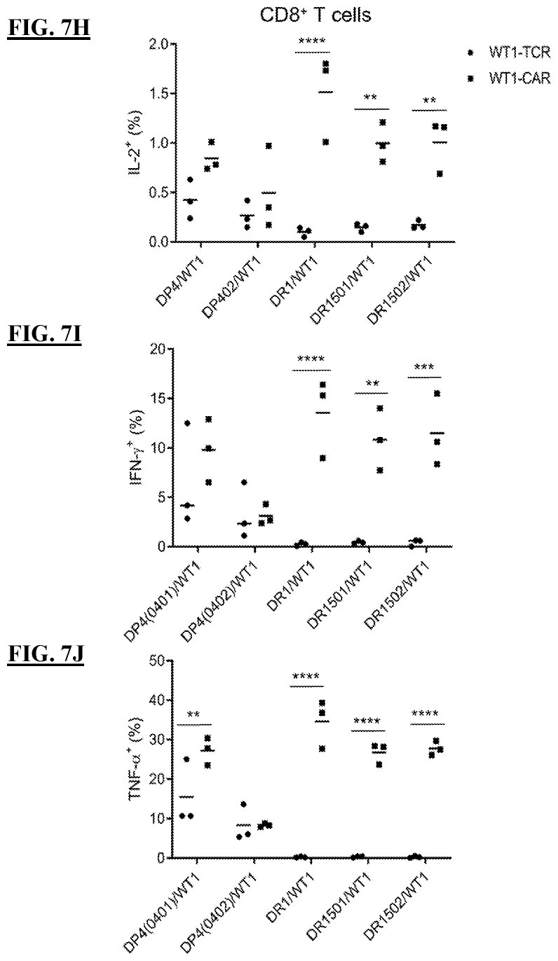

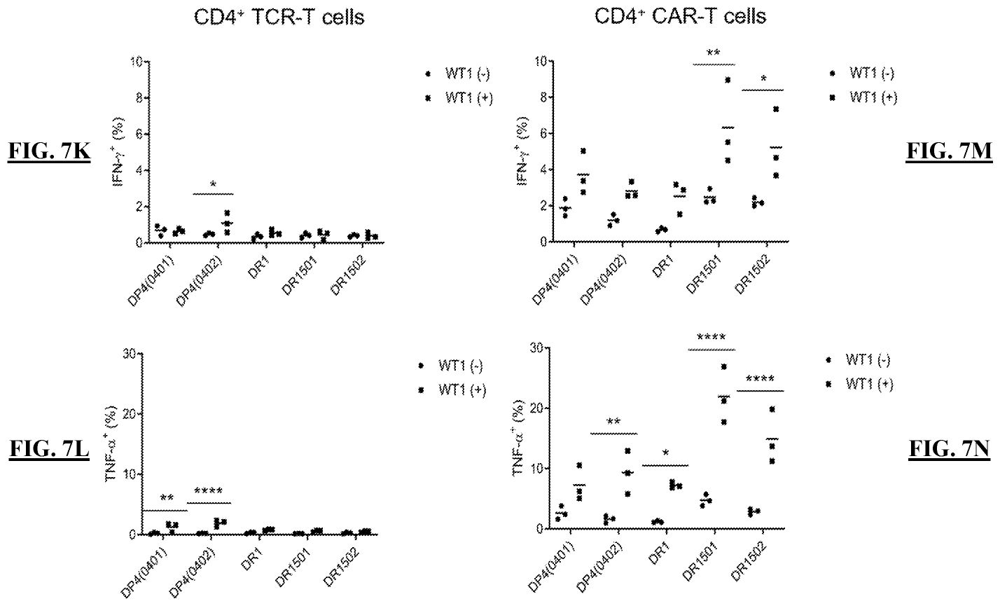

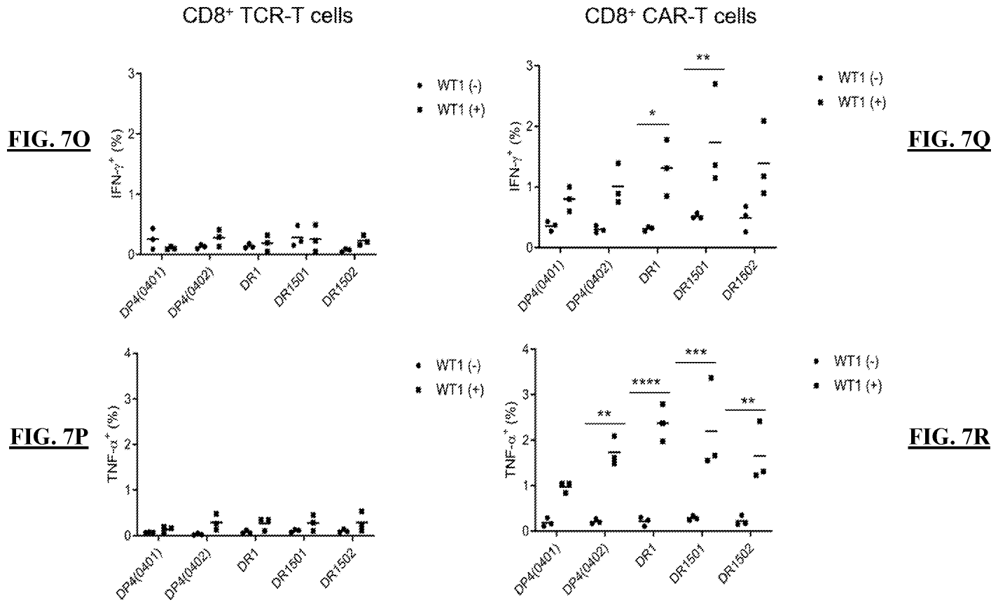

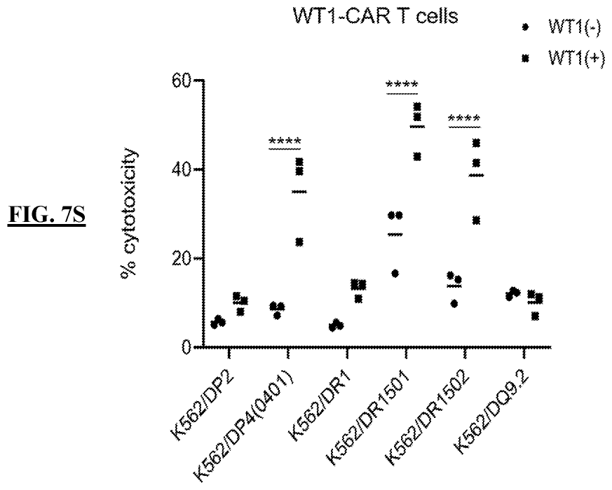

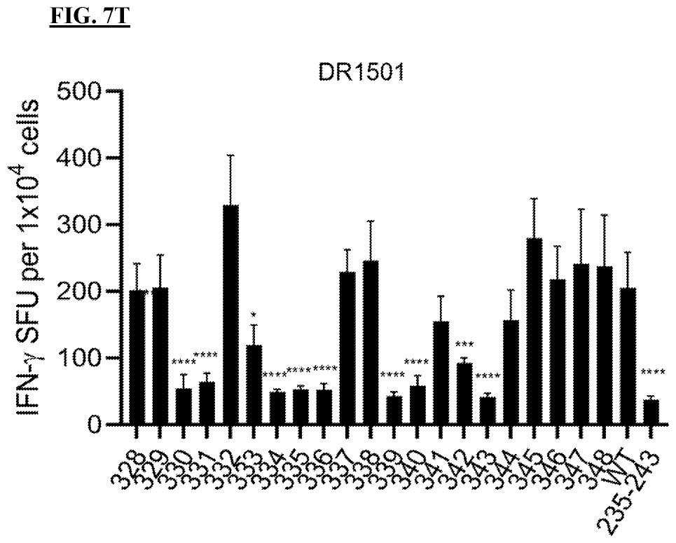

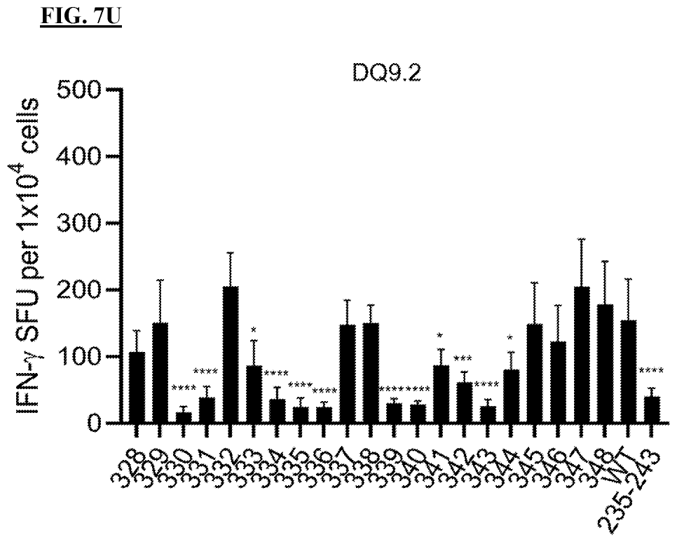

Our class II WT1 CAR was derived from mAb raised against WT1 peptide itself without context of class II. If the same or similar WT1 peptide with different ends are presented by diverse class II molecules, our peptide-centric WT1 CAR-T cells can be broadly used for cancer patients with various class II by overcoming the HLA class II barrier. We first investigated whether WT1₃₂₈₋₃₄₈ peptide binds to various HLA-DP, DR and DQ molecules in silico. NetMHC 3.2 algorithm predicted that WT1₃₂₈₋₃₄₈ peptide possessed binding affinity towards multiple DP and DR molecules comparable to DP2 and DP4 (Table 2) which were validated in vitro. It also showed predicted binding affinity towards DQ molecules albeit weaker. We then performed in vitro cytotoxicity assays and studied the ability of WT1 CAR-T cells to recognize WT1 peptide presented by different DP, DR and DQ molecules. While WT1 TCR-T cells only demonstrated cytotoxicity towards WT1₃₂₈₋₃₄₈-pulsed K562 cells expressing DP4 (0401 and 0402), WT1 CAR-T cells demonstrated strong cytotoxicity towards K562 cells expressing various class II pulsed with WT 1328-348 (FIGS. 7A, 7B). More importantly, WT1 CAR-T cells were able to kill WT1-transduced K562 cells individually expressing a broad panel of DPs, DRs and DQs, whereas as WT1 TCR-T cells were only able to kill WT1-transduced K562 cells individually expressing DP2 and DP4 (0401 and 0402) (FIGS. 7C, 7D). Cytokine secretion assays showed that CD4⁺ and CD8⁺ WT1 CAR-T cells secreted significantly higher level of IL-2, IFN-γ, and TNF-α towards various K562/DR cells ectopically expressing WT1 but not WT1 TCR-T cells (FIGS. 7E-7J). Since K562 cells express endogenous WT1, we also tested if WT1 CAR-T cells were able to recognize K562/class II⁺ cells without ectopic WT1 expression. The results showed that WT1 CAR-T cells secreted significantly greater cytokines and were more cytotoxic towards a series of DPs and DRs compared to their WT1-knockout counterparts (FIGS. 7K-7S). Collectively, these results demonstrate that WT1 CAR-T cells recognize WT1 peptide presented by a wide array of BLA class II molecules across DPs, DRs and DQs. WT1 TCR-T cells, on the other hand, are strictly bound by BILA restriction and are much less promiscuous than the WT1 CAR-T cells.

TABLE 2
Binding affinity and core binding sequence of WT1₃₂₈₋₃₄₈
for various HLA-DP, DR and DQ predicted by NetMHCII 3.2.

| | | Calculated |
|---|---|---|
| Allele | Predicted binding core sequence | affinity (nM) |
| DR1 | ₃₃₄YFKLSHLQM₃₄₂ | 71.07 |
| DR3 | ₃₃₅FKLSHLQMH₃₄₃ | 1342 |
| DR4 | ₃₃₅FKLSHLQMH₃₄₃ | 252.52 |
| DR7 | ₃₃₅FKLSHLQMH₃₄₃ | 220.05 |
| DR8 | ₃₃₅FKLSHLQMH₃₄₃ | 120.47 |
| DR9 | ₃₃₅FKLSHLQMH₃₄₃ | 237.57 |
| DR11 | ₃₃₄YFKLSHLQM₃₄₂ | 47.9 |
| DR13 | ₃₃₄YFKLSHLQM₃₄₂ | 111.65 |
| DR14 | ₃₃₅FKLSHLQMH₃₄₃ | 312.11 |
| DR1501 | ₃₃₅FKLSHLQMH₃₄₃ | 185.85 |
| DR1502 | ₃₃₅FKLSHLQMH₃₄₃ | 425.89 |
| DR16 | ₃₃₄YFKLSHLQM₃₄₂ | 70.84 |
| DR51b | ₃₃₅FKLSHLQMH₃₄₃ | 608 |
| DR52b | ₃₃₅FKLSHLQMH₃₄₃ | 364.08 |
| DP1 | ₃₃₅FKLSHLQMH₃₄₃ | 130.75 |
| DP2 | ₃₃₅FKLSHLQMH₃₄₃ | 125.5 |
| DP4 (0401) | ₃₃₅FKLSHLQMH₃₄₃ | 138.35 |
| DP4 (0402) | ₃₃₅FKLSHLQMH₃₄₃ | 208.9 |
| DP5 | ₃₃₅FKLSHLQMH₃₄₃ | 102.35 |
| DP13 | ₃₃₅FKLSHLQMH₃₄₃ | 187.6 |
| DP104 | ₃₃₅FKLSHLQMH₃₄₃ | 182.17 |
| DQ2.2 | ₃₃₅FKLSHLQMH₃₄₃ | 9241.11 |
| DQ5.2 | ₃₃₅FKLSHLQMH₃₄₃ | 2064.13 |
| DQ5.3 | ₃₃₅FKLSHLQMH₃₄₃ | 2294.13 |
| DQ9.2 | ₃₃₅FKLSHLQMH₃₄₃ | 2862.16 |

Since WT1 CAR-T cells recognize WT1 peptide presented by different class II molecules, we investigated the underlying mode of target recognition. To include one representative class II molecule from each of the three main class II types, we chose DP4 (0401), DR1501, and DQ9.2, all of which are common BILA alleles and were able to present WT1 peptide and induce WT1 CAR-T response. Alanine scanning, as described earlier, was performed to identify the amino acid residues of WT1₃₂₈₋₃₄₈ critical for recognition by WT1 CAR-T cells when presented by respective class II molecules. The results showed that the residues at positions 330, 331, 334, 335, 336, 339, 340, 343 were essential when WT1₃₂₈₋₃₄₈ was presented by any of the three class II molecule (FIGS. 6R, 7T, 7U). Unlike DP4 (0401), residues at position 333, 342 and 344 did not meet our predefined criteria to be considered critical when presented by DR1501 and DQ9.2, though they did lead to significantly weaker CAR-T response. Overall, the largely overlapping critical residues suggest that WT1 CAR-T cells recognize WT 1328-348 presented by these three representative class II molecules in a similar manner.

WT1 CAR-T Cells Recognize Various WT1⁺/Class II⁺ Leukemia and Lymphoma Cell Lines and Primary Leukemic Samples

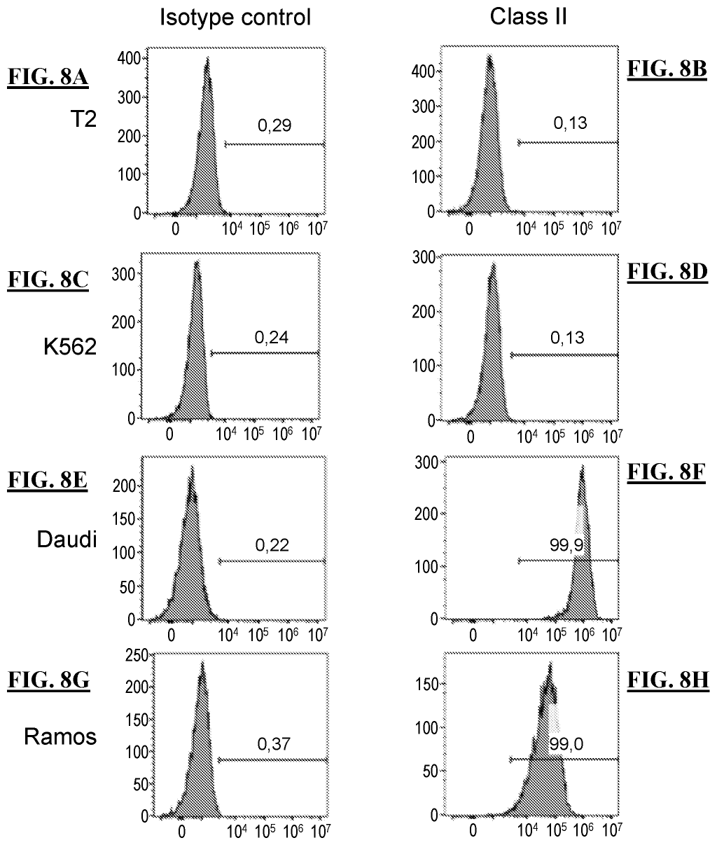

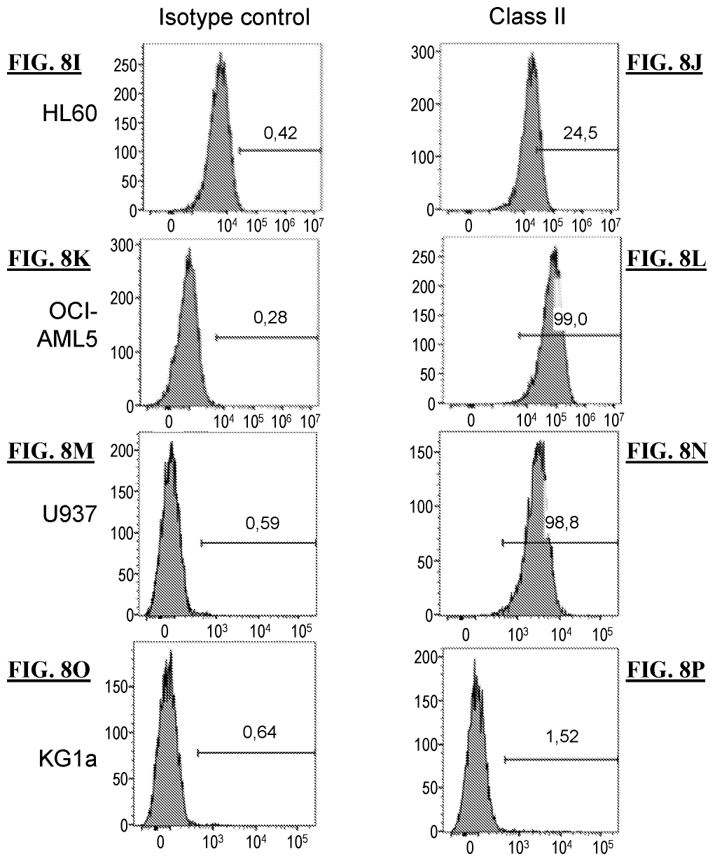

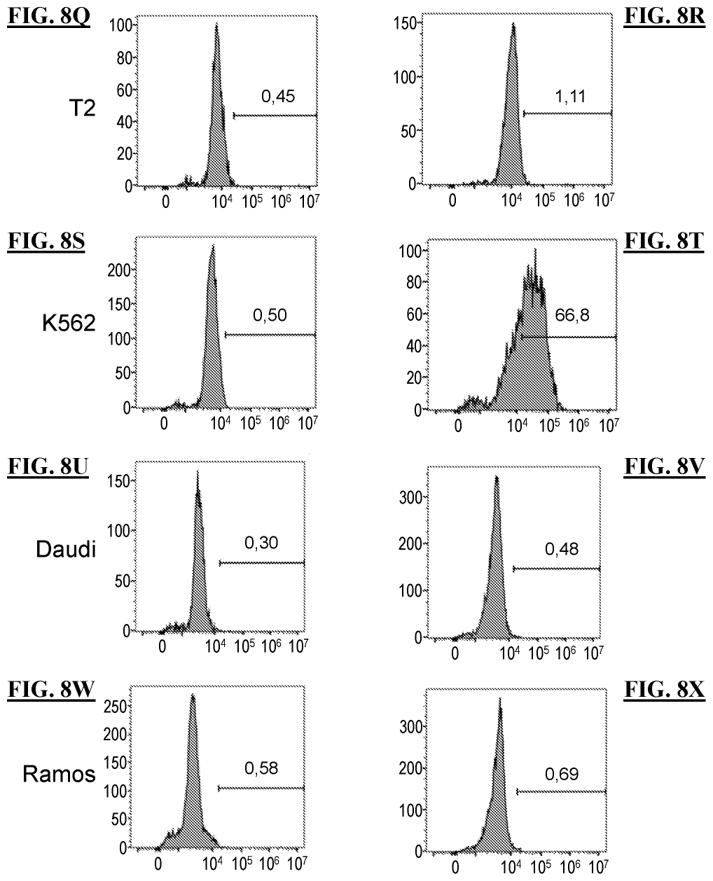

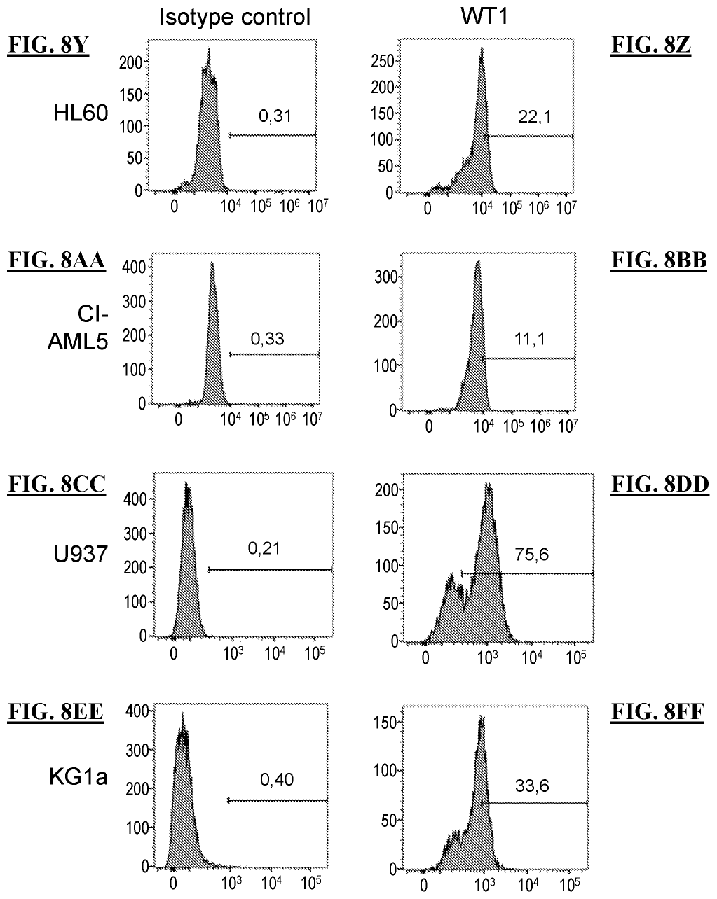

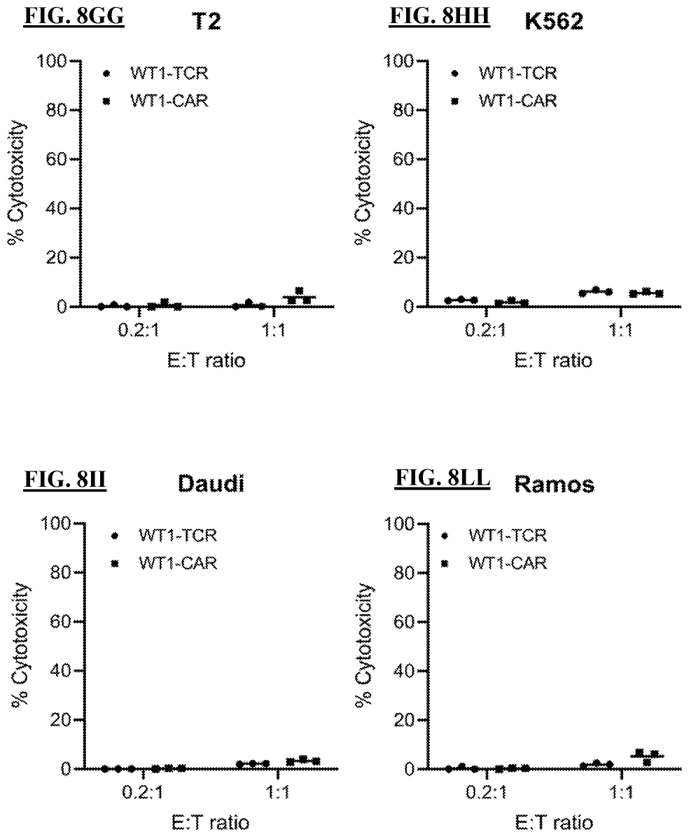

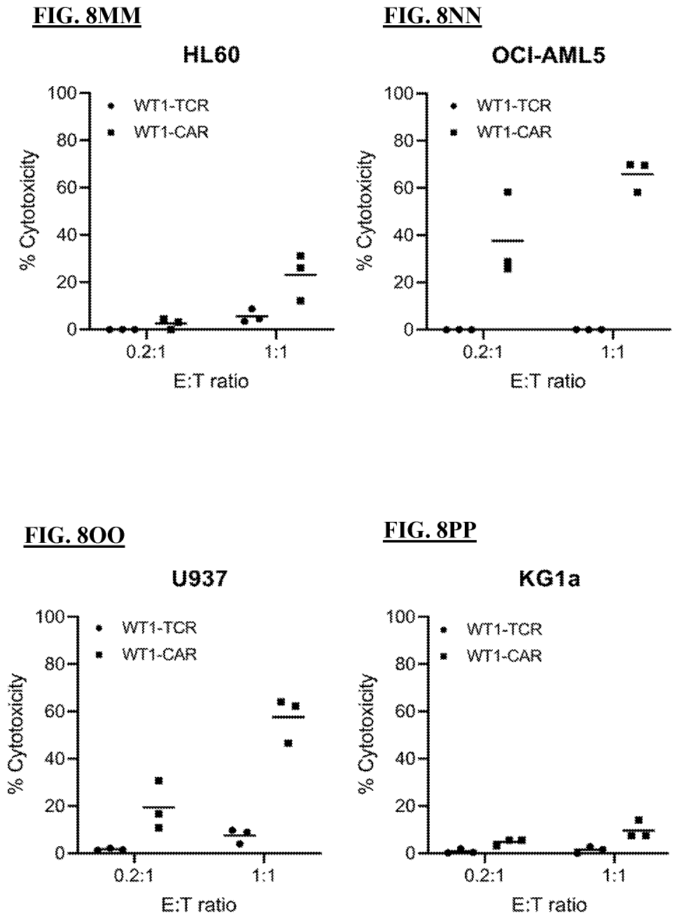

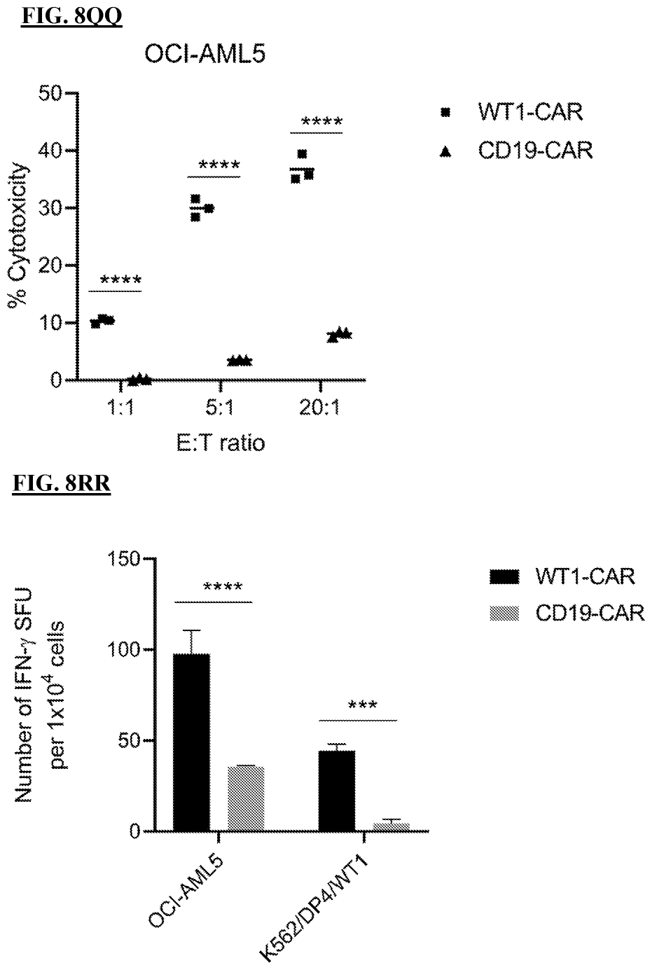

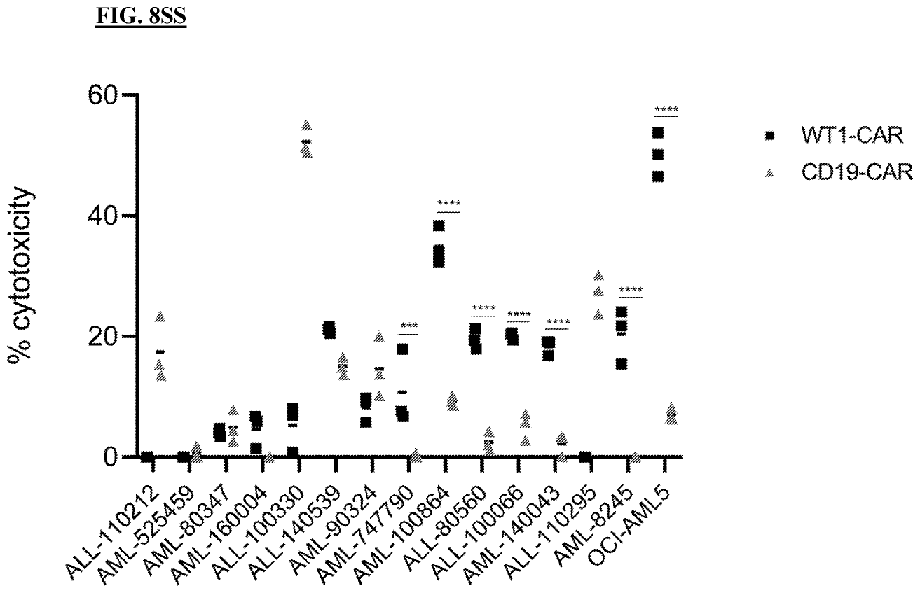

We then investigated the ability of WT1 CAR-T cells to recognize a panel of leukemia and lymphoma cell lines with varying class II and WT1 expressions. Class II and WT1 expressions were studied by flow cytometric analysis (FIGS. 8A-8FF; Table 3). WT1 CAR-T cells demonstrated cytotoxicity towards WT1⁺/class II+ cell lines such as HL-60, OCI-AML5 and U937 but not the cell lines lacking either WT1 or class II expression (FIGS. 8GG-8PP). WT1 TCR-T cells failed to recognize any of the cell lines regardless of their WT1 expression level probably because of HLA-mismatch. Among the WT1⁺/class II+ cell lines studied, WT1 CAR-T cells were most cytotoxic towards OCI-AML5. We confirmed that WT1 CAR-T cells were in fact reactive to OCI-AML5 using CD19 CAR-T cells as a control (FIGS. 8QQ, 8RR).

TABLE 3
A summary of HLA class II and WT1 expression of a
panel of leukemic cell lines. Class II and WT1 expression
were measured by flow cytometric analysis.

| | HLA class II | WT1 |
|---|---|---|
| T2 | − | − |
| K562 | − | +++ |
| Daudi | +++ | − |
| Ramos | +++ | − |
| HL60 | + | + |
| OCI-AML5 | +++ | + |
| U937 | +++ | +++ |
| KG1a | − | ++ |

−: <3% positive; +: 3-33% positive; ++: 33-66% positive; +++: >66% positive.

TABLE 4
A summary of HLA class II and WT1 expression of a panel
of primary ALL or AML samples. Class II and WT1 expression
were measured by flow cytometric analysis.

| | HLA class II | WT1 | |
|---|---|---|---|
| Sample ID | (Protein) | (Protein) | AML/ALL |
| 110212 | − | − | ALL |
| 525459 | − | + | AML |
| 80347 | − | ++ | AML |
| 160004 | + | − | AML |
| 100330 | ++ | − | ALL |
| 140539 | +++ | − | ALL |
| 90324 | + | + | AML |
| 747790 | + | + | AML |
| 100864 | + | + | AML |
| 80560 | + | ++ | ALL |
| 100066 | ++ | ++ | ALL |
| 140043 | ++ | ++ | AML |
| 110295 | +++ | ++ | ALL |
| 8245 | +++ | ++ | AML |

−: <3% positive; +: 3-33% positive; ++: 33-66% positive; +++: >66% positive.

In addition to the cell lines, we co-cultured primary AML or ALL samples with WT1 or CD19 CAR-T cells (Table 4) and showed that WT1 CAR-T cells were able to recognize primary leukemic samples tested in a class II-restricted and WT1-specific manner (FIG. 8SS). These results further confirm that WT1 CAR-T cells can recognize WT1 peptide presented by various class II molecules.

Assessing Potential on-Target and Off-Target Reactivity of WT1 Car-T Cells

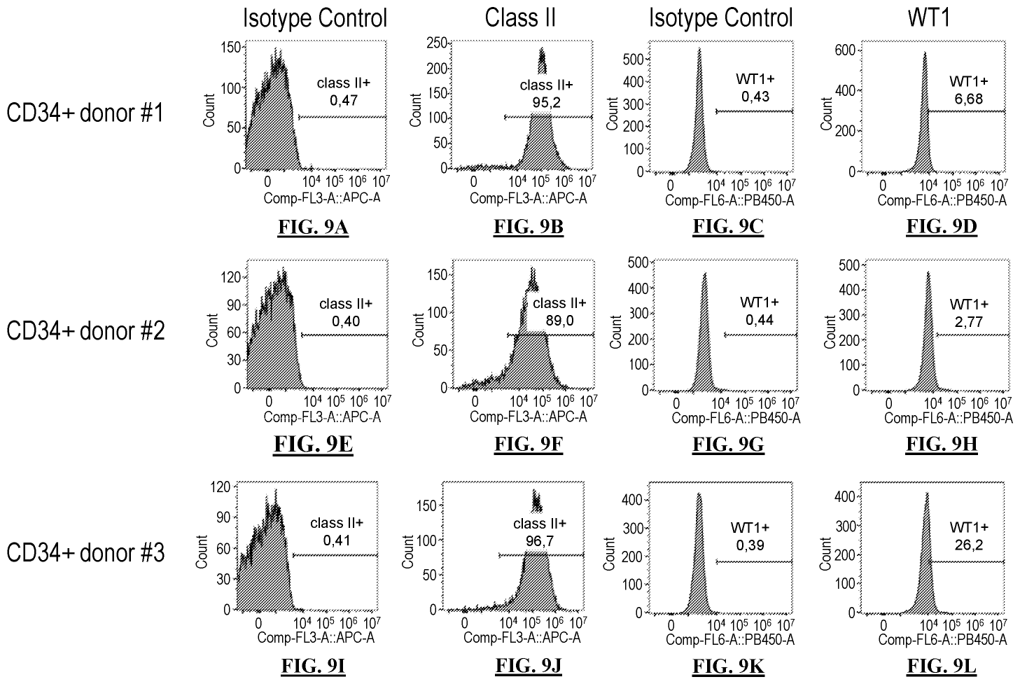

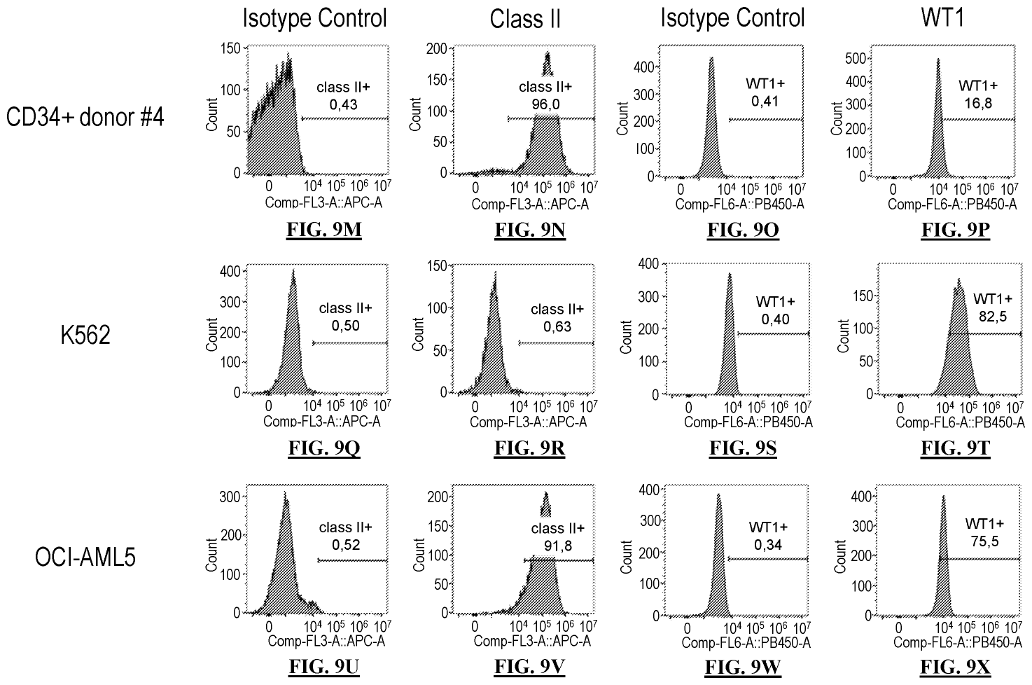

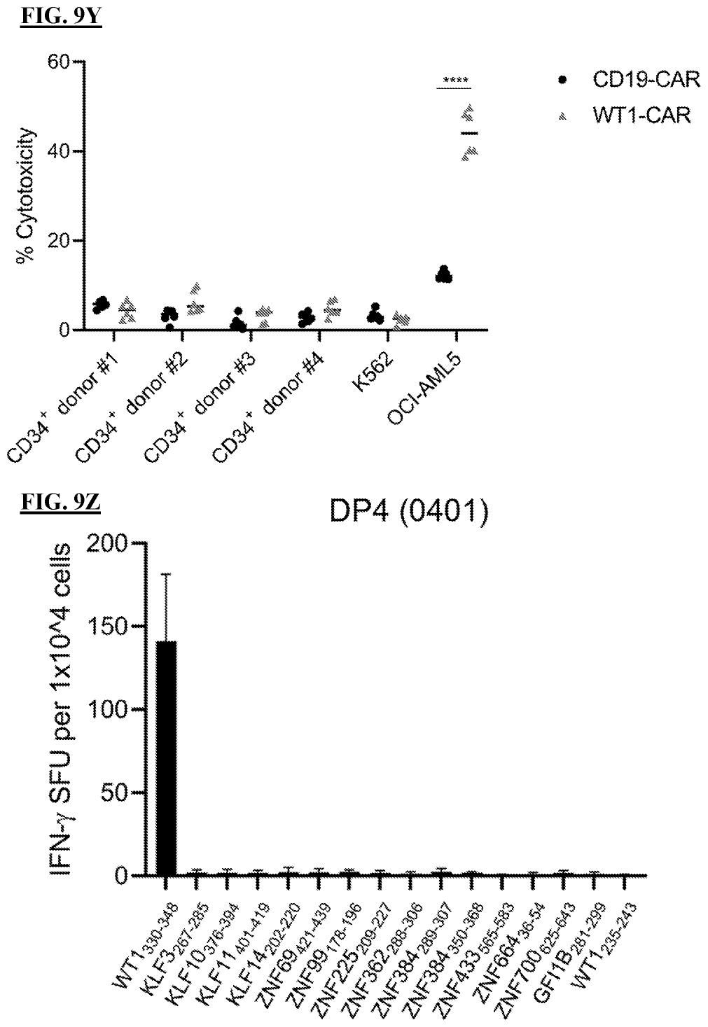

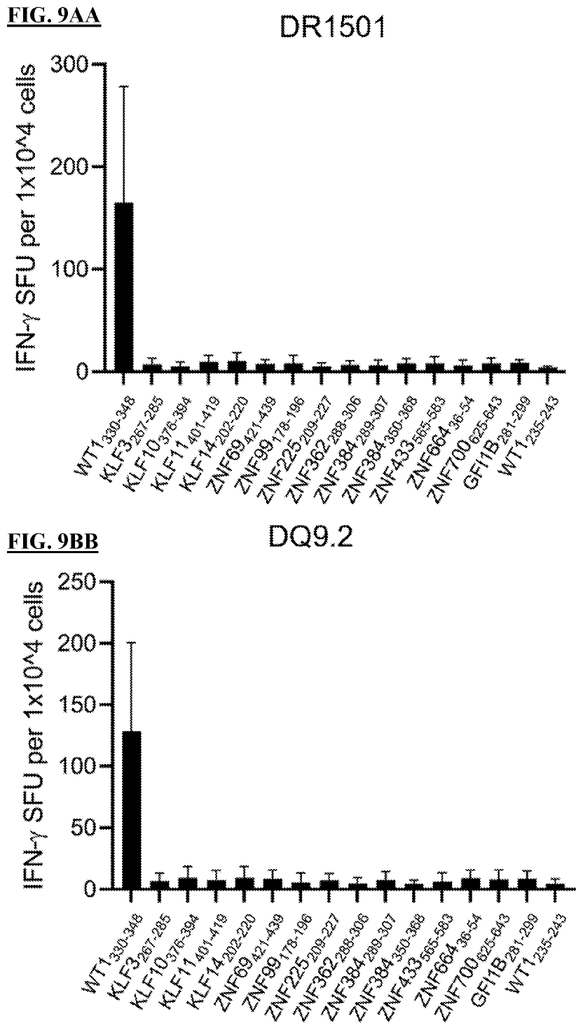

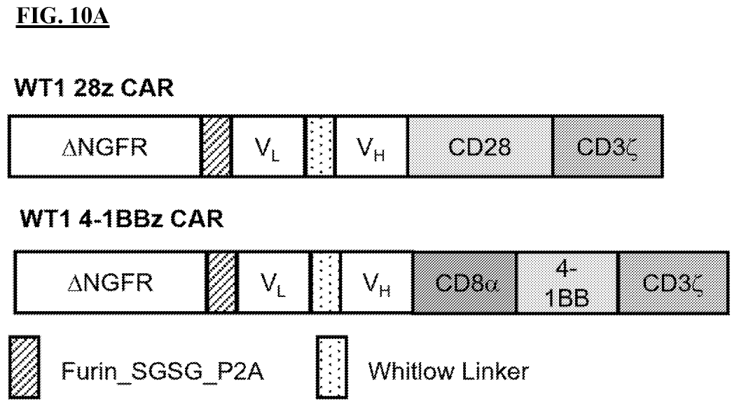

One of the major challenges of TCR and CAR-T therapy is on-target and off-target reactivity, which can lead to undesired toxicity (L. A. Johnson & June, 2017; Morgan et al., 2010). To address potential off-tumor, on-target toxicity, we investigated whether WT1 CAR-T cells could recognize and kill CD34⁺ hematopoietic cells, which are known to express WT1. We first confirmed that primary human CD34⁺ hematopoietic cells purified from 4 different individuals indeed expressed both class II molecules and WT1 (FIGS. 9A-9X). However, cytotoxicity assays showed that WT1 CAR-T cells did not recognize CD34⁺ hematopoietic cells (FIG. 9Y). These results suggest that CD34⁺ hematopoietic cells will not be a target of our WT1 CAR T cell therapy.

To assess off-target cross-reactivity of WT1 CAR-T cells, we first identified peptides which have sequence homology with WT1₃₃₀₋₃₄₈ (Table 5) in silico by using BLAST search. All the peptides identified were derived from transcription factors containing zinc finger domains such as zinc finger proteins (ZNF), Kruppel-like factors (KLF) and growth factor independence 1B (GFI1B), although majority of these peptides showed lower predicted binding affinity for DP4 (0401), DR1501 and DQ9.2, compared to WT1₃₃₀₋₃₄₈. T2 cells individually expressing DP4 (0401), DR1501 and DQ9.2 cells were pulsed with WT1₃₃₀₋₃₄₈ or identified peptides and co-cultured with WT1 CAR T cells in IFN-γELISPOT assays. None of the cross-reactive peptide candidates was able to induce IFN-γ secretion (FIGS. 9Z-9BB). Notably, none of the cross-reactive peptide candidates possessed all the amino acid residues identified in FIGS. 6S, 6T, 7T, and 7U which were critical for recognition by WT1 CAR-T cells, which may explain the lack of off-target reactivity towards these peptides.

TABLE 5
Putative cross-reactive peptides. Potential cross-reactive peptide
candidates were identified by BLAST, and their respective binding
affinities for DP4 (0401), DR1501 and DQ9.2 were predicted by NetMHC3.2.
Amino acid residues of WT1₃₃₀₋₃₄₈ critical for WT1 CAR-T cell recognition
were shown in red. Amino acid residues shared between cross-reactive peptide
candidates and WT1₃₃₀₋₃₄₈ were shown in green.

| | NetMHC prediction (nM) | | | |
|---|---|---|---|---|
| Peptides | DP4 (0401) | DR1501 | DQ9.2 | Peptide sequence |
| WT1₃₃₀₋₃₄₈ | 169.76 | 144.98 | 2940.41 | CNKRYFKLSHLQMHSRKHT |
| KLF3₂₆₇₋₂₈₅ | 506.71 | 1629.77 | 3908.46 | CNKVYTKSSHLKAHRRTHT |
| KLF10₃₇₈₋₃₉₄ | 298.95 | 331.65 | 2277.56 | CGKTYFKSSHLKAHTRTHT |
| KLF11₄₀₁₋₄₁₉ | 188.09 | 190.09 | 1959.39 | CRKTYFKSSHLKAHLRTHT |
| KLF14₂₀₂₋₂₂₀ | 359.91 | 938.81 | 5192.5 | CTKAYYKSSHLKSHQRTHT |
| ZNF69₄₂₁₋₄₃₉ | 408.5 | 303.4 | 1510.91 | CGKAFRSSSHLQLHGRTHT |
| ZNF99₁₇₈₋₁₉₆ | 233.46 | 145.04 | 1935.44 | CSKSFFMLSHLIQHKRIHT |
| ZNF225₂₀₉₋₂₂₇ | 969.96 | 817.88 | 2792.24 | CGKEFNQSSHLQIHQRIHT |
| ZNF362₂₅₈₋₃₀₆ | 1153.13 | 1681.12 | 6463.88 | CDKSFRQLSHLQQHTRIHT |
| ZNF384₂₈₉₋₃₀₇ | 1075.43 | 1472.75 | 6102.39 | CEKSFRQLSHLQQHTRIHS |
| ZNF384₃₅₀₋₃₆₈ | 819.47 | 1285.96 | 5960.36 | CQKAFRQLSHLQQHTRIHT |
| ZNF433₅₆₇₋₅₈₅ | 1411.52 | 1468.71 | 638.73 | CGKAFGSASHLQMHGRTHT |
| ZNF664₃₆₋₅₄ | 377.58 | 143.12 | 2433.14 | CDKGFFHISELHIHWRDHT |
| ZNF700₆₂₅₋₆₄₃ | 179.3 | 679.92 | 508.2 | CGKAFRSASNLQMHERTHT |
| GFI1B₂₈₁₋₂₉₉ | 726.16 | 1323.43 | 860.75 | CGKAFSQSSNLITHSRKHT |

### Comparison Between WT1/28Z and WT1/4-1BBZ Car-T Cells

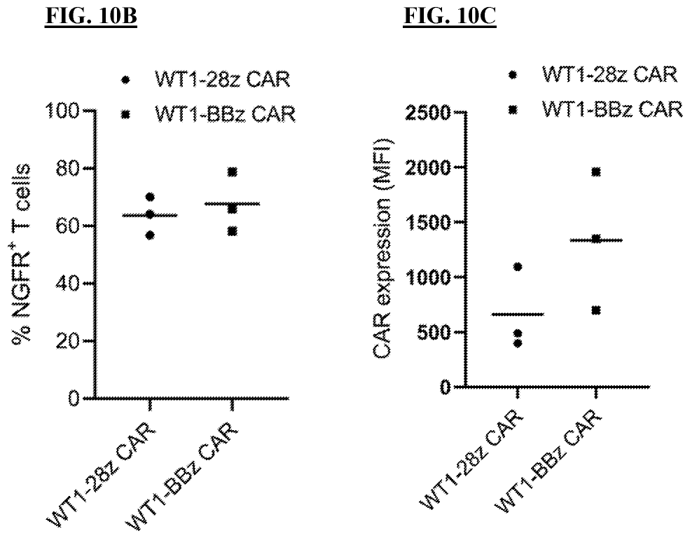

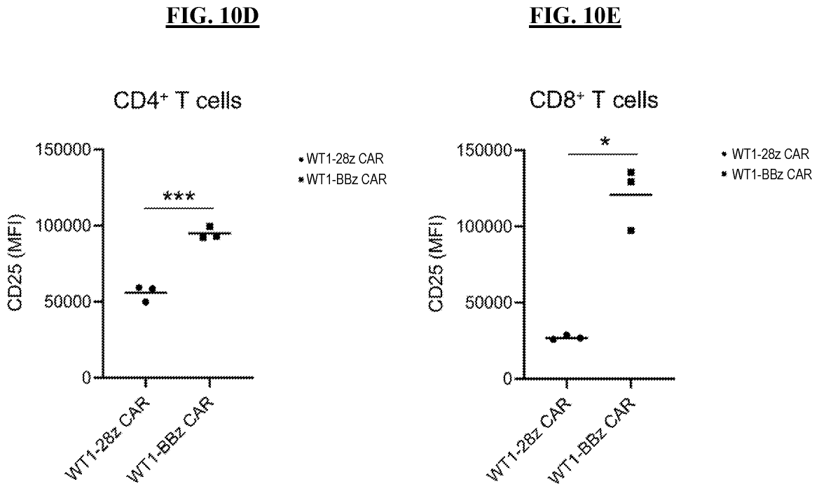

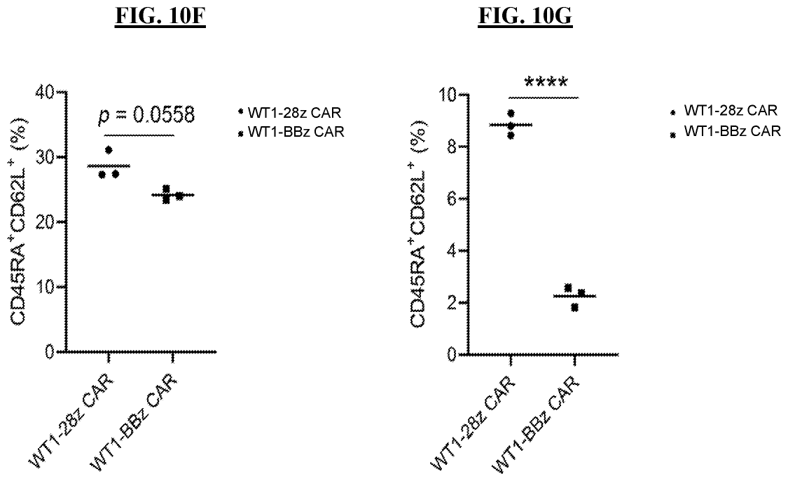

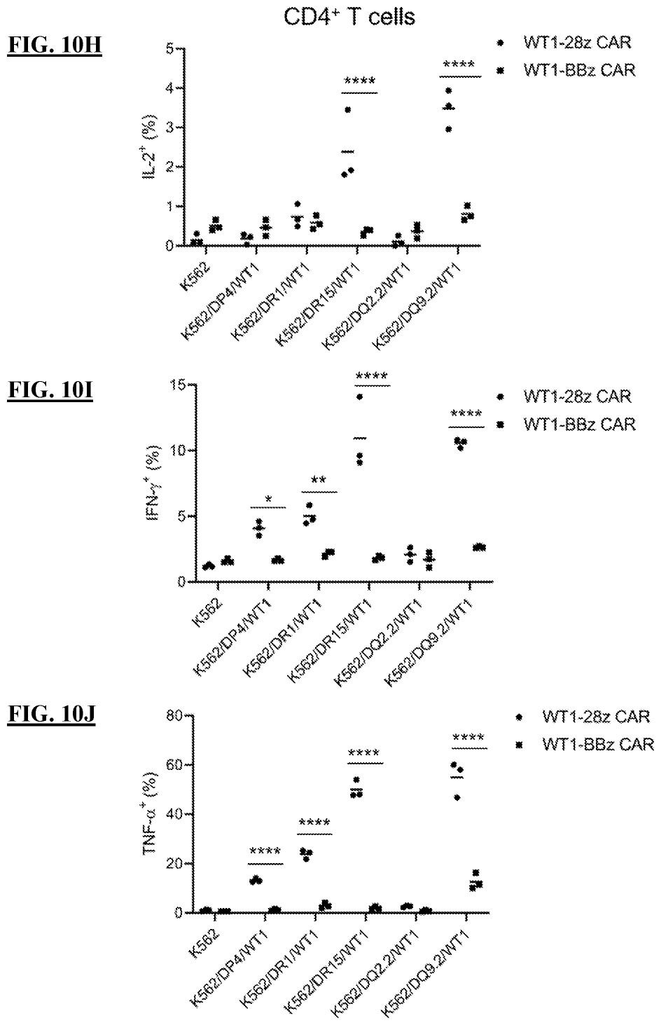

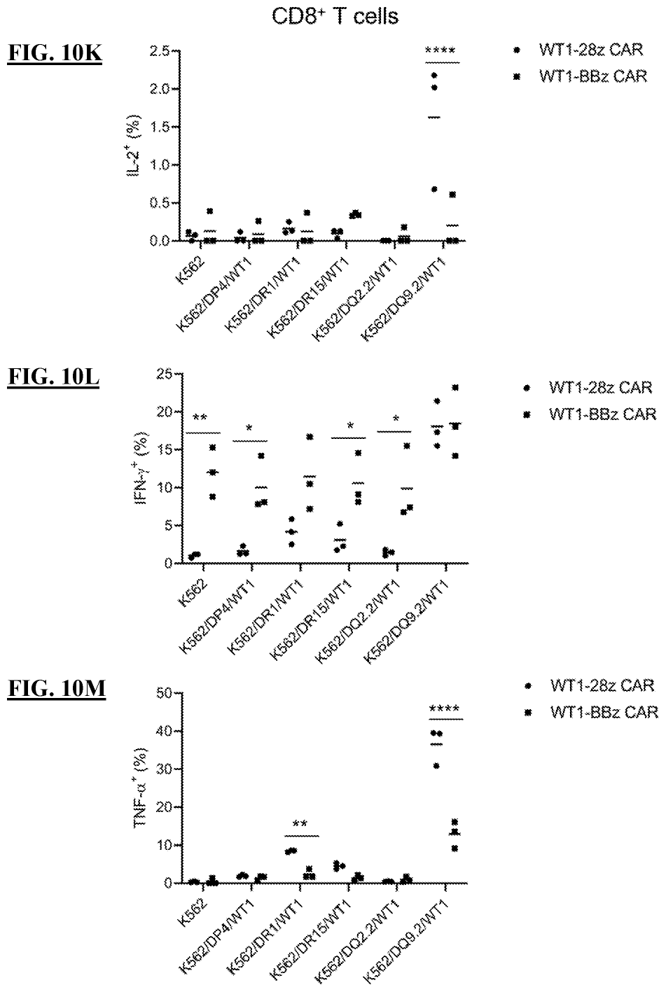

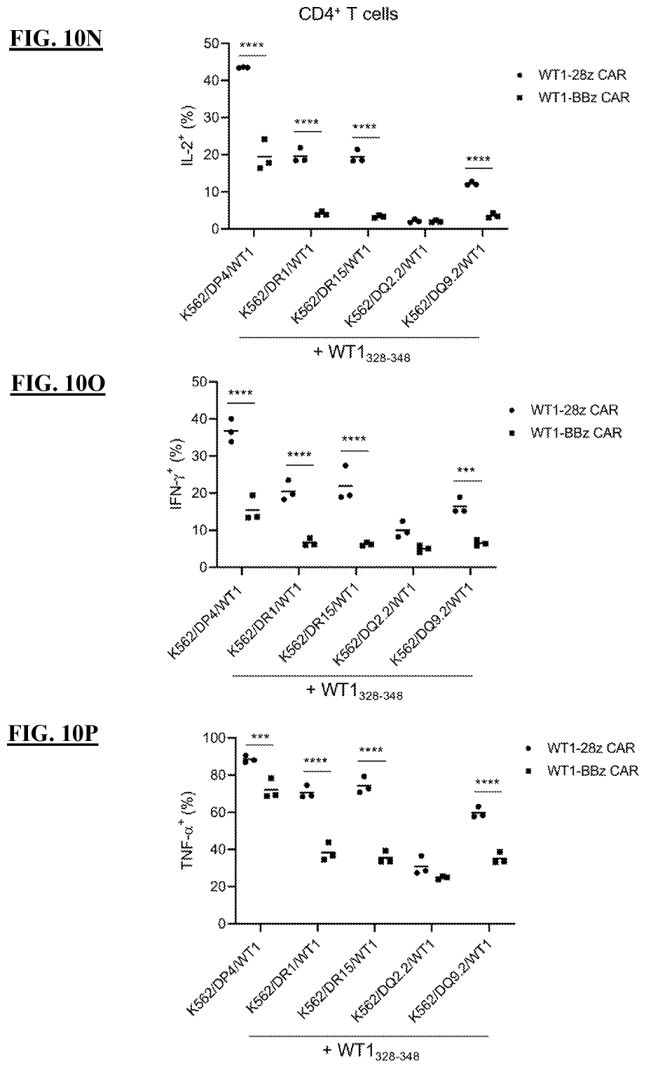

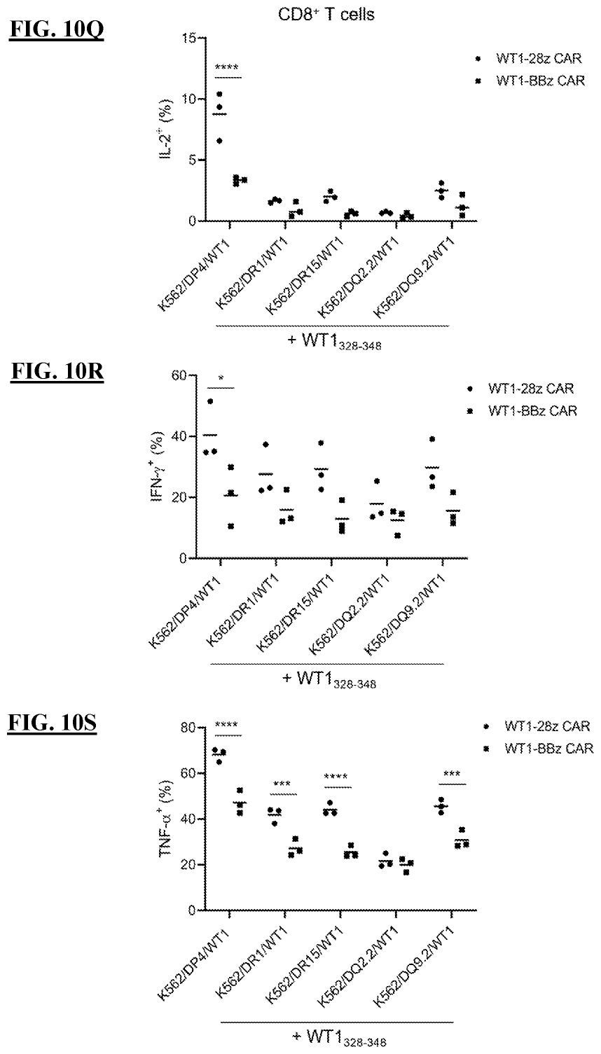

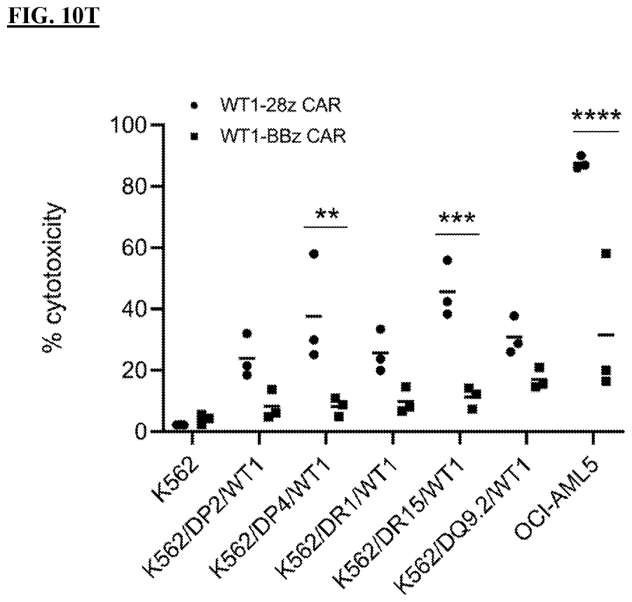

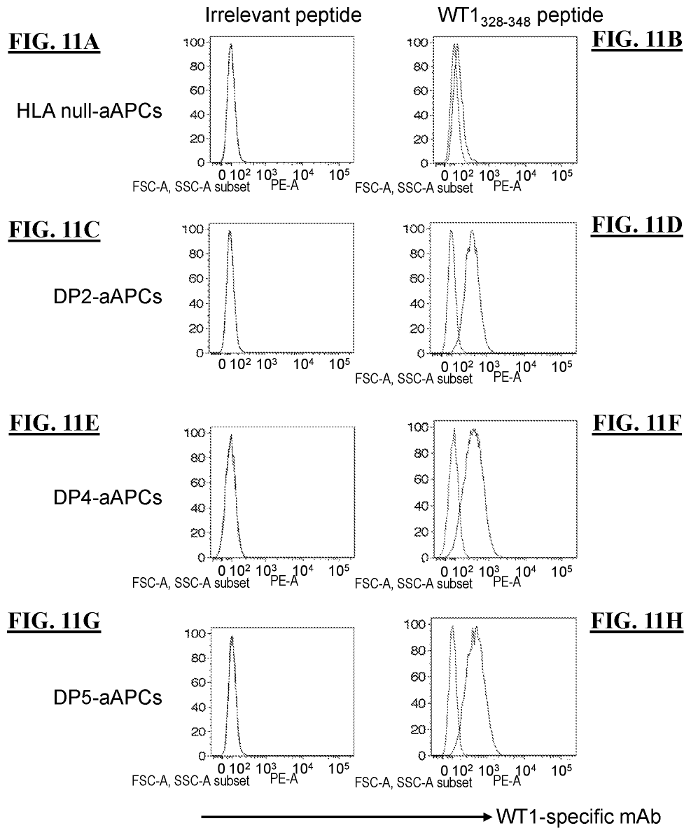

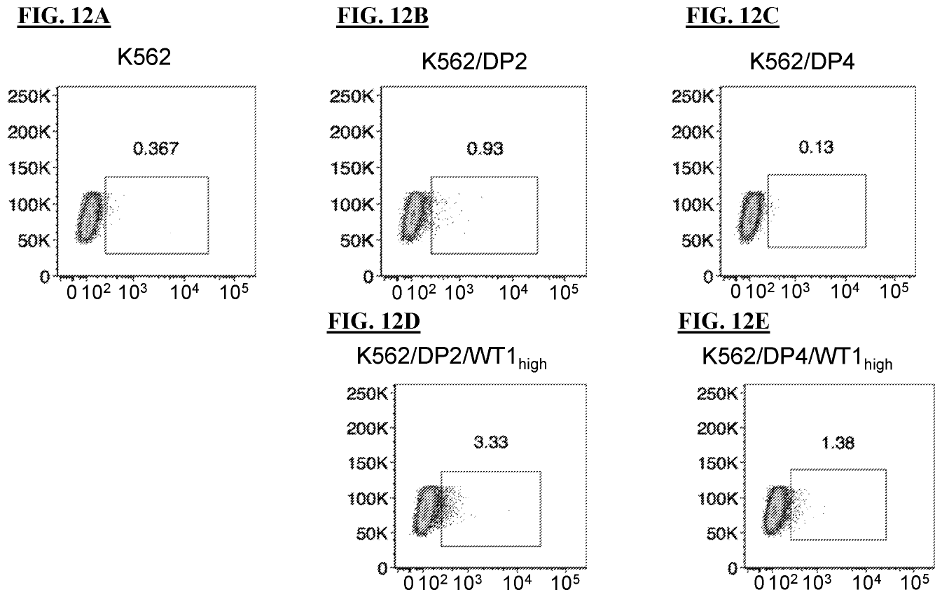

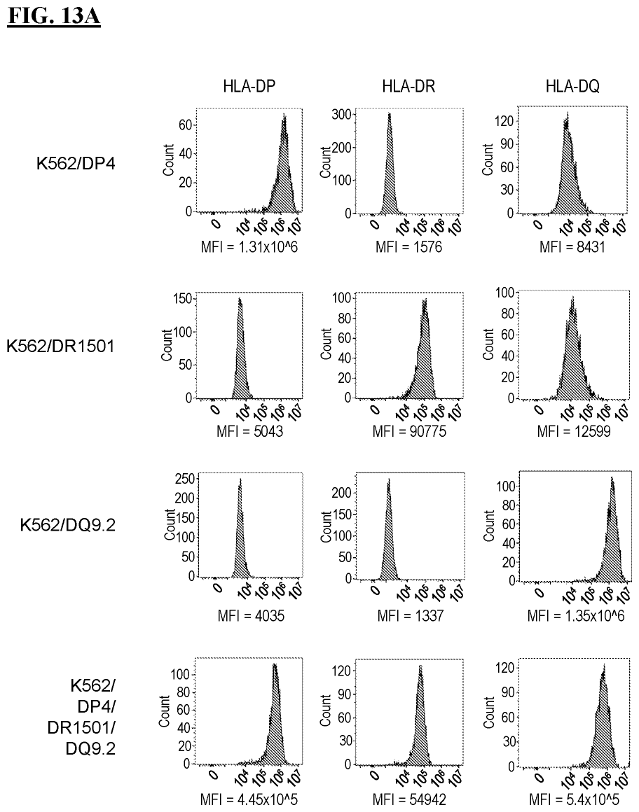

In all the studies above, we used the second generation 28z CAR-T cells. In addition to 28z CAR-T cells, 4-1BB CAR T cells have been approved and clinically tested. Thus, we generated and compared WT1 4-1BBz CAR with WT1 28z CAR (FIG. 10A). When transduced into primary T cells, both WT1 28z and 4-1BBz CAR-T cells showed similar transduction efficiency, indicated by NGFR expression, though 4-1BBz CAR was expressed at higher level on the cell surface when stained by protein L (FIGS. 10B, 10C). Previous studies have reported that higher level of CAR expression could lead to greater ligand-independent tonic signaling, which in some cases contributed to reduced stemness and functionality of CAR-T cells (Gomes-Silva et al., 2017; Long et al., 2015; E. W. Weber et al., 2021). Indeed, we observed higher level of T cell activation marker, CD25, and lower frequency of stem cell-like memory T cells (CD45RA+/CD62L+) in CD4⁺ and CD8⁺ WT1 4-1BBz CAR-T cells compared to 28z CAR-T cells 7 days after transduction (FIGS. 10D-10G). When stimulated with K562-derived class II transfectants ectopically expressing WT1 or pulsed with WT1₃₂₈₋₃₄₈, WT1 4-1BBz CAR-T cells also exhibited inferior functionality characterized by lower percentage of cytokine-secreting population and weaker cytotoxicity compared to WT1 28z CAR-T cells (FIGS. 10H-10T). These results suggest that, in the context of short-term in vitro culture, WT1 28z CAR-T cells demonstrate superior ability to recognize WT1 peptide presented by class II molecules. Furthermore, lower tonic signaling and greater stemness of WT1 28z CAR-T cells may indicate their superiority in the long term.

### Conclusions

Adoptive T cell therapy redirected by class I-restricted TCR has shown promise in the clinic. However, normal tissues can become vulnerable to therapies targeting class I-presented peptide due to the ubiquitous expression of HLA class I molecules, and off-tumor toxicities have been observed in some clinical trials. In contrast, expression of HLA class II molecules is limited to certain cell types; thus, targeting class II-restricted peptide can be a safer alternative. To date, there has only been one published clinical study on class II-restricted TCR-T cell therapy (Lu et al., 2017), though several other groups have identified promising TCR candidates targeting class II-restricted peptide (Dillard et al., 2021; Poncette, Chen, Lorenz, & Blankenstein, 2019), with preparation for clinical trials underway. Most of these identified TCRs, however, have not been shown to be capable of targeting peptides presented by class II molecules across different subsets, despite ample evidence demonstrating that class II-restricted peptides can promiscuously bind to diverse class II molecules (Fujiki et al., 2008; Y. Hu et al., 2014; Kobayashi et al., 2000; Kudela et al., 2007; Panina-Bordignon et al., 1989). In our study, WT1₃₂₈₋₃₄₈ was predicted to bind to various class II molecules including different HLA-DP, DR and DQ molecules and indeed our WT1 CAR-T cells were able to recognize the peptide presented by all the DP, DR, and DQ molecules tested. In contrast, WT1 TCR-T cells only targeted WT1 peptide presented by DP2, DP4, and DP5, but not any of the tested DR or DQ molecules. Since TCR binds to pMHC, structural differences across various class II molecules probably hindered its ability to recognize the same WT1 peptide promiscuously presented by the class II molecules studied. The antibody used for our WT1 CAR was raised against peptide alone instead of pMHC, potentially allowing WT1 CAR-T cells to promiscuously recognize WT1 peptide presented by any class II. The promise of overcoming HLA restriction provides an obvious advantage for the peptide-centric WT1 CAR over WT1 TCR since it will surely broaden the population of patients who can benefit from the therapy. The concept of raising mAb against peptide itself presented by various class II molecules was best exemplified when Denzin et al developed CerCLIP.1, a widely-used mAb capable of recognizing the CLIP peptide presented by various class II molecules decades ago (Denzin et al., 1994). The potential therapeutic benefits of peptide-centric mAb have not been sufficiently explored until Yarmarkovich et al recently showcased CAR-T cell therapy targeting PHOX2B-derived peptide presented by several class I molecules such as HLA-A*24:02, HLA-A*23:01 and HLA-B*14:02 (Yarmarkovich et al., 2021). Prior to their findings, several other groups have developed similar TCR-like mAb-based approaches targeting class I-restricted, intracellular tumor antigen-derived peptide. However, none of them was peptide-centric and instead targeting of MHC as well as the MHC-bound peptide was required (Ataie et al., 2016; Douglass et al., 2021; Hsiue et al., 2021; Rafiq et al., 2017). Interestingly, structural studies conducted by Ataie et al and Hsiue et al showed that HLA-A2/WT1-specific antibody and HLA-A2/P53^{R175H}-specific antibody make 85% and 68%, respectively, of the total contacts with the A2 molecules. In the current study, we not only generated a novel CAR against tumor antigenic peptide presented by class II molecules, but also demonstrated the potential of applying our WT1 CAR-T cell therapy to a large population of cancer patients with different class II genotypes.

The affinity of the antibody used for our WT1-CAR was 50 nM, which lies within the typical range of KD values (1-100 nM) for an antibody. It is possible that, by enhancing the affinity of CAR, WT1 CAR-T cells can recognize class II⁺/WT1^{low} cancer cell lines or primary patient samples more efficiently. However, several studies have suggested that high-affinity CAR can negatively impact the functionality and specificity of CAR-T cells, an observation that is likely dependent on target antigen density (Ghorashian et al., 2019; Oren et al., 2014; S. Park et al., 2017). Furthermore, CARs derived from high affinity antibodies have greater propensity to target normal tissues with low surface antigen density, posing significant risk of on-target, off-tumor toxicity (Caruso et al., 2015; X. Liu et al., 2015). Many groups have demonstrated that CARs utilizing endogenous CD3 signaling machinery, such as synthetic T cell receptor and antigen receptor (STAR), T cell antigen coupler (TAC), antibody-T-cell receptor (AbTCR) and T cell receptor fusion construct (TRuC), can be more sensitive than 2^{nd}generation CARs in general (Baeuerle et al., 2019; Helsen et al., 2018; Y. Liu et al., 2021; Xu et al., 2018). A prototypic CD28 CAR construct has 3 immunoreceptor tyrosine-rich activation domains (ITAMs), whereas a native CD3 signaling complex contains 10 ITAMs, allowing stronger amplification of downstream signaling and hence greater sensitivity. It has been reported that advantages of incorporating TCR signaling complex include reduced tonic signaling, greater in vivo persistence, and more favorable cytokine secretion profile (Baeuerle et al., 2019; Helsen et al., 2018; Y. Liu et al., 2021; Xu et al., 2018). Indeed, we showed that compared to WT1 TCR-T cells, WT1 CAR-T cells had stronger tonic signaling, greater exhaustion, and slower proliferation upon chronic antigen stimulation, which could possibly translate into poorer persistence and tumor control in vivo. Linking our WT 1-specific scFv with the TCR signaling machinery is, therefore, an avenue worth exploring to develop T cell therapies that combine the merits of broader applicability and superior functionality.

Currently, majority of the active clinical trials are evaluating CAR-T cell therapies incorporating second generation 28z or 4-1BBz CAR, which remains the most commonly tested CAR constructs to date. Many studies, mostly still preclinical, have been conducted to directly compare the two approaches. Our results indicate that WT128z CAR-T cells were superior to WT1 4-1BBz CAR-T cells, with reduced tonic signaling, enhanced stemness and greater functionality in vitro. However, it has been reported by several groups that compared to 28z CAR, 4-1BBz CAR-T cells tend to display a more favorable phenotype associated with less exhaustion and enhanced proliferative capacity in vitro, and better tumor control in vivo (Kawalekar et al., 2016; Long et al., 2015; Milone et al., 2009; Salter et al., 2018). Findings from head-to-head clinical trials comparing the two constructs also suggest that 4-1BBz CAR-T cells may be safer and more effective (Ying et al., 2019; Zhao et al., 2020). Studies have revealed that CAR molecules highly expressed on the cell surface are prone to self-aggregate, driving antigen-independent tonic signaling and ensuing functional impairment (Ajina & Maher, 2018; Gomes-Silva et al., 2017; Long et al., 2015; E. W. Weber et al., 2021). The inferior functionality of WT1 4-1BBz CAR-T cells observed in our study could be indeed ascribed to higher level of surface WT1 4-1BBz CAR expression resulting in enhanced clustering of scFvs and stronger tonic signaling. Furthermore, when retroviral vector with long terminal repeats (LTR) is employed for CAR transduction, as the one utilized in the current study, NFκB pathway activated by 4-1BBz CAR can further enhance CAR expression and tonic signaling through a positive feedback loop, undermining its antitumor function (Gomes-Silva et al., 2017). It is, therefore, imperative to determine the level of surface CAR expression for optimal antitumor efficacy. Strategies to modify CAR expression such as the use of alternative promoters or the insertion of internal ribosome entry site (IRES) could be applied to WT1 4-1BBz CAR to improve its in vitro and in vivo functionality. In addition to the second generation 28z and 4-1BBz CAR, many other novel constructs have been developed. For example, we previously illustrated that a CD19-targeting CAR incorporating a JAK-STAT signaling domain exhibited superior antitumor effects in vitro and in vivo compared with 28z or 4-1BBz CAR (Kagoya et al., 2018). Further studies are warranted to identify the optimal construct to fully realize the potential of our WT1-targeting CAR.

One of the major concerns associated with T cell gene therapy is on-target and off-target toxicities. While studies on TCR-like antibody-derived therapeutics targeting class I-presented peptides processed from neoantigens or TSA did not show obvious on- and off-target toxicities (Douglass et al., 2021; Hsiue et al., 2021; Yarmarkovich et al., 2021), TCR-like antibodies targeting HLA-A2/WT1 have been shown to cross-react with non-WT1 derived peptides presented by HLA-A2 molecules (Ataie et al., 2016; Oren et al., 2014). Although WT1 CAR-T cells did not show cross-reactivity to a list of analogous peptides tested, the potential of off-target toxicity in humans cannot be completely excluded with these limited studies. To address possible on-target reactivity to WT1^{low} normal tissues, we demonstrated that WT1 CAR-T cells also did not recognize class II⁺ CD34⁺ hematopoietic cells, approximately 1% of which have been shown to be WT1⁺ (Hosen et al., 2002). This is possibly due to the low level of WT1 expression or different processing and presentation of WT1 peptide in the normal CD34⁺ hematopoietic cells. Cells in gonads, uterus, mesothelium, and kidney also express WT1 (Gulyas, Dobra, & Hjerpe, 1999; Mundlos et al., 1993). Hence, further studies will be necessary to assess potential on-target toxicity towards these normal tissues.

In summary, this study presents the first proof-of-concept that antibody-based modalities can be developed against tumor antigenic peptides presented by diverse HLA class II molecules. Three significant features set our approach apart from other existing antibody or cell-based antitumor therapies. First, we have selected WT1, which is an intracellular oncogenic protein highly expressed in many types of cancer, whereas most, if not all, current antibody-based therapeutics target surface non-oncogenic proteins. Second, the limited class II expression on normal tissues reduces the risk of on-target toxicity, compared to most TCR-T therapy or TCR-like antibody targeting peptides presented by the ubiquitously-expressed class I molecules. Third, the peptide-specific nature of our antibody, combined with the promiscuity of class II-presented peptide, allows class II-agnostic approach to potentially benefit a broad range of patients. The research described here should pave the way for further development of similar approach which can be applied to other class II-restricted tumor antigen peptides expressed in a wide variety of tumors.

Presented herein are novel antitumor therapeutic strategies based on immune receptors targeting WT1 peptides presented by HLA class II molecules. The advantages of the proposed strategies are manifold, with the most important of which potentially being the favorable safety profile and broad applicability. Antibody-based therapeutics targeting surface molecules in an HLA-independent manner, such as CAR-T cell therapy or BiTE targeting CD19 in blood cancer, have yielded unprecedented clinical responses with minimal toxicities (Larson & Maus, 2021). Since B cells are the only cell type that is known to highly express CD19, patients treated with CD19-targeting therapies usually develop B cell aplasia, a non-serious condition that can be easily managed along with intravenous immunoglobulin supplementation. BCMA-targeting T cell therapies have also shown promise in the clinic, though a recent study reported the presence of BCMA in neurons, leading to parkinsonism observed in several patients (Van Oekelen et al., 2021). For solid tumors, the development of similar strategies has been considerably impeded by the lack of appropriate tumor-specific surface proteins. Thus, the application of CAR-T cell therapy and BiTE targeting tumor antigens independently of HLA molecules have been limited to blood cancer. To expand the target repertoire for antitumor treatments, TCR-T cell therapies and TCR-like antibodies have been developed, which recognize peptides derived from intracellular tumor antigens presented in the context of HLA class I molecules. However, the ubiquitous expression of class I molecules in virtually all cell types poses significant risk of on-target and off-target toxicities towards normal tissues, which have been observed in several clinical trials, resulting in severe adverse events and patient deaths (L. A. Johnson & June, 2017). Given that class II molecules are absent in most cell types, except for TECs, activated T cells, and APCs such as DCs, B cells, monocytes, and macrophages, we believe that targeting peptides presented by class II molecules is less likely to induce undesired on-target or off-target reactivities against normal tissues. The observations that several cancer types, including leukemia and some solid tumors, express high level of class II molecules (Axelrod et al., 2018), further strengthen the rationale for developing strategies directed towards class II-presented peptides to specifically target tumor cells. Indeed, we showed that WT1 CAR-T cells are able to kill class II+/WT1⁺ primary ALL and AML samples. Further studies are needed to evaluate the types of solid tumors that express class II and can also be targeted by the class II-directed TCR or CAR-T cell strategies described herein. It remains to be seen in future clinical studies if the proposed therapeutics can indeed avoid toxicities similar to those observed in class I-directed trials. It is possible that in cancer patients, some normal tissues might upregulate class II molecules in vivo in the presence of abnormally high level of certain hormones or cytokines, such as IFN-γ, rendering them vulnerable to our class II-directed therapies.

The second advantage of the proposed strategies lies in their potential to overcome the HLA barrier. Despite the polymorphism of class II genes, binding pockets of different class II molecules can have shared preference for certain anchor residues, resulting in overlapping repertories of binding peptides (Greenbaum et al., 2011). This allows the same peptide to be promiscuously presented by various class II molecules, whether they belong to the same or different supertypes. This is supported by the observation of promiscuous T cell response mediated by class II-restricted TCR towards the same peptide presented by different class II molecules, as exemplified by clone 9 TCR described in previous studies (Lin et al., 2013) and in the present examples. Since a TCR makes contacts with both the class II molecule and the class II-bound peptide, it is restricted by structurally similar class II proteins and is unlikely to recognize the same peptide promiscuously presented by other structurally distinct class II molecules. In contrast, it is possible that a receptor targeting peptide itself, instead of pMIIC, can potentially overcome the HLA barrier. Very recently, Yarmarkovich et al described a peptide-centric TCR-like antibody capable of binding to peptide presented by several HLA class I molecules (Yarmarkovich et al., 2021). However, they also showed that structural hindrance imposed by HLA-C*07:02 significantly block the interaction between the antibody and the antigenic peptide. Unlike class I molecules, the binding groove of class II molecules is open-ended and can typically accommodate longer peptides of 13-25 amino acids, with part of the peptides extending beyond the groove, forming the peptide-flanking residues (PFRs) (Godkin et al., 2001; Stern et al., 1994). We posit that our WT1-specific antibody might partially, at least, target the PFRs of WT1 peptide, allowing it to circumvent HLA-mediated structural impedance and recognize the WT1 peptide promiscuously presented by a wide variety of class II molecules. We show that WT1 TCR and CAR-T cells display different modes of recognition of WT1 peptide presented by HLA-DP4, where the WT1 epitope of CAR-T cells potentially span beyond the peptide binding cleft (Table 6). We also show that WT1 CAR-T cells recognize and kill K562 cells individually expressing a broad panel of DPs, DRs, and DQs, as well as various class II+/WT1 cancer cell lines and primary ALL and AIL samples. The results reaffirm our hypothesis that a peptide-specific, class II-agnostic receptor can be developed and applied to cancer patients with diverse class II genotypes.

TABLE 6
WT1 amino acid mapping for TCR and CAR-T cells. Amino
acid residues of WT1₃₂₈₋₃₄₈ presented by DP4 critical
for recognition by WT1 TCR or CAR-T cells were shown (*).
DP4-binding core residues of WT1₃₂₈₋₃₄₈ predicted
by NetMHCII 3.2 were shown in blue.

| | |
|---|---|
| TCR | ******* * |
| CAR | ** **** ** *** |
| | ₃₂₈PGCNKRYFKLSHLQMHSRKHT₃₄₈ |

### Potential Mechanisms of Resistance to the Proposed WT1-Targeting Strategy

It is well-known that antitumor T cell therapies can impose a strong selective pressure on tumor cells, which often gives rise to those that are treatment-resistant. Antigen loss or mutations are predominant mechanisms of resistance frequently reported in the clinic (Larson & Maus, 2021). For instance, after initial clinical response, a subset of patients who received CD19-targeting CAR-T cell therapy relapsed due to the emergence of leukemic blasts which lacked CD19 or expressed mutant CD19 devoid of the epitope for CAR-T cells (Larson & Maus, 2021). It has also been frequently observed that cancer cells can downregulate HLA class I molecules or other molecules involved int the class I presentation pathway to avoid recognition by endogenous antitumor CD8⁺ T cells and engineered T cells directed by class I-restricted TCRs (Dhatchinamoorthy, Colbert, & Rock, 2021). Since class I molecules are considered non-essential for tumor cells, the downregulation of which allows them to escape immunosurveillance without impairing their ability to grow. Similar mechanisms of resistance to the strategies proposed herein could occur when HLA class II molecules or WT1 proteins are downregulated or mutated in the tumors. For example, IL-10, the suppressive cytokine which is often upregulated in the tumor microenvironment, can downregulate class II expression on APCs (Mittal & Roche, 2015), and potentially tumor cells. In addition, certain types of cancer cells can repress class II expression through genomic alteration or epigenetic silencing of CIITA, which has been reported in lymphoma and several solid tumors (Axelrod et al., 2018). In contrast to class II molecules, which are dispensable for tumor growth, WT1 is less likely to be downregulated in tumors due to its oncogenic role in the survival, proliferation, and metastasis of cancer cells (Sugiyama, 2010). Indeed, WT1 downregulation has yet to be shown to contribute to relapse experienced by patients who received WT1 peptide vaccines or TCR-T cell therapy directed towards WT1 peptide in the context of class I molecules. Although WT1 mutations have been identified in a small percentage of AML patients, their impact on prognosis remains unclear (Krauth et al., 2015). There has also been no report showing that WT1-targeting therapies induced WT1 mutations in patients, suggesting that potential loss-of-function mutation might be as detrimental to the fitness of tumor cells as immune responses directed towards wild type WT1. The natural DP2 and DP4-restricted WT1 epitope, WT1₃₂₈-348, identified herein forms part of the zinc finger domain 1, which is critical for the functions of WT1 such as target DNA binding. Thus, it is unlikely that the selective pressure exerted by the proposed WT1 TCR-T or CAR-T cell therapies will favor WT1 mutants which lack the epitope. In addition to downregulation and mutation of class II molecules or WT1, other potential mechanisms of resistance are common to T cell therapies such as lack of T cell persistence, poor access to solid tumors and the suppressive tumor microenvironment. These will be discussed further in the sections below.

### Affinity of WT1-Specific TCR and Antibody

We showed that clone 9 WT1 TCR-T cells recognize K562/DP4 cells that ectopically express WT1. Although K562 cells express endogenous WT1, WT1-untransduced K562/DP4 cannot stimulate WT1 TCR-transduced T cells. We also observed lack of cytotoxicity of WT1 TCR-transduced T cells when stimulated with class II+/WT1⁺ cancer cell lines, even when some of which have been genotyped and identified as DP4*. The lack of recognition could be due to a low density of DP4-restricted WT1 epitope for the TCR that is below the threshold needed to activate T cells. The affinity of TCRs, along with the target antigen density, are the two predominant factors determining the avidity of TCR-T cells. Although we did not measure the affinity of WT1 TCR, it is presumably low since TCRs with high affinity for self-antigens are usually eliminated by means of central and peripheral tolerance. Since DP4 is the most prevalent class II genotype in many ethnic groups, it is imperative to optimize the affinity of TCRs against DP4/WT1 for the development of TCR gene therapy. Various strategies have been employed by others to enhance affinity of TCRs for greater antitumor efficacy, such as phage display (Y. Li et al., 2005; Robbins et al., 2008; Schmitt et al., 2013). We have previous reported a novel alternative for TCR affinity maturation by exploiting TCR chain centricity (Nakatsugawa et al., 2015; Ochi et al., 2015). When pairing the chain-centric TCR hemi-chain with various non-chain-centric counter chains, we were able to generate TCR pairs with optimal affinity against A2/MART1₂₇₋₃₅ or A24/WT1₂₃₅₋₂₄₃ complexes. This strategy also allows us to properly select TCR pairs that possess minimal cross-reactivity, which is critical given that some affinity-matured TCRs tested in the clinic have led to severe adverse events caused by TCR cross-reactivity. Thus, we will employ this method to first determine the chain centricity of the WT1 TCR, generating a pool of TCR counter chains, and identify the TCR pairs with optimal affinity against DP4/WT1 complexes. This would allow us to obtain antitumor T cells with suitable avidity towards DP4⁺ tumor cells with a wide range of WT1 expression.

Like TCR-T cells, the avidity of CAR-T cells can be primarily influenced by antigen density and scFv affinity. We identified the affinity of the W T1-specific antibody to be 50 nM, which lies within the typical range of KD values (1-100 nM) for an antibody. Since T cells with higher avidity tend to have greater sensitivity for low antigen density, WT1 CAR-T cells with increased scFv affinity, hence greater T cell avidity, might recognize class II cancer cell lines or primary leukemic samples with low WT1 expression more effectively. Multiple methods for affinity maturation of antibody or scFv have been described to date, such as error-prone PCR, site-directed mutagenesis, or AID-mediated in vitro somatic hypermutation. However, in the context of high antigen density, such as HER2 or EGFR on solid tumors, CAR-T cells with enhanced scFv affinity can become overly sensitive to low level of antigens displayed in normal tissues, leading to off-tumor toxicities (Caruso et al., 2015; X. Liu et al., 2015). This might be less of an issue for our WT1-CAR-T cells, as both WT1 and class II molecules are either absent or expressed at low level in small subsets normal tissues. The optimal range of affinity for CAR is still very controversial. Potential off-tumor toxicity needs to be carefully monitored if we conduct affinity maturation studies in the future. Even when on-target, off-tumor reactivity is not a concern-as is the case for CAR-T cells targeting CD19-T cells transduced high affinity scFv has been shown to have inferior in vitro and in vivo antitumor functionality. This can be explained by the serial triggering model, which suggests that a TCR needs to have fast off-rate when binding to its cognate pMHC, thereby allowing a single pMHC to serially engage multiple TCRs for efficient T cell activation (Valitutti, Muller, Cella, Padovan, & Lanzavecchia, 1995). The model can be applied to CARs expressed on the surface of T cells, and their cognate antigens on target cells. Thus, it is crucial to optimize the affinity of WT1-specific scFv to maximize the antitumor efficacy of WT1 CAR-T cells.

### Optimization of WT1-Specific CAR Construct

In addition to antigen density and scFv affinity, surface CAR expression and other parts of a CAR construct such as hinge, transmembrane domain, and signaling domain, can all affect a CAR-T cell's response towards the target cells. Although higher CAR expression on cell surface can improve the sensitivity of CAR-T cells for low antigen density, it can also increase the likelihood of antigen-independent clustering of scFv, leading to tonic signaling, T cell exhaustion, and functional impairment (Ajina & Maher, 2018). This effect has been observed in both 28z and 4-1BBz CAR-T cells and can be alleviated by lowering surface CAR expression (Gomes-Silva et al., 2017; Long et al., 2015). We observed higher expression of 4-1BBz CAR on the cell surface compared to 28z CAR, which possibly explain stronger tonic signaling, less favorable T cell phenotype, and inferior antitumor efficacy observed in 4-1BBz CAR-T cells. It will be interesting to see if 4-1BBz CAR-T cells with decreased CAR expression on surface can mediate greater antitumor response in vitro and in vivo compared to their 28z counterparts. For hinge and transmembrane domains, the optimal combination might depend on the structure and density of the target antigens. It has also been shown that the appropriate hinge length can be determined by how distal or proximal the target antigen epitope is to the target cell membrane to form an immunological synapse for effective antitumor T cell response (Larson & Maus, 2021). Many published studies have described various modifications of the intracellular signaling domains to improve CAR-T cell response towards tumor cells. Co-stimulatory domains such as CD28, 4-1BB, OX40, ICOS have been extensively tested, and their effects on the in vitro and in vivo functionality of CAR-T cells appear to be context-dependent (Larson & Maus, 2021). Instead of the synthetic signaling domains incorporated in most CAR constructs, a promising strategy demonstrated in several recent studies combines scFv or separate VH/VL with the endogenous CD3 signaling machinery of a TCR. Since the native CD3 signaling complex contains more ITAMs than a prototypic CAR construct, T cells transduced with the hybrid constructs are more sensitive to their targets due to greater amplification of downstream signals. Reported strategies include TRuc (scFv linked to CD3F), TAC (scFv linked to CD3F-targeting domain and co-receptor domain), and STAR (VH/VL linked to TCRα/β constant regions), all of which have displayed advantages associated with CD3 signaling machinery such as reduced tonic signaling, lower risk of CRS and superior in vivo antitumor control related to enhanced T cell persistence (Baeuerle et al., 2019; Helsen et al., 2018; Y. Liu et al., 2021). We showed that WT1 CAR-T cells have stronger tonic signaling compared to WT1 TCR-T cells. It is possible that for T cells expressing the same WT1-targeting scFvs, those utilizing the endogenous CD3 signaling machinery might experience weaker tonic signaling and exhaustion and mediate more effective and sustained antitumor response in vitro and in vivo. Further studies are thus warranted to develop constructs which integrate the advantages of our peptide-centric, class II-agnostic antigen targeting domain with the optimal functionality provided by the suitable signaling domains.

Since the antibody described herein was developed from mice, humanization of the scFv sequences might be helpful to minimize potential xenogeneic response which could reduce clinical efficacy or even induce rejection in patients. Other modifications of our WT1 CAR can also be incorporated to overcome challenges associated with targeting solid tumors. For example, CAR-T cells can be engineered to express chemokines or proinflammatory cytokines to facilitate trafficking to solid tumors and to counter the suppressive tumor microenvironment, respectively. They can also be engineered to secrete checkpoint inhibitors to reduce T cell exhaustion and prolong persistence. WT1 is high expressed in various solid tumors.

Development of Relevant Strategies Targeting Class II-Presented Peptides Derived from Other Tumor Antigens

We chose WT1 as a target in this study since it has been well-established and possesses key attributes for an ideal tumor antigen for immunotherapy. The proposed peptide-specific, class II-agnostic approach can also be applied to other TAAs or cancer germline antigens if certain criteria are met. First, the antigens need to be highly expressed in tumor cells, but with low to no expression in normal tissues, to avoid on-target, off-tumor toxicity. Second, the tumor cells expressing the antigens of interest must also express class II molecules capable of presenting the peptides derived from the intracellular antigens on the cell surface to allow direct target cell recognition by antibodies or T cells. Third, the same antigenic peptide needs to be promiscuously presented by various class II molecules so an antibody specific for the antigenic peptide can overcome the HLA barrier and be applied to diverse patient subsets. Two of the most promising target antigens that satisfy the above criteria are MAGE-A3 and NY-ESO1, both of which are well-known cancer germline antigens. MAGE-A3 is highly expressed in melanoma, non-small cell lung cancer, breast cancer, ovarian cancer, and colon cancer, all of which have been shown to express class II molecules (Axelrod et al., 2018; Zajac et al., 2017). The feasibility of targeting MAGE-A3-derived peptide presented by class II molecules was recently demonstrated by Rosenberg's group, as they reported promising data from a TCR-T cell therapy targeting DP4/MAGE-A3 in patients with solid tumors such as melanoma and breast cancer (Lu et al., 2017). An earlier study also identified MAGE-A3-derived peptide that can be promiscuously presented by various HLA-DR molecules (Kobayashi et al., 2000). NY-ESO1 is also known to be expressed in class II tumors such as melanoma and ovarian cancer (Thomas et al., 2018). Although most of the past or current NY-ESO1 clinical trials have studied peptides presented by class I molecules, the promiscuous NY-ESO1 peptide capable of binding to multiple DP and DR molecules has been identified (Kudela et al., 2007), which serves as a promising candidate for the strategies described herein. Importantly, the expression of both MAGE-A3 and NY-ESO1 in normal tissues are mainly restricted to germline cells and trophoblastic cells devoid of the class II expression, lowering the possibility of treatment-induced on-target, off-tumor toxicity (Coulie et al., 2014). Thus, we believe that the peptide-centric, class II-agnostic approach illustrated herein can be applied to antigens other than WT1, including but not limited to MAGE-A3 and NY-ESO1.

We have identified the natural WT1 epitope in the context of the highly prevalent ILA-DP2 and DP4 and demonstrated the potential of TCR-T cell therapy directed towards DP4⁺/WT1⁺ tumor cells. Built on these findings, we have described the development and highlighted the efficacy and safety of a novel CAR-T cell therapy targeting WT1 peptide presented by various HLA class II molecules. Unlike TCRs or other TCR-like antibodies, the proposed WT1 CAR display unique mode of antigen recognition by targeting the peptide itself, rather than pMHC, enabling it to overcome conventional HLA restriction and substantially broaden its clinical reach to more diverse patient populations. These examples provide important proof-of-concept for the future development of relevant therapeutics towards many other promising tumor antigens, representing an exciting and innovative class of anticancer treatment.

### Example 3—Characterization of CAR-T Cell Therapy Targeting WT1 Peptide

### Materials and Methods

### Impedance-Based Cytotoxicity Assay

The long-term, continuous CAR-T cell-mediated killing of K562 transfectants was monitored using an xCELLigence Real-Time Cell Analyzer-Multiple Plate instrument (Agilent Technologies). On day 1, 50 μl of tethering reagent (anti-CD71 antibody; 4 ug/ml) was added to each well of a 96-well plate. The plate was then incubated at 4° C. overnight. On day 2, the plate was washed twice with PBS before adding 50 μl of culture media into each well. The plate was then placed into the instrument and let equilibrate at 37° C. for one hour before taking background measurement. Target cells in 50 μl of culture media were then added to each well and the plate was placed back into the instrument to measure impedance every 15 minutes for 24 hours. On day 3, CAR-T cells in 100 μl of culture media were added to each well and the plate was placed back into the instrument to measure impedance every 15 minutes for 84 hours. Variation in electrical impedance measured was represented as the cell index, which was calculated using the RTCA software. Cell index values were then converted to % cytotoxicity.

$$\% Cytotoxicity = [ \text{} ( cell {index}_{without T cells} - cell {index}_{with T cells} ) / cell \text{} {index}_{without T cells} ] \times 100 \% .$$

### Mouse Experiments

Six- to eight-week-old male NOD-scid-IL2Rg^{null} (NNSG) mice were used. Mice were first irradiated with 1.5 Gy using X-RAD 320 before the transplantation of leukemic cells to enhance engraftment. 0.1 million PR9 leukemia cells that had been transduced with pMX-EGFP-firefly luciferase were injected intravenously via tail vein of the NSG mice. CD3⁺ T cells were retrovirally transduced with WT1 or MSLN CAR-encoding gene and expanded for one week. Five million CAR-T cells were administered via tail vein to the NSG mice two days after the transplantation of PR9 cells. The leukemia burden was analyzed with Xenogen IVIS Spectrum (PerkinElmer). Mice were injected intraperitoneally with 200 L of 15 mg/mL D-luciferin in PBS and imaged 20 to 30 minutes post-injection. Mice were monitored at least four times per week and euthanized by C02 inhalation after they became moribund due to the leukemia progression or reached endpoints outlined in institutional guidelines. The mice were randomly assigned to treatment groups in each experiment. No statistical methods were used to predetermine sample size. The investigators were not blinded to allocation during experiments and outcome assessment.

### Time-of-Flight Mass Cytometry (CyTOF)

CyTOF staining was performed as previously described (Gadalla et al., 2022). The Briefly, live-cell barcoding using CD45 antibodies conjugated to metal isotopes combined with DNA-based palladium barcoding were first applied to samples. Samples were then fixed and permeabilized prior to surface and intracellular immunostaining. All antibodies were used at concentrations previously determined by titration. Samples were acquired on a Fluidigm Helios mass cytometer at the PMCC Tumor Immunotherapy Profiling Facility. Data were exported and de-barcoded into individual FCS files and analyzed using Cytobank (Cytobank, Inc). The CyTOF data was clustered and analyzed using FlowSOM algorithm together with the R implementation of UMAP. Heatmaps were generated from Z score-normalized values of mean metal intensity (MMI).

### Results

### WT1 CAR-T Cells Effectively Recognize Target Cells Simultaneously Presenting WT1 Peptide on Multiple Class II Molecules

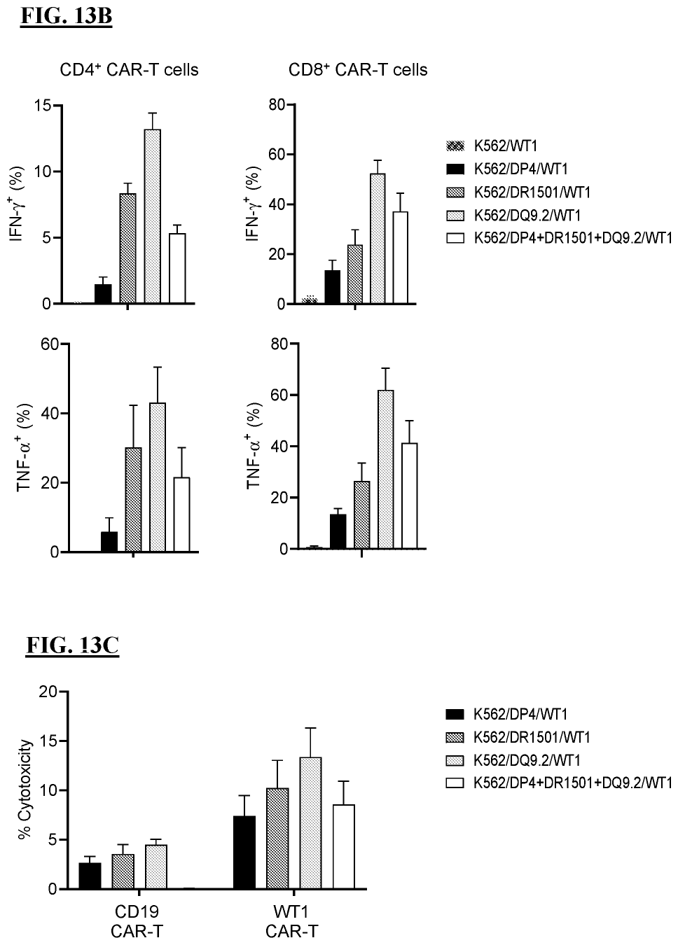

HLA class II+ tumor cells can express more than one type of class II molecules on the cell surface. Different class II molecules can potentially present endogenously-derived WT1 peptides with different length and conformation, inducing distinct CAR-T response. K562 transfectants were generated co-expressing DP4 (DPB1*04:01), DR1501, DQ9.2 to investigate if the presence of diverse class II/WT1 complexes on target cells stimulates WT1 CAR-T cells differently compared to the K562 counterparts expressing only one type of class II. To ensure similar number of class II molecules on the cell surface, we confirmed that triple class II+ transfectants expressed approximately one-third of individual class II molecules compared to the single class II+ transfectants (FIG. 13A). In a five-hour co-culture assay, WT1 CAR-T cells showed intermediate cytotoxicity and cytokine response against triple class II+K562 transfectants (FIGS. 13A-13C).

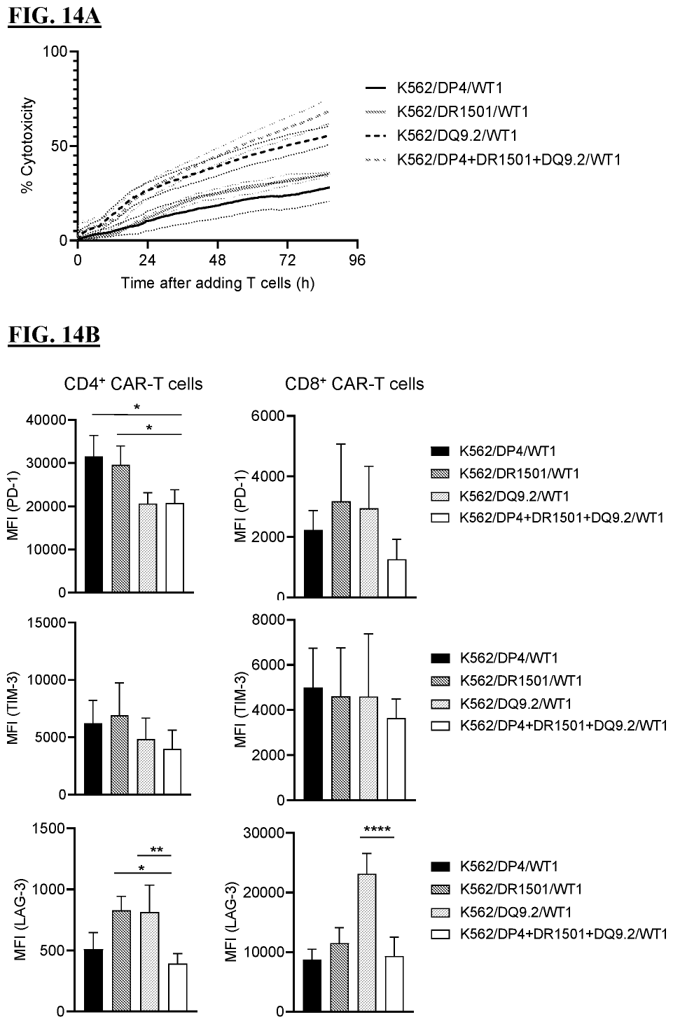

Next, to investigate antitumor response of WT1 CAR-T cells in the context of chronic stimulation, a four-day impedance assay was run where target cells were cultured in excess with CAR-T cells. Interestingly, WT1 CAR-T cells demonstrated the greatest long-term cytotoxicity against triple class II+ transfectants (FIG. 14A). By the end of the impedance assay, both CD4⁺ and CD8⁺ WT1 CAR-T cells co-cultured with triple class II+ transfectants also expressed noticeably lower exhaustion markers such as PD-1, TIM-3 and LAG-3 (FIG. 14B), suggesting their superior long-term antitumor response might be attributed to a more favorable T cell phenotype.

### WT1 CAR-T Cells Recognize a WT⁺ Class II+ Leukemia Cell Line

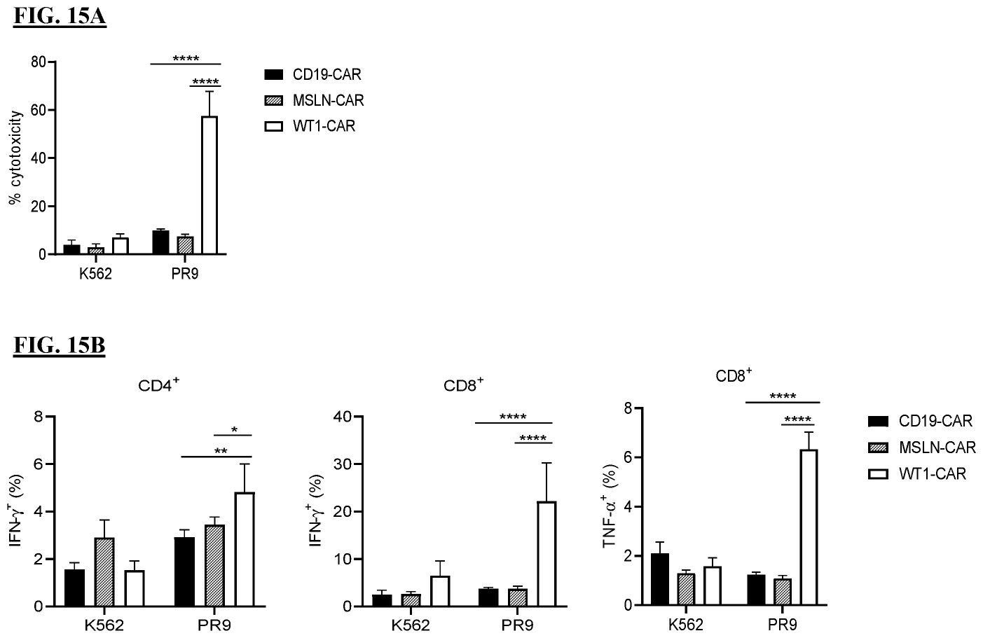

We have so far demonstrated that WT1 CAR-T cells can recognize class II-restricted WT1 peptides on K562 cells. Since WT1 can potentially be processed differently by other cell types, the ability of WT1 CAR-T cells to target another WT1⁺/class II+ AML cell line, PR9, was investigated. WT1 CAR-T cells exhibited significantly greater cytotoxic and cytokine response than CD19 or mesothelin (MSLN)-specific CAR-T cells when stimulated with PR9 (FIGS. 15A-15B). Wild-type K562 cells, which lack class II expression (FIG. 15A), induced minimal cytotoxic and cytokine response from WT1 CAR-T cells.

### Antitumor Activity of WT1 CAR-T Cells In Vivo

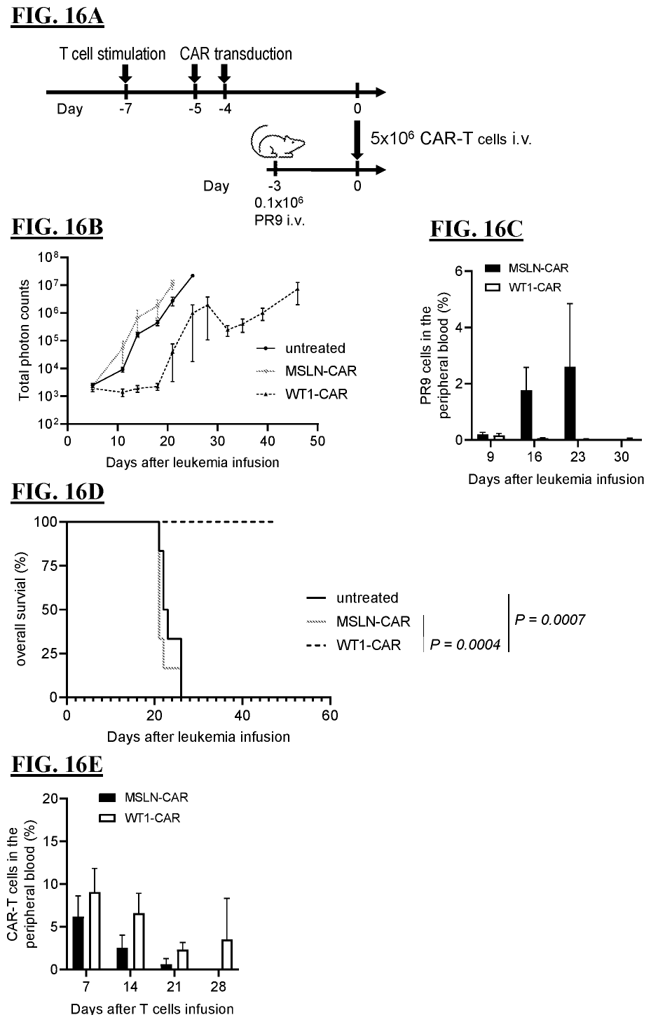

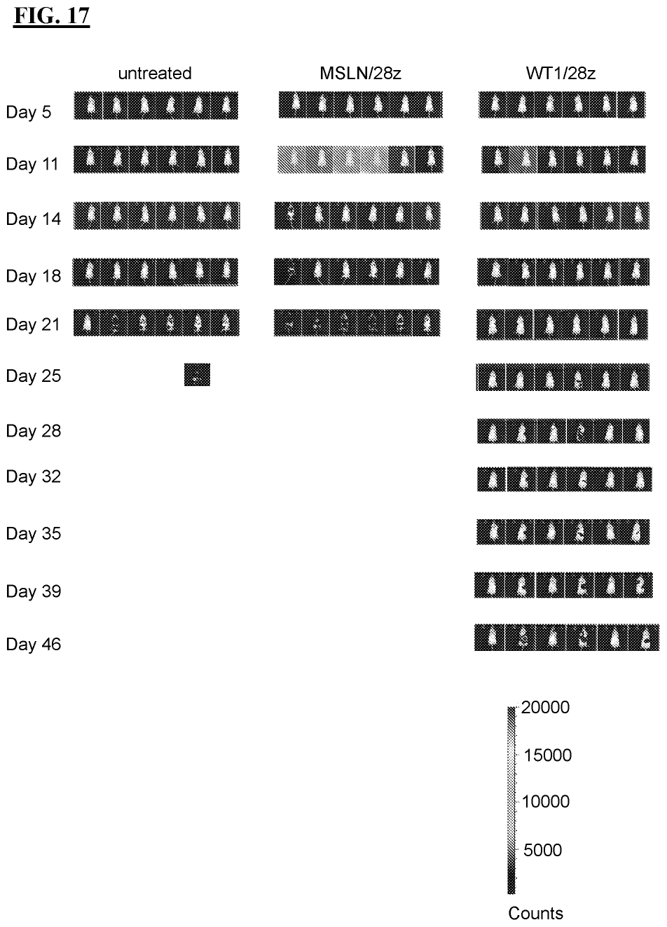

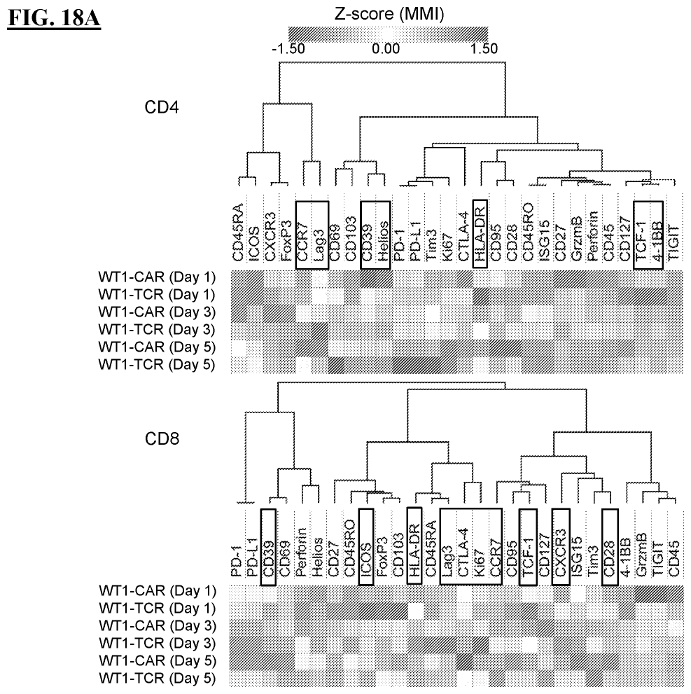

To determine whether WT1 CAR-T cells were able to control tumor growth in vivo, WT1V/class II⁺ AML cell line PR9 was first transduced with a construct expressing EGFP-firefly luciferase fusion protein and engrafted into NOD-scid IL2Rg^{null} (NSG) mice. Two days later, tumor-bearing mice were treated with WT1 CAR-T cells or control T cells, which were either untransduced or which expressed MSLN CAR (FIG. 16A). WT1 CAR-T cells markedly suppressed leukemic progression compared to control T cells (FIGS. 16B and 17). While PR9 cells were present in the peripheral blood of the control mice two weeks after tumor engraftment, they remained undetectable in the peripheral blood of WT1 CAR-T treated mice for more than four weeks (FIG. 16C). Mice treated with WT1 CAR-T cells also had significantly longer overall survival (FIG. 16D). Notably, WT1 CAR-T cells demonstrated significantly greater persistence in the peripheral blood compared to MSLN CAR-T cell (FIG. 16E).

### CyTOF-Based Analysis of WT1 TCR or CAR-T Cells Targeting WT1 Peptide Presented by HLA-DP4

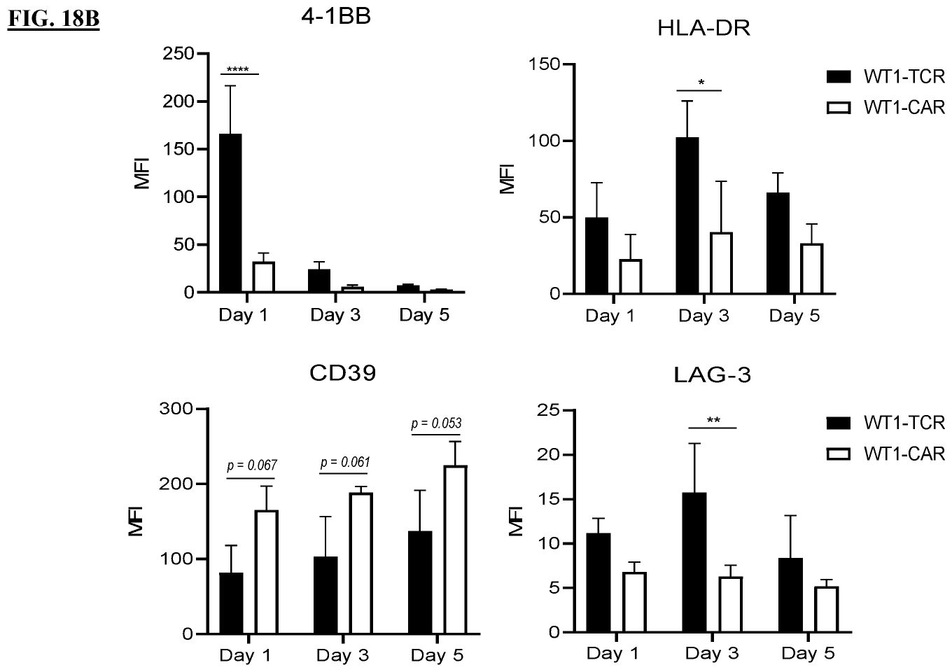

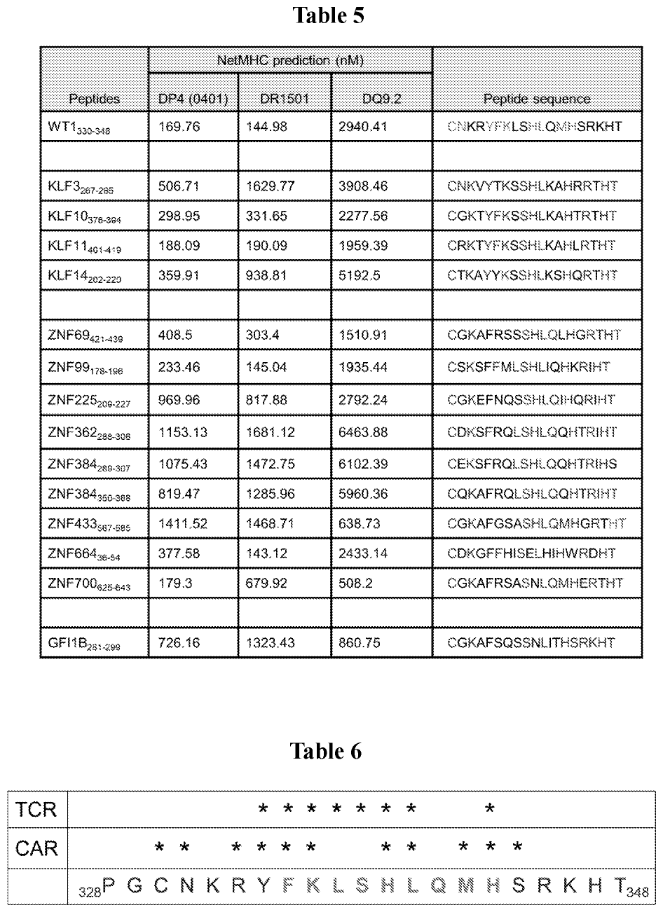

We have shown that WT1 CAR-T and TCR-T cells demonstrate comparable cytotoxic and cytokine response against WT1 peptide presented by HLA-DP4. Here, WT1 CAR-T and TCR-T cells were stimulated with WT1-overexpressing K562/DP4 cells and their phenotypes were comprehensively analysed using mass cytometry (CyTOF) at various time points (FIG. 18A). CD4⁺ WT1 TCR-T cells showed higher level of 4-1BB and HLA-DR, whereas CD8⁺ WT1 CAR-T cells showed higher level of CD39 and lower level of LAG-3 (FIG. 18B). Overall, we did not observe major phenotypic differences between WT1-TCR and WT1-CAR T cells upon stimulation with DP4⁺/WT1⁺ target cells.

### REFERENCES

Ajina, A., & Maher, J. (2018). Strategies to Address Chimeric Antigen Receptor Tonic Signaling. Mol Cancer Ther, 17(9), 1795-1815. doi:10.1158/1535-7163.MCT-17-1097
Anczurowski, M., & Hirano, N. (2018). Mechanisms of HLA-DP Antigen Processing and Presentation Revisited. Trends Immunol, 39(12), 960-964. doi:10.1016/j.it.2018.10.008
Anczurowski, M., Yamashita, Y., Nakatsugawa, M., Ochi, T., Kagoya, Y., Guo, T., . . . Hirano, N. (2018). Mechanisms underlying the lack of endogenous processing and CLIP-mediated binding of the invariant chain by HLA-DP(84Gly). Sci Rep, 8(1), 4804. doi:10.1038/s41598-018-22931-4
Ataie, N., Xiang, J., Cheng, N., Brea, E. J., Lu, W., Scheinberg, D. A., . . . Ng, H. L. (2016). Structure of a TCR-Mimic Antibody with Target Predicts Pharmacogenetics. J Mol Biol, 428(1), 194-205. doi:10.1016/j.jmb.2015.12.002
Axelrod, M. L., Cook, R. S., Johnson, D. B., & Balko, J. M. (2018). Biological Consequences of MHC-II Expression by Tumor Cells in Cancer. Clin Cancer Res. doi:10.1158/1078-0432.CCR-18-3200
Baeuerle, P. A., Ding, J., Patel, E., Thorausch, N., Horton, H., Gierut, J., . . . Hofmeister, R. (2019). Synthetic TRuC receptors engaging the complete T cell receptor for potent anti-tumor response. Nat Commun, 10(1), 2087. doi:10.1038/s41467-019-10097-0
Butler, M. O., Ansen, S., Tanaka, M., Imataki, O., Berezovskaya, A., Mooney, M. M., . . . Hirano, N. (2010). A panel of human cell-based artificial APC enables the expansion of long-lived antigen-specific CD4⁺ T cells restricted by prevalent HLA-DR alleles. Int Immunol, 22(11), 863-873. doi:10.1093/intimm/dxq440
Butler, M. O., & Hirano, N. (2014). Human cell-based artificial antigen-presenting cells for cancer immunotherapy. Immunol Rev, 257(1), 191-209. doi:10.1111/imr.12129
Butler, M. O., Lee, J. S., Ansen, S., Neuberg, D., Hodi, F. S., Murray, A. P., . . . Hirano, N. (2007). Long-lived antitumor CD8⁺ lymphocytes for adoptive therapy generated using an artificial antigen-presenting cell. Clin Cancer Res, 13(6), 1857-1867. doi:10.1158/1078-0432.CCR-06-1905
Caruso, H. G., Hurton, L. V., Najjar, A., Rushworth, D., Ang, S., Olivares, S., . . . Cooper, L. J. (2015). Tuning Sensitivity of CAR to EGFR Density Limits Recognition of Normal Tissue While Maintaining Potent Antitumor Activity. Cancer Res, 75(17), 3505-3518. doi:10.1158/0008-5472.CAN-15-0139
Castelli, F. A., Buhot, C., Sanson, A., Zarour, H., Pouvelle-Moratille, S., Nonn, C., . . . Maillere, B. (2002). HLA-DP4, the most frequent HLA II molecule, defines a new supertype of peptide-binding specificity. J Immunol, 169(12), 6928-6934.
Cooper, L. J., Topp, M. S., Serrano, L. M., Gonzalez, S., Chang, W. C., Naranjo, A., . . . Jensen, M. C. (2003). T-cell clones can be rendered specific for CD19: toward the selective augmentation of the graft-versus-B-lineage leukemia effect. Blood, 101(4), 1637-1644. doi:10.1182/blood-2002-07-1989
Coulie, P. G., Van den Eynde, B. J., van der Bruggen, P., & Boon, T. (2014). Tumour antigens recognized by T lymphocytes: at the core of cancer immunotherapy. Nat Rev Cancer, 14(2), 135-146. doi:10.1038/nrc3670
Denzin, L. K., Robbins, N. F., Carboy-Newcomb, C., & Cresswell, P. (1994). Assembly and intracellular transport of HLA-DM and correction of the class II antigen-processing defect in T2 cells. Immunity, 1(7), 595-606.
Dhatchinamoorthy, K., Colbert, J. D., & Rock, K. L. (2021). Cancer Immune Evasion Through Loss of MHC Class I Antigen Presentation. Front Immunol, 12, 636568. doi:10.3389/fimmu.2021.636568
Dillard, P., Koksal, H., Maggadottir, S. M., Winge-Main, A., Pollmann, S., Menard, M., . . . Inderberg, E. M. (2021). Targeting Telomerase with an HLA Class II-Restricted TCR for Cancer Immunotherapy. Mol Ther, 29(3), 1199-1213. doi:10.1016/j.ymthe.2020.11.019
Douglass, J., Hsiue, E. H., Mog, B. J., Hwang, M. S., DiNapoli, S. R., Pearlman, A. H., . . . Zhou, S. (2021). Bispecific antibodies targeting mutant RAS neoantigens. Sci Immunol, 6(57). doi:10.1126/sciimmunol.abd5515
Forero, A., Li, Y., Chen, D., Grizzle, W. E., Updike, K. L., Merz, N. D., . . . Varley, K. E. (2016). Expression of the MHC Class II Pathway in Triple-Negative Breast Cancer Tumor Cells Is Associated with a Good Prognosis and Infiltrating Lymphocytes. Cancer Immunol Res, 4(5), 390-399. doi:10.1158/2326-6066.CIR-15-0243
Fujiki, F., Oka, Y., Kawakatsu, M., Tsuboi, A., Nakajima, H., Elisseeva, O. A., . . . Sugiyama, H. (2008). A WT1 protein-derived, naturally processed 16-mer peptide, WT1(332), is a promiscuous helper peptide for induction of WT1-specific Th1-type CD4(+) T cells. Microbiol Immunol, 52(12), 591-600. doi:10.1111/j.1348-0421.2008.00080.x
Fujiki, F., Oka, Y., Tsuboi, A., Kawakami, M., Kawakatsu, M., Nakajima, H., . . . Sugiyama, H. (2007). Identification and characterization of a WT1 (Wilms Tumor Gene) protein-derived HLA-DRB1*0405-restricted 16-mer helper peptide that promotes the induction and activation of WT1-specific cytotoxic T lymphocytes. J Immunother, 30(3), 282-293. doi:10.1097/01.cji.0000211337.91513.94
Ghorashian, S., Kramer, A. M., Onuoha, S., Wright, G., Bartram, J., Richardson, R., . . . Amrolia, P. J. (2019). Enhanced CAR T cell expansion and prolonged persistence in pediatric patients with ALL treated with a low-affinity CD19 CAR. Nat Med, 25(9), 1408-1414. doi:10.1038/s41591-019-0549-5
Godkin, A. J., Smith, K. J., Willis, A., Tejada-Simon, M. V., Zhang, J., Elliott, T., & Hill, A. V. (2001). Naturally processed HLA class II peptides reveal highly conserved immunogenic flanking region sequence preferences that reflect antigen processing rather than peptide-MHC interactions. J Immunol, 166(11), 6720-6727. doi:10.4049/jimmunol.166.11.6720
Gomes-Silva, D., Mukherjee, M., Srinivasan, M., Krenciute, G., Dakhova, O., Zheng, Y., . . . Mamonkin, M. (2017). Tonic 4-1BB Costimulation in Chimeric Antigen Receptors Impedes T Cell Survival and Is Vector-Dependent. Cell Rep, 21(1), 17-26. doi:10.1016/j.celrep.2017.09.015
Greenbaum, J., Sidney, J., Chung, J., Brander, C., Peters, B., & Sette, A. (2011). Functional classification of class II human leukocyte antigen (HLA) molecules reveals seven different supertypes and a surprising degree of repertoire sharing across supertypes. Immunogenetics, 63(6), 325-335. doi:10.1007/s00251-011-0513-0
Gulyas, M., Dobra, K., & Hjerpe, A. (1999). Expression of genes coding for proteoglycans and Wilms' tumour susceptibility gene 1 (WT1) by variously differentiated benign human mesothelial cells. Differentiation, 65(2), 89-96. doi:10.1046/j.1432-0436.1999.6520089.x
Helsen, C. W., Hammill, J. A., Lau, V. W. C., Mwawasi, K. A., Afsahi, A., Bezverbnaya, K., . . . Bramson, J. L. (2018). The chimeric TAC receptor co-opts the T cell receptor yielding robust anti-tumor activity without toxicity. Nat Commun, 9(1), 3049. doi:10.1038/s41467-018-05395-y
Henne, C., Schwenk, F., Koch, N., & Moller, P. (1995). Surface expression of the invariant chain (CD74) is independent of concomitant expression of major histocompatibility complex class II antigens. Immunology, 84(2), 177-182.
Hirano, N., Butler, M. O., Von Bergwelt-Baildon, M. S., Maecker, B., Schultze, J. L., O'Connor, K. C., . . . Nadler, L. M. (2003). Autoantibodies frequently detected in patients with aplastic anemia. Blood, 102(13), 4567-4575. doi:10.1182/blood-2002-11-3409
Hirano, N., Butler, M. O., Xia, Z., Ansen, S., von Bergwelt-Baildon, M. S., Neuberg, D., . . . . Nadler, L. M. (2006). Engagement of CD83 ligand induces prolonged expansion of CD8+ T cells and preferential enrichment for antigen specificity. Blood, 107(4), 1528-1536. doi:10.1182/blood-2005-05-2073
Hosen, N., Sonoda, Y., Oji, Y., Kimura, T., Minamiguchi, H., Tamaki, H., . . . Sugiyama, H. (2002). Very low frequencies of human normal CD34⁺ haematopoietic progenitor cells express the Wilms' tumour gene WT1 at levels similar to those in leukaemia cells. Br J Haematol, 116(2), 409-420. doi:10.1046/j.1365-2141.2002.03261.x
Hsiue, E. H., Wright, K. M., Douglass, J., Hwang, M. S., Mog, B. J., Pearlman, A. H., . . . Zhou, S. (2021). Targeting a neoantigen derived from a common TP53 mutation. Science, 371(6533). doi:10.1126/science.abc8697
Hu, Y., Petroni, G. R., Olson, W. C., Czarkowski, A., Smolkin, M. E., Grosh, W. W., . . . Slingluff, C. L., Jr. (2014). Immunologic hierarchy, class II MHC promiscuity, and epitope spreading of a melanoma helper peptide vaccine. Cancer Immunol Immunother, 63(8), 779-786. doi:10.1007/s00262-014-1551-x
Johnson, D. B., Estrada, M. V., Salgado, R., Sanchez, V., Doxie, D. B., Opalenik, S. R., . . . Balko, J. M. (2016). Melanoma-specific MHC-II expression represents a tumour-autonomous phenotype and predicts response to anti-PD-1/PD-L1 therapy. Nat Commun, 7, 10582. doi:10.1038/ncomms10582
Johnson, L. A., & June, C. H. (2017). Driving gene-engineered T cell immunotherapy of cancer. Cell Res, 27(1), 38-58. doi:10.1038/cr.2016.154
Kagoya, Y., Tanaka, S., Guo, T., Anczurowski, M., Wang, C. H., Saso, K., . . . Hirano, N. (2018). A novel chimeric antigen receptor containing a JAK-STAT signaling domain mediates superior antitumor effects. Nat Med, 24(3), 352-359. doi:10.1038/nm.4478
Kawalekar, O. U., R S, O. C., Fraietta, J. A., Guo, L., McGettigan, S. E., Posey, A. D., Jr., . . . June, C. H. (2016). Distinct Signaling of Coreceptors Regulates Specific Metabolism Pathways and Impacts Memory Development in CAR T Cells. Immunity, 44(3), 712. doi:10.1016/j.immuni.2016.02.023
Kobayashi, H., Wood, M., Song, Y., Appella, E., & Celis, E. (2000). Defining promiscuous MHC class II helper T-cell epitopes for the HER2/neu tumor antigen. Cancer Res, 60(18), 5228-5236.
Krauth, M. T., Alpermann, T., Bacher, U., Eder, C., Dicker, F., Ulke, M., . . . Schnittger, S. (2015). WT1 mutations are secondary events in AML, show varying frequencies and impact on prognosis between genetic subgroups. Leukemia, 29(3), 660-667. doi:10.1038/leu.2014.243
Kudela, P., Janjic, B., Fourcade, J., Castelli, F., Andrade, P., Kirkwood, J. M., . . . Zarour, H. M. (2007). Cross-reactive CD4⁺ T cells against one immunodominant tumor-derived epitope in melanoma patients. J Immunol, 179(11), 7932-7940. doi:10.4049/jimmunol.179.11.7932
Larson, R. C., & Maus, M. V. (2021). Recent advances and discoveries in the mechanisms and functions of CAR T cells. Nat Rev Cancer, 21(3), 145-161. doi:10.1038/s41568-020-00323-z
Li, Y., Moysey, R., Molloy, P. E., Vuidepot, A. L., Mahon, T., Baston, E., . . . Boulter, J. M. (2005). Directed evolution of human T-cell receptors with picomolar affinities by phage display. Nat Biotechnol, 23(3), 349-354. doi:10.1038/nbt1070
Lin, Y., Fujiki, F., Katsuhara, A., Oka, Y., Tsuboi, A., Aoyama, N., . . . Sugiyama, H. (2013). HLA-DPB1*05: 01-restricted WT1₃₃₂-specific TCR-transduced CD4⁺ T lymphocytes display a helper activity for WT1-specific CTL induction and a cytotoxicity against leukemia cells. J Immunother, 36(3), 159-170. doi:10.1097/CJI.0b013e3182873581
Liu, X., Jiang, S., Fang, C., Yang, S., Olalere, D., Pequignot, E. C., . . . Zhao, Y. (2015). Affinity-Tuned ErbB2 or EGFR Chimeric Antigen Receptor T Cells Exhibit an Increased Therapeutic Index against Tumors in Mice. Cancer Res, 75(17), 3596-3607. doi:10.1 158/0008-5472.CAN-15-0159
Liu, Y., Liu, G., Wang, J., Zheng, Z. Y., Jia, L., Rui, W., . . . Lin, X. (2021). Chimeric STAR receptors using TCR machinery mediate robust responses against solid tumors. Sci Transl Med, 13(586). doi:10.1126/scitranslmed.abb5191
Long, A. H., Haso, W. M., Shern, J. F., Wanhainen, K. M., Murgai, M., Ingaramo, M., . . . Mackall, C. L. (2015). 4-1BB costimulation ameliorates T cell exhaustion induced by tonic signaling of chimeric antigen receptors. Nat Med, 21(6), 581-590. doi:10.1038/nm.3838
Lu, Y. C., Parker, L. L., Lu, T., Zheng, Z., Toomey, M. A., White, D. E., . . . Rosenberg, S. A. (2017). Treatment of Patients With Metastatic Cancer Using a Major Histocompatibility Complex Class II-Restricted T-Cell Receptor Targeting the Cancer Germline Antigen MAGE-A3. J Clin Oncol, 35(29), 3322-3329. doi:10.1200/JCO.2017.74.5463
May, R. J., Dao, T., Pinilla-Ibarz, J., Korontsvit, T., Zakhaleva, V., Zhang, R. H., . . . Scheinberg, D. A. (2007). Peptide epitopes from the Wilms' tumor 1 oncoprotein stimulate CD4⁺ and CD8⁺ T cells that recognize and kill human malignant mesothelioma tumor cells. Clin Cancer Res, 13(15 Pt 1), 4547-4555. doi:10.1158/1078-0432.CCR-07-0708
Milone, M. C., Fish, J. D., Carpenito, C., Carroll, R. G., Binder, G. K., Teachey, D., . . . June, C. H. (2009). Chimeric receptors containing CD137 signal transduction domains mediate enhanced survival of T cells and increased antileukemic efficacy in vivo. Mol Ther, 17(8), 1453-1464. doi:10.1038/mt.2009.83
Mittal, S. K., & Roche, P. A. (2015). Suppression of antigen presentation by IL-10. Curr Opin Immunol, 34, 22-27. doi:10.1016/j.coi.2014.12.009
Mohan, J. F., & Unanue, E. R. (2012). Unconventional recognition of peptides by T cells and the implications for autoimmunity. Nat Rev Immunol, 12(10), 721-728. doi:10.1038/nri3294
Morgan, R. A., Yang, J. C., Kitano, M., Dudley, M. E., Laurencot, C. M., & Rosenberg, S. A. (2010). Case report of a serious adverse event following the administration of T cells transduced with a chimeric antigen receptor recognizing ERBB2. Mol Ther, 18(4), 843-851. doi:10.1038/mt.2010.24
Mundlos, S., Pelletier, J., Darveau, A., Bachmann, M., Winterpacht, A., & Zabel, B. (1993). Nuclear localization of the protein encoded by the Wilms' tumor gene WT1 in embryonic and adult tissues. Development, 119(4), 1329-1341.
Nakatsugawa, M., Yamashita, Y., Ochi, T., Tanaka, S., Chamoto, K., Guo, T., . . . Hirano, N. (2015). Specific roles of each TCR hemichain in generating functional chain-centric TCR. J Immunol, 194(7), 3487-3500. doi:10.4049/jimmunol.1401717
Ochi, T., Nakatsugawa, M., Chamoto, K., Tanaka, S., Yamashita, Y., Guo, T., . . . Hirano, N. (2015). Optimization of T-cell Reactivity by Exploiting TCR Chain Centricity for the Purpose of Safe and Effective Antitumor TCR Gene Therapy. Cancer Immunol Res, 3(9), 1070-1081. doi:10.1158/2326-6066.CIR-14-0222
Oren, R., Hod-Marco, M., Haus-Cohen, M., Thomas, S., Blat, D., Duvshani, N., . . . Reiter, Y. (2014). Functional comparison of engineered T cells carrying a native TCR versus TCR-like antibody-based chimeric antigen receptors indicates affinity/avidity thresholds. J Immunol, 193(11), 5733-5743. doi:10.4049/jimmunol.1301769
Panina-Bordignon, P., Tan, A., Termijtelen, A., Demotz, S., Corradin, G., & Lanzavecchia, A. (1989). Universally immunogenic T cell epitopes: promiscuous binding to human MHC class II and promiscuous recognition by T cells. Eur J Immunol, 19(12), 2237-2242. doi:10.1002/eji.1830191209
Park, I. A., Hwang, S. H., Song, I. H., Heo, S. H., Kim, Y. A., Bang, W. S., . . . Lee, H. J. (2017). Expression of the MHC class II in triple-negative breast cancer is associated with tumor-infiltrating lymphocytes and interferon signaling. PLoS One, 12(8), e0182786. doi:10.1371/journal.pone.0182786
Park, S., Shevlin, E., Vedvyas, Y., Zaman, M., Park, S., Hsu, Y. S., . . . Jin, M. M. (2017). Micromolar affinity CAR T cells to ICAM-1 achieves rapid tumor elimination while avoiding systemic toxicity. Sci Rep, 7(1), 14366. doi:10.1038/s41598-017-14749-3
Poncette, L., Chen, X., Lorenz, F. K., & Blankenstein, T. (2019). Effective NY-ESO-1-specific MHC II-restricted T cell receptors from antigen-negative hosts enhance tumor regression. J Clin Invest, 129(1), 324-335. doi:10.1172/JCI120391
Quezada, S. A., Simpson, T. R., Peggs, K. S., Merghoub, T., Vider, J., Fan, X., . . . Allison, J. P. (2010). Tumor-reactive CD4(+) T cells develop cytotoxic activity and eradicate large established melanoma after transfer into lymphopenic hosts. J Exp Med, 207(3), 637-650. doi:10.1084/jem.20091918
Rafiq, S., Purdon, T. J., Daniyan, A. F., Koneru, M., Dao, T., Liu, C., . . . Brentjens, R. J. (2017). Optimized T-cell receptor-mimic chimeric antigen receptor T cells directed toward the intracellular Wilms Tumor 1 antigen. Leukemia, 31(8), 1788-1797. doi:10.1038/leu.2016.373
Riberdy, J. M., & Cresswell, P. (1992). The antigen-processing mutant T2 suggests a role for MHC-linked genes in class II antigen presentation. J Immunol, 148(8), 2586-2590.
Robbins, P. F., Li, Y. F., El-Gamil, M., Zhao, Y., Wargo, J. A., Zheng, Z., . . . Rosenberg, S. A. (2008). Single and dual amino acid substitutions in TCR CDRs can enhance antigen-specific T cell functions. J Immunol, 180(9), 6116-6131.
Rodig, S. J., Gusenleitner, D., Jackson, D. G., Gjini, E., Giobbie-Hurder, A., Jin, C., . . . Hodi, F. S. (2018). MHC proteins confer differential sensitivity to CTLA-4 and PD-1 blockade in untreated metastatic melanoma. Sci Transl Med, 10(450). doi:10.1 126/scitranslmed.aar3342
Roemer, M. G. M., Redd, R. A., Cader, F. Z., Pak, C. J., Abdelrahman, S., Ouyang, J., . . . Shipp, M. A. (2018). Major Histocompatibility Complex Class II and Programmed Death Ligand 1 Expression Predict Outcome After Programmed Death 1 Blockade in Classic Hodgkin Lymphoma. J Clin Oncol, 36(10), 942-950. doi:10.1200/JCO.2017.77.3994
Salter, A. I., Ivey, R. G., Kennedy, J. J., Voillet, V., Rajan, A., Alderman, E. J., . . . Riddell, S. R. (2018). Phosphoproteomic analysis of chimeric antigen receptor signaling reveals kinetic and quantitative differences that affect cell function. Sci Signal, 11(544). doi:10.1126/scisignal.aat6753
Schmitt, T. M., Aggen, D. H., Stromnes, I. M., Dossett, M. L., Richman, S. A., Kranz, D. M., & Greenberg, P. D. (2013). Enhanced-affinity murine T-cell receptors for tumor/self-antigens can be safe in gene therapy despite surpassing the threshold for thymic selection. Blood, 122(3), 348-356. doi:10.1182/blood-2013-01-478164
Stern, L. J., Brown, J. H., Jardetzky, T. S., Gorga, J. C., Urban, R. G., Strominger, J. L., & Wiley, D. C. (1994). Crystal structure of the human class II MHC protein HLA-DR1 complexed with an influenza virus peptide. Nature, 368(6468), 215-221. doi:10.1038/368215a0
Sugata, K., Matsunaga, Y., Yamashita, Y., Nakatsugawa, M., Guo, T., Halabelian, L., . . . Hirano, N. (2021). Affinity-matured HLA class II dimers for robust staining of antigen-specific CD4(+) T cells. Nat Biotechnol, 39(8), 958-967. doi:10.1038/s41587-021-00836-4
Sugiyama, H. (2010). WT1 (Wilms' tumor gene 1): biology and cancer immunotherapy. Jpn J Clin Oncol, 40(5), 377-387. doi:10.1093/jjco/hyp194
Svedberg, H., Chylicki, K., & Gullberg, U. (1999). Downregulation of Wilms' tumor gene (WT1) is not a prerequisite for erythroid or megakaryocytic differentiation of the leukemic cell line K562. Exp Hematol, 27(6), 1057-1062.
Tanaka, M., Butler, M. O., Ansen, S., Imataki, O., Berezovskaya, A., Nadler, L. M., & Hirano, N. (2011). Induction of HLA-DP4-restricted anti-survivin Th1 and Th2 responses using an artificial antigen-presenting cell. Clin Cancer Res, 17(16), 5392-5401. doi:10.1158/1078-0432.CCR-10-3083
Thomas, R., Al-Khadairi, G., Roelands, J., Hendrickx, W., Dermime, S., Bedognetti, D., & Decock, J. (2018). NY-ESO-1 Based Immunotherapy of Cancer: Current Perspectives. Front Immunol, 9, 947. doi:10.3389/fimmu.2018.00947
Valitutti, S., Muller, S., Cella, M., Padovan, E., & Lanzavecchia, A. (1995). Serial triggering of many T-cell receptors by a few peptide-MHC complexes. Nature, 375(6527), 148-151. doi:10.1038/375148a0
Van Oekelen, O., Aleman, A., Upadhyaya, B., Schnakenberg, S., Madduri, D., Gavane, S., . . . Parekh, S. (2021). Neurocognitive and hypokinetic movement disorder with features of parkinsonism after BCMA-targeting CAR-T cell therapy. Nat Med, 27(12), 2099-2103. doi:10.1038/s41591-021-01564-7
Weber, E. W., Parker, K. R., Sotillo, E., Lynn, R. C., Anbunathan, H., Lattin, J., . . . Mackall, C. L. (2021). Transient rest restores functionality in exhausted CAR-T cells through epigenetic remodeling. Science, 372(6537). doi:10.1126/science.abal786
Xu, Y., Yang, Z., Horan, L. H., Zhang, P., Liu, L., Zimdahl, B., . . . Liu, C. (2018). A novel antibody-TCR (AbTCR) platform combines Fab-based antigen recognition with gamma/delta-TCR signaling to facilitate T-cell cytotoxicity with low cytokine release. Cell Discov, 4, 62. doi:10.1038/s41421-018-0066-6
Yamashita, Y., Anczurowski, M., Nakatsugawa, M., Tanaka, M., Kagoya, Y., Sinha, A., . . . . Hirano, N. (2017). HLA-DP(84Gly) constitutively presents endogenous peptides generated by the class I antigen processing pathway. Nat Commun, 8, 15244. doi:10.1038/ncommsl5244
Yarmarkovich, M., Marshall, Q. F., Warrington, J. M., Premaratne, R., Farrel, A., Groff, D., . . . Maris, J. M. (2021). Cross-HLA targeting of intracellular oncoproteins with peptide-centric CARs. Nature, 599(7885), 477-484. doi:10.1038/s41586-021-04061-6
Ying, Z., He, T., Wang, X., Zheng, W., Lin, N., Tu, M., . . . Zhu, J. (2019). Parallel Comparison of 4-1BB or CD28 Co-stimulated CD19-Targeted CAR-T Cells for B Cell Non-Hodgkin's Lymphoma. Mol Ther Oncolytics, 15, 60-68. doi:10.1016/j.omto.2019.08.002
Zajac, P., Schultz-Thater, E., Tornillo, L., Sadowski, C., Trella, E., Mengus, C., . . . Spagnoli, G. C. (2017). MAGE-A Antigens and Cancer Immunotherapy. Front Med (Lausanne), 4, 18. doi:10.3389/fmed.2017.00018
Zhao, X., Yang, J., Zhang, X., Lu, X. A., Xiong, M., Zhang, J., . . . Huang, X. (2020). Efficacy and Safety of CD28- or 4-1BB-Based CD19 CAR-T Cells in B Cell Acute Lymphoblastic Leukemia. Mol Ther Oncolytics, 18, 272-281. doi:10.1016/j.omto.2020.06.016
Zheng, Z., Chinnasamy, N., & Morgan, R. A. (2012). Protein L: a novel reagent for the detection of chimeric antigen receptor (CAR) expression by flow cytometry. J Transl Med, 10, 29. doi:10.1186/1479-5876-10-29

### Antigen Binding Sequences

TABLE 7

| SEQ | | |
|---|---|---|
| ID | | |
| NO: | Description | Sequence |
| 1 | 1B1-VH-CDR1 | GFTFSNYA |
| 2 | 1B1-VH-CDR2 | ISSGGNT |
| 3 | 1B1-VH-CDR3 | ARIDDGYFVF |
| 4 | 1B1-VL-CDR1 | QSIDYDGDSY |
| 5 | 1B1-VL-CDR2 | AAS |
| 6 | 1B1-VL-CDR3 | QQSYEDPPT |
| 7 | 1B1-VH | EVKLVESGGGLVKPGESLKLACAASGFTFSNYAMSWVRQTPEKRLEWVASISSGGNT |
| | | YFPDSVKGRFTISRDNARNTLYLQMNSLRSEDTAMYYCARIDDGYFVFGGQGTLVTV |
| | | SA |
| 8 | 1B1-VL | DIVLTQSPASLAVSLGQRATISCKASQSIDYDGDSYMNWYQQKPGQPPKLLIYAASNL |
| | | ESGIPARFSGSGSGTDFTLNIHPVEEEDAATYYCQQSYEDPPTFGGGTKLEIK |
| 9 | 1B1-VH Nuc | GAAGTGAAGCTGGTGGAGTCTGGGGGAGGCTTAGTGAAGCCTGGAGAGTCCCTG |
| | | AAACTCGCCTGTGCAGCCTCTGGATTCACTTTCAGTAACTATGCCATGTCTTGGGT |
| | | TCGCCAGACTCCAGAGAAGAGGCTGGAGTGGGTCGCATCCATTAGTAGTGGTGG |
| | | TAACACCTACTTTCCAGACAGTGTGAAGGGCCGATTCACCATCTCCAGAGATAATG |
| | | CCAGGAACACCCTGTACCTGCAAATGAACAGTCTGAGGTCTGAGGACACGGCCAT |
| | | GTATTACTGTGCAAGAATAGACGATGGTTACTTCGTTTTCGGGGGCCAAGGGACT |
| | | CTGGTCACTGTCTCTGCA |
| 10 | 1B1-VL Nuc | GACATTGTGCTGACCCAATCTCCAGCTTCTTTGGCTGTGTCTCTAGGGCAGAGGGC |
| | | CACCATCTCCTGCAAGGCCAGCCAAAGTATTGATTATGATGGTGATAGTTATATGA |
| | | ACTGGTACCAACAGAAACCAGGACAGCCACCCAAACTCCTCATCTATGCTGCATCC |
| | | AATCTAGAATCTGGGATCCCAGCCAGGTTTAGTGGCAGTGGGTCTGGGACAGACT |
| | | TCACCCTCAACATCCATCCTGTGGAGGAGGAGGATGCTGCAACCTATTACTGTCAG |
| | | CAAAGTTATGAGGATCCTCCGACGTTCGGTGGAGGCACCAAGCTGGAAATCAAA |
| 11 | 2E7-VH-CDR1 | GFTFSNYA |
| 12 | 2E7-VH-CDR2 | ISSGGNT |
| 13 | 2E7-VH-CDR3 | ARIDDGYFVF |
| 14 | 2E7-VL-CDR1 | QSIDYDGDSY |
| 15 | 2E7-VL-CDR2 | AAS |
| 16 | 2E7-VL-CDR3 | QQSYEDPPT |
| 17 | 2E7-VH | EVKLVESGGGLVKPGESLKLACAASGFTFSNYAMSWVRQTPEKRLEWVASISSGGNT |
| | | YFPDSVKGRFTISRDNARNTLYLQMNSLRSEDTAMYYCARIDDGYFVFGGQGTLVTV |
| | | SA |
| 18 | 2E7-VL | DIVLTQSPASLAVSLGQRATISCKASQSIDYDGDSYMNWYQQKPGQPPKLLIYAASNL |
| | | ESGIPARFSGSGSGTDFTLNIHPVEEEDAATYYCQQSYEDPPTFGGGTKLEIK |
| 19 | 2E7-VH Nuc | GAAGTGAAGCTGGTGGAGTCTGGGGGAGGCTTAGTGAAGCCTGGAGAGTCCCTG |
| | | AAACTCGCCTGTGCAGCCTCTGGATTCACTTTCAGTAACTATGCCATGTCTTGGGT |
| | | TCGCCAGACTCCAGAGAAGAGGCTGGAGTGGGTCGCATCCATTAGTAGTGGTGG |
| | | TAACACCTACTTTCCAGACAGTGTGAAGGGCCGATTCACCATCTCCAGAGATAATG |
| | | CCAGGAACACCCTGTACCTGCAAATGAACAGTCTGAGGTCTGAGGACACGGCCAT |
| | | GTATTACTGTGCAAGAATAGACGATGGTTACTTCGTTTTCGGGGGCCAAGGGACT |
| | | CTGGTCACTGTCTCTGCA |
| 20 | 2E7-VL Nuc | GACATTGTGCTGACCCAATCTCCAGCTTCTTTGGCTGTGTCTCTAGGGCAGAGGGC |
| | | CACCATCTCCTGCAAGGCCAGCCAAAGTATTGATTATGATGGTGATAGTTATATGA |
| | | ACTGGTACCAACAGAAACCAGGACAGCCACCCAAACTCCTCATCTATGCTGCATCC |
| | | AATCTAGAATCTGGGATCCCAGCCAGGTTTAGTGGCAGTGGGTCTGGGACAGACT |
| | | TCACCCTCAACATCCATCCTGTGGAGGAGGAGGATGCTGCAACCTATTACTGTCAG |
| | | CAAAGTTATGAGGATCCTCCGACGTTCGGTGGAGGCACCAAGCTGGAAATCAAA |
| 21 | 3C3-VH-CDR1 | GYSITSGYS |
| 22 | 3C3-VH-CDR2 | IHYSGST |
| 23 | 3C3-VH-CDR3 | ASHDFDY |
| 24 | 3C3-VL-CDR1 | QSLLDSDGKTY |
| 25 | 3C3-VL-CDR2 | LVS |
| 26 | 3C3-VL-CDR3 | WQGTHLPYT |
| 27 | 3C3-VH | DVQLQESGPDLVKPSQSLSLTCTVTGYSITSGYSWPWIRQFPGNKLEWMGYIHYSGS |
| | | TNYNPSLKSRISITRDTSKNQFFLQLNSVTTEDTATYYCASHDFDYWGQGTTLTVSS |
| 28 | 3C3-VL | DVVMTQTPLTLSVTIGQPASVSCKSSQSLLDSDGKTYLNWLLQRPGQSPKRLIYLVSKL |
| | | DSGVPDRLTGSGSGTDFTLKISRVEAEDLGVYYCWQGTHLPYTFGGGTKLEIK |
| 29 | 3C3-VH Nuc | GATGTGCAGCTTCAGGAGTCAGGACCTGACCTGGTGAAACCTTCTCAGTCACTTTC |
| | | ACTCACCTGCACTGTCACTGGCTACTCCATCACCAGTGGTTATAGCTGGCCCTGGA |
| | | TCCGGCAGTTTCCAGGAAACAAACTGGAATGGATGGGCTACATACACTACAGTGG |
| | | TAGCACTAACTACAACCCATCTCTCAAAAGTCGAATCTCTATCACTCGAGACACAT |
| | | CCAAGAACCAGTTCTTCCTGCAATTGAATTCTGTGACTACTGAGGACACAGCCACA |
| | | TATTACTGTGCAAGCCACGACTTTGACTACTGGGGCCAAGGCACCACTCTCACAGT |
| | | CTCCTCA |
| 30 | 3C3-VL Nuc | GATGTTGTGATGACCCAGACTCCACTCACTTTGTCGGTTACCATTGGACAACCAGC |
| | | CTCCGTCTCTTGCAAGTCAAGTCAGAGCCTCTTAGATAGTGATGGAAAGACATATT |
| | | TGAATTGGTTGTTACAGAGGCCAGGCCAGTCTCCAAAACGCCTAATATATCTGGT |
| | | GTCTAAACTGGACTCTGGAGTCCCTGACAGGCTCACTGGCAGTGGATCAGGGACA |
| | | GATTTCACACTGAAAATCAGCAGAGTGGAGGCTGAGGATTTGGGAGTTTATTATT |
| | | GTTGGCAAGGCACACATCTTCCGTACACGTTCGGAGGGGGGACCAAGCTGGAAA |
| | | TAAAA |
| 31 | 5H2-VH-CDR1 | GFNIKDYY |
| 32 | 5H2-VH-CDR2 | IDPENGNT |
| 33 | 5H2-VH-CDR3 | TRDGYYMVFAY |
| 34 | 5H2-VL-CDR1 | QDINKC |
| 35 | 5H2-VL-CDR2 | YTS |
| 36 | 5H2-VL-CDR3 | LQYDNLYT |
| 37 | 5H2-VH | EVQLQQSGAELVRPGALVKLSCKASGFNIKDYYMHWVKQRPEQGLEWIGWIDPENG |
| | | NTIYDPKFQGKASITADTSSNTAYLQLSSLTSGDTAVYYCTRDGYYMVFAYWGQGTLV |
| | | TVSA |
| 38 | 5H2-VL | DIQMTQSPSSLSASLGGKVTITCKASQDINKCIAWYKHKPGKGPRLLIHYTSILQPGI |
| | | PSRFSGSGSGRDYSFSISNLEPEDIATYYCLQYDNLYTFGGGTKLEIK |
| 39 | 5H2-VH Nuc | GAGGTTCAGCTGCAGCAGTCTGGGGCTGAGCTTGTGAGGCCAGGGGCCTTAGTC |
| | | AAGTTGTCCTGCAAAGCTTCTGGCTTCAACATTAAAGACTACTATATGCACTGGGT |
| | | GAAGCAGAGGCCTGAACAGGGCCTGGAGTGGATTGGATGGATTGATCCTGAGAA |
| | | TGGTAATACTATATATGACCCGAAGTTCCAGGGCAAGGCCAGTATAACAGCAGAC |
| | | ACATCCTCCAATACAGCCTACCTGCAACTCAGCAGCCTGACATCTGGGGACACTGC |
| | | CGTCTATTACTGTACTAGAGATGGTTACTACATGGTTTTTGCTTACTGGGGCCAAG |
| | | GGACTCTGGTCACTGTCTCTGCA |
| 40 | 5H2-VL Nuc | GACATCCAGATGACGCAGTCTCCATCCTCACTGTCTGCATCTCTGGGAGGCAAAGT |
| | | CACCATCACTTGCAAGGCAAGCCAAGACATTAACAAGTGTATAGCTTGGTACAAA |
| | | CACAAGCCTGGAAAAGGTCCTAGGCTGCTCATTCATTACACATCTATATTACAGCC |
| | | AGGCATCCCATCAAGGTTCAGTGGAAGTGGGTCTGGGAGAGATTATTCCTTCAGC |
| | | ATCAGCAACCTGGAGCCTGAAGATATTGCAACTTATTATTGTCTACAGTATGATAA |
| | | TCTGTACACGTTCGGAGGGGGGACCAAGCTGGAAATAAAA |
| 41 | 6B7-VH-CDR1 | GFTFSSYA |
| 42 | 6B7-VH-CDR2 | ISSGGST |
| 43 | 6B7-VH-CDR3 | ARVGEGAMDY |
| 44 | 6B7-VL-CDR1 | QSVDYDGDSY |
| 45 | 6B7-VL-CDR2 | AAS |
| 46 | 6B7-VL-CDR3 | QQSNEDPPT |
| 47 | 6B7-VH | EVKLVESGGGLVKPGGSLKLSCAASGFTFSSYAMSWVRQTPEKRLEWVASISSGGSTY |
| | | YPDSVKGRFTISRDNTRNILYLQMSSLRSEDTAMYSCARVGEGAMDYWGQGTSVTV |
| | | SS |
| 48 | 6B7-VL | DIVLTQSPASLAVSLGQRATISCKASQSVDYDGDSYMNWYQQKPGQPPKLLIYAASNL |
| | | ESGIPARFSGSGSGTDFTLNIHPVEEEDAATYYCQQSNEDPPTFGGGTKLEIK |
| 49 | 6B7-VH Nuc | GAAGTGAAGCTGGTGGAGTCTGGGGGAGGCTTAGTGAAGCCTGGAGGGTCCCTG |
| | | AAACTCTCCTGTGCAGCCTCTGGATTCACTTTCAGTAGCTATGCCATGTCTTGGGTT |
| | | CGCCAGACTCCAGAGAAGAGGCTGGAGTGGGTCGCATCCATTAGTAGTGGTGGT |
| | | AGCACCTACTATCCAGACAGTGTGAAGGGCCGATTCACCATCTCCAGAGATAATA |
| | | CCAGGAACATCCTGTACCTGCAAATGAGCAGTCTGAGGTCTGAGGACACGGCCAT |
| | | GTATTCCTGTGCAAGAGTGGGGGAAGGAGCTATGGACTACTGGGGTCAAGGAAC |
| | | CTCAGTCACCGTCTCCTCA |
| 50 | 6B7-VL Nuc | GACATTGTGCTGACCCAATCTCCAGCTTCTTTGGCTGTATCTCTAGGGCAGAGGGC |
| | | CACCATCTCCTGCAAGGCCAGCCAAAGTGTTGATTATGATGGTGATAGTTATATGA |
| | | ACTGGTACCAACAGAAACCAGGACAGCCACCCAAACTCCTCATCTATGCTGCATCC |
| | | AATCTAGAATCTGGGATCCCAGCCAGGTTTAGTGGCAGTGGGTCTGGGACAGACT |
| | | TCACCCTCAACATCCATCCTGTGGAGGAGGAGGATGCTGCAACCTATTACTGTCAG |
| | | CAAAGTAATGAGGATCCTCCGACGTTCGGTGGAGGCACCAAGCTGGAAATCAAA |
| 51 | Epitope-WT1 | FKLSHLQMH |
| | fragment | |
| 52 | Epitope-WT1 | CNKRYFKLSHLQMHSRKHT |
| | fragment | |
| 53 | Peptide | AYKRKSRESD |
| 54 | Peptide | FNNFTVSFWLRVPKVSASHLE |
| 55 | Peptide | LPKPPKPVSKMRMATPLLMQALPM |
| 56 | Peptide | PGCNKRYFKLSHLQMHSRKHT |
| 57 | Peptide | KLLTQHFVQENYLEY |

1.-6. (canceled)

7. An antibody or an antigen-binding portion thereof that specifically binds WT1, wherein the antibody comprises (i) a heavy chain variable region (VH) comprising a VH complementarity determining region (CDR)-1, a VH-CDR2, and a VH-CDR3; and (ii) a light chain variable region (VL) comprising a VL CDR1, a VL-CDR2, and a VL-CDR3; wherein the VH-CDR1, VH-CDR2, VH-CDR3, VL-CDR1, VL-CDR2, and VL-CDR3 comprise
(a) the amino acid sequences set forth in SEQ ID NOs: 1, 2, 3, 4, 5, and 6 respectively;
(b) the amino acid sequence set forth in SEQ ID NOs: 11, 12, 13, 14, 15, and 16, respectively;
(c) the amino acid sequence set forth in SEQ ID NOs: 21, 22, 23, 24, 25, and 26, respectively;
(d) the amino acid sequence set forth in SEQ ID NOs: 31, 32, 33, 34, 35, and 36, respectively;
(e) the amino acid sequence set forth in SEQ ID NOs: 41, 42, 43, 44, 45, and 46, respectively; or
(f) the amino acid sequence set forth in SEQ ID NOs: 51, 52, 53, 54, 55, and 56, respectively.

8.-13. (canceled)

14. The antibody or antigen-binding portion thereof of claim 7, wherein the VH comprises an amino acid sequence having at least about 80% sequence identity to an amino acid sequence set forth in SEQ ID NO: 7, 17, 27, 37, 47, or 57.

15. The antibody or antigen-binding portion thereof of claim 7, wherein the VH comprises an amino acid sequence set forth in SEQ ID NOs: 7, 17, 27, 37, 47, or 57.

16. The antibody or antigen-binding portion thereof of claim 7, wherein the VL comprises an amino acid sequence having at least about 80% sequence identity to an amino acid sequence set forth in SEQ ID NO: 8, 18, 28, 38, 48, or 58.

17. The antibody or antigen-binding portion thereof of claim 7, wherein the VL comprises an amino acid sequence set forth in SEQ ID NO: 8, 18, 28, 38, 48, or 58.

18. The antibody or antigen-binding portion thereof of claim 7, wherein
(a) the VH comprises the amino acid sequence set forth in SEQ ID NO: 7, and the VL comprises the amino acid sequence set forth in SEQ ID NO: 8;
(b) the VH comprises the amino acid sequence set forth in SEQ ID NO: 17, and the VL comprises the amino acid sequence set forth in SEQ ID NO: 18;
(c) the VH comprises the amino acid sequence set forth in SEQ ID NO: 27, and the VL comprises the amino acid sequence set forth in SEQ ID NO: 28;
(d) the VH comprises the amino acid sequence set forth in SEQ ID NO: 37, and the VL comprises the amino acid sequence set forth in SEQ ID NO: 38;
(e) the VH comprises the amino acid sequence set forth in SEQ ID NO: 47, and the VL comprises the amino acid sequence set forth in SEQ ID NO: 48; or
(f) the VH comprises the amino acid sequence set forth in SEQ ID NO: 57, and the VL comprises the amino acid sequence set forth in SEQ ID NO: 58.

19. The antibody or antigen-binding portion thereof of claim 7, which binds a peptide fragment of WT1 comprising the amino acid sequence set forth in SEQ ID NO: 51.

20. (canceled)

21. The antibody or antigen-binding portion thereof of claim 19, wherein the peptide fragment is presented by an MHC Class II molecule.

22. (canceled)

23. The antibody or antigen-binding portion thereof of claim 21, wherein the MHC Class II molecule comprises:
(i) a DP beta chain and a DP alpha chain, wherein
(a) the beta chain of the MHC class II molecule is a DP1, DP2, DP3, DP4, DP5, DP6, DP8, or DP9 allele;
(b) the alpha chain of the MHC class II molecule comprises an HLA-DPA1*01, HLA-DPA1*02, HLA-DPA1*03, or HLA-DPA1*04 allele; or
(c) both (a) and (b);
(ii) a DR beta chain and a DR alpha chain, wherein
(a) the beta chain of the MHC class II molecule comprises a DR2, DR3, DR4, DR5, DR6, DR7, DR8, DR9, DR10, DR11, DR12, DR13, DR14, DR15, or DR16 allele;
(b) the alpha chain of the MHC class II molecule comprises an HLA-DRA1*01 allele; or
(c) both (a) and (b); or
(iii) a DQ beta chain and a DQ alpha chain, wherein
(a) the beta chain of the MHC class II molecule comprises a DQ2, DQ3, DQ4, DQ5, or DQ6 allele;
(b) the alpha chain of the MHC class II molecule comprises an HLA-DQA1*01, HLA-DQA1*02, HLA-DQA1*03, HLA-DQA1*04, HLA-DQA1*05, or HLA-DQA1*06 allele; or
(c) both (a) and (b).

24.-29. (canceled)

30. An antibody or an antigen-binding portion thereof that specifically binds a peptide fragment of a tumor antigen, wherein the peptide fragment is presented by a first major histocompatibility complex class II molecule and a second MHC class II molecule, wherein the first and the second MHC class II molecules are different,
wherein the tumor antigen comprises NY-ESO-1, gp100, Tyr, MAGE-A1, MAGE-A3, SSX2, CCND1, MUC5C, WT1, or any combination thereof.

31.-45. (canceled)

46. A chimeric antigen receptor (CAR) comprising an antigen-binding domain, wherein the antigen-binding domain comprises the antibody or antigen-binding portion thereof of claim 7.

47.-53. (canceled)

54. A bispecific antibody comprising a first antigen-binding domain and a second antigen-binding domain, wherein the first antigen-binding domain, the second antigen-binding domain, or both comprises the antibody or antigen-binding portion thereof of claim 7.

55. (canceled)

56. A bi-specific T cell engager (BiTE) comprising (i) a first antigen-binding domain that specifically binds CD3 receptor and (ii) a second antigen-binding domain that specifically binds a peptide fragment of WT1, wherein second antigen-binding domain comprises the antibody or antigen-binding portion thereof of claim 7.

57.-63. (canceled)

64. A tri-specific T cell engager (TriTE) comprising (i) a first antigen-binding domain that specifically binds CD3 receptor, (ii) a second antigen-binding domain that specifically binds a peptide fragment of WT1, and (iii) a third antigen-binding domain; wherein second antigen-binding domain comprises the antibody or antigen-binding portion thereof of claim 7.

65.-71. (canceled)

72. A nucleic acid molecule or a set of nucleic acid molecules encoding the antibody or antigen-binding portion thereof of claim 7.

73. A vector or a set of vectors comprising the nucleic acid molecule or the set of nucleic acid molecules of claim 72.

74-75. (canceled)

76. A cell comprising the antibody or antigen-binding portion thereof of claim 7.

77.-78. (canceled)

79. A pharmaceutical composition comprising the antibody or antigen-binding portion thereof of claim 7.

80. A method of treating a disease or condition in a subject in need thereof, comprising administering to the subject the antibody or antigen-binding portion thereof of claim 7.

81.-85. (canceled)

86. A method of generating an antibody or an antigen-binding portion thereof that binds a peptide fragment complexed with an MHC Class II molecule comprising: (i) identifying a peptide fragment associated with an MHC Class II molecule, and (ii) raising an antibody against the peptide fragment; wherein the antibody is not raised against the MHC Class II molecule or a complex of the MHC Class II molecule and the peptide fragment.

87.-91. (canceled)
